(19) United States
(12) Patent Application Publication
(10) Pub. No.: US 2004/0005558 A1
(43) Pub. Date: Jan. 8, 2004

(54) Proteins, polynucleotides ecoding them and methods of using the same

(76) Inventors: David W. Anderson, Branford, CT (US); Catherine E. Burgess, Wethersfield, CT (US); Stacie J. Casman, North Haven, CT (US); Steven D. Colman, Guilford, CT (US); Shlomit R. Edinger, New Haven, CT (US); Karen Ellerman, Branford, CT (US); Valerie Gerlach, Branford, CT (US); Erik Gunther, Branford, CT (US); Ramesh Kekuda, Stamford, CT (US); John R. MacDougall, Hamden, CT (US); Fuad Mehraban, Trumbull, CT (US); Meera Patturajan, Branford, CT (US); Mark Rothenberg, Clinton, CT (US); Richard A. Shimkets, Guilford, CT (US); Glennda Smithson, Guilford, CT (US); Kimberly A. Spytek, New Haven, CT (US); David J. Stone, Guilford, CT (US); Corine A.M. Vernet, Branford, CT (US); Bryan D. Zerhusen, Branford, CT (US)

Correspondence Address: Ivor R. Elrifi, MINTZ, LEVIN, COHN, FERRIS,, GLOVSKY and POPEO, P.C., One Financial Center, Boston, MA, 02111, US

(21) Appl. No.: 10/052,648

(22) Filed: Jan. 18, 2002

Related U.S. Application Data
(60) Provisional application No. 60/262,454, filed on Jan. 18, 2001.
(60) Provisional application No. 60/272,920, filed on Mar. 2, 2001.
(60) Provisional application No. 60/284,549, filed on Apr. 18, 2001.
(60) Provisional application No. 60/303,229, filed on Jul. 5, 2001.
(60) Provisional application No. 60/262,892, filed on Jan. 19, 2001.
(60) Provisional application No. 60/263,605, filed on Jan. 23, 2001.
(60) Provisional application No. 60/269,098, filed on Feb. 15, 2001.
(60) Provisional application No. 60/264,159, filed on Jan. 25, 2001.
(60) Provisional application No. 60/265,517, filed on Jan. 31, 2001.
(60) Provisional application No. 60/271,855, filed on Feb. 27, 2001.
(60) Provisional application No. 60/267,057, filed on Feb. 7, 2001.
(60) Provisional application No. 60/286,287, filed on Apr. 25, 2001.

(51) Int. Cl. C12Q 1/68; G01N 33/53; G01N 33/567; A61K 38/17; A61K 48/00; C12P 21/02; C12N 5/06; C07K 14/47; C07K 16/18

(52) U.S. Cl. 435/6; 435/7.2; 435/69.1; 435/320.1; 435/325; 514/12; 514/44; 530/350; 530/388.1

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority to U.S. Ser. No. 60/262,454 (Attorney Ref.: 21402-250), filed Jan. 18, 2001; U.S. Ser. No. 60/272,920 (Attorney Ref.: 21402-250G1), filed Mar. 2, 2001; U.S. Ser. No. 60/284,549 (Attorney Ref.: 21402-250G2), filed Apr. 18, 2001; U.S. Ser. No. 60/303,229 (Attorney Ref.: 21402-250G3), filed Jul. 5, 2001; U.S. Ser. No. 60/262,892 (Attorney Ref.: 21402-251), filed Jan. 19, 2001; U.S. Ser. No. 60/263,605 (Attorney Ref.: 21402-252), filed Jan. 23, 2001, U.S. Ser. No. 60/269,098 (Attorney Ref.: 21402-252A), filed Feb. 15, 2001, U.S. Ser .No. 60/264,159 (Attorney Ref.: 21402-254), filed Jan. 25, 2001, U.S. Ser. No. 60/265,517 (Attorney Ref.: 21402-259), filed Jan. 31, 2001, U.S. Ser. No. 60/271,855 (Attorney Ref.: 21402-259B) filed Feb. 27, 2001, U.S. Ser. No. 60/267,057 (Attorney Ref.: 21402-266), filed Feb. 7, 2001, and U.S. Ser. No. 60/286,287 (Attorney Ref.: 21402-250G2 REV), filed Apr. 25, 2001, each of which is incorporated by reference in its entirety.

### FIELD OF THE INVENTION

The invention relates to polynucleotides and the polypeptides encoded by such polynucleotides, as well as vectors, host cells, antibodies and recombinant methods for producing the polypeptides and polynucleotides, as well as methods for using the same.

### BACKGROUND OF THE INVENTION

The present invention is based in part on nucleic acids encoding proteins that are new members of the following protein families: delta serrate ligand receptors, protein kinases, G-protein coupled receptors (GPCR), ankyrin repeat containing proteins, TNF intracellular domain interacting proteins, secretory proteins and dual specificity phosphatases. More particularly, the invention relates to nucleic acids encoding novel polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, and NOV9 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS: 1, 3, 5, 7, 9, 11, 13, 15, 17, 19, 21, 23, 25, 27 and 29. Protein phosphorylation is a fundamental process for the regulation of cellular functions. The coordinated action of both protein kinases and phosphatases controls the levels of phosphorylation and, hence, the activity of specific target proteins. One of the predominant roles of protein phosphorylation is in signal transduction, where extracellular signals are amplified and propagated by a cascade of protein phosphorylation and dephosphorylation events. Eukaryotic protein kinases are enzymes that belong to a very extensive family of proteins which share a conserved catalytic core common with both serine/threonine and tyrosine protein kinases. There are a number of conserved regions in the catalytic domain of protein kinases. In the N-terminal extremity of the catalytic domain there is a glycine-rich stretch of residues in the vicinity of a lysine residue, which has been shown to be involved in ATP binding. In the central part of the catalytic domain there is a conserved aspartic acid residue which is important for the catalytic activity of the enzyme. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS: 2, 4, 6, 8, 10, 12, 14, 16, 18, 20, 22, 24, 26, 28 and 30. The nucleic acid can be, for example, a genomic DNA fragment or a cDNA molecule that includes the nucleic acid sequence of any of SEQ ID NOS: 1, 3, 5, 7, 9, 11, 13, 15, 17, 19, 21, 23, 25, 27 and 29.

Also included in the invention is an oligonucleotide, e.g., an oligonucleotide which includes at least 6 contiguous nucleotides of a NOVX nucleic acid (e.g., SEQ ID NOS: 1, 3, 5, 7, 9, 11, 13, 15, 17, 19, 21, 23, 25, 27 and 29) or a complement of said oligonucleotide. Also included in the invention are substantially purified NOVX polypeptides (SEQ ID NOS: 2, 4, 6, 8, 10, 12, 14, 16, 18, 20, 22, 24, 26, 28 and 30). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., trauma, regeneration (in vitro and in vivo), viral/bacterial/parasitic infections, Von Hippel-Lindau (VHL) syndrome, Alzheimer's disease, stroke, Tuberous sclerosis, hypercalceimia, Parkinson's disease, Huntington's disease, Cerebral palsy, Epilepsy, Lesch-Nyhan syndrome, multiple sclerosis, Ataxia-telangiectasia, leukodystrophies, behavioral disorders, addiction, anxiety, pain, actinic keratosis, acne, hair growth diseases, allopecia, pigmentation disorders, endocrine disorders, connective tissue disorders, such as severe neonatal Marfan syndrome, dominant ectopia lentis, familial ascending aortic aneurysm, isolated skeletal features of Marfan syndrome, Shprintzen-Goldberg syndrome, genodermatoses, contractural arachnodactyly, inflammatory disorders such as osteo- and rheumatoid-arthritis, inflammatory bowel disease, Crohn's disease; immunological disorders, AIDS; cancers including but not limited to lung cancer, colon cancer, neoplasm; adenocarcinoma; lymphoma; prostate cancer; uterus cancer, leukemia or pancreatic cancer; blood disorders; asthma; psoriasis; vascular disorders, hypertension, skin disorders, renal disorders including Alport syndrome, immunological disorders, tissue injury, fibrosis disorders, bone diseases, Ehlers-Danlos syndrome type VI, VII, type IV, S-linked cutis laxa and Ehlers-Danlos syndrome type V, osteogenesis imperfecta, neurologic diseases, brain and/or autoimmune disorders like encephalomyelitis, neurodegenerative disorders, immune disorders, hematopoietic disorders, muscle disorders, inflammation and wound repair, bacterial, fungal, protozoal and viral infections (particularly infections caused by HIV-1 or HIV-2), pain, acute heart failure, hypotension, hypertension, urinary retention, osteoporosis, treatment of Albright hereditary ostocodystrophy, angina pectoris, myocardial infarction, ulcers, benign prostatic hypertrophy, arthrogryposis multiplex congenita, osteogenesis imperfecta, keratoconus, scoliosis, duodenal atresia, esophageal atresia, intestinal malrotation, pancreatitis, obesity systemic lupus erythematosus, autoimmune disease, emphysema, scleroderma, allergy, ARDS, neuroprotection, fertility Myasthenia gravis, diabetes, obesity, growth and reproductive disorders hemophilia, hypercoagulation, idiopathic thrombocytopenic purpura, immunodeficiencies, graft vesus host, adrenoleukodystrophy, congenital adrenal hyperplasia, endometriosis, xerostomia, ulcers, cirrhosis, transplantation, diverticular disease, Hirschsprung's disease, appendicitis, arthritis, ankylosing spondylitis, tendinitis, renal artery stenosis, interstitial nephritis, glomerulonephritis, polycystic kidney disease, erythematosus, renal tubular acidosis, IgA nephropathy, anorexia, bulimia, psychotic disorders, including anxiety, schizophrenia, manic depression, delirium, dementia, severe mental retardation and dyskinesias, such as Huntington's disease and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

NOVX nucleic acids and polypeptides are further useful in the generation of antibodies that bind immuno-specifically to the novel NOVX substances for use in therapeutic or diagnostic methods. These NOVX antibodies may be generated according to methods known in the art, using prediction from hydrophobicity charts, as described in the “Anti-NOVX Antibodies” section below. The disclosed NOVX proteins have multiple hydrophilic regions, each of which can be used as an immunogen. These NOVX proteins can be used in assay systems for functional analysis of various human disorders, which will help in understanding of pathology of the disease and development of new drug targets for various disorders.

The NOVX nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their encoded polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE 1
Sequences and Corresponding SEQ ID Numbers

| | | | Nucleic | Poly- |
|---|---|---|---|---|
| | | | Acid | peptide |
| NOVX | | | SEQ ID | SEQ ID |
| No. | Internal Acc. No. | Homology | NO. | NO. |
| 1a | COR87920446_A | Delta serrate ligand | 1 | 2 |
| | | receptor | | |
| 1b | CG57012-01 | Delta serrate ligand | 3 | 4 |
| | | receptor | | |
| 1c | CG57012-02 | Delta serrate ligand | 5 | 6 |
| | | receptor | | |
| 1d | CG57012-03 | Delta serrate ligand | 7 | 8 |
| | | receptor | | |
| 1e | CG57012-04 | Delta serrate ligand | 9 | 10 |
| | | receptor | | |
| 2 | COR87940554 | Protein kinase | 11 | 12 |
| 3 | COR100339661 | GPCR | 13 | 14 |
| 4a | COR87934767 | Ankyrin repeat | 15 | 16 |
| | | containing protein | | |
| 4b | CG57238-01 | Ankyrin repeat | 17 | 18 |
| | | containing protein | | |
| 5 | COR100396092 | Ankyrin repeat | 19 | 20 |
| | | containing protein | | |
| 6 | COR87941483 | TNF intracellular | 21 | 22 |
| | | domain interacting | | |
| | | protein | | |
| 7 | COR101716725 | Secretory protein | 23 | 24 |
| 8a | CG56663-01 | GPCR | 25 | 26 |
| 8b | CG56663-02 | GPCR | 27 | 28 |
| 9 | CG56787_01 | Dual specificity | 29 | 30 |
| | | phosphatase | | |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

NOV1a to NOV1e are homologous to the Delta serrate ligand receptor family of proteins. Thus, the NOV1a to NOV1e nucleic acids, polypeptides, antibodies and related compounds according to the invention are useful in potential diagnostic and therapeutic applications implicated in, for example, cardiovascular disease, Alagille syndrome, neural development defects, other developmental defects and other diseases, disorders and conditions of the like.

NOV2 is homologous to Protein kinases. Therefore, the nucleic acids and proteins of the invention are useful in potential diagnostic and therapeutic applications implicated in, for example, Hypercalceimia, Ulcers, Hemophilia, hypercoagulation, idiopathic thrombocytopenic purpura, autoimmume disease, allergies, immunodeficiencies, transplantation, Graft versus host disease (GVHD), Lymphaedema, Systemic lupus erythematosus, Autoimmune disease, Asthma, Emphysema, Scleroderma, allergy, Diabetes, Autoimmune disease, Renal artery stenosis, Interstitial nephritis, Glomerulonephritis, Polycystic kidney disease, Systemic lupus erythematosus, Renal tubular acidosis, IgA nephropathy, Cardiovascular disease, Hypercalceimia, Lesch-Nyhan syndrome, Fertility, Cancer and other diseases, disorders and conditions of the like.

NOV3, NOV8a and NOV8b are homologous to GPCRs. Thus, the NOV3, NOV8a and NOV8b nucleic acids and polypeptides, antibodies and related compounds according to the invention will be useful in therapeutic and diagnostic applications implicated in, for example, Von Hippel-Lindau (VHL) syndrome, Cirrhosis,Transplantation, Hemophilia, Hypercoagulation, Idiopathic thrombocytopenic purpura, Immunodeficiencies, Graft versus host disorders and other diseases, disorders and conditions of the like.

NOV4a, NOV4b and NOV5 are homologous to the Ankyrin repeat containing proteins. Thus, NOV4a, NOV4b and NOV5 nucleic acids, polypeptides, antibodies and related compounds according to the invention will be useful in therapeutic and diagnostic applications implicated in, for example, Endometriosis, Fertility, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Systemic lupus erythematosus, Autoimmune disease, Asthma, Emphysema, Scleroderma, allergy, and other diseases, disorders and conditions of the like.

NOV6 is homologous to the TNF intracellular domain interaction proteins. Thus NOV6 nucleic acids, polypeptides, antibodies and related compounds according to the invention will be useful in therapeutic and diagnostic applications implicated in, for example, cardiovascular disorders, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Systemic lupus erythematosus, Autoimmune disease, Asthma, Emphysema, Scleroderma, allergy, Diabetes, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome and other diseases, disorders and conditions of the like.

NOV7 is homologous to Secretory proteins. Thus, the NOV7 nucleic acids, polypeptides, antibodies and related compounds according to the invention will be useful in therapeutic and diagnostic applications implicated in, for example, cardiovascular diseases, Cardiomyopathy, Atherosclerosis, Hypertension, Congenital heart defects, Aortic stenosis, Atrial septal defect (ASD), Atrioventricular (A-V) canal defect, Ductus arteriosus , Pulmonary stenosis , Subaortic stenosis, Ventricular septal defect (VSD), valve diseases, Tuberous sclerosis, Scleroderma, Obesity, Transplantation, Systemic lupus erythematosus , Autoimmune disease, Asthma, Emphysema, Scleroderma, allergy, Diabetes, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome and other diseases, disorders and conditions of the like.

NOV9 is homologous to Dual specificity phosphatase. Thus, the NOV9 nucleic acids, polypeptides, antibodies and related compounds according to the invention will be useful in therapeutic and diagnostic applications implicated in, for example, the treatment of patients suffering from: brain disorders including epilepsy, eating disorders, schizophrenia, ADD, and cancer; heart disease; blood disorders, kidney disorders, liver diseases, inflammation and autoimmune disorders including Crohn's disease, IBD, allergies, rheumatoid and osteoarthritis, inflammatory skin disorders, allergies, blood disorders; psoriasis; colon-, ovarian-, testicular-, lymphatic-, brain-, and pancreatic cancers; leukemia AIDS; thalamus disorders; metabolic disorders including diabetes and obesity; lung diseases such as asthma, emphysema, cystic fibrosis, and cancer; pancreatic disorders including pancreatic insufficiency; and prostate disorders including prostate cancer and other diseases, disorders and conditions of the like.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

One NOVX protein of the invention, referred to herein as NOV 1, includes five delta serrate ligand receptors. The disclosed proteins have been named NOV1a, NOV1b, NOV1c, NOV1d and NOV1e.

NOV1a

A disclosed NOV1a (designated CuraGen Ace. No. COR87920446_A), which encodes a novel delta serrate ligand receptor and includes the 3063 nucleotide sequence (SEQ ID NO: 1) is shown in Table 1A. An open reading frame for the mature protein was identified beginning with an ATG initiation codon at nucleotides 1-3 and ending with a TGA codon at nucleotides 3061-3063. Putative untranslated regions, if any, are found upstream from the initiation codon and downstream from the termination codon and are underlined in Table 1A, and the start and stop codons are in bold letters.

TABLE 1A
NOV1a Nucleotide Sequence

| | |
|---|---|
| ATGTCACCGCCTCTGTGTCCCCTCCTTCTCCTGGCTGTGGGCCTGCGGCTGGCTGGAACTCTCAACC | (SEQ ID NO:1) |
| CCAGTGATCCCAATACCTGCAGCTTCTGGGAAAGCTTCACTACCACCACCAAGGAGTCCCACTCCC | |
| GCCCCTTCAGCCTGCTCCCCTCAGAGCCCTGCGAGCGGCCCTGGGAGGGCCCCCATACTTGCCCCC | |
| AGCCCACGGTTGTATACCGGACCGTGTACCGTCAGGTGGTGAAGACGGACCACCGCCAGCGCCTGC | |
| AGTGCTGCCATGGCTTCTATGAGAGCAGGGGGTTCTGTGTCCCGCTCTGTGCCCAGGAGTGTGTCC | |
| ATGGCCGTTGTGTGGCACCCAATCAGTGCCAATGTGTGCCAGGCTGGCGGGGCGACGACTGTTCCA | |
| GTGAGTGTGCCCCAGGAATGTGGGGGCCACAGTGTGACAAGCCCTGCAGCTGCGGCAACAACAGC | |
| TCGTGTGATCCCAAGAGTGGGGTATGTTCTTGCCCTTCTGGTCTGCAGCCCCCGAACTGCCTTCAGC | |
| CCTGTACCCCTGGCTACTATGGCCCTGCCTGCCAGTTCCGCTGCCAGTGCCATGGGGCACCCTGCGA | |
| TCCCCAGACTGGAGCCTGCTTCTGCCCCGCAGAGAGAACTGGGCCCAGCTGTGACGTGTCCTGTTC | |
| CCAGGGCACTTCTGGCTTCTTCTGCCCCAGCACCCATTCTTGCCAAAATGGAGGTGTCTTCCAAACC | |
| CCACAGGGCTCCTGCAGCTGCCCCCCTGGCTGGATGGTATGGAGGGTGGGGCCTGTGGGCATGGGG | |
| TGTGGGTCTGGGGAGAATTCTGTGGGTGGTGCTAAGCAGGGCTCCAAGGGCACCATCTGCTCCCTG | |
| CCCTGCCCAGAGGGCTTTCACGGACCCAACTGCTCCCAGGAATGTCGCTGCCACAACGGCGGCCTC | |
| TGTGACCGATTCACTGGGCAGTGCCGCTGCGCTCCGGGTTACACTGGGGATCGGTGCCGGGAGGAG | |
| TGCCCGGTGGGCCGCTTTGGGCAGGACTGTGCTGAGACGTGCGACTGCGCCCCGGACGCCCGTTGC | |
| TTCCCGGCCAACGGCGCATGTCTGTGCGAACACGGCTTCACTGGGGACCGCTGCACGGATCGCCTC | |
| TGCCCCGACGGCTTCTACGGTCTCAGCTGCCAGGCCCCCTGCACCTGCGACCGGGAGCACAGCCTC | |
| AGCTGCCACCCGATGAACGGGGAGTGCTCCTGCCTGCCGGGCTGGGCGGGCCTCCACTGCAACGA | |
| GAGCTGCCCGCAGGACACGCATGGGCCAGGGTGCCAGGAGCACTGTCTCTGCCTGCACGGTGGCG | |
| TCTGCCAGGCTACCAGCGGCCTCTGTCAGTGCGCGCCGGGTTACACGGGCCCTCACTGTGCTAGTC | |
| TTTGTCCTCCTGACACCTACGGTGTCAACTGTTCTGCACGCTGCTCATGTGAAAATGCCATCGCCTG | |
| CTCACCCATCGACGGCGAGTGCGTCTGCAAGGAAGGTTGGCAGCGTGGTAACTGCTCTGTGCCCTG | |
| CCCACCCGGAACCTGGGGCTTCAGTTGCAATGCCAGCTGCCAGTGTGCCCATGAGGCAGTCTGCAG | |
| CCCCCAAACTGGAGCCTGTACCTGCACCCCTGGGTGGCATGGGGCCCACTGCCAGCTGCCCTGTCC | |
| GAAGGGGCAGTTTGGAGAAGGTTGTGCCAGTCGCTGTGACTGTGACCACTCTGATGGCTGTGACCC | |
| TGTTCATGGACGCTGTCAGTGCCAGGCTGGCTGGATGGGTGCCCGCTGCCACCTGTCCTGCCCTGA | |
| GGGCTTATGGGGAGTCAACTGTAGCAACACCTGCACCTGCAAGAATGGGGGCACCTGTCTCCCTGA | |
| GAATGGCAACTGCGTGTGTGCACCCGGATTCCGGGGCCCCTCCTGCCAGAGATCCTGTCAGCCTGG | |
| CCGCTATGGCAAACGCTGTGTGCCCTGCAAGTGCGCTAACCACTCCTTCTGCCACCCCTCGAACGG | |
| GACCTGCTACTGCCTGGCTGGCTGGACAGGCCCCGACTGCTCCCAGCGCTGCCCTCTGGGGACATT | |
| TGGTGCTAACTGCTCCCAGCCATGCCAGTGTGGTCCTGGAGAAAAGTGCCACCCAGAGACTGGGGC | |
| CTGTGTATGTCCCCCAGGGCACAGTGGTGCACCTTGCAGGATTGGAATCCAGGAGCCCTTTACTGT | |
| GATGCCGACCACTCCAGTAGCGTATAACTCGCTGGGTGCAGTGATTGGCATTGCAGTGCTGGGGTC | |
| CCTTGTGGTAGCCCTGGTGGCACTGTTCATTGGCTATCGGCACTGGCAAAAAGGCAAGGAGCACCA | |
| CCACCTGGCTGTGGCTTACAGCAGCGGGCGCCTGGACGGCTCCGAGTATGTCATGCCAGATGTCCC | |
| TCCCAGCTACAGTCACTACTACTCCAACCCCAGCTACCACACCCTGTCGCAGTGCTCCCCAAACCCC | |
| CCACCCCCTAACAAGGTTCCAGGCCCGCTCTTTGCCAGCCTGCAGAAACCTGAGCGGCCAGGTGGG | |
| GCCCAAGGGCATGATAACCACACCACCCTGCCTGCTGACTGGAAGCACCGCCGGGAGCCCCCTCCA | |
| GGGCCTCTGGACAGGGGGAGCAGCCGCCTGGACCGAAGCTACAGCTATAGCTACAGCAATGGCCC | |
| AGGCCCATTCTACAATAAAGGGCTCATCTCTGAAGAGGAGCTCGGGGCCAGTGTGGCTTCCCTGAG | |
| CAGTGAGAACCCATATGCCACCATCCGGGACCTGCCCAGCTTGCCAGGGGGCCCCCGGGAGAGCA | |
| GCTACATGGAGATGAAAGGCCCTCCCTCAGGATCTCCCCCCAGGCAGCCTCCTCAGTTCTGGGACA | |
| GCCAGAGGCGGCGGCAACCCCAGCCACAGAGAGACAGTGGCACCTACGAGCAGCCCAGCCCCCTG | |
| ATCCATGACCGAGACTCTGTGGGCTCCCAGCCCCCTCTGCCTCCGGGCCTACCCCCCGGCCACTATG | |
| ACTCACCCAAGAACAGCCACATCCCTGGACATTATGACTTGCCTCCAGTACGGCATCCCCCATCAC | |
| CTCCACTTCGACGCCAGGACCGTTGA | |

The disclosed NOV1a nucleic acid sequence maps to chromosome 1 and has 1120 of 1951 bases (57%) identical to a gb:GENBANK-ID:AB011532|acc:AB011532.1 mRNA from Rattus norvegicus (Rattus norvegicus mRNA for MEGF6, complete cds).

The NOV1 a polypeptide (SEQ ID NO: 2) is 1020 amino acid residues in length and is presented using the one-letter amino acid code in Table 1B. The SignalP, Psort and/or Hydropathy results predict that NOV1 a has a signal peptide and is likely to be localized on the plasma membrane with a certainty of 0.6760. In alternative embodiments, a NOV1a polypeptide is located outside the cell with a certainty of 0.1000, in the endoplasmic reticulum (membrane) with a certainty of 0.1000, or in the endoplasmic reticulum (lumen) with a certainty of 0.1000. The SignalP predicts a likely cleavage site for a NOV1a peptide between amino acid positions 20 and 21, i.e., at the dash in the sequence AGT-LN.

TABLE 1B
Encoded NOV1a Protein Sequence

(SEQ ID NO:2)

MSPPLCPLLLLAVGLRLAGTLNPSDPNTCSFWESFTTTTKESHSRPFSLLPSEPCERPWEGPHTCPQPTV
VYRTVYRQVVKTDHRQRLQCCHGFYESRGFCVPLCAQECVHGRCVAPNQCQCVPGWRGDDCSSECA
PGMWGPQCDKPCSCGNNSSCDPKSGVCSCPSGLQPPNCLQPCTPGYYGPACQFRCQCHGAPCDPQTG
ACFCPAERTGPSCDVSCSQGTSGFFCPSTHSCQNGGVFQTPQGSCSCPPGWMVWRVGPVGMGCGSGE
NSVGGAKQGSKGTICSLPCPEGFHGPNCSQECRCHNGGLCDRFTGQCRCAPGYTGDRCREECPVGRFG
QDCAETCDCAPDARCFPANGACLCEHGFTGDRCTDRLCPDGFYGLSCQAPCTCDREHSLSCHPMNGE
CSCLPGWAGLHCNESCPQDTHGPGCQEHCLCLHGGVCQATSGLCQCAPGYTGPHCASLCPPDTYGVN
CSARCSENAIACSPIDGECVCKEGWQRGNCSVPCPPGTWGFSCNASQCAHEAVCSPQTGACTCTPG
WHGAHCQLPCPKGQFGEGCASRCDCDHSDGCDPVHGRCQCQAGWMGARCHLSCPEGLWGVNCSNT
CTCKNGGTCLPENGNCVCAPGFRGPSCQRSCQPGRYGKRCVPCKCANHSFCHPSNGTCYCLAGWTGP
DCSQRCPLGTFGANCSQPCQCGPGEKCHPETGACVCPPGHSGAPCRIGIQEPFTVMPTTPVAYNSLGAV
IGIAVLGSLVVALVALFIGYRHWQKGKEHHHLAVAYSSGRLDGSEYVMPDVPPSYSHYYSNPSYHTLS
QCSPNPPPPNKVPGPLFASLQKPERPGGAQGHDNHTTLPADWKHRREPPPGPLDRGSSRLDRSYSYSYS
NGPGPFYNKGLISEEELGASVASLSSENPYATIRDLPSLPGGPRESSYMEMKGPPSGSPPRQPPQFWDSQ
RRRQPQPQRDSGTYEQPSPLIHDRDSVGSQPPLPPGLPPGHYDSPKNSHIPGHYDLPPVRHPPSPPLRRQ
DR

The NOV1a amino acid sequence has 834 of 1064 amino acid residues (78%) identical to, and 881 of 1064 amino acid residues (82%) similar to, the 1034 amino acid residue gi|17386053|gb|AAL38571.1 AF444274_{—}1 (AF444274) Jedi protein [Mus musculus] (E=0.0).

Possible small nucleotide polymorphisms (SNPs) found for NOV1a are listed in Table 1C.

TABLE 1C
SNPs for NOV1a

| | Nucleotide | Base | Amino Acid | Base |
|---|---|---|---|---|
| Variant | Position | Change | Position | Change |
| 13374399 | 447 | C > T | NA | NA |
| 13374400 | 934 | C > A | NA | NA |
| 13374401 | 975 | G > A | NA | NA |
| 13374402 | 984 | C > T | NA | NA |
| 13374403 | 1011 | T > C | NA | NA |
| 13374404 | 1269 | G > A | NA | NA |
| 13374405 | 1278 | T > C | NA | NA |
| 13374406 | 1297 | C > T | 433 | His > Tyr |
| 13374407 | 1298 | A > G | 433 | His > Arg |
| 13374408 | 1398 | T > A | NA | NA |
| 13374409 | 1585 | A > G | 529 | Ser > Gly |
| 13374410 | 1595 | C > T | 532 | Thr > Ile |
| 13374411 | 1701 | C > T | NA | NA |
| 13374413 | 2300 | G > A | 767 | Gly > Asp |
| 13374414 | 2361 | T > C | NA | NA |

NOV1a is expressed in at least the following tissues: testis. This information was derived by determining the tissue sources of the sequences that were included in the invention including but not limited to SeqCalling sources, Public EST sources, Literature sources, and/or RACE sources

NOV1b

A disclosed NOV1b (designated CuraGen Ace. No. CG57012-01), which includes the 2919 nucleotide sequence (SEQ ID NO: 3) shown in Table 1D. An open reading frame for the mature protein was identified beginning with an ATG codon at nucleotides 83-85 and ending with a TGA codon at nucleotides 2867-2869. The start and stop codons of the open reading frame are highlighted in bold type. Putative untranslated regions are underlined.

TABLE 1D
NOV1b Nucleotide Sequence

(SEQ ID NO:3)

AGATCTCTGCAGACAGGTCCTCCAGGCTGCTGGCTGCAGCGCCACTGCCCACTCTGCGCCGGTCTTGCTGCAG
GCCTCTGCAATGTCACCGCCTCTGTGTCCCCTCCTTCTCCTGGCTGTGGGCCTGCGGCTGGCTGGAACTCTCA
ACCCCAGTGATCCCAATACCTGCAGCTTCTGGGAAAGCTTCACTACCACCACCAAGGAGTCCCACTCCCGCCC
CTTCAGCCTGCTCCCCTCAGAGCCCTGCGAGCGGCCCTGGGAGGGCCCCCATACTTGCCCCCAGCCCACGGTT
GTATACCGGACCGTGTACCGTCAGGTGGTGAAGACGGACCACCGCCAGCGCCTGCAGTGCTGCCATGGCTTCT
ATGAGAGCAGGGGGTTCTGTGTCCCGCTCTGTGCCCAGGAGTGTGTCCATGGCCGTTGTGTGGCACCCAATCA
GTGCCAATGTGTGCCAGGCTGGCGGGGCGACGACTGTTCCAGTGAGTGTGCCCCAGGAATGTGGGGGCCACAG
TGTGACAAGCCCTGCAGCTGCGGCAACAACAGCTCGTGTGATCCCAAGAGTGGGGTATGTTCTTGCCCTTCTG
GTCTGCAGCCCCCGAACTGCCTTCAGCCCTGTACCCCTGGCTACTATGGCCCTGCCTGCCAGTTCCGCTGCCA
GTGCCATGGGGCACCCTGCGATCCCCAGACTGGAGCCTGCTTCTGCCCCGCAGAGAGAACTGGGCCCAGCTGT
GACGTGTCCTGTTCCCAGGGCACTTCTGGCTTCTTCTGCCCCAGCACCCATTCTTGCCAAAATGGAGGTGTCT
TCCAAACCCCACAGGGCTCCTGCAGCTGCCCCCCTGGCTGGATGGTATGGAGGGTGGGGCCTGTGGGCATGGG
GTGTGGGTCTGGGGAGAATTCTGTGGGTGGTGCTAAGCAGGGCTCCAAGGGCACCATCTGCTCCCTGCCCTGC
CCAGAGGGCTTTCACGGACCCAACTGCTCCCAGGAATGTCGCTGCCACAACGGCGGCCTCTGTGACCGATTCA
CTGGGCAGTGCCGCTGCGCTCCGGGTTACACTGGGGATCGGTGCCGGGAGGAGTGCCCGGTGGGCCGCTTTGG
GCAGGACTGTGCTGAGACGTGCGACTGCGCCCCGGACGCCCGTTGCTTCCCGGCCAACGGCGCATGTCTGTGC
GAACACGGCTTCACTGGGGACCGCTGCACGGATCGCCTCTGCCCCGACGGCTTCTACGGTCTCAGCTGCCAGG
CCCCCCGCACCTGCGACCGGGAGCACAGCCTCAGCTGCCACCCGATGAACGGGGAGTGCTCCTGCCTGCCGGG
CTGGGCGGGCCTCCACTGCAACGAGAGCTGCCCGCAGGACACGCATGGGCCAGGGTGCCAGGAGCACTGTCTC
TGCCTGCACGGTGGCGTCTGCCAGGCTACCAGCGGCCTCTGTCAGTGCGCGCCGGGTTACACGGGCCCTCACT
GTGCTAGTCTTTGTCCTCCTGACACCTACGGTGTCAACTGTTCTGCACGCTGCTCATGTGAAAATGCCATCGC
CTGCTCACCCATCGACGGCGAGTGCGTCTGCAAGGAAGGTTGGCAGCGTGGTAACTGCTCTGTGCCCTGCCCA
CCCGGAACCTGGGGCTTCAGTTGCAATGCCAGCTGCCAGTGTGCCCATGAGGCAGTCTGCAGCCCCCAAACTG
GAGCCTGTACCTGCACCCCTGGGTGGCATGGGGCCCACTGCCAGCTGCCCTGTCCGAAGGGGCAGTTTGGAGA
AGGTTGTGCCAGTCGCTGTGACTGTGACCACTCTGATGGCTGTGACCCTGTTCATGGACGCTGTCAGTGCCAG
GCTGGCTGGATGGGTGCCCGCTGCCACCTGTCCTGCCCTGAGGGCTTATGGGGAGTCAACTGTAGCAACACCT
GCACCTGCAAGAATGGGGGCACCTGTCTCCCTGAGAATGGCAACTGCGTGTGTGCGCCCGGATTCCGGGGCCC
CTCCTGCCAGAGATCCTGTCAGCCTGGCCGCTATGGCAAACGCTGTGTGCCCTGCAAGTGCGCTAACCACTCC
TTCTGCCACCCCTCGAACGGGGCCTGCTACTGCCTGGCTGGCTGGACAGGCCCCGACTGCTCCCAGCCATGCC
CTCCAGGACACTGGGGAGAAAACTGTGCCCAGACCTGCCAATGTCACCATGGTGGGACCTGCCATCCCCAGGA
TGGGAGCTGTATCTGCCCCCTAGGCTGGACTGGACACCACTGCTTAGAAGGCTGCCCTCTGGGGACATTTGGT
GCTAACTGCTCCCAGCCATGCCAGTGTGGTCCTGGAGAAAAGTGCCACCCAGAGACTGGGGCCTGTGTATGTC
CCCCAGGGCACAGTGGTGCACCTTGCAGGATTGGAATCCAGGAGCCCTTTACTGTGATGCCGACCACTCCAGT
AGCGTATAACTCGCTGGGTGCAGTGATTGGCATTGCAGTGCTGGGGTCCCTTGTGGTAGCCCTGGTGGCACTG
TTCATTGGCTATCGGCACTGGCAAAAAGACAAGGAGCACCACCACCTGGCTGTGGCTTACAGCAGCGGGCGCC
TGGACGGCTCCGAGTATGTCATGCCAGATGTCCCTCCGAGCTACAGTCACTACTACTCCAACCCCAGCTACCA
CACCCTGTCGCAGTGCTCCCCAAACCCCCCACCCCCTAACAAGGTTCCAGGCCCGCTCTTTGCCAGCCTGCAG
AACCCTGAGCGGCCAGGTGGGGCCCAAGGGCATGATAACCACACCACCCTGCCTGCTGACTGGAAGCACCGCC
GGGAGCCCCCTCCAGGGCCTCTGGACAGGGGTAGGTGCCGGGAGGCCAGGGTCTCTGGCGCGGGTGGATGTGT
GCAGCCCAGATGCCGCGTCTGAGTGTGTGTGTCTGGAGACGGGGGCTCTGGGCCCCATTTCTAGAGGAAGTG

The disclosed NOV1b nucleic acid sequence maps to chromosome 1 and has 853 of 1409 bases (60%) identical to a gb:GENBANK-ID:AB011532|acc:AB011532.1 mRNA from Rattus norvegicus (Rattus norvegicus mRNA for MEGF6, complete cds).

The NOV1b polypeptide (SEQ ID NO: 4) is 928 amino acid residues in length and is presented using the one-letter amino acid code in Table 1E. The SignalP, Psort and/or Hydropathy results predict that NOV1 b has a signal peptide and is likely to be localized to the plasma membrane with a certainty of 0.6760. In alternative embodiments, a NOV1b polypeptide is located to the outside of the cell with a certainty of 0.1000, the endoplasmic reticulum (membrane) with a certainly of 0.1000, or the endoplasmic reticulum (lumen) with a certainty of 0.1000. The SignalP predicts a likely cleavage site for a NOV1b peptide between amino acid positions 20 and 21, i.e., at the dash in the sequence AGT-LN.

TABLE 1E
Encoded NOV1b Protein Sequence

(SEQ ID NO:4)

MSPPLCPLLLLAVGLRLAGTLNPSDPNTCSFWESFTTTTKESHSRPFSLLPSEPCERPWEGPHTCPQPTVVYRT
VYRQVVKTDHRQRLQCCHGFYESRGFCVPLCAQECVHGRCVAPNQCQCVPGWRGDDCSSECAPGMWGPQCDKPC
SCGNNSSCDPKSGVCSCPSGLQPPNCLQPCTPGYYGPACQFRCQCHGAPCDPQTGACFCPAERTGPSCDVSCSQ
GTSGFFCPSTHSCQNGGVFQTPQGSCSCPPGWMVWRVGPVGMGCGSGENSVGGAKQGSKGTICSLPCPEGFHGP
NCSQECRCHNGGLCDRFTGQCRCAPGYTGDRCREECPVGRFGQDCAETCDCAPDARCFPANGACLCEHGFTGDR
CTDRLCPDGFYGLSCQAPRTCDREHSLSCHPMNGECSCLPGWAGLHCNESCPQDTHGPGCQEHCLCLHGGVCQA
TSGLCQCAPGYTGPHCASLCPPDTYGVNCSARCSCENAIACSPIDGECVCKEGWQRGNCSVPCPPGTWGFSCNA
SCQCAHEAVCSPQTGACTCTPGWHGAHCQLPCPKGQFGEGCASRCDCDHSDGCDPVHGRCQCQAGWMGARCHLS
CPEGLWGVNCSNTCTCKNGGTCLPENGNCVCAPGFRGPSCQRSCQPGRYGKRCVPCKCANHSFCHPSNGACYCL
AGWTGPDCSQPCPPGHWGENCAQTCQCHHGGTCHPQDGSCICPLGWTGHHCLEGCPLGTFGANCSQPCQCGPGE
KCHPETGACVCPPGHSGAPCRIGIQEPFTVMPTTPVAYNSLGAVIGIAVLGSLVVALVALFIGYRHWQKDKEHH
HLAVAYSSGRLDGSEYVMPDVPPSYSHYYSNPSYHTLSQCSPNPPPPNKVPGPLFASLQNPERPGGAQGHDNHT
TLPADWKHRREPPPGPLDRGRCREARVSGAGGCVQPRCRV

The NOV1b amino acid sequence 834 of 1064 amino acid residues (78%) identical to, and 881 of 1064 amino acid residues (82%) similar to, the 1034 amino acid residue gi|17386053|gb|AAL38571.1|AF444274_{—}1 (AF444274) Jedi protein [Mus musculus] (E=0.0).

Possible small nucleotide polymorphisms (SNPs) found for NOV1a are listed in Table 1F.

TABLE 1F
SNPs for NOV1b

| | Nucleotide | Base | Amino Acid | Base |
|---|---|---|---|---|
| Variant | Position | Change | Position | Change |
| 13374399 | 529 | C > T | NA | NA |
| 13374400 | 1016 | C > A | NA | NA |
| 13374401 | 1057 | G > A | NA | NA |
| 13374402 | 1066 | C > T | NA | NA |
| 13374403 | 1003 | T > C | NA | NA |
| 13374408 | 1480 | T > A | NA | NA |
| 13374409 | 1667 | A > G | 529 | Ser > Gly |
| 13374410 | 1677 | C > T | 532 | Thr > Ile |
| 13374411 | 1783 | C > T | NA | NA |
| 13374413 | 2511 | A > G | 810 | Asp > Gly |
| 13374414 | 2572 | T > C | NA | NA |

NOV1c

A disclosed NOV1c (designated CuraGen Ace. No. CG57012-02), which includes the 2919 nucleotide sequence (SEQ ID NO: 5) shown in Table 1G. An open reading frame for the mature protein was identified beginning with an ATG codon at nucleotides 83-85 and ending with a TGA codon at nucleotides 2867-2869. The start and stop codons of the open reading frame are highlighted in bold type. Putative untranslated regions are underlined.

TABLE 1G
NOV1c Nucleotide Sequence

(SEQ ID NO:5)

AGATCTCTGCAGACAGGTCCTCCAGGCTGCTGGCTGCAGCGCCACTGCCCACTCTGCGCCGGTCTTGCTGCAG
GCCTCTGCAATGTCACCGCCTCTGTGTCCCCTCCTTCTCCTGGCTGTGGGCCTGCGGCTGGCTGGAACTCTCA
ACCCCAGTGATCCCAATACCTGCAGCTTCTGGGAAAGCTTCACTACCACCACCAAGGAGTCCCACTCCCGCCC
CTTCAGCCTGCTCCCCTCAGAGCCCTGCGAGCGGCCCTGGGAGGGCCCCCATACTTGCCCCCAGCCCACGGTT
GTATACCGGACCGTGTACCGTCAGGTGGTGAAGACGGACCACCGCCAGCGCCTGCAGTGCTGCCATGGCTTCT
ATGAGAGCAGGGGGTTCTGTGTCCCGCTCTGTGCCCAGGAGTGTGTCCATGGCCGTTGTGTGGCACCCAATCA
GTGCCAATGTGTGCCAGGCTGGCGGGGCGACGACTGTTCCAGTGAGTGTGCCCCAGGAATGTGGGGGCCACAG
TGTGACAAGCCCTGCAGCTGCGGCAACAACAGCTCGTGTGATCCCAAGAGTGGGGTATGTTCTTGCCCTTCTG
GTCTGCAGCCCCCGAACTGCCTTCAGCCCTGTACCCCTGGCTACTATGGCCCTGCCTGCCAGTTCCGCTGCCA
GTGCCATGGGGCACCCTGCGATCCCCAGACTGGAGCCTGCTTCTGCCCCGCAGAGAGAACTGGGCCCAGCTGT
GACGTGTCCTGTTCCCAGGGCACTTCTGGCTTCTTCTGCCCCAGCACCCATTCTTGCCAAAATGGAGGTGTCT
TCCAAACCCCACAGGGCTCCTGCAGCTGCCCCCCTGGCTGGATGGTATGGAGGGTGGGGCCTGTGGGCATGGG
GTGTGGGTCTGGGGAGAATTCTGTGGGTGGTGCTAAGCAGGGCTCCAAGGGCACCATCTGCTCCCTGCCCTGC
CCAGAGGGCTTTCACGGACCCAACTGCTCCCAGGAATGTCGCTGCCACAACGGCGGCCTCTGTGACCGATTCA
CTGGGCAGTGCCGCTGCGCTCCGGGTTACACTGGGGATCGGTGCCGGGAGGAGTGCCCGGTGGGCCGCTTTGG
GCAGGACTGTGCTGAGACGTGCGACTGCGCCCCGGACGCCCGTTGCTTCCCGGCCAACGGCGCATGTCTGTGC
GAACACGGCTTCACTGGGGACCGCTGCACGGATCGCCTCTGCCCCGACGGCTTCTACGGTCTCAGCTGCCAGG
CCCCCCGCACCTGCGACCGGGAGCACAGCCTCAGCTGCCACCCGATGAACGGGGAGTGCTCCTGCCTGCCGGG
CTGGGCGGGCCTCCACTGCAACGAGAGCTGCCCGCAGGACACGCATGGGCCAGGGTGCCAGGAGCGCTGTCTC
TGCCTGCACGGTGGCGTCTGCCAGGCTACCAGCGGCCTCTGTCAGTGCGCGCCGGGTTACACGGGCCCTCACT
GTGCTAGTCTTTGTCCTCCTGACACCTACGGTGTCAACTGTTCTGCACGCTGCTCATGTGAAAATGCCATCGC
CTGCTCACCCATCGACGGCGAGTGCGTCTGCAAGGAAGGTTGGCAGCGTGGTAACTGCTCTGTGCCCTGCCCA
CCCGGAACCTGGGGCTTCAGTTGCAATGCCAGCTGCCAGTGTGCCCATGAGGCAGTCTGCAGCCCCCAAACTG
GAGCCTGTACCTGCACCCCTGGGTGGCATGGGGCCCACTGCCAGCTGCCCTGTCCGAAGGGGCAGTTTGGAGA
AGGTTGTGCCAGTCGCTGTGACTGTGACCACTCTGATGGCTGTGACCCTGTTCATGGACGCTGTCAGTGCCAG
GCTGGCTGGATGGGTGCCCGCTGCCACCTGTCCTGCCCTGAGGGCTTATGGGGAGTCAACTGTAGCAACACCT
GCACCTGCAAGAATGGGGGCACCTGTCTCCCTGAGAATGGCAACTGCGTGTGTGCGCCCGGATTCCGGGGCCC
CTCCTGCCAGAGATCCTGTCAGCCTGGCCGCTATGGCAAACGCTGTGTGCCCTGCAAGTGCGCTAACCACTCC
TTCTGCCACCCCTCGAACGGGGCCTGCTACTGCCTGGCTGGCTGGACAGGCCCCGACTGCTCCCAGCCATGCC
CTCCAGGACACTGGGGAGAAAACTGTGCCCAGACCTGCCAATGTCACCATGGTGGGACCTGCCATCCCCAGGA
TGGGAGCTGTATCTGCCCCCTAGGCTGGACTGGACACCACTGCTTAGAAGGCTGCCCTCTGGGGACATTTGGT
GCTAACTGCTCCCAGCCATGCCAGTGTGGTCCTGGAGAAAAGTGCCACCCAGAGACTGGGGCCTGTGTATGTC
CCCCAGGGCACAGTGGTGCACCTTGCAGGATTGGAATCCAGGAGCCCTTTACTGTGATGCCGACCACTCCAGT
AGCGTATAACTCGCTGGGTGCAGTGATTGGCATTGCAGTGCTGGGGTCCCTTGTGGTAGCCCTGGTGGCACTG
TTCATTGGCTATCGGCACTGGCAAAAAGACAAGGAGCACCACCACCTGGCTGTGGCTTACAGCAGCGGGCGCC
TGGACGGCTCCGAGTATGTCATGCCAGATGTCCCTCCGAGCTACAGTCACTACTACTCCAACCCCAGCTACCA
CACCCTGTCGCAGTGCTCCCCAAACCCCCCACCCCCTAACAAGGTTCCAGGCCCGCTCTTTGCCAGCCTGCAG
AACCCTGAGCGGCCAGGTGGGGCCCAAGGGCATGATAACCACACCACCCTGCCTGCTGACTGGAAGCACCGCC
GGGAGCCCCCTCCAGGGCCTCTGGACAGGGGTAGGTGCCGGGAGGCCAGGGTCTCTGGCGCGGGTGGATGTGT
GCAGCCCAGATGCCGCGTCTGAGTGTGTGTGTCTGGAGACGGGGGCTCTGGGCCCCATTTCTAGAGGAAGTG

The nucleic acid sequence of NOV1c maps to chromosome 1 and has 852 of 1409 bases (60%) identical to a gb:GENBANK-ID:AB011532|acc:AB011532.1 mRNA from Rattus norvegicus (Rattus norvegicus mRNA for MEGF6, complete cds).

The NOV1c polypeptide (SEQ ID NO: 6) is 928 amino acid residues in length and is presented using the one-letter amino acid code in Table 1H. The SignalP, Psort and/or Hydropathy results predict that NOV1c has a signal peptide and is likely to be localized to the plasma membrane with a certainty of 0.6760. In alternative embodiments, a NOV1c polypeptide is located to the outside of the cell with a certainty of 0.1000, the endoplasmic reticulum (membrane) with a certainty of 0.1000, or the endoplasmic reticulum (lumen) with a certainty of 0.1000. The SignalP predicts a likely cleavage site for a NOV1c peptide between amino acid positions 20 and 21, i.e. at the dash in the sequence AGT-LN.

TABLE 1H
Encoded NOV1c Protein Sequence

(SEQ ID NO:6)

MSPPLCPLLLLAVGLRLAGTLNPSDPNTCSFWESFTTTTKESHSRPFSLLPSEPCERPWEGPHTCPQPTVVYRT
VYRQVVKTDHRQRLQCCHGFYESRGFCVPLCAQECVHGRCVAPNQCQCVPGWRGDDCSSECAPGMWGPQCDKPC
SCGNNSSCDPKSGVCSCPSGLQPPNCLQPCTPGYYGPACQFRCQCHGAPCDPQTGACFCPAERTGPSCDVSCSQ
GTSGFFCPSTHSCQNGGVFQTPQGSCSCPPGWMVWRVGPVGMGCGSGENSVGGAKQGSKGTICSLPCPEGFHGP
NCSQECRCHNGGLCDRFTGQCRCAPGYTGDRCREECPVGRFGQDCAETCDCAPDARCFPANGACLCEHGFTGDR
CTDRLCPDGFYGLSCQAPRTCDREHSLSCHPMNGECSCLPGWAGLHCNESCPQDTHGPGCQERCLCLHGGVCQA
TSGLCQCAPGYTGPHCASLCPPDTYGVNCSARCSCENAIACSPIDGECVCKEGWQRGNCSVPCPPGTWGFSCNA
SCQCAHEAVCSPQTGACTCTPGWHGAHCQLPCPKGQFGEGCASRCDCDHSDGCDPVHGRCQCQAGWMGARCHLS
CPEGLWGVNCSNTCTCKNGGTCLPENGNCVCAPGFRGPSCQRSCQPGRYGKRCVPCKCANHSFCHPSNGACYCL
AGWTGPDCSQPCPPGHWGENCAQTCQCHHGGTCHPQDGSCICPLGWTGHHCLEGCPLGTFGANCSQPCQCGPGE
KCHPETGACVCPPGHSGAPCRIGIQEPFTVMPTTPVAYNSLGAVIGIAVLGSLVVALVALFIGYRHWQKDKEHH
HLAVAYSSGRLDGSEYVMPDVPPSYSHYYSNPSYHTLSQCSPNPPPPNKVPGPLFASLQNPERPGGAQGHDNHT
TLPADWKHRREPPPGPLDRGRCREARVSGAGGCVQPRCRV

The NOV1c amino acid sequence has 834 of 1064 amino acid residues (78%) identical to, and 881 of 1064 amino acid residues (82%) similar to, the 1034 amino acid residue gi|17386053|gb|AAL38571.1|AF444274_{—}1 (AF444274) Jedi protein [Mus musculus] (E=0.0).

NOV1d

A disclosed NOV1d (designated CuraGen Acc. No. CG57012-03), which includes the 5000 nucleotide sequence (SEQ ID NO: 7) shown in Table 1I. An open reading frame for the mature protein was identified beginning with an ATG codon at nucleotides 83-85 and ending with a TGA codon at nucleotides 3194-3196. The start and stop codons of the open reading frame are highlighted in bold type.

TABLE 1I
NOV1d Nucleotide Sequence

| | |
|---|---|
| AGATCTCTGCAGACAGGTCCTCCAGGCTGCTGGCTGCAGCGCCACTGCCCACTCTGCGCCGGTCT | (SEQ ID NO:7) |
| TGCTGCAGGCCTCTGCAATGTCACCGCCTCTGTGTCCCCTCCTTCTCCTGGCTGTGGGCCTGCGG | |
| CTGGCTGGAACTCTCAACCCCAGTGATCCCAATACCTGCAGCTTCTGGGAAAGCTTCACTACCAC | |
| CACCAAGGAGTCCCACTCCCGCCCCTTCAGCCTGCTCCCCTCAGAGCCCTGCGAGCGGCCCTGGG | |
| AGGGCCCCCATACTTGCCCCCAGCCCACGGTTGTATACCGGACCGTGTACCGTCAGGTGGTGAA | |
| GACGGACCACCGCCAGCGCCTGCAGTGCTGCCATGGCTTCTATGAGAGCAGGGGGTTCTGTGTC | |
| CCGCTCTGTGCCCAGGAGTGTGTCCATGGCCGTTGTGTGGCACCCAATCAGTGCCAATGTGTGCC | |
| AGGCTGGCGGGGCGACGACTGTTCCAGTGAGTGTGCCCCAGGAATGTGGGGGCCACAGTGTGAC | |
| AAGCCCTGCAGCTGCGGCAACAACAGCTCGTGTGATCCCAAGAGTGGGGTATGTTCTTGCCCTTC | |
| TGGTCTGCAGCCCCCGAACTGCCTTCAGCCCTGTACCCCTGGCTACTATGGCCCTGCCTGCCAGT | |
| TCCGCTGCCAGTGCCATGGGGCACCCTGCGATCCCCAGACTGGAGCCTGCTTCTGCCCCGCAGA | |
| GAGAACTGGGCCCAGCTGTGACGTGTCCTGTTCCCAGGGCACTTCTGGCTTCTTCTGCCCCAGCA | |
| CCCATCCTTGCCAAAATGGAGGTGTCTTCCAAACCCCACAGGGCTCCTGCAGCTGCCCCCCTGGC | |
| TGGATGGGCACCATCTGCTCCCTGCCCTGCCCAGAGGGCTTTCACGGACCCAACTGCTCCCAGGA | |
| ATGTCGCTGCCACAACGGCGGCCTCTGTGACCGATTCACTGGGCAGTGCCGCTGCGCTCCGGGTT | |
| ACACTGGGGATCGGTGCCGGGAGGAGTGCCCGGTGGGCCGCTTTGGGCAGGACTGTGCTGAGAC | |
| GTGCGACTGCGCCCCGGACGCCCGTTGCTTCCCGGCCAACGGCGCATGTCTGTGCGAACACGGC | |
| TTCACTGGGGACCGCTGCACGGATCGCCTCTGCCCCGACGGCTTCTACGGTCTCAGCTGCCAGGC | |
| CCCCCGCACCTGCGACCGGGAGCACAGCCTCAGCTGCCACCCGATGAACGGGGAGTGCTCCTGC | |
| CTGCCGGGCTGGGCGGGCCTCCACTGCAACGAGAGCTGCCCGCAGGACACGCATGGGCCAGGGT | |
| GCCAGGAGCACTGTCTCTGCCTGCACGGTGGCGTCTGCCAGGCTACCAGCGGCCTCTGTCAGTGC | |
| GCGCCGGGTTACACGGGCCCTCACTGTGCTAGTCTTTGTCCTCCTGACACCTACGGTGTCAACTG | |
| TTCTGCACGCTGCTCATGTGAAAATGCCATCGCCTGCTCACCCATCGACGGCGAGTGCGTCTGCA | |
| AGGAAGGTTGGCAGCGTGGTAACTGCTCTGTGCCCTGCCCACCCGGAACCTGGGGCTTCAGTTG | |
| CAATGCCAGCTGCCAGTGTGCCCATGAGGCAGTCTGCAGCCCCCAAACTGGAGCCTGTACCTGC | |
| ACCCCTGGGTGGCATGGGGCCCACTGCCAGCTGCCCTGTCCGAAGGGGCAGTTTGGAGAAGGTT | |
| GTGCCAGTCGCTGTGACTGTGACCACTCTGATGGCTGTGACCCTGTTCATGGACGCTGTCAGTGC | |
| CAGGCTGGCTGGATGGGTGCCCGCTGCCACCTGTCCTGCCCTGAGGGCTTATGGGGAGTCAACT | |
| GTAGCAACACCTGCACCTGCAAGAATGGGGGCACCTGTCTCCCTGAGAATGGCAACTGCGTGTG | |
| TGCGCCCGGATTCCGGGGCCCCTCCTGCCAGAGATCCTGTCAGCCTGGCCGCTATGGCAAACGCT | |
| GTGTGCCCTGCAAGTGCGCTAACCACTCCTTCTGCCACCCCTCGAACGGGACCTGCTACTGCCTG | |
| GCTGGCTGGACAGGCCCCGACTGCTCCCAGCCATGCCCTCCAGGACACTGGGGAGAAAACTGTG | |
| CCCAGACCTGCCAATGTCACCATGGTGGGACCTGCCATCCCCAGGATGGGAGCTGTATCTGCCC | |
| CCTAGGCTGGACTGGACACCACTGCTTAGAAGGCTGCCCTCTGGGGACATTTGGTGCTAACTGCT | |
| CCCAGCCATGCCAGTGTGGTCCTGGAGAAAAGTGCCACCCAGAGACTGGGGCCTGTGTATGTCC | |
| CCCAGGGCACAGTGGTGCACCTTGCAGGATTGGAATCCAGGAGCCCTTTACTGTGATGCCGACC | |
| ACTCCAGTAGCGTATAACTCGCTGGGTGCAGTGATTGGCATTGCAGTGCTGGGGTCCCTTGTGGT | |
| AGCCCTGGTGGCACTGTTCATTGGCTATCGGCACTGGCAAAAAGACAAGGAGCACCACCACCTG | |
| GCTGTGGCTTACAGCAGCGGGCGCCTGGACGGCTCCGAGTATGTCATGCCAGATGTCCCTCCGA | |
| GCTACAGTCACTACTACTCCAACCCCAGCTACCACACCCTGTCGCAGTGCTCCCCAAACCCCCCA | |
| CCCCCTAACAAGGTTCCAGGCCCGCTCTTTGCCAGCCTGCAGAACCCTGAGCGGCCAGGTGGGG | |
| CCCAAGGGCATGATAACCACACCACCCTGCCTGCTGACTGGAAGCACCGCCGGGAGCCCCCTCC | |
| AGGGCCTCTGGACAGGGGGAGCAGCCGCCTGGACCGAAGCTACAGCTATAGCTACAGCAATGG | |
| CCCAGGCCCATTCTACAATAAAGGGCTCATCTCTGAAGAGGAGCTCTGGGCCAGTGTGGCTTCC | |
| CTGAGCAGTGAGAACCCATATGCCACCATCCGGGACCTGCCCAGCTTGCCAGGGGGCCCCCGGG | |
| AGAGCAGCTACATGGAGATGAAAGGCCCTCCCTCAGGATCTCCCCCCAGGCAGCCTCCTCAGTT | |
| CTGGGACAGCCAGAGGCGGCGGCAACCCCAGCCACAGAGAGACAGTGGCACCTACGAGCAGCC | |
| CAGCCCCCTGATCCATGACCGAGACTCTGTGGGCTCCCAGCCCCCTCTGCCTCCGGGCCTACCCC | |
| CCGGCCACTATGACTCACCCAAGAACAGCCACATCCCTGGACATTATGACTTGCCTCCAGTACG | |
| GCATCCCCCATCACCTCCACTTCGACGCCAGGACCGTTGAGGAGCCAGGATGGTATGGCAGAGG | |
| CCAGCACACCTGGCTGTTGCTGCTCAAGGCTGGGGACAGAGCCTAGTGTACCCCTGCCAGGAGC | |
| AGGGAGTGGACCGGCAGGCTGTGAACATGAACAACGCTTAACAGAGCAAGTGATGGGAGCCTT | |
| GTTCCTGGGTTCTACCATGGGAGACGCTGATCAGCAGGATGCCTGGCTCCCTTTCCCAACCCACT | |
| GCTCCCAAGGCCTCCAGGGCCCTGTGTACATAAACTGGTGGGTTGGAAGTTGCTGGGTAACTCT | |
| GATTTCAGACATGCGTGTGGGGTACCTTTTCTGTGCATGCTCAGCCTGGGCTCTGTGCGTGTGTG | |
| TGTTTCTGTGATTTTAGAAGGGTACCAGGCACAGGTTCTGTCCTAGGGCACTTACCATTTAGTAG | |
| GGAGATGGAACCAACCCAATTAACTCTAGCAATAGCCTCCTAACTGGCCTCCTCCATTGATTCAG | |
| TGAACCTTCCAATGCATGGCTCATAATTTCAAAATACAGGCTGGTTAGTTACTCCCTACCTGAAA | |
| GCCTTCATAGGTGCCTCTTTGCTCTTCTGCCAGTATCAAAACTTTTGAAGGCCTTAAAGGCCCTG | |
| CTTTGCCTGGCCCATCTGTCTCTCCAGCCTCACCTTGAACTGTGTTCCTGTCACTGCACGCCAGTC | |
| ACACCGGCCTCTAGGTCCTCCTGTAGGCCACTCTTCTTTCTGGCACAGGGACCTGCACACCTGGA | |
| GTGCCCTTCCTCCCCCACTCGCCTGTTCACCCCTGCTTTTCCTTTACACCTCCTCCTCAGGGAAGT | |
| GCCCACCCTCCGTACATCTTTCACAGCCCTGATTGCAGCTGTGTTCACTCACCAGGTACCTGCAG | |
| AAGGCCTACAGGGTGCCAGGCACTTCTTTAATGGGTTCTTTCTTTATGTGATTATTTGATTAATCT | |
| CTGCCTCCCCCACTAGACTGTAAGCTCCCTGAAGGCAAGAATCCTGTGCTTATGCTCAATATTAG | |
| CTCTCCCTTGGCACAGAGTAGGCACTCAACAAATGCTCCCCAAAAGGCTGAGTGGCTGACTGAA | |
| TTAAGTACCAGTGACATGCAGTAACTGCTAAGATAGATGAGCCATCTGTATGCTCTGACAGTTAC | |
| AGACTGAATAAGTTGGAGACTTCCCTAAAGGGTGGCATTTCCCCAGGGTAACAACGCAGAGCTC | |
| AGGTGTGGGAAGGTGCCAGGGGCAGGGGTGCAGAGGGGCTGAGGCTGAGGGGGGTGCAGAGG | |
| CTGGAGAAAGGATAACAGGAGAGAGTATACAGGCATGCCTTGATTTATTGCACTTCACAGGTAG | |
| CAGAATTTTTAAAGAAATTGAAGGTTTTGGGACATATATGTGACAGCAATAGGTTAAGAAAAGC | |
| AAAGCAGAGAAATTGAAGATTTGTGTCAACACTGCTTTAAGCAAATCTGTTGGCACCATTTTTCC | |
| AATAGCATGTGCCCATTTTGGGTCTCTACATTGCATTTTGGTAATTGCTTGCAATATTTCAAGCAT | |
| TTTCATTGTTATTATATGTGTTATAGTGATCTGTGATCAGTGATCTTTGATATATTATTGTAATTG | |
| TTTCGGGGCGCCATGAACCGCACCCATATAACACGGTAAACTTAATCAGCAAAAAAAAAAAAA | |
| AAAAAAAAACCCGGAAAAATTTTAGAATTGAAAAATATGAAAAACCCCCGGGGGGGTCTTTTCA | |
| GGGGGGGGCGGGGCCCCCAATTTAAATTTTTTTTTTTTTAACAAGGGTAAGTCCCTTTTTTGAGA | |
| AAAAAATCCTCCTGAAAGATTAAATTTGGGGGCC | |

The nucleic acid sequence of NOV1d has 414 of 421 bases (98%) identical to a gb:GENBANK-ID:AX071876|acc:AX071876.1 mRNA from Homo sapiens (Sequence 2348 from Patent WO0102568).

The NOV1d polypeptide (SEQ ID NO: 8) is 1037 amino acid residues in length and is presented using the one-letter amino acid code in Table 1J. The SignalP, Psort and/or Hydropathy results predict that NOV1d has a signal peptide and is likely to be localized to the plasma membrane with a certainty of 0.6760. In alternative embodiments, a NOV1d polypeptide is located to the outside of the cell with a certainty of 0.1000, the endoplasmic reticulum (membrane) with a certainty of 0.1000, or the endoplasmic reticulum (lumen) with a certainty of 0.1000. The SignalP predicts a likely cleavage site for a NOV1d peptide between amino acid positions 20 and 21, i.e., at the dash in the sequence AGT-LN.

TABLE 1J
Encoded NOV1d Protein Sequence

(SEQ ID NO:8)

MSPPLCPLLLLAVGLRLAGTLNPSDPNTCSFWESFTTTTKESHSRPFSLLPSEPCERPWEGPHTCPQPTV
VYRTVYRQVVKTDHRQRLQCCHGFYESRGFCVPLCAQECVHGRCVAPNQCQCVPGWRGDDCSSEC
APGMWGPQCDKPCSCGNNSSCDPKSGVCSCPSGLQPPNCLQPCTPGYYGPACQFRCQCHGAPCDPQT
GACFCPAERTGPSCDVSCSQGTSGFFCPSTHPCQNGGVFQTPQGSCSCPPGWMGTICSLPCPEGFHGPN
CSQECRCHNGGLCDRFTGQCRCAPGYTGDRCREECPVGRFGQDCAETCDCAPDARCFPANGACLCEH
GFTGDRCTDRLCPDGFYGLSCQAPRTCDREHSLSCHPMNGECSCLPGWAGLHCNESCPQDTHGPGCQ
EHCLCLHGGVCQATSGLCQCAPGYTGPHCASLCPPDTYGVNCSARCSCENAIACSPIDGECVCKEGW
QRGNCSVPCPPGTWGFSCNASCQCAHEAVCSPQTGACTCTPGWHGAHCQLPCPKGQFGEGCASRCD
CDHSDGCDPVHGRCQCQAGWMGARCHLSCPEGLWGVNCSNTCTCKNGGTCLPENGNCVCAPGFRG
PSCQRSCQPGRYGKRCVPCKCANHSFCHPSNGTCYCLAGWTGPDCSQPCPPGHWGENCAQTCQCHH
GGTCHPQDGSCICPLGWTGHHCLEGCPLGTFGANCSQPCQCGPGEKCHPETGACVCPPGHSGAPCRIG
IQEPFTVMPTTPVAYNSLGAVIGIAVLGSLVVALVALFIGYRHWQKDKEHHHLAVAYSSGRLDGSEYV
MPDVPPSYSHYYSNPSYHTLSQCSPNPPPPNKVPGPLFASLQNPERGGAQGHDNHTTLPADWKHRRE
PPPGPLDRGSSRLDRSYSYSYSNGPGPFYNKGLISEEELWASVASLSSENPYATIRDLPSLPGGPRESSY
MEMKGPPSGSPPRQPPQFWDSQRRRQPQPQRDSGTYEQPSPLIHDRDSVGSQPPLPPGLPPGHYDSPKN
SHIPGHYDLPPVRHPPSPPLRRQDR

NOV1e

A disclosed NOV1e (designated CuraGen Acc. No. CG57012-04), which includes the 3114 nucleotide sequence (SEQ ID NO: 9) shown in Table 1K. An open reading frame for the mature protein was identified beginning with an ATG codon at nucleotides 1-3 and ending with a TGA codon at nucleotides 3112-3114 The start and stop codons of the open reading frame are highlighted in bold type.

TABLE 1K
NOV1e Nucleotide Sequence

(SEQ ID NO:9)

ATGTCACCGCCTCTGTGTCCCCTCCTTCTCCTGGCTGTGGGCCTGCGGCTGGCTGGAACTCTCAACCCCAGTG
ATCCCAATACCTGCAGCTTCTGGGAAAGCTTCACTACCACCACCAAGGAGTCCCACTCCCGCCCCTTCAGCCT
GCTCCCCTCAGAGCCCTGCGAGCGGCCCTGGGAGGGCCCCCATACTTGCCCCCAGCCCACGGTTGTATACCGG
ACCGTGTACCGTCAGGTGGTGAAGACGGACCACCGCCAGCGCCTGCAGTGCTGCCATGGCTTCTATGAGAGCA
GGGAGTTCTGTGTCCCGCTCTGTGCCCAGGAGTGTGTCCATGGCCGTTGTGTGGCACCCAATCAGTGCCAATG
TGTGCCAGGCTGGCGGGGCGACGACTGTTCCAGTGAGTGTGCCCCAGGAATGTGGGGGCCACAGTGTGACAAG
CCCTGCAGTTGCGGCAACAACAGCTCGTGTGATCCCAAGAGTGGGGTATGTTCTTGCCCTTCTGGTCTGCAGC
CCCCGAACTGCCTTCAGCCCTGTACCCCTGGCTACTATGGCCCTGCCTGCCAGTTCCGCTGCCAGTGCCATGG
GGCACCCTGCGATCCCCAGACTGGAGCCTGCTTCTGCCCCGCAGAGAGAACTGGGCCCAGCTGTGACGTGTCC
TGTTCCCAGGGCACTTCTGGCTTCTTCTGCCCCAGCACCCATCCTTGCCAAAATGGAGGTGTCTTCCAAACCC
CACAGGGCTCCTGCAGCTGCCCCCCTGGCTGGATGGGCACCATCTGCTCCCTGCCCTGCCCAGAGGGCTTTCA
CGGACCCAACTGCTCCCAGGAATGTCGCTGCCACAACGGCGGCCTCTGTGACCGATTCACTGGGCAGTGCCGC
TGCGCTCCGGGTTACACTGGGGATCGGTGCCGGGAGGAGTGCCCGGTGGGCCGCTTTGGGCAGGACTGTGCTG
AGACGTGCGACTGCGCCCCGGACGCCCGTTGCTTCCCGGCCAACGGCGCATGTCTGTGCGAACACGGCTTCAC
TGGGGACCGCTGCACGGATCGCCTCTGCCCCGACGGCTTCTACGGTCTCAGCTGCCAGGCCCCCTGCACCTGC
GACCGGGAGCACAGCCTCAGCTGCCACCCGATGAACGGGGAGTGCTCCTGCCTGCCGGGCTGGGCGGGCCTCC
ACTGCAACGAGAGCTGCCCGCAGGACACGCATGGGCCAGGGTGCCAGGAGTACTGTCTCTGCCTGCACGGTGG
CGTCTGCCAGGCTACCAGCGGCCTCTGTCAGTGCGCGCCGGGTTACACGGGCCCTCACTGTGCTAGTCTTTGT
CCTCCTGACACCTACGGTGTCAACTGTTCTGCACGCTGCTCATGTGAAAATGCCATCGCCTGCTCACCCATCG
ACGGCGAGTGCGTCTGCAAGGAAGGTTGGCAGCGTGGTAACTGCTCTGTGCCCTGCCCACCCGGAACCTGGGG
CTTCAGTTGCAATGCCAGCTGCCAGTGTGCCCATGAGGCAGTCTGCAGCCCCCAAACTGGAGCCTGTACCTGC
ACCCCTGGGTGGCATGGGGCCCACTGCCAGCTGCCCTGTCCGAAGGGGCAGTTTGGAGAAGGTTGTGCCAGTC
GCTGTGACTGTGACCACTCTGATGGCTGTGACCCTGTTCATGGACGCTGTCAGTGCCAGGCTGGCTGGATGGG
TGCCCGCTGCCACCTGTCCTGCCCTGAGGGCTTATGGGGAGTCAACTGTAGCAACACCTGCACCTGCAAGAAT
GGGGGCACCTGTCTCCCTGAGAATGGCAACTGCGTGTGTGCACCCGGATTCCGGGGCCCCTCCTGCCAGAGAT
CCTGTCAGCCTGGCCGCTATGGCAAACGCTGTGTGCCCTGCAAGTGCGCTAACCACTCCTTCTGCCACCCCTC
GAACGGGACCTGCTACTGCCTGGCTGGCTGGACAGGCCCCGACTGCTCCCAGCCATGCCCTCCAGGACACTGG
GGAGAAAACTGTGCCCAGACCTGCCAATGTCACCATGGTGGGACCTGCCATCCCCAGGATGGGAGCTGTATCT
GCCCCCTAGGCTGGACTGGACACCACTGCTTAGAAGGCTGCCCTCTGGGGACATTTGGTGCTAACTGCTCCCA
GCCATGCCAGTGTGGTCCTGGAGAAAAGTGCCACCCAGAGACTGGGGCCTGTGTATGTCCCCCAGGGCACAGT
GGTGCACCTTGCAGGATTGGAATCCAGGAGCCCTTTACTGTGATGCCGACCACTCCAGTAGCGTATAACTCGC
TGGGTGCAGTGATTGGCATTGCAGTGCTGGGGTCCCTTGTGGTAGCCCTGGTGGCACTGTTCATTGGCTATCG
GCACTGGCAAAAAGGCAAGGAGCACCACCACCTGGCTGTGGCTTACAGCAGCGGGCGCCTGGACGGCTCCGAG
TATGTCATGCCAGATGTCCCTCCGAGCTACAGTCACTACTACTCCAACCCCAGCTACCACACCCTGTCGCAGT
GCTCCCCAAACCCCCCACCCCCTAACAAGGTTCCAGGCCCGCTCTTTGCCAGCCTGCAGAACCCTGAGCGGCC
AGGTGGGGCCCAAGGGCATGATAACCACACCACCCTGCCTGCTGACTGGAAGCACCGCCGGGAGCCCCCTCCA
GGGCCTCTGGACAGGGGGAGCAGCCACCTGGACCGAAGCTACAGCTATAGCTACAGCAATGGCCCAGGCCCAT
TCTACGATAAAGGGCTCATCTCTGAAGAGGAGCTCGGGGCCAGTGTGACTTCCCTGAGCAGTGAGAACCCATA
TGCCACCATCCGGGACCTGCCCAGCTTGCCAGGGGGCCCCCGGGAGAGCAGCTACATGGAGATGAAAGGCCCT
CCCTCAGGATCTCCCCCCAGGCAGCCTCCTCAGTTCTGGGACAGCCAGAGGCGGCGGCAACCCCAGCCACAGA
GAGACAGTGGCACCTACGAGCAGCCCAGCCCCCTGATCCATGACCGAGACTCTGTGGGCTCCCAGCCCCCTCT
GCCTCCGGGCCTACCCCCCGGCCACTATGACTCACCCAAGAACAGCCACATCCCTGGACATTATGACTTGCCT
CCAGTACGGCATCCCCCATCACCTCCACTTCGACGCCAGGACCGTTGA

The NOV1e polypeptide (SEQ ID NO: 10) is 1037 amino acid residues in length and is presented using the one-letter amino acid code in Table 1L. The SignalP, Psort and/or Hydropathy results predict that NOV1 e has a signal peptide and is likely to be localized to the plasma membrane with a certainty of 0.6760. In alternative embodiments, a NOV1e polypeptide is located to the outside of the cell with a certainty of 0.1000, the endoplasmic reticulum (membrane) with a certainty of 0.1000, or the endoplasmic reticulum (lumen) with a certainty of 0.1000. The SignalP predicts a likely cleavage site for a NOV1e peptide between amino acid positions 20 and 21, i.e., at the dash in the sequence AGT-LN.

TABLE 1L
Encoded NOV1e Protein Sequence

(SEQ ID NO:10)

MSPPLCPLLLLAVGLRLAGTLNPSDPNTCSFWESFTTTTKESHSRPFSLLPSEPCERPWEGPHTCPQPTV
VYRTVYRQVVKTDHRQRLQCCHGFYESREFCVPLCAQECVHGRCVAPNQCQCVPGWRGDDCSSECA
PGMWGPQCDKPCSCGNNSSCDPKSGVCSCPSGLQPPNCLQPCTPGYYGPACQFRCQCHGAPCDPQTG
ACFCPAERTGPSCDVSCSQGTSGFFCPSTHPCQNGGVFQTPQGSCSCPPGWMGTICSLPCPEGFHGPNC
SQECRCHNGGLCDRFTGQCRCAPGYTGDRCREECPVGRFGQDCAETCDCAPDARCFPANGACLCEH
GFTGDRCTDRLCPDGFYGLSCQAPCTCDREHSLSCHPMNGECSCLPGWAGLHCNESCPQDTHGPGCQ
EYCLCLHGGVCQATSGLCQCAPGYTGPHCASLCPPDTYGVNCSARCSCENAIACSPIDGECVCKEGW
QRGNCSVPCPPGTWGFSCNASCQCAHEAVCSPQTGACTCTPGWHGAHCQLPCPKGQFGEGCASRCD
CDHSDGCDPVHGRCQCQAGWMGARCHLSCPEGLWGVNCSNTCTCKNGGTCLPENGNCVCAPGFRG
PSCQRSCQPGRYGKRCVPCKCANHSFCHPSNGTCYCLAGWTGPDCSQPCPPGHWGENCAQTCQCHH
GGTCHPQDGSCICPLGWTGHHCLEGCPLGTFGANCSQPCQCGPGEKCHPETGACVCPPGHSGAPCRIG
IQEPFTVMPTTPVAYNSLGAVIGIAVLGSLVVALVALFIGYRHWQKGKEHHHLAVAYSSGRLDGSEYV
MPDVPPSYSHYYSNPSYHTLSQCSPNPPPPNKVPGPLFASLQNPERPGGAQGHDNHTTLPADWKHRRE
PPPGPLDRGSSHLDRSYSYSYSNGPGPFYDKGLISEEELGASVTSLSSENPYATIRDLPSLPGGPRESSYM
EMKGPPSGSPPRQPPQFWDSQRRRQPQPQRDSGTYEQPSPLIHDRDSVGSQPPLPPGLPPGHYDSPKNS
HIPGHYDLPPVRHPPSPPLRRQDR

One or more consensus positions (Cons. Pos.) of the nucleotide sequence have been identified as SNPs as shown in Table 1 M. “Depth” represents the number of clones covering the region of the SNP. The Putative Allele Frequency (Putative Allele Freq.) is the fraction of all the clones containing the SNP. A dash (“—”), when shown, means that a base is not present. The sign “>” means “is changed to”.

TABLE 1M
SNPs of NOV1e

| | | | Putative | |
|---|---|---|---|---|
| Cons. | | | Allele | |
| Pos. | Depth | Change | Freq. | Fragment Listing |
| 2716 | 10 | G > A | 0.200 | 163608053(−, i, 119650936) Fpos: |
| | | | | 482 |
| | | | | 163610839(−, i, 119650936) Fpos: |
| | | | | 485 |
| 2758 | 9 | G > A | 0.333 | 172614573(+, i, −1) Fpos: 132 |
| | | | | 172614575(+, i, −1) Fpos: 14q8 |
| | | | | 172614579(+, i, −1) Fpos: 146 |

The NOV1 amino acid sequence has 834 of 1064 amino acid residues (78%) identical to, and 881 of 1064 amino acid residues (82%) similar to, the 1034 amino acid residue gi|1738605|gb|AAL38571.1 AF444274_{—}1 (AF444274) Jedi protein [Mus musculus] (E=0.0).

NOV1b, NOV1c and NOV1d are expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. NOV1e is expressed in at least the following tissues: adipose, heart, aorta, umbilical vein, pancreas, parathyroid gland, thyroid, stomach, liver, colon, bone marrow, peripheral blood, bone, cartilage, synovium/synovial membrane, brain, thalamus, cervix, placenta, amnion, vulva, testis, lung, kidney, skin, epidermis and dermis. Expression information was derived from the tissue sources of the sequences that were included in the derivation of each of the sequences of NOV1.

NOV1a, NOV1b, NOV1c, NOV1d and NOV1e are very closely homologous as is shown in the amino acid alignment in Table 1N.

TABLE 1N
Amino Acid Alignment of NOV1a, NOV1b, NOV1c, NOV1d and NOV1e

| | |
|---|---|
| | 10 20 30 40 50 |
| | ....\|....\|....\|....\|....\|....\|....\|....\|....\|....\| |
| COR87920446_A | |
| CG57012-01 | |
| CG57012-02 | |
| CG57012-03 | |
| CG57012-04 | |
| | 60 70 80 90 100 |
| | ....\|....\|....\|....\|....\|....\|....\|....\|....\|....\| |
| COR87920446_A | |
| CG57012-01 | |
| CG57012-02 | |
| CG57012-03 | |
| CG57012-04 | E |
| | 110 120 130 140 150 |
| | ....\|....\|....\|....\|....\|....\|....\|....\|....\|....\| |
| COR87920446_A | |
| CG57012-01 | |
| CG57012-02 | |
| CG57012-03 | |
| CG57012-04 | |
| | 160 170 180 190 200 |
| | ....\|....\|....\|....\|....\|....\|....\|....\|....\|....\| |
| COR87920446_A | |
| CG57012-01 | |
| CG57012-02 | |
| CG57012-03 | |
| CG57012-04 | |
| | 210 220 230 240 250 |
| | ....\|....\|....\|....\|....\|....\|....\|....\|....\|....\| |
| COR87920446_A | |
| CG57012-01 | |
| CG57012-02 | |
| CG57012-03 | P |
| CG57012-04 | P |
| | 260 270 280 290 300 |
| | ....\|....\|....\|....\|....\|....\|....\|....\|....\|....\| |
| COR87920446_A | |
| CG57012-01 | |
| CG57012-02 | |
| CG57012-03 | --------------------------- |
| CG57012-04 | --------------------------- |
| | 310 320 330 340 350 |
| | ....\|....\|....\|....\|....\|....\|....\|....\|....\|....\| |
| COR87920446_A | |
| CG57012-01 | |
| CG57012-02 | |
| CG57012-03 | |
| CG57012-04 | |
| | 360 370 380 390 400 |
| | ....\|....\|....\|....\|....\|....\|....\|....\|....\|....\| |
| COR87920446_A | C |
| CG57012-01 | |
| CG57012-02 | |
| CG57012-03 | |
| CG57012-04 | C |
| | 410 420 430 440 450 |
| | ....\|....\|....\|....\|....\|....\|....\|....\|....\|....\| |
| COR87920446_A | |
| CG57012-01 | |
| CG57012-02 | R |
| CG57012-03 | |
| CG57012-04 | Y |
| | 460 470 480 490 500 |
| | ....\|....\|....\|....\|....\|....\|....\|....\|....\|....\| |
| COR87920446_A | |
| CG57012-01 | |
| CG57012-02 | |
| CG57012-03 | |
| CG57012-04 | |
| | 510 520 530 540 550 |
| | ....\|....\|....\|....\|....\|....\|....\|....\|....\|....\| |
| COR87920446_A | |
| CG57012-01 | |
| CG57012-02 | |
| CG57012-03 | |
| CG57012-04 | |
| | 560 570 580 590 600 |
| | ....\|....\|....\|....\|....\|....\|....\|....\|....\|....\| |
| COR87920446_A | |
| CG57012-01 | |
| CG57012-02 | |
| CG57012-03 | |
| CG57012-04 | |
| | 610 620 630 640 650 |
| | ....\|....\|....\|....\|....\|....\|....\|....\|....\|....\| |
| COR87920446_A | |
| CG57012-01 | |
| CG57012-02 | |
| CG57012-03 | |
| CG57012-04 | |
| | 660 670 680 690 700 |
| | ....\|....\|....\|....\|....\|....\|....\|....\|....\|....\| |
| COR87920446_A | R --------------------- |
| CG57012-01 | A |
| CG57012-02 | A |
| CG57012-03 | |
| CG57012-04 | |
| | 710 720 730 740 750 |
| | ....\|....\|....\|....\|....\|....\|....\|....\|....\|....\| |
| COR87920446_A | ---------------------- |
| CG57012-01 | |
| CG57012-02 | |
| CG57012-03 | |
| CG57012-04 | |
| | 760 770 780 790 800 |
| | ....\|....\|....\|....\|....\|....\|....\|....\|....\|....\| |
| COR87920446_A | |
| CG57012-01 | |
| CG57012-02 | |
| CG57012-03 | |
| CG57012-04 | |
| | 810 820 830 840 850 |
| | ....\|....\|....\|....\|....\|....\|....\|....\|....\|....\| |
| COR87920446_A | G |
| CG57012-01 | |
| CG57012-02 | |
| CG57012-03 | |
| CG57012-04 | G |
| | 860 870 880 890 900 |
| | ....\|....\|....\|....\|....\|....\|....\|....\|....\|....\| |
| COR87920446_A | K |
| CG55012-01 | |
| CG57012-02 | |
| CG57012-03 | |
| CG57012-04 | |
| | 910 920 930 940 950 |
| | ....\|....\|....\|....\|....\|....\|....\|....\|....\|....\| |
| COR87920446_A | N G A |
| CG57012-01 | RC EA ------VS A --------------------GCVQ |
| CG57012-02 | RC EA ------VS A --------------------GCVQ |
| CG57012-03 | N W A |
| CG57012-04 | H D G T |
| | 960 970 980 990 1000 |
| | ....\|....\|....\|....\|....\|....\|....\|....\|....\|....\| |
| COR87920446_A | |
| CG57012-01 | RCRV---------------------------------------------- |
| CG57012-02 | RCRV---------------------------------------------- |
| CG57012-03 | |
| CG57012-04 | |
| | 1010 1020 1030 1040 1050 |
| | ....\|....\|....\|....\|....\|....\|....\|....\|....\|....\| |
| COR87920446_A | |
| CG57012-01 | -------------------------------------------------- |
| CG57012-02 | -------------------------------------------------- |
| CG57012-03 | |
| CG57012-04 | |
| | 1060 |
| | ...\|....\|... |
| COR87920446_A | |
| CG57012-01 | ------------- |
| CG57012-02 | ------------- |
| CG57012-03 | |
| CG57012-04 | |

Homologies to any of the above NOV1 proteins will be shared by the other NOV1 proteins insofar as they are homologous to each other as shown above. Any reference to NOV1 is assumed to refer to both of the NOV1 proteins in general, unless otherwise noted.

NOV1 also has homology to the amino acid sequences shown in the BLASTP data listed in Table 1O.

TABLE 1O
BLAST results for NOV1

| Gene Index/ | Protein/ | Length | Identity | Positives | |
|---|---|---|---|---|---|
| Identifier | Organism | (aa) | (%) | (%) | Expect |
| gi\|17386053\|gb\| | Jedi protein | 1034 | 834/1064 | 881/1064 | 0.0 |
| AAI,38571.1\|AF444274_1 | [Mus | | (78%) | (82%) | |
| (AF444274) | musculus] | | | | |
| gi\|17017251 gb\| | MEGF12 [Mus | 1034 | 836/1064 | 882/1064 | 0.0 |
| AAL33583.1 AF440279_1 | musculus] | | (78%) | (82%) | |
| (AF440279) | | | | | |
| gi\|14192943\|ref NP_ | MEGF10 | 1140 | 349/713 | 422/713 | e-163 |
| 115822.1\| | protein [Homo | | (48%) | (58%) | |
| (NM_032446) | sapiens] | | | | |
| gi\|14724016\|ref\|XP_ | MEGF10 | 1140 | 349/713 | 422/713 | e-163 |
| 030163.1\| | protein [Homo | | (48%) | (58%) | |
| (XM_030163) | sapiens] | | | | |
| gi\|14017777\|dbj\| | MEGF10 | 1140 | 349/713 | 422/713 | e-163 |
| BAB47409.1(AB058676) | protein [Homo | | (48%) | (58%) | |
| | sapiens] | | | | |

The homology of these sequences is shown graphically in the ClustalW analysis shown in Table 1P.

TABLE 1P
ClustalW Analysis of NOV1

| | |
|---|---|
| 1) NOV1a | (SEQ ID NO:2) |
| 2) NOV1b | (SEQ ID NO:4) |
| 3) NOV1c | (SEQ ID NO:5) |
| 4) NOV1e | (SEQ ID NO:10) |
| 5) NOV1d | (SEQ ID NO:8) |
| 6) gi\|17386053\|gb\|AAL38571.1\|AF444274 1 (AF444274) Jedi | (SEQ ID NO:31) |
| protein [Mus musculus] | |
| 7) gi\|7017251\|gb\|AAL33588.1\|AF440279 1 (AF440279) MEGF12 | (SEQ ID NO:32) |
| [Mus musculus] | |
| 8) gi\|14192943\|ref\|NP 115822.1\| (NM_032446) MEGF10 | (SEQ ID NO:33) |
| protein [Homo sapiens] | |
| 9) gi\|14192941\|ref NP 115821.1 (NM_032445) MEGF11 | (SEQ ID NO:34) |
| protein [Homo sapiens] | |
| 10) gi\|1616114\|ref\|XP 050906.2\| (XM_050906) MEGF11 | (SEQ ID NO:35) |
| protein [Homo sapiens] | |

| | |
|---|---|
| | 10 20 30 40 50 |
| | ....\|....\|....\|....\|....\|....\|....\|....\|....\|....\| |
| NOV1a COR87920446_A | -----M P V PS T S |
| NOV1b CG57012-01 | -----M P V PS T S |
| NOV1c CG57012-02 | -----M P V PS T S |
| NOV1e CG57012-04 | -----M P V PS T S |
| NOV1d CG57012-03 | -----M P V PS T S |
| gi\|17386053\| | -------M L T SN V T L |
| gi\|17017251\| | -------M L T SN V T L |
| gi\|14192943\| | MVISLN CLSFIC CHWIGT SP LE V H YSV VQ YPH |
| gi\|14192941\| | -------------------------------------------------- |
| gi\|16161114\| | -------------------------------------------------- |
| | 60 70 80 90 100 |
| | ....\|....\|....\|....\|....\|....\|....\|....\|....\|....\| |
| NOV1a COR87920446_A | S P E G P H Q H |
| NOV1b CG57012-01 | S P E G P H Q H |
| NOV1c CG57012-02 | S P E G P H Q H |
| NOV1e CG57012-04 | S P E G P H Q H |
| NOV1d CG57012-03 | S P E G P H Q H |
| gi\|17386053\| | A S H D A M S P R Y |
| gi\|17017251\| | A S H D A M S P R Y |
| gi\|14192943\| | DQIYYTS TDILN-WFK TRHR S A HGE MY RKS P |
| gi\|14192941\| | ---------------------MH PSI -SITHDAQ SSTGSS--AP -- |
| gi\|16161114\| | ---------------------MH PSI -SITHDAQ SSTGSS--AP -- |
| | 110 120 130 140 150 |
| | ....\|....\|....\|....\|....\|....\|....\|....\|....\|....\| |
| NOV1a COR87920446_A | F V D |
| NOV1b CG57012-01 | F V D |
| NOV1c CG57012-02 | F V D |
| NOV1e CG57012-04 | EF V D |
| NOV1d CG57012-03 | F V D |
| gi\|17386053\| | A A G |
| gi\|17017251\| | A A G |
| gi\|14192943\| | GEM H DK I T E G TN A DGDH H T |
| gi\|14192941\| | ------TA TE S DT H E G P G DSDH H S |
| gi\|16161114\| | ------TA TE S DT H E G P G DSDH H S |
| | 160 170 180 190 200 |
| | ....\|....\|....\|....\|....\|....\|....\|....\|....\|....\| |
| NOV1a COR87920446_A | P S V S T Y - |
| NOV1b CG57012-01 | P S V S T Y - |
| NOV1c CG57012-02 | P S V S T Y - |
| MOV1e CG57012-04 | P S V S T Y - |
| NOV1d CG57012-03 | P S V S T Y - |
| gi\|17386053\| | F H A F PA H D -Y |
| gi\|17017251\| | F H T F PA H D -Y |
| gi\|14192943\| | SR Q K GAL N IT A H AA FRGWR EDR EQ T ND NQ QN |
| gi\|14192941\| | NR Q Q GAL N IT A V AA FRGWR EEL A TH KG LP R |
| gi\|16161114\| | NR Q Q GAL N IT A V AA FRGWR EEL A TH KG LP R |
| | 210 220 230 240 250 |
| | ....\|....\|....\|....\|....\|....\|....\|....\|....\|....\| |
| NOV1a COR87920446_A | P AE D S S S HS F TP |
| NOV1b CG57012-01 | P AE D S S S HS F TP |
| NOV1c CG57012-02 | P AE D S S S HS F TP |
| NOV1e CG57012-04 | P AE D S S S H F TP |
| NOV1d CG57012-03 | P AE D S S S H F TP |
| gi\|17386053\| | S D PG A N P D R Y P GS |
| gi\|17017251\| | S D PG A N P D R Y P GS |
| gi\|14192943\| | T HV E R PGY AF EDL PP KH PQ EQRC CHHV |
| gi\|14192941\| | S RA E L APGY VY EEL PP SH AH ELRC TCHHI |
| gi\|16161114\| | S RA E L APGY VY EEL PP SH AH ELRC TCHHI |
| | 260 270 280 290 300 |
| | ....\|....\|....\|....\|....\|....\|....\|....\|....\|....\| |
| NOV1a COR87920446_A | VWRVGPVGMGCGSGENSVGGAKQGSK |
| NOV1b CG57012-01 | VWRVGPVGMGCGSGENSVGGAKQGSK |
| NOV1c CG57012-02 | VWRVGPVGMGCGSGENSVGGAKQGSK |
| NOV1e CG57012-04 | -------------------------- |
| NOV1d CG57012-03 | -------------------------- |
| gi\|17386053\| | -------------------------- V |
| gi\|17017251\| | -------------------------- V |
| gi\|14192943\| | T E S -------------------------- V GQ RF |
| gi\|14192941\| | T E A T-------------------------- AV AQ P TF |
| gi\|16161114\| | T E A T-------------------------- AV AQ P TF |
| | 310 320 330 340 350 |
| | ....\|....\|....\|....\|....\|....\|....\|....\|....\|....\| |
| NOV1a COR87920446_A | R R |
| NOV1b CG57012-01 | R R |
| NOV1c CG57012-02 | R R |
| NOV1e CG57012-04 | R R |
| NOV1d CG57012-03 | R R |
| gi\|17386053\| | T H I Q |
| gi\|17017251\| | T H I Q |
| gi\|14192943\| | K Q T AA H S E QD TY VL |
| gi\|14192941\| | Q D P H Q HV H TA M Q F S FQ SQR |
| gi\|16161114\| | Q D P H Q HV H TA M Q F S FQ SQH |
| | 360 370 380 390 400 |
| | ....\|....\|....\|....\|....\|....\|....\|....\|....\|....\| |
| NOV1a COR87920446_A | D D F A R |
| NOV1b CG57012-01 | D D F A R R |
| NOV1c CG57012-02 | D D F A R R |
| MOV1e CG57012-04 | D D F A R |
| NOV1d CG57012-03 | D D F A R R |
| gi\|17386053\| | G E R E P |
| gi\|17017251\| | G E R E P |
| gi\|14192943\| | Q VNGGK YHVS A A E EA E L IK DKR P HL |
| gi\|14192941\| | HNGGQ S TT E P YK P QE E LH PG TL P AD |
| gi\|16161114\| | HNGGQ S TT E P YK P QE E LH PG TL P AD |
| | 410 420 430 440 450 |
| | ....\|....\|....\|....\|....\|....\|....\|....\|....\|....\| |
| NOV1a COR87920446_A | N L H V Q |
| NOV1b CG57012-01 | N L H V Q |
| NOV1c CG57012-02 | N L R V Q |
| NOV1e CG57012-04 | N L Y V Q |
| NOV1d CG57012-03 | N L H V Q |
| gi\|17386053\| | H Q H L L |
| gi\|17017251\| | H Q H L L |
| gi\|14192943\| | NTH S A K S Y T SPGFY EA QI S QN AD DS |
| gi\|14192941\| | NTI VT A T Q S H VGYY D LP T QN AD HS |
| gi\|16161114\| | NTI VT A T Q S H VGYY D LP T QN AD HS |
| | 460 470 480 490 500 |
| | ....\|....\|....\|....\|....\|....\|....\|....\|....\|....\| |
| NOV1a COR87920446_A | T Q S V A E V |
| NOV1b CG57012-01 | T Q S V A E V |
| NOV1c CG57012-02 | T Q S V A E V |
| NOV1e CG57012-04 | T Q S V A E V |
| NOV1d CG57012-03 | T Q S V A E V |
| gi\|17386053\| | D R N I S T I |
| gi\|17017251\| | D R N I S T I |
| gi\|14192943\| | VT K T FK ID STP LG I S G K DAV V S T |
| gi\|14192941\| | IT G T FM EV VS AAG P SI N GGT V S T |
| gi\|16161114\| | IT G T FM EV VS AAG P SI N GGT V S T |
| | 510 520 530 540 550 |
| | ....\|....\|....\|....\|....\|....\|....\|....\|....\|....\| |
| NOV1a COR87920446_A | P S E |
| NOV1b CG57012-01 | P S E |
| NOV1c CG57012-02 | P S E |
| NOV1e CG57012-04 | P S E |
| NOV1d CG57012-03 | P S E |
| gi\|17386053\| | L N DG |
| gi\|17017251\| | L N DG |
| gi\|14192943\| | A HGVD IR S G LT LNGGA NTLD T A R E |
| gi\|14192941\| | GLD TL S LN E T NG A ID S S L D |
| gi\|16161114\| | GLD TL S LN E T NG A ID S S L D |
| | 560 570 580 590 600 |
| | ....\|....\|....\|....\|....\|....\|....\|....\|....\|....\| |
| NOV1a COR87920446_A | R Q A S |
| NOV1b CG57012-01 | R Q A S |
| NOV1c CG57012-02 | R Q A S |
| NOV1e CG57012-04 | R Q A S |
| NOV1d CG57012-03 | R Q A S |
| gi\|17386053\| | V Q R T P |
| gi\|17017251\| | V Q R T P |
| gi\|14192943\| | K E QD TY LN E S A H TT H R LP S VH DSV A |
| gi\|14192941\| | T E D T LN SEH S A T H C L T I DST |
| gi\|16161114\| | T E D T LN SEH S A T H C L T I DST |
| | 610 620 630 640 650 |
| | ....\|....\|....\|....\|....\|....\|....\|....\|....\|....\| |
| NOV1a COR87920446_A | L V L S Q |
| NOV1b CG57012-01 | L V L S Q |
| NOV1c CG57012-02 | L V L S Q |
| NOV1e CG57012-04 | L V L S Q |
| NOV1d CG57012-03 | L V L S Q |
| gi\|17386053\| | F A VS P P |
| gi\|17017251\| | F A VS P P |
| gi\|14192943\| | R P LP Y AS S DD I E TT I S F H |
| gi\|14192941\| | P R P VS S E S S D S E L I P F HG |
| gi\|16161114\| | P R P VS S E S S D S E L I P F HG |
| | 660 670 680 690 700 |
| | ....\|....\|....\|....\|....\|....\|....\|....\|....\|....\| |
| NOV1a COR87920446_A | P-- A - F N T Y R ------------ |
| NOV1b CG57012-01 | P-- A - F N A Y P EN T |
| NOV1c CG57012-02 | P-- A - F N A Y P EN T |
| NOV1e CG57012-04 | P-- A - F N T Y P EN T |
| NOV1d CG57012-03 | P-- A - F N T Y P EN T |
| gi\|17386053\| | Q-- N N S D T S EA LK S L |
| gi\|17017251\| | Q-- N N S D T S EA LK S L |
| gi\|14192943\| | SQTCPQ VHSSGP HIT L D P F AL NEV S RF KN GI |
| gi\|14192941\| | AQPCPL VHSSRP HIS I E P FS AL N V AG YF QD L |
| gi\|16161114\| | AQPCPL VHSSRP HIS I E P FS AL N V AG YF QD L |
| | 710 720 730 740 750 |
| | ....\|....\|....\|....\|....\|....\|....\|....\|....\|....\| |
| NOV1a COR87920446_A | -------------------------------L T A P GP K |
| NOV1b CG57012-01 | PL HH L T A P GP K |
| NOV1c CG57012-02 | PL HH L T A P GP K |
| NOV1e CG57012-04 | PL HH L T A P GP K |
| NOV1d CG57012-03 | PL HH L T A P GP K |
| gi\|17386053\| | TP PN PRM V L DL M |
| gi\|17017251\| | TP PN PRM V L DL M |
| gi\|14192943\| | T TNN N I R Q YP I SD SQP PAHW P IHT N HN AF |
| gi\|14192941\| | S ANN S I Q FP I KD SQA P FW PA FHA S HN AS |
| gi\|16161114\| | S ANN S I Q FP I KD SQA P FW PA FHA S HN AS |
| | 760 770 780 790 800 |
| | ....\|....\|....\|....\|....\|....\|....\|....\|....\|....\| |
| NOV1a COR87920446_A | P RI I P V T AY - |
| NOV1b CG57012-01 | P RI I P V T AY - |
| NOV1c CG57012-02 | P RI I P V T AY - |
| NOV1e CG57012-04 | P RI I P V T AY - |
| NOV1d CG57012-03 | P RI I P V T AY - |
| gi\|17386053\| | Q D KM S S I S TH - |
| gi\|17017251\| | D KM S S I S TH - |
| gi\|14192943\| | SAYD E K T WT LY TQRCPLG YGKDCALICQCQN DCDH SGQ |
| gi\|14192941\| | SA D H T WT LF TQRCPAA FGKDCGR CQCQN SCDH SGK |
| gi\|16161114\| | SA D H T WT LF TQRCPAA FGKDCGR CQCQN SCDH SGK |
| | 810 820 830 840 850 |
| | ....\|....\|....\|....\|....\|....\|....\|....\|....\|....\| |
| NOV1a COR87920446_A | S V H -- H S --- E |
| NOV1b CG57012-01 | S V H -- D H S --- E |
| NOV1c CG57012-02 | S V H -- D H S --- E |
| NOV1e CG57012-04 | S V H -- H S --- E |
| NOV1d CG57012-03 | S V H -- D H S --- E |
| gi\|17386053\| | T I Q -- E T --- D |
| gi\|17017251\| | T I Q -- E T --- D |
| gi\|14192943\| | CTCRTGFMGRHCEQKCPSGTY YGCRQICDCLNNSTC HIT TC CS GW |
| gi\|14192941\| | CTCRTGFTGQHCEQ CAPGTF YGCQQ CECMNNSTC HVT TC CS GF |
| gi\|16161114\| | CTCRTGFTGQHCEQ CAPGTF YGCQQ CECMNNSTC HVT TC CS GF |
| | 860 870 880 890 900 |
| | ....\|....\|....\|....\|....\|....\|....\|....\|....\|....\| |
| NOV1a COR87920446_A | P --- ---------------- |
| NOV1b CG57012-01 | P --- ---------------- |
| NOV1c CG57012-02 | P --- ---------------- |
| NOV1e CG57012-04 | P --- ---------------- |
| NOV1d CG57012-03 | P --- ---------------- |
| gi\|17386053\| | S --- S--------------- |
| gi\|17017251\| | S --- S--------------- |
| gi\|14192943\| | KGARCDQAGVIIVGNLNS RT TAL ADSYQI AIAGIIILVLVVLFLL |
| gi\|14192941\| | KGIRCDQA-ALMMEELNPYTKI ALGAERHSV AVTGIMLLLFFIVVLL |
| gi\|16161114\| | KGIRCDQA-ALMMEELNPYTKI ALGAERHSV AVTGIMLLLFLIVVLL |
| | 910 920 930 940 950 |
| | ....\|....\|....\|....\|....\|....\|....\|....\|....\|....\| |
| NOV1a COR87920446_A | P L K G Q HD PPG------------- |
| NOV1b CG57012-01 | P L N G Q HD PPG------------- |
| NOV1c CG57012-02 | P L N G Q HD PPG------------- |
| NOV1e CG57012-04 | P L N G Q HD PPG------------- |
| NOV1d CG57012-03 | P L N G Q HD PPG------------- |
| gi\|17386053\| | Q V S A SR H RE V ---------------- |
| gi\|17017251\| | Q V S A SR H RE V ---------------- |
| gi\|14192943\| | A IIYRHKQKGK-ESSMPAV YT MRVVNADYTISGTLPHSNGGNANS |
| gi\|14192941\| | G WHRRRQKEK RDLAPRVSYT MRMTSTDYSLS------------- |
| gi\|16161114\| | G WHRRRQKEK RDLAPRVSYT MRMTSTDYSLS------------- |
| | 960 970 980 990 1000 |
| | ....\|....\|....\|....\|....\|....\|....\|....\|....\|....\| |
| NOV1a COR87920446_A | -----------------------------------------------PLD |
| NOV1b CG57012-01 | -----------------------------------------------PLD |
| NOV1c CG57012-02 | -----------------------------------------------PLD |
| NOV1e CG57012-04 | -----------------------------------------------PLD |
| NOV1d CG57012-03 | -----------------------------------------------PLD |
| gi\|17386053\| | ------------------------------------------------HE |
| gi\|17017251\| | ------------------------------------------------HE |
| gi\|14192943\| | HYFTNPSYHTLTQCATSPHVNNRDRMTVTKSKNNQLFVNLKNVNPGKRGP |
| gi\|14192941\| | -------------------------------------------------- |
| gi\|16161114\| | -------------------------------------------------- |
| | 1010 1020 1030 1040 1050 |
| | ....\|....\|....\|....\|....\|....\|....\|....\|....\|....\| |
| NOV1a COR87920446_A | SSR----L SY--S SY----------SN P PFYN GLIS EELGA |
| NOV1b CG57012-01 | RCR----EA ------------------VS A --------------- |
| NOV1c CG57012-02 | RCR----EA ------------------VS A --------------- |
| NOV1e CG57012-04 | SSH----L SY--S SY----------SN P PFYD GLIS EELGA |
| NOV1d CG57012-03 | SSR----L SY--S SY----------SN P PFYN GLIS EELWA |
| gi\|17386053\| | ASH----L SYSCS SH----------RN P PFCH GPIS EGLGA |
| gi\|17017251\| | ASH----L SYSCS SH----------RN P PFCH GPIS EGLGA |
| gi\|14192943\| | V DCTGTLPA WKHG-G LNELGAFGLDRSYM KS---L DLGKNSEYNS |
| gi\|14192941\| | - ACG----M RQN-T I------------MDK ---F DYMK SVCSS |
| gi\|16161114\| | - ACG----M RQN-T I------------MDK ---F XXXXXXXXXX |
| | 1060 1070 1080 1090 1100 |
| | ....\|....\|....\|....\|....\|....\|....\|....\|....\|....\| |
| NOV1a COR87920446_A | VA - R L S PGGPR S M G PSGSP RQPPQFWD |
| NOV1b CG57012-01 | ------GCVQ RCRV----------------------------------- |
| NOV1c CG57012-02 | ------GCVQ RCRV----------------------------------- |
| NOV1e CG57012-04 | VT - R L S PGGPR S M G PSGSP RQPPQFWD |
| NOV1d CG57012-03 | VA - R L S PGGPR S M G PSGSP RQPPQFWD |
| gi\|17386053\| | VM - R L S PGEPR G V G PSVSP RQSLHLRD |
| gi\|17017251\| | VM - R L S PGEPR G V G PSVSP RQSLHLRD |
| gi\|14192943\| | NC S K P V IPKSS CG V S ARRDS YAEINNST |
| gi\|14192941\| | TC N K P I TCKLP S V S VHMGS YTDVPSLS |
| gi\|16161114\| | XXXXXXXXXXXXXXXXXXXXXXXXXXXXXXXX S VHMGS YTDVPSLS |
| | 1110 1120 1130 1140 1150 |
| | ....\|....\|....\|....\|....\|....\|....\|....\|....\|....\| |
| NOV1a COR87920446_A | SQR RQPQPQRDSGT QPSPLIHDRDSV SQPP PPGLPPGH S |
| NOV1b CG57012-01 | -------------------------------------------------- |
| NOV1c CG57012-02 | -------------------------------------------------- |
| NOV1e CG57012-04 | SQR RQPQPQRDSGT QPSPLIHDRDSV SQPP PPGLPPGH S |
| NOV1d CG57012-03 | SQR RQPQPQRDSGT QPSPLIHDRDSV SQPP PPGLPPGH S |
| gi\|17386053\| | RQQ -QLQPQRDSGT QPSPLSHNEESL STPP PPGLPPGQ S |
| gi\|17017251\| | RQQ -QLQPQRDSGT QPSPLSHNEESL STPP PPGLPPGH S |
| gi\|14192943\| | SAN ---------NV VEPTVSVVQGVFSNNGR SQ----DP L |
| gi\|14192941\| | TSNK---------NI VEPTVSVVQEGC HNSSYIQ----NA L R |
| gi\|16161114\| | TSNK---------NI VEPTVSVVQEGC HNSSYIQ----NA L R |
| | 1160 1170 1180 |
| | ....\|....\|....\|....\|....\|....\|....\| |
| NOV1a COR87920446_A | P HP P-------- LRRQDR--- |
| NOV1b CG57012-01 | ----------------------------------- |
| NOV1c CG57012-02 | ----------------------------------- |
| NOV1e CG57012-04 | P HP P-------- LRRQDR--- |
| NOV1d CG57012-03 | P HP P-------- LRRQDR--- |
| gi\|17386053\| | P HP P-------- SRRQDR--- |
| gi\|17017251\| | P HP P-------- SRRQDR--- |
| gi\|14192943\| | C L DSS SPKQEDSGGSSSNSSSSSE |
| gi\|14192941\| | L QS AN-------G SQDKQS--- |
| gi\|16161114\| | L QS AN-------G SQDKQS--- |

A sequence of about thirty to forty amino-acid residues long found in the sequence of epidermal growth factor (EGF) has been shown to be present, in a more or less conserved form, in a large number of other, mostly animal proteins. The list of proteins currently known to contain one or more copies of an EGF-like pattern is large and varied. The functional significance of EGF domains in what appear to be unrelated proteins is not yet clear. However, a common feature is that these repeats are found in the extracellular domain of membrane-bound proteins or in proteins known to be secreted (exception: prostaglandin G/H synthase). The EGF domain includes six cysteine residues which have been shown (in EGF) to be involved in disulfide bonds. The main structure is a two-stranded beta-sheet followed by a loop to a C-terminal short two-stranded sheet. Subdomains between the conserved cysteines vary in length.

Ligands of the Delta/Serrate/lag-2 (DSL) family and their receptors, members of the lin-12/Notch family, mediate cell-cell interactions that specify cell fate in invertebrates and vertebrates. In C. elegans, two DSL genes, lag-2 and apx-1, influence different cell fate decisions during development. Molecular interaction between Notch and Serrate, another EGF-homologous transmembrane protein containing a region of striking similarity to Delta, has been shown and the same two EGF repeats of Notch may also constitute a Serrate binding domain.

The Notch signaling pathway is a conserved intercellular signaling mechanism that is essential for proper embryonic development in numerous metazoan organisms. Members of the Notch gene family encode transmembrane receptors that are critical for various cell fate decisions. Multiple ligands that activate Notch and related receptors have been identified, including Serrate and Delta in Drosophila and JAG1 in vertebrates. By searching for human brain expressed sequence tags (ESTs) homologous to Serrate and Delta, (Luo et al. (1997) Molec. Cell. Biol. 17: 6057-6067) identified a cDNA which they called Jagged-2 (JAG2). The predicted 1,238-amino acid JAG2 protein has several recognizable motifs, including a signal peptide, 16 EGF-like repeats, a transmembrane domain, and a short cytoplasmic domain. The amino acid sequence of human JAG2 is 89% identical to that of rat Jag2. Northern blot analysis and in situ hybridization showed expression of Jag2 in various murine tissues. Immunohistochemistry revealed coexpression of Jag2 and Notch1 within murine fetal thymus and other murine fetal tissues. Coculture of fibroblasts expressing human JAG2 with murine C2C12 myoblasts inhibited myogenic differentiation. This effect was simulated by expression of constitutively active Notch1, suggesting that JAG2 engages the Notch1 pathway of signal transduction.

Jiang et al. (1998) (Genes Dev. 12: 1046-1057) examined the in vivo role of the Jag2 gene by making a targeted mutation that removed a domain of the Jagged-2 protein required for receptor interaction. Mice homozygous for this deletion died perinatally because of defects in craniofacial morphogenesis. The mutant homozygotes exhibited cleft palate and fusion of the tongue with the palatal shelves. They also exhibited syndactyly of the fore- and hindlimbs. The apical ectodermal ridge (AER) of the limb buds of the mutant homozygotes was hyperplastic, and Jiang et al. (1998) (Genes Dev. 12: 1046-1057) observed an expanded domain of Fgf8 expression in the AER. In the foot plates of the mutant homozygotes, both Bmp2 and Bmp7 expression and apoptotic interdigital cell death were reduced. Mutant homozygotes also displayed defects in thymic development, exhibiting altered thymic morphology and impaired differentiation of T cells of the gamma/delta lineage. These results demonstrated that Notch signaling mediated by Jag2 plays an essential role during limb, craniofacial, and thymic development in mice.

Lanford et al. (1999) (Nature Genet. 21: 289-292) showed that the genes encoding the receptor protein Notch1 and its ligand, Jag2, are expressed in alternating cell types in the developing sensory epithelium of the mammalian cochlea (the organ of Corti). The sensory epithelium contains 4 rows of mechanosensory hair cells: a single row of inner hair cells and 3 rows of outer hair cells. Each hair cell is separated from the next by an interceding supporting cell, forming an invariant and alternating mosaic that extends the length of the cochlear duct. Previous results had suggested that determination of cell fates in the cochlear mosaic occurs via inhibitory interactions between adjacent progenitor cells. Cells populating the cochlear epithelium appear to constitute a developmental equivalence group in which developing hair cells suppress differentiation in their immediate neighbors through lateral inhibition. Lanford et al. (1999) (Nature Genet. 21: 289-292) also found that genetic deletion of Jag2 results in a significant increase in sensory hair cells, presumably as the result of a decrease in Notch activation. These results provided direct evidence for Notch-mediated lateral inhibition in a mammalian system and supported a role for Notch in the development of the cochlear mosaic.

The protein similarity information, expression pattern, and map location for the NOV1 proteins and nucleic acids disclosed herein suggest that it may have important structural and/or physiological functions characteristic of the family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: cardiovascular disease, Alagille syndrome, neural development defects, other developmental defects and other diseases, disorders and conditions of the like.

NOV2

A disclosed NOV2 nucleic acid (designated as CuraGen Acc. No. COR87940554), which encodes a novel secretin receptor precursor-like protein includes the 1833 nucleotide sequence (SEQ ID NO: 11) shown in Table 2A. An open reading frame for the mature protein was identified beginning with an ATG codon at nucleotides 74-76 and ending with a TGA codon at nucleotides 1745-1747. Putative untranslated regions are underlined in Table 2A, and the start and stop codons are in bold letters.

TABLE 2A
NOV2 Nucleotide Sequence

(SEQ ID NO:11)

AGCGAGTCCGTCTGTCAGGCCGCCTCCTCTCCGGCCGTCTGATTTTCTACCCTTCGGCGCCCTGCTCTTCCTCAT
GTTGGCATCCCCGGCCACGGAGACCACCGTCCTCATGTCCCAGACTGAGGCCGACCTGGCCCTGCGGCCCCCGCC
TCCTCTTGGCACCGCGGGGCAGCCCCGCCTCGGGCCCCCTCCTCGCCGAGCGCGCCGCTTCTCCGGGAAGGCTGA
GCCCCGGCCGCGCTCTTCGAGACCTAGCCGCCGCAGCTCAGTCGATCTGGGACTGCTGAGCTCCTGGTCTCAACC
AGCCTCACTCCTTCCGGAACCCCCGGATCCTCCAGACTCCGCTGGCCCCACGAGGAGCCCACCTTCAAGCTCTAA
AGAACCCCCCGAGGGCACATGGATGGGGGCAGCTCCCGTGAAGGCTGTGGACTCTGCATGTCCTGAGCTTACGGG
ATCTTCAGGGGGCCCGGGGTCCAGGGAGCCGCTAAGGGTCCCTGAAGCTGTGGCCCTAGAGCGGCGGCGGGAGCA
GGAAGAAAAGGAGGACATGGAGACCCAGGCTGTGGCAACGTCCCCCGATGGCCGATACCTCAAGTTTGACATCGA
GATTGGACGTGGCTCCTTCAAGACGGTGTATCGAGGGCTAGACACCGACACCACAGTGGAGGTGGCCTGGTGTGA
GCTGCAGACTCGGAAACTGTCTAGAGCTGAGCGGCAGCGCTTCTCAGAGGAGGTGGAGATGCTCAAGGGGCTGCA
GCACCCCAACATCGTCCGCTTCTATGATTCGTGGAAGTCGGTGCTGAGGGGCCAGGTTTGCATCGTGCTGGTCAC
CGAACTCATGACCTCGGGCACGCTCAAGACGTACCTGAGGCGGTTCCGGGAGATGAAGCCGCGGGTCCTTCAGCG
CTGGAGCCGCCAAATCCTGCGGGGACTTCATTTCCTACACTCCCGGGTTCCTCCCATCCTGCACCGGGATCTCAA
GTGCGACAATGTCTTTATCACGGGACCTACTGGCTCTGTCAAAATCGGGGACCTGGGCCTGGCCACGCTCAAGCG
CGCCTCCTTTGCCAAGAGTGTCATCGGGACCCCGGAATTCATGGCCCCCGAGATGTACGAGGAAAAGTACGATGA
GGCCGTGGACGTGTACGCGTTCGGCATGTGCATGCTGGAGATGGCCACCTCTGAGTACCCGTACTCCGAGTGCCA
GAATGCCGCGCAAATCTACCGCAAGGTCACTTCGGGCAGAAAGCCGAACAGCTTCCACAAGGTGAAGATACCCGA
GGTGAAGGAGATCATTGAAGGCTGCATCCGCACGGATAAGAACGAGAGGTTCACCATCCAGGACCTCCTGGCCCA
CGCCTTCTTCCGCGAGGAGCGCGGTGTGCACGTGGAACTAGCGGAGGAGGACGACGGCGAGAAGCCGGGCCTCAA
GCTCTGGCTGCGCATGGAGGACGCGCGGCGCGGGGGGCGCCCACGGGACAACCAGGCCATCGAGTTCCTGTTCCA
GCTGGGCCGGGACGCGGCCGAGGAGGTGGCACAGGAGATGGTGGCTCTGGGCTTGGTCTGTGAAGCCGATTACCA
GCCAGTGGCCCGTGCAGTACGTGAACGGGTTGCTGCCATCCAGCGAAAGCGTGAGAAGCTGCGTAAAGCAAGGGA
ATTGGAGGCACTCCCACCAGAGCCAGGACCTCCACCAGCAACTGTGCCCATGGACCCCGGTCCACCAACAGATGT
CTATCCACCCCATGAGACCTGAGGAGCAAGAGGCAAGACCAGAACACAGCACCTTCCTTATTACAGACACGCCAA
GCTACTCATCTACCACTTCGGATTGCGAGACTG

The nucleic acid sequence of NOV2 maps to chromosome 17 and has 1025 of 1464 bases (70%) identical to a gb:GENBANK-ID:AB044546|acc:AB044546.1 mRNA from Homo sapiens (Homo sapiens P/OKc1.13 mRNA for mitogen-activated protein kinase kinase kinase, partial cds).

The NOV2 polypeptide (SEQ ID NO: 12) is 557 amino acid residues in length and is presented using the one-letter amino acid code in Table 2B. The SignalP, Psort and/or Hydropathy results predict that NOV2 is likely to be localized in the nucleus with a certainty of 0.6000. In alternative embodiments, a NOV2 polypeptide is located in the mitochondrial matrix space with a certainty of 0.3600 or the lysosome (lumen) with a certainty of 0.1000.

TABLE 2B
Encoded NOV2 Protein Sequence

(SEQ ID NO:12)

MLASPATETTVLMSQTEADLALRPPPPLGTAGQPRLGPPPRRARRFSGKAEPRPRSSRPSRRSSVDLGLLSSWS
QPASLLPEPPDPPDSAGPTRSPPSSSKEPPEGTWMGAAPVKAVDSACPELTGSSGGPGSREPLRVPEAVALERR
REQEEKEDMETQAVATSPDGRYLKFDIEIGRGSFKTVYRGLDTDTTVEVAWCELQTRKLSRAERQRFSEEVEML
KGLQHPNIVRFYDSWKSVLRGQVCIVLVTELMTSGTLKTYLRRFREMKPRVLQRWSRQILRGLHFLHSRVPPIL
HRDLKCDNVFITGPTGSVKIGDLGLATLKRASFAKSVIGTPEFMAPEMYEEKYDEAVDVYAFGMCMLEMATSEY
PYSECQNAAQIYRKVTSGRKPNSFHKVKIPEVKEIIEGCIRTDKNERFTIQDLLAHAFFREERGVHVELAEEDD
GEKPGLKLWLRMEDARRGGRPRDNQAIEFLFQLGRDAAEEVAQEMVALGLVCEADYQPVARAVRERVAAIQRKR
EKLRKARELEALPPEPGPPPATVPMDPGPPTDVYPPHET

The NOV2 amino acid sequence to 521 of 552 amino acid residues (94%) identical to, and 524 of 552 amino acid residues (94%) similar to, the 1243 amino acid residue gi|15212448|gb|AAK91995.1 AF390018_{—}1 (AF390018) protein from Homo sapiens (PUTATIVE PROTEIN KINASE WNK4) (E=0.0).

NOV2 is expressed in at least the following: blood, lymphocyte, breast, tonsil, colon, lymph, stomach, adrenal gland, kidney, testis, lung.

NOV2 also has homology to the amino acid sequences shown in the BLASTP data listed in Table 2C.

TABLE 2C
BLAST results for NOV2

| Gene Index/ | Protein/ | Length | Identity | Positives | |
|---|---|---|---|---|---|
| Identifier | Organism | (aa) | (%) | (%) | Expect |
| gi\|15212448\|gb\| | putative | 1243 | 521/552 | 524/552 | 0.0 |
| AAK91995.1 AF390018_1 | protein | | (94%) | (94%) | |
| (AF390018) | kinase WNK4 | | | | |
| | [Homo | | | | |
| | sapiens] | | | | |
| gi\|15277312\|ref\|NP_ | putative | 1231 | 509/540 | 512/540 | 0.0 |
| 115763.1\| | protein | | (94%) | (94%) | |
| (NM_032387) | kinase WNK4 | | | | |
| | [Homo | | | | |
| | sapiens] | | | | |
| gi\|15131540\|emb\| | serine/threon | 1231 | 509/540 | 512/540 | 0.0 |
| CAC48387.1(AJ316534) | ine protein | | (94%) | (94%) | |
| | kinase [Homo | | | | |
| | sapiens] | | | | |
| gi\|6933864\|gb\| | kinase | 670 | 309/479 | 372/479 | e−159 |
| AAF31483.1(AF061944) | deficient | | (64%) | (77%) | |
| | protein KDP | | | | |
| | [Homo | | | | |
| | sapiens] | | | | |
| gi\|16758634\|ref\|NP_ | protein | 2126 | 304/476 | 363/476 | e−153 |
| 446246.1\| | kinase, | | (63%) | (75%) | |
| (NM_053794) | lysine | | | | |
| | deficient 1 | | | | |
| | [Rattus | | | | |
| | norvegicus] | | | | |
| gi\|8272557\|gb\| | protein | 2126 | 304/476 | 363/476 | e−153 |
| AAF74258.1\|AF227741.1\| | kinase WNK1 | | (63%) | (75%) | |
| (AF227741) | [Rattus | | | | |
| | norvegicus] | | | | |
| gi\|1271160\|ref\|NP_ | protein | 2382 | 309/479 | 372/479 | e−153 |
| 061852.1\| | kinase, | | (64%) | 77%) | |
| (NM_018979) | lysine | | | | |
| | deficient 1; | | | | |
| | kinase | | | | |
| | deficient | | | | |
| gi\|11125348\|emb\| | putative | 2382 | 309/479 | 372/479 | e−153 |
| CAC15069.1(AJ296290) | protein | | (64%) | (77%) | |
| | kinase [Homo | | | | |
| | sapiens] | | | | |

The homology of these sequences is shown graphically in the ClustalW analysis shown in Table 2D.

TABLE 2D
ClustalW Analysis for NOV2

| | |
|---|---|
| 1) NOV2 | (SEQ ID NO:12) |
| 2) gi\|15212448\|gb\|AAK91995.1\|AF390018.1 (AF390018) | (SEQ ID NO:36) |
| putative protein kinase WNK4 [Homo sapiens] | |
| 3) gi\|15277312\|ref\|NP 115763.1\| (NM_032387) putative | (SEQ ID NO:37) |
| protein kinase WNK4 [Homo sapiens] | |
| 4) gi\|6933864\|gb\|AAF31483.1 (AF061944) kinase | (SEQ ID NO:38) |
| deficient protein KDP [Homo sapiens] | |
| 5) gi\|16758634 ref NP 446246.1 (NM_053794) protein | (SEQ ID NO:39) |
| kinase, lysine deficient 1 [Rattus norvegicus] | |
| 6) gi\|12711660\|ref NP 061852.1 (NM_018979) protein | (SEQ ID NO:40) |
| kinase, lysine deficient 1; kinase deficient protein | |
| [Homo sapiens] | |

| | |
|---|---|
| | 10 20 30 40 50 |
| | ....\|....\|....\|....\|....\|....\|....\|....\|....\|....\| |
| NOV2 COR87940554 | MLASPATETTVLM Q EAD A R P LGTA QP-------R PPPR-- |
| gi\|15212448\| | MLASPATETTVLM Q EAD A R P LGTA QP-------R PPPR-- |
| gi\|15277312\| | ------------M Q EAD A R P LGTA QP-------R PPPR-- |
| gi\|6933864\| | ----MSGGAAEKQ S PGS F S A APKN SSSDSSVGEK AAAADA |
| gi\|16758634\| | ----MSDGTAEKQ G PG--F S A VPKN SSSDSSVGEK AAVADS |
| gi\|12711660\| | ----MSGGAAEKQ S PGS F S A APKN SSSDSSVGEK AAAADA |
| | 60 70 80 90 100 |
| | ....\|....\|....\|....\|....\|....\|....\|....\|....\|....\| |
| NOV2 COR87940554 | -------- A FSGKAEP ------- RSS PS SVDLGLLSSWSQ |
| gi\|15212448\| | -------- A FSGKAEP ------- RSS LS SVDLGLLSSWS |
| gi\|15277312\| | -------- A FSGKAEP ------- RSS LS SVDLGLLSSWS |
| gi\|6933864\| | VTGRTEEY R HTMDKDS GAAATTTTTEH FF VICDSNATALE |
| gi\|16758634\| | GIGRTEEY R HTMDKDS GAAATTT TEH FF VICDSNATALE |
| gi\|12711660\| | VTGRTEEY R HTMDKDS GAAATTTTTEH FF VICDSNATALE |
| | 110 120 130 140 150 |
| | ....\|....\|....\|....\|....\|....\|....\|....\|....\|....\| |
| NOV2 COR87940554 | ASL -- E PD --- D G-- T SP------------- SSSKE P G |
| gi\|15212448\| | AS A-- D PD --- D G G A SP------------- SSKE P G |
| gi\|15277312\| | AS A-- D PD --- D G G A SP------------- SSKE P G |
| gi\|6933864\| | GL SL Q SI AAV Q P E H EETVTATATSQVAQQ AAAA G Q |
| gi\|16758634\| | GL SL Q SV AVV Q P E H EETLTATVASQVSQQ SAAAS G Q |
| gi\|12711660\| | GL SL Q SI AAV Q P E H EETVTATATSQVAQQ AAAA G Q |
| | 160 170 180 190 200 |
| | ....\|....\|....\|....\|....\|....\|....\|....\|....\|....\| |
| NOV2 COR87940554 | TWM A -- KAVD-SAC ELTG SG ---- G- EPLR----VPEAVA |
| gi\|15212448\| | TWTEG -- KAAEDSA ELPD A ---- G- EPLR----VPEAVA |
| gi\|15277312\| | TWTEG -- KAAEDSA ELPD A ---- G- EPLR----VPEAVA |
| gi\|6933864\| | AVA P ST PSSTSKD VSQP L SKEE PPA SGSG--GGSAKEPQ |
| gi\|16758634\| | AVV S TAT PSSTSKD VSQP L SKEE PP SGSGSGGASAKEPQ |
| gi\|12711660\| | AVA P ST PSSTSKD VSQP L SKEE PPA SGSG--GGSAKEPQ |
| | 210 220 230 240 250 |
| | ....\|....\|....\|....\|....\|....\|....\|....\|....\|....\| |
| NOV2 COR87940554 | L RRE EEK DM Q AT P Y R D |
| gi\|15212448\| | L RRE EEK DM Q AT P Y R D |
| gi\|15277312\| | L RRE EEK DM Q AT P Y R D |
| gi\|6933864\| | E SQQ DDI EL K GM N F K E |
| gi\|16758634\| | E NQQ DDI EL K GM N F K E |
| gi\|12711660\| | E SQQ DDI EL K GM N F K E |
| | 260 270 280 290 300 |
| | ....\|....\|....\|....\|....\|....\|....\|....\|....\|....\| |
| NOV2 COR87940554 | T SRA S V K VLR Q |
| gi\|15212448\| | T SRA S V K VLR Q |
| gi\|15277312\| | T SRA S V K VLR Q |
| gi\|6933864\| | D TKS K A E TVK K |
| gi\|16758634\| | D TKS K A E TVK K |
| gi\|12711660\| | D TKS K A E TVK K |
| | 310 320 330 340 350 |
| | ....\|....\|....\|....\|....\|....\|....\|....\|....\|....\| |
| NOV2 COR87940554 | V R RE PR QR S R H S V |
| gi\|15212448\| | V R RE PR QR S R H S V |
| gi\|15277312\| | V R RE PR QR S R H S V |
| gi\|6933864\| | K K KV IK RS C K Q T T |
| gi\|16758634\| | K K KV IK RS C K Q T T |
| gi\|12711660\| | K K KV IK RS C K Q T T |
| | 360 370 380 390 400 |
| | ....\|....\|....\|....\|....\|....\|....\|....\|....\|....\| |
| NOV2 COR87940554 | L V |
| gi\|15212448\| | L V |
| gi\|15277312\| | L V |
| gi\|6933864\| | I I |
| gi\|16758634\| | I I |
| gi\|12711660\| | I I |
| | 410 420 430 440 450 |
| | ....\|....\|....\|....\|....\|....\|....\|....\|....\|....\| |
| NOV2 COR87940554 | A K R N |
| gi\|15212448\| | A K R N |
| gi\|15277312\| | A K R N |
| gi\|6933864\| | S R V A |
| gi\|16758634\| | S R V A |
| gi\|12711660\| | S R V A |
| | 460 470 480 490 500 |
| | ....\|....\|....\|....\|....\|....\|....\|....\|....\|....\| |
| NOV2 COR87940554 | H K TD N FT Q A R R H |
| gi\|15212448\| | H K TD N FT Q A R R H |
| gi\|15277312\| | H K TD N FT Q A R R H |
| gi\|6933864\| | D A QN D YS K N Q T R |
| gi\|16758634\| | D A QN D YW K N Q T R |
| gi\|12711660\| | D A QN D YW K N Q T R |
| | 510 520 530 540 550 |
| | ....\|....\|....\|....\|....\|....\|....\|....\|....\|....\| |
| NOV2 COR87940554 | PGL M ARR-G RPR Q L Q G AA E AL |
| gi\|15212448\| | PGL M ARR-G RPR Q L Q G AA E AL |
| gi\|15277312\| | PGL M ARR-G RPR Q L Q G AA E AL |
| gi\|6933864\| | IAI I IKKLK KYK E S D E VP D ES |
| gi\|16758634\| | IAI I IKKLK KYK E S D E VP D ES |
| gi\|12711660\| | IAI I IKKLK KYK E S D E VP D ES |
| | 560 570 580 590 600 |
| | ....\|....\|....\|....\|....\|....\|....\|....\|....\|....\| |
| NOV2 COR87940554 | L A YQPV R VRE AA Q KLRKA - L ALPP PG------- |
| gi\|15212448\| | L A YQPV R VRE AA Q KLRKA - L ALPP PG------- |
| gi\|15277312\| | L A YQPV R VRE AA Q KLRKA - L ALPP PG------- |
| gi\|6933864\| | Y G HKTM K IKD SL K QRQLV E Q KKKQ ESSLKQQVE |
| gi\|16758634\| | Y G HKTM K IKD SL K QRQLV E Q KRKQ ESSFKQQNE |
| gi\|12711660\| | Y G HKTM K IKD SL K QRQLV E Q KKKQ ESSLKQQVE |
| | 610 620 630 640 650 |
| | ....\|....\|....\|....\|....\|....\|....\|....\|....\|....\| |
| NOV2 COR87940554 | ---PPPA -----------V MD GPPTD YP------ --HET------ |
| gi\|15212448\| | ---PPPA -----------V M GPPSVFPP-- PFLFR |
| gi\|15277312\| | ---PPPA -----------V M GPPSVFPP-- PFLFR |
| gi\|6933864\| | Q-SSASQ GIKQLPSASTGI T STTSAS STQV QLQYQ |
| gi\|16758634\| | QQASVSQAGIQPLSVASTGI T TTSAS STQV QLQYQ |
| gi\|12711660\| | Q-SSASQ GIKQLPSASTGI T STTSAS STQV QLQYQ |
| | 660 670 680 690 700 |
| | ....\|....\|....\|....\|....\|....\|....\|....\|....\|....\| |
| NOV2 COR87940554 | -------------------------------------------------- |
| gi\|15212448\| | HA Y STT CET GYLS G LDASDPAL ----------------P |
| gi\|15277312\| | HA Y STT CET GYLS G LDASDPAL ----------------P |
| gi\|6933864\| | QP I -VL GTV SGQG V TESRGG---------------------- |
| gi\|16758634\| | QP I -VL GTV SGQG V TESRVSSQ TVSYGSQHEQAHSIGTA |
| gi\|12711660\| | QP I -VL GTV SGQG V TESRVSSQ TVSYGSQHEQAHSTGTV |
| | 710 720 730 740 750 |
| | ....\|....\|....\|....\|....\|....\|....\|....\|....\|....\| |
| NOV2 COR87940554 | -------------------------------------------------- |
| gi\|15212448\| | GVP SLAES----HLCL AF LSIPRSG G D----------------- |
| gi\|15277312\| | GVP SLAES----HLCL AF LSIPRSG G D----------------- |
| gi\|6933864\| | -------------------------------------------------- |
| gi\|16758634\| | HTV SIQAQSQPHGVYP SM QGQNQGQ S S-LAGVLSSQPVQHPQQQ |
| gi\|12711660\| | HTV TVQAQSQPHGVYP SV QGQSQGQ S SSLTGVSSSQPIQHPQQQ |
| | 760 770 780 790 800 |
| | ....\|....\|....\|....\|....\|....\|....\|....\|....\|....\| |
| NOV2 COR87940554 | -------------------------------------------------- |
| gi\|15212448\| | -------------------------------------------F P --- |
| gi\|15277312\| | -------------------------------------------F P --- |
| gi\|6933864\| | -------------------------------------------------- |
| gi\|16758634\| | -GIQPTVPPQQAVQYSLPQAASSSEQ-TVQPVSQPQ-------V A --- |
| gi\|12711660\| | QGIQQTAPPQQTVQYSLSQTSTSSEATTAQPVSQPQAPQVLPQV A KQL |
| | 810 820 830 840 850 |
| | ....\|....\|....\|....\|....\|....\|....\|....\|....\|....\| |
| NOV2 COR87940554 | -------------------------------------------------- |
| gi\|15212448\| | -------------------------------------------------- |
| gi\|15277312\| | -------------------------------------------------- |
| gi\|6933864\| | -------------------------------------------------- |
| gi\|16758634\| | --TQS--------------------------------------------- |
| gi\|12711660\| | PVSQPVPTIQGEPQIPVATQPSVVPVHSGAHFLPVGQPLPTPLLPQYPVS |
| | 860 870 880 890 900 |
| | ....\|....\|....\|....\|....\|....\|....\|....\|....\|....\| |
| NOV2 COR87940554 | -------------------------------------------------- |
| gi\|15212448\| | -------------------------------------------------- |
| gi\|15277312\| | -------------------------------------------------- |
| gi\|6933864\| | -------------------------------------------------- |
| gi\|16758634\| | -------------------------------------------------- |
| gi\|12711660\| | QIPISTPHVSTAQTGFSSLPITMAAGITQPLLTLASSATTAAIPGVSTVV |
| | 910 920 930 940 950 |
| | ....\|....\|....\|....\|....\|....\|....\|....\|....\|....\| |
| NOV2 COR87940554 | -------------------------------------------------- |
| gi\|15212448\| | -------------------------------------------------- |
| gi\|15277312\| | -------------------------------------------------- |
| gi\|6933864\| | -------------------------------------------------- |
| gi\|16758634\| | -------------------------------------------------- |
| gi\|12711660\| | PSQLPTLLQPVTQLPSQVHPQLLQPAVQSMGIPANLGQAAEVPLSSGDVL |
| | 960 970 980 990 1000 |
| | ....\|....\|....\|....\|....\|....\|....\|....\|....\|....\| |
| NOV2 COR87940554 | -------------------------------------------------- |
| gi\|15212448\| | -------------------------------------------------- |
| gi\|15277312\| | -------------------------------------------------- |
| gi\|6933864\| | -------------------------------------------------- |
| gi\|16758634\| | -------------------------------------------------- |
| gi\|12711660\| | YQGFPPRLPPQYPGDSNIAPSSNVASVCIHSTVLSPPMPTEVLATPGYFP |
| | 1010 1020 1030 1040 1050 |
| | ....\|....\|....\|....\|....\|....\|....\|....\|....\|....\| |
| NOV2 COR87940554 | -------------------------------------------------- |
| gi\|15212448\| | -------------------------------------------------- |
| gi\|15277312\| | -------------------------------------------------- |
| gi\|6933864\| | -------------------------------------------------- |
| gi\|16758634\| | ---------------------------------------------STQGV |
| gi\|12711660\| | TVVQPYVESNLLVPMGGVGGQVQVSQPGGSLAQAPTTSSQQAVLESTQGV |
| | 1060 1070 1080 1090 1100 |
| | ....\|....\|....\|....\|....\|....\|....\|....\|....\|....\| |
| NOV2 COR87940554 | -------------------------------------------------- |
| gi\|15212448\| | -------------------------- YA A L VG GMG----- |
| gi\|15277312\| | -------------------------- YA A L VG GMG----- |
| gi\|6933864\| | -------------------------------------------------- |
| gi\|16758634\| | SQAAPPEQTPITQSQPTQPVPLVSSV AH V M GN NAPSSSGR |
| gi\|12711660\| | SQVAPAEPVAVAQPQATQPTTLASSV AH V M GN NVPSSSGR |
| | 1110 1120 1130 1140 1150 |
| | ....\|....\|....\|....\|....\|....\|....\|....\|....\|....\| |
| NOV2 COR87940554 | -------------------------------------------------- |
| gi\|15212448\| | QMR PPG NL RRP ---------- VTS DQN Q S |
| gi\|15277312\| | QMR PPG NL RRP ---------- VTS DQN Q S |
| gi\|6933864\| | -------------------------------------------------- |
| gi\|16758634\| | HEG TTK HY KSV SRHEKTSRPK ILN NKG E R |
| gi\|12711660\| | HEG TTK HY KSV SRHEKTSRPK ILN NKG E R |
| | 1160 1170 1180 1190 1200 |
| | ....\|....\|....\|....\|....\|....\|....\|....\|....\|....\| |
| NOV2 COR87940554 | -------------------------------------------------- |
| gi\|15212448\| | R S AA Y E PS DG LRRI QRVETL |
| gi\|15277312\| | R S AA Y E PS DG LRRI QRVETL |
| gi\|6933864\| | -------------------------------------------------- |
| gi\|16758634\| | K N TI N D AI ES VAQV EKADEM |
| gi\|12711660\| | K N TI N D AI ES VDQV EKADEM |
| | 1210 1220 1230 1240 1250 |
| | ....\|....\|....\|....\|....\|....\|....\|....\|....\|....\| |
| NOV2 COR87940554 | -------------------------------------------------- |
| gi\|15212448\| | KR TGPMEAAEDT SPQE------------------E APLPAL VPLPD |
| gi\|15277312\| | KR TGPMEAAEDT SPQE------------------E APLPAL VPLPD |
| gi\|6933864\| | -------------------------------------------------- |
| gi\|16758634\| | SE VSVEPEGDQG ESLQGKDDYGFPGSQKLEGEFKQ IAVSSM QQIGV |
| gi\|12711660\| | SE VSVEPEGDQG ESLQGKDDYGFSGSQKLEGEFKQ IPASSM QQIGI |
| | 1260 1270 1280 1290 1300 |
| | ....\|....\|....\|....\|....\|....\|....\|....\|....\|....\| |
| NOV2 COR87940554 | -------------------------------------------------- |
| gi\|15212448\| | SNEEL SST LEH --------------------S-WTAFSTSSSS T |
| gi\|15277312\| | SNEEL SST LEH --------------------S-WTAFSTSSSS T |
| gi\|6933864\| | -------------------------------------------------- |
| gi\|16758634\| | TSSLT VVH AGR FIVSPVPESRLRESKIFTSEIPDPVAASTSQG M |
| gi\|12711660\| | TSSLT VVH AGR FIVSPVPESRLRESKVFPSEITDTVAASTAQS M |
| | 1310 1320 1330 1340 1350 |
| | ....\|....\|....\|....\|....\|....\|....\|....\|....\|....\| |
| NOV2 COR87940554 | -------------------------------------------------- |
| gi\|15212448\| | -------P P-------------- NPFS GTPISP I P-------- |
| gi\|15277312\| | -------P P-------------- NPFS GTPISP I P-------- |
| gi\|6933864\| | -------------------------------------------------- |
| gi\|16758634\| | NLSHSASS LQQAFSELKHGQMTE PNTA PNFNHP T S---PFLTS |
| gi\|12711660\| | NLSHSASS LQQAFSELRRAQMTE PNTA PNFSHT T PVVPPFLSS |
| | 1360 1370 1380 1390 1400 |
| | ....\|....\|....\|....\|....\|....\|....\|....\|....\|....\| |
| NOV2 COR87940554 | -------------------------------------------------- |
| gi\|15212448\| | -------------------------------------------------- |
| gi\|15277312\| | -------------------------------------------------- |
| gi\|6933864\| | -------------------------------------------------- |
| gi\|16758634\| | IAGVQTVAASTPSVSVPITSSPLNDISTSVMQSEGALPTDKGIGGVTTST |
| gi\|12711660\| | IAGVPTTAAAT--APVPATSSPPNDISTSVIQSEVTVPTEEGIAGVATST |
| | 1410 1420 1430 1440 1450 |
| | ....\|....\|....\|....\|....\|....\|....\|....\|....\|....\| |
| NOV2 COR87940554 | -------------------------------------------------- |
| gi\|15212448\| | -------ITSPPCHPS SPF PI ---------QVS NPSPHP SP-- |
| gi\|15277312\| | -------ITSPPCHPS SPF PI ---------QVS NPSPHP SP-- |
| gi\|6933864\| | -------------------------------------------------- |
| gi\|16758634\| | GVVASGGLTTLSVSET TLS AV STAPAVVTVSTT QPVQAF GS-- |
| gi\|12711660\| | GVVTSGGLPIPPVSES VLS VV ITIPAVVSISTT PSLQVP TSEI |
| | 1460 1470 1480 1490 1500 |
| | ....\|....\|....\|....\|....\|....\|....\|....\|....\|....\| |
| NOV2 COR87940554 | -------------------------------------------------- |
| gi\|15212448\| | ------------------------------------------------LP |
| gi\|15277312\| | ------------------------------------------------LP |
| gi\|6933864\| | -------------------------------------------------- |
| gi\|16758634\| | IASSTGSFPSGTFSTTTGTTVSSVAVPNAKPPTVLLQQVAGNTAGVAIVT |
| gi\|12711660\| | VVSSTALYPSVTVSATSASAGGSTATPGPKPPAVVSQQAAGSTTVGATLT |
| | 1510 1520 1530 1540 1550 |
| | ....\|....\|....\|....\|....\|....\|....\|....\|....\|....\| |
| NOV2 COR87940554 | -------------------------------------------------- |
| gi\|15212448\| | FS S PE VPL CPWSSLPT P FSP----T---------C QVT |
| gi\|15277312\| | FS S PE VPL CPWSSLPT P FSP----T---------C QVT |
| gi\|6933864\| | -------------------------------------------------- |
| gi\|16758634\| | SV T TP AMA PSLPLGSS A LAETVVVSAHSLDKASHS TAG |
| gi\|12711660\| | SV T TS STA LSIQLSSS T LAETVVVSAHSLDKTSHS TTG |
| | 1560 1570 1580 1590 1600 |
| | ....\|....\|....\|....\|....\|....\|....\|....\|....\|....\| |
| NOV2 COR87940554 | -------------------------------------------------- |
| gi\|15212448\| | SSPFFP CP T S-------------------------F ST------ A |
| gi\|15277312\| | SSPFFP CP T S-------------------------F ST------ A |
| gi\|6933864\| | -------------------------------------------------- |
| gi\|16758634\| | GLSFCA SS S SGTAVSSSVSQPGIVHPLVISSAIAST VLPQPAVP S |
| gi\|12711660\| | AFSLSA SS S PGAGVSSYISQPGGLHPLVIPSVIAST ILPQAAGP S |
| | 1610 1620 1630 1640 1650 |
| | ....\|....\|....\|....\|....\|....\|....\|....\|....\|....\| |
| NOV2 COR87940554 | -------------------------------------------------- |
| gi\|15212448\| | A SLASAFSLA MT ------ S LS-- S G SQS P-------- |
| gi\|15277312\| | A SLASAFSLA MT ------ S LS-- S G SQS P-------- |
| gi\|6933864\| | -------------------------------------------------- |
| gi\|16758634\| | T PQVPNIPPL QP NVPAVQ T IHSQ Q A PNQ HTHCPEMDA |
| gi\|12711660\| | T PQVPSIPPL QP NVPAVQ T IHSQ Q A PNQ HTHCPEVDS |
| | 1660 1670 1680 1690 1700 |
| | ....\|....\|....\|....\|....\|....\|....\|....\|....\|....\| |
| NOV2 COR87940554 | -------------------------------------------------- |
| gi\|15212448\| | -------------------------------------------------- |
| gi\|15277312\| | -------------------------------------------------- |
| gi\|6933864\| | -------------------------------------------------- |
| gi\|16758634\| | DTQSKAPGIDDIKTLEEKLRSLFSEHSSSGTQHASVSLETPLVVET-VTP |
| gi\|12711660\| | DTQPKAPGIDDIKTLEEKLRSLFSEHSSSGAQHASVSLETSLVIESTVTP |
| | 1710 1720 1730 1740 1750 |
| | ....\|....\|....\|....\|....\|....\|....\|....\|....\|....\| |
| NOV2 COR87940554 | -------------------------------------------------- |
| gi\|15212448\| | --------------------A SP S----L LP PVA GQES --- |
| gi\|15277312\| | --------------------A SP S----L LP PVA GQES --- |
| gi\|6933864\| | -------------------------------------------------- |
| gi\|16758634\| | GIPTTAVAPSKLMTSTTSTCL TN LGTAGM VM VGT QVST GTH |
| gi\|12711660\| | GIPTTAVAPSKLLTSTTSTCL TN LGTVAL VT VVT QVST --- |
| | 1760 1770 1780 1790 1800 |
| | ....\|....\|....\|....\|....\|....\|....\|....\|....\|....\| |
| NOV2 COR87940554 | -------------------------------------------------- |
| gi\|15212448\| | ----SPHTAEVESEAS PPAR------------------------ L - |
| gi\|15277312\| | ----SPHTAEVESEAS PPAR------------------------ L - |
| gi\|6933864\| | -------------------------------------------------- |
| gi\|16758634\| | ASAPASTATGAKPGTT PKPSLTKTVVPPVGTELSAGTVPCEQLP F P |
| gi\|12711660\| | ---VSTTTSGVKPGTA SKPPLTKAPVLPVGTELPAGTLPSEQLP F P |
| | 1810 1820 1830 1840 1850 |
| | ....\|....\|....\|....\|....\|....\|....\|....\|....\|....\| |
| NOV2 COR87940554 | -------------------------------------------------- |
| gi\|15212448\| | --------------EA -----------------L--AP--IS E -K |
| gi\|15277312\| | --------------EA -----------------L--AP--IS E -K |
| gi\|6933864\| | -------------------------------------------------- |
| gi\|16758634\| | SLIQTQQPLEDLDAQL RTLSPETIPVTPAVGPLSTMSSTAVT A SQ |
| gi\|12711660\| | SLTQSQQPLEDLDAQL RTLSPEIITVTSAVGPVSMAAPTAIT A TQ |
| | 1860 1870 1880 1890 1900 |
| | ....\|....\|....\|....\|....\|....\|....\|....\|....\|....\| |
| NOV2 COR87940554 | -------------------------------------------------- |
| gi\|15212448\| | ---------------------LV TSSKEP EPLPLQPTSPTL GS |
| gi\|15277312\| | ---------------------LV TSSKEP EPLPLQPTSPTL GQ |
| gi\|6933864\| | -------------------------------------------------- |
| gi\|16758634\| | KDGTEVH---VTASSSGAGVVKM SVTMDD QKERKNRSEDTK VH |
| gi\|12711660\| | KGVSQVKEGPVLATSSGAGVFKM SVAADG QKEGKNKSEDAK VH |
| | 1910 1920 1930 1940 1950 |
| | ....\|....\|....\|....\|....\|....\|....\|....\|....\|....\| |
| NOV2 COR87940554 | -------------------------------------------------- |
| gi\|15212448\| | PKP PQLTSE DTED AGGG ---- REALAESDRAAEGLGAGV EE |
| gi\|15277312\| | PKP PQLTSE DTED AGGG ---- REALAESDRAAEGLGAGV EE |
| gi\|6933864\| | -------------------------------------------------- |
| gi\|16758634\| | FES SESSVL SSPE TLVK PNGI VSGISLDVPDSTHRTPTP AK |
| gi\|12711660\| | FES SESSVL SSPE TLVK PNGI IPGISSDVPESAHKTTAS AK |
| | 1960 1970 1980 1990 2000 |
| | ....\|....\|....\|....\|....\|....\|....\|....\|....\|....\| |
| NOV2 COR87940554 | -------------------------------------------------- |
| gi\|15212448\| | GDD KEPQ ---------------------------------------- |
| gi\|15277312\| | GDD KEPQ ---------------------------------------- |
| gi\|6933864\| | -------------------------------------------------- |
| gi\|16758634\| | SET QPTK RFQVTTTANKVGRFSVSRTEDKVTELKKEGPVTSP-FRDS |
| gi\|12711660\| | SDT QPTK RFQVTTTANKVGRFSVSKTEDKITDTKKEGPVASPPFMDL |
| | 2010 2020 2030 2040 2050 |
| | ....\|....\|....\|....\|....\|....\|....\|....\|....\|....\| |
| NOV2 COR87940554 | -------------------------------------------------- |
| gi\|15212448\| | -------------------------------------------- QPLS |
| gi\|15277312\| | -------------------------------------------- QPLS |
| gi\|6933864\| | -------------------------------------------------- |
| gi\|16758634\| | EQTVIPAAIPKKEKPELAEPSHLN PSSDLEAAFLSRGGEDGSG HSPP |
| gi\|12711660\| | EQAVLPAVIPKKEKPELSEPSHLN PSSDPEAAFLSRDVDDGSG HSPH |
| | 2060 2070 2080 2090 2100 |
| | ....\|....\|....\|....\|....\|....\|....\|....\|....\|....\| |
| NOV2 COR87940554 | -------------------------------------------------- |
| gi\|15212448\| | HPSPVWMNYSYS LC -------- EES SG EFWA QS Q |
| gi\|15277312\| | HPSPVWMNYSYS LC -------- EES SG EFWA QS Q |
| gi\|6933864\| | -------------------------------------------------- |
| gi\|16758634\| | HLCSKSLPIQTL QS NSFNSSYM SDN DI DLRL RR E |
| gi\|12711660\| | QLSSKSLPSQNL QS NSFNSSYM SDN DI DLKL RR D |
| | 2110 2120 2130 2140 2150 |
| | ....\|....\|....\|....\|....\|....\|....\|....\|....\|....\| |
| NOV2 COR87940554 | -------------------------------------------------- |
| gi\|15212448\| | S VET TL K D SR Q PG VA M S Q LS GSFPT |
| gi\|15277312\| | S VET TL K D SR Q PG VA M S Q LS GSFPT |
| gi\|6933864\| | -------------------------------------------------- |
| gi\|16758634\| | K IQD SR H S TK V AV IP P G R PT SKGSK |
| gi\|12711660\| | K IQD SR H S TK V AV IP P G R PT SKGSK |
| | 2160 2170 2180 2190 2200 |
| | ....\|....\|....\|....\|....\|....\|....\|....\|....\|....\| |
| NOV2 COR87940554 | -------------------------------------------------- |
| gi\|15212448\| | R N ------------------------------------------ R |
| gi\|15277312\| | R N ------------------------------------------ R |
| gi\|6933864\| | -------------------------------------------------- |
| gi\|16758634\| | S S SLGNKSPQLSGNLSGQSGTSVLNPQQTLHPPGNTPETGHNQL P |
| gi\|12711660\| | S S SLGNKSPQLSGNLSGQSAASVLHPQQTLHPPGNIPESGQNQL P |
| | 2210 2220 2230 2240 2250 |
| | ....\|....\|....\|....\|....\|....\|....\|....\|....\|....\| |
| NOV2 COR87940554 | -------------------------------------------------- |
| gi\|15212448\| | SE ------------------------ P IMRR SLSG--S TGS E |
| gi\|15277312\| | SE ------------------------ P IMRR SLSG--S TGS E |
| gi\|6933864\| | -------------------------------------------------- |
| gi\|16758634\| | LK SPSSDNLYSAFTSDGAISIPSLSA Q TSST TVGGTVS QAA A |
| gi\|12711660\| | LK SPSSDNLYSAFTSDGAISVPSLSA Q TSST TVGATVN QAA A |
| | 2260 2270 2280 2290 2300 |
| | ....\|....\|....\|....\|....\|....\|....\|....\|....\|....\| |
| NOV2 COR87940554 | -------------------------------------------------- |
| gi\|15212448\| | ---R-A KGV AG VGRM------------------------------- |
| gi\|15277312\| | ---R-A KGV AG VGRM------------------------------- |
| gi\|6933864\| | -------------------------------------------------- |
| gi\|16758634\| | PPAMTS RKG TD LHKLVDNWARDAMNLSGRRGSKGHMNYEGPGMARK |
| gi\|12711660\| | PPAMTS RKG TD LHKLVDNWARDAMNLSGRRGSKGHMNYEGPGMARK |
| | 2310 2320 2330 2340 2350 |
| | ....\|....\|....\|....\|....\|....\|....\|....\|....\|....\| |
| NOV2 COR87940554 | -------------------------------------------------- |
| gi\|15212448\| | -------------------------------------------------- |
| gi\|15277312\| | -------------------------------------------------- |
| gi\|6933864\| | -------------------------------------------------- |
| gi\|16758634\| | FSAPGQLCISMTSNMGGSTPISAASATSLGHFTKSMCPPQQYGFPAAPFG |
| gi\|12711660\| | FSAPGQLCISMTSNLGGSAPISAASATSLGHFTKSMCPPQQYGFPATPFG |
| | 2360 2370 2380 2390 |
| | ....\|....\|....\|....\|....\|....\|....\|....\|....\|... |
| NOV2 COR87940554 | ------------------------------------------------ |
| gi\|15212448\| | ------------------------------------------------ |
| gi\|15277312\| | ------------------------------------------------ |
| gi\|6933864\| | ------------------------------------------------ |
| gi\|16758634\| | TQWSGTGGPAPQPLGQFQPVGTTSLQNFNISNLQKSISNPPSSNLRTT |
| gi\|12711660\| | AQWSGTGGPAPQPLGQFQPVGTASLQNFNISNLQKSISNPPGSNLRTT |

Tables 2E, 2F and 2G list the domain description from DOMAIN analysis results against NOV2. This indicates that the NOV2 sequence has properties similar to those of other proteins known to contain these domains.

TABLE 2E
Domain Analysis of NOV2

gnl|Smart|smart00220, S_TKc, Serine/Threonine protein
kinases, catalytic domain; Phosphotransferases. Serine or
threonine-specific kinase subfamily.
(SEQ ID NO:41)
CD-Length = 256 residues, 98.0% aligned
Score = 221 bits (562), Expect = 1e−58

| | | | |
|---|---|---|---|
| NOV 2: | 176 | EIGRGSFKTVYRGLDTDTTVEVAWCELQTRKLSRAERQRFSEEVEMLKGLQHPNIVRFYD | 235 |
| | | +\|+\|+\| \|\| \| \| \|\| ++ \|\| + +\|+\| \|+++\|\| \| \|\|\|\|\|+ \|\| | |
| Sbjct: | 6 | VLGKGAFGKVYLARDKKTGKLVAIKVIKKEKLKKKKRERILREIKILKKLDHPNIVKLYD | 65 |
| NOV 2: | 236 | SWKSVLRGQVCIVLVTELMTSGTLKTYLRRFREMKPRVLQRWSRQILRGLHFLHSRVPPI | 295 |
| | | ++ + \|\| \| \| \| \|++ + + ++\|\|\|\| \| +\|\|\|+ \| | |
| Sbjct: | 66 | VFED----DDKLYLVMEYCEGGDLFDLLKKRGRLSEDEARFYARQILSALEYLHSQ--GI | 119 |
| NOV 2: | 296 | LHRDLKCDNVFITGPTGSVKIGDLGLATL--KRASFAKSVIGTPEFMAPEMY-EEKYDEA | 352 |
| | | +\|\|\|\|\| +\|+ + \| \|\|+ \| \|\|\| + + +\|\|\|\|+\|\|\|\|+ + \| +\| | |
| Sbjct: | 120 | IHRDLKPENILLD-SDGHVKLADFGLAKQLDSGGTLLTTFVGTPEYMAPEVLLGKGYGKA | 178 |
| NOV 2: | 353 | VDVYAFGMCMLEMATSEYPYSECQNAAQIYRKVTSGRKPNSFHKVKI-PEVKEIIEGCIR | 411 |
| | | \|\|+++ \|+ + \|+ \| + \|+ +++\|+ \| + \|\| \|\| \|++\|+ + | |
| Sbjct: | 179 | VDIWSLGVILYELLTGKPPFPGDDQLLALFKKIGKPPPPFPPPEWKISPEAKDLIKKLLV | 238 |
| NOV 2: | 412 | TDKNERFTIQDLLAHAFF | 429 |
| | | \| +\| \| ++ \| \| \|\| | |
| Sbjct: | 239 | KDPEKRLTAEEALEHPFF | 256 |

TABLE 2F
Domain Analysis of NOV2

gnl|Pfam|pfam00069, pkinase, Protein kinase domain.
(SEQ ID NO:42)
CD-Length = 256 residues, 98.0% aligned
Score = 197 bits (500) , Expect = 2e−51

| | | | |
|---|---|---|---|
| NOV 2: | 176 | EIGRGSFKTVYRGLDTDTTVEVAWCELQTRKLSRAERQRFSEEVEMLKGLQHPNIVRFYD | 235 |
| | | ++\| \|+\| \|\|+\| \|\| \|\| \|+ \| \|\| +++\|\| \|+++\|+ \| \|\|\|\|\|\| | |
| Sbjct: | 6 | KLGSGAFGKVYKGKHKDTGEIVAIKILKKRSLS-EKKKRFLREIQILRRLSHPNIVRLLG | 64 |
| NOV 2: | 236 | SWKSVLRGQVCIVLVTELMTSGTLKTYLRR-FREMKPRVLQRWSRQILRGLHFLHSRVPP | 294 |
| | | ++ + \|\| \| \| \| \| \|\|\|\| + + ++ + \|\|\|\|\|\| +\|\|\|\| | |
| Sbjct: | 65 | VFEE----DDHLYLVMEYMEGGDLFDYLRRNGLLLSEKEAKKIALQILRGLEYLHSRG-- | 118 |
| NOV 2: | 295 | ILHRDLKCDNVFITGPTGSVKIGDLGLATLKRA---SFAKSVIGTPEFMAPE-MYEEKYD | 350 |
| | | \|+\|\|\|\|\| +\|+ + \|+\|\|\| \| \|\|\| + + +\|\|\|\|+\|\|\|\| + \| | |
| Sbjct: | 119 | IVHRDLKPENILLDEN-GTVKIADFGLARKLESSSYEKLTTFVGTPEYMAPEVLEGRGYS | 177 |
| NOV 2: | 351 | EAVDVYAFGMCMLEMATSEYPYSECQNAAQIYRKVTSGRKPNSFHKVKIPEVKEIIEGCI | 410 |
| | | \|\|\|++ \|+ + \|+ \| + \|+ +++\| \| \|+\|++\|+ \|+ | |
| Sbjct: | 178 | SKVDVWSLGVILYELLTGKLPFPGIDPLEELFRIKERPRLRLPLPPNCSEELKDLIKKCL | 237 |
| NOV 2: | 411 | RTDKNERFTIQDLLAHAFF | 429 |
| | | \| +\| \| +++\| \| +\| | |
| Sbjct: | 238 | NKDPEKRPTAKEILNHPWF | 256 |

TABLE 2G
Domain Analysis of NOV2

gnl|Smart|smart00219, TyrKc, Tyrosine kinase, catalytic domain; Phospho-
transferases. Tyrosine-specific kinase subfamily.
(SEQ ID NO:43)
CD-Length = 258 residues, 98.4% aligned
Score = 143 bits (361) Expect = 2e−35

| | | | |
|---|---|---|---|
| NOV 2: | 171 | LKFDIEIGRGSFKTVYRGL---DTDTTVEVAWCELQTRKLSRAERQRFSEEVEMLKGLQH | 227 |
| | | \| ++\| \|+\| \|\|+\| \|\|\|\| \|+ \| + + \| \| +++ \| \| | |
| Sbjct: | 1 | LTLGKKLGEGAFGEVYKGTLKGKGGVEVEVAVKTLKEDA-SEQQIEEFLREARLMRKLDH | 59 |
| NOV 2: | 228 | PNIVRFYDSWKSVLRGQVCIVLVTELMTSGTLKTYLR--RFREMKPRVLQRWSRQILRGL | 285 |
| | | \|\|\|\|+ \| + +++\| \| \| \| \| \|\|\| \| +\|+ \| ++ \|\| \|\|+ | |
| Sbjct: | 60 | PNIVKLL----GVCTEEEPLMIVMEYMEGGDLLDYLRKNRPKELSLSDLLSFALQIARGM | 115 |
| NOV 2: | 286 | HFLHSRVPPILHRDLKCDNVFITGPTGSVKIGDLGLATLKRASFAKSVIGTPE----FMA | 341 |
| | | +\| \|+ +\|\|\|\| \| + +\|\|\| \| \|\|\| +\| +\|\| | |
| Sbjct: | 116 | EYLESK--NFVHRDLAARNCLVGEN-KTVKIADFGLARDLYDDDYYRKKKSPRLPIRWMA | 172 |
| NOV 2: | 342 | PEMYEE-KYDEAVDVYAFGMCMLEMAT-SEYPYSECQNAAQIYRKVTSG---RKPNSFHK | 396 |
| | | \|\| ++ \|+ \|\|++\|\|+ + \|+ \| \| \|\| \| ++ + \| +\| + | |
| Sbjct: | 173 | PESLKDGKFTSKSDVWSFGVLLWEIFTLGESPYPGMSN-EEVLEYLKKGYRLPQPPNCP- | 230 |
| NOV 2: | 397 | VKIPEVKEIIEGCIRTDKNERFTIQDL | 423 |
| | | \|+ +++ \| \| +\| \| +\| | |
| Sbjct: | 231 | ---DEIYDLMLQCWAEDPEDRPTFSEL | 254 |

The protein similarity information, expression pattern, and map location for the NOV2 protein and nucleic acid disclosed herein suggest that it may have important structural and/or physiological functions characteristic of the family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: Hypercalceimia, Ulcers, Hemophilia, hypercoagulation, idiopathic thrombocytopenic purpura, autoimmume disease, allergies, immunodeficiencies, transplantation, Graft versus host disease (GVHD), Lymphaedema, Systemic lupus erythematosus, Autoimmune disease, Asthma, Emphysema, Scleroderma, allergy, Diabetes, Autoimmune disease, Renal artery stenosis, Interstitial nephritis, Glomerulonephritis, Polycystic kidney disease, Systemic lupus erythematosus, Renal tubular acidosis, IgA nephropathy, Cardiovascular disease, Hypercalceimia, Lesch-Nyhan syndrome, Fertility, Cancer and other diseases, disorders and conditions of the like.

Protein phosphorylation is a fundamental process for the regulation of cellular functions. The coordinated action of both protein kinases and phosphatases controls the levels of phosphorylation and, hence, the activity of specific target proteins. One of the predominant roles of protein phosphorylation is in signal transduction, where extracellular signals are amplified and propagated by a cascade of protein phosphorylation and dephosphorylation events. Eukaryotic protein kinases are enzymes that belong to a very extensive family of proteins which share a conserved catalytic core common with both serine/threonine and tyrosine protein kinases. There are a number of conserved regions in the catalytic domain of protein kinases. In the N-terminal extremity of the catalytic domain there is a glycine-rich stretch of residues in the vicinity of a lysine residue, which has been shown to be involved in ATP binding. In the central part of the catalytic domain there is a conserved aspartic acid residue which is important for the catalytic activity of the enzyme.

NOV3

A disclosed NOV3 nucleic acid (designated as CuraGen Acc. No. COR100339661), which encodes a novel GPCR-like protein and includes the 2646 nucleotide sequence (SEQ ID NO: 13) shown in Table 3A. An open reading frame for the mature protein was identified beginning with an ATG codon at nucleotides 800-802 and ending with a TAA codon at nucleotides 1766-1768. Putative untranslated regions downstream from the termination codon and upstream from the initiation codon are underlined in Table 3A, and the start and stop codons are in bold letters.

TABLE 3A
NOV3 Nucleotide Sequence

(SEQ ID NO:13)

AAACTCACTAAAAATAACAAAAGGACAGAATGTGTCCCGTGGGTCCAAGGCAAAGCATGGTTCGTTTGCTCCAGAT
ATGGTGCAGTGCTTCAGCTGCTCACTGAAGTTCCCTCTGGCAGGAACGGTGCTCTAGATGGGTTTTGTCAATCTAG
ATAAACTTTAATGGTTTACAGTAGATTTCTCTATATTTTGCAGTAGATTATAAAATACATAATGTATATATACAGT
CTATATATTTGTAAAAAAAAATTAAAGATATTTCTAGGTAACACCAGTCTGTCCTTGAATTACCAAATTTTCAAAA
GTCTCTAAAGAAAAACCCAGCAAATTTATTTTCAAATACATCTGTGTGTGAGCCAATCCAAGTGGGCTCACATGGG
TGATGTCCACATTTCCCATCTGCTGTGCTGGGCATGTTCAAATGCTCTGGGTTGATTATGCAGGGCTGGATGCTGG
GCATGTTCAAATGCTCTGGGTTGATTATGCAGGGCTGGATTTTGTGCTCTTTGCCTTTGGACAGGAGCTTGGGATT
GTGGGTCTGGAGAGAATCAAAATCTGGACCACAGCACAGTTCATCTCTTGCTTCATGGAATTAGAGGCAAGACTAG
AGCAAGTGAAGCAGAAACAAAGCATCAATTGCTAGGTTCAAAGACAACCATGTCCTGTTTCTCCGTATGACATCTG
ACTTGCCATATACATGACGCAGTTTGCTTATCTGTCAGAGTTACTACATGGTTGTTGGAACTAAACAAGTAATAAA
TAATTGAAGTTCTGTCCTCTCCCATCACTGTCAGTATTGATGTCTTCCTCAGGTGCAGTAGAGATGGGAGCAACCA
ATGACAGCACCTTCAGCCATTTCATCCTTATAGGCTTCTCTGACCGGCCCGAGCTGGAGAGGGTCCTCTTCGCCAT
CATCCTGCCCGCCTACCTCCTAACCCTGCTGGGCAACAGCATCATCATCCTGGTATCCAGGCTGGACCCGCACCTT
CACACCCCCATGTACTTCTTTCTCACACACCTGTCCTTCCTTGACCTCAGCTTCACCAGTAGCTCCATCCCACAGC
TACTCTATAACCTCAGCGGGCCGGACAAGACCATCAGCTATGTGGGCTGTGCTCTGCAGCTGGTCCTGTTCCTGGG
CCTGGGGGGTGTGGAGTGCCTGCTGCTGGCTGTCATGGCCTATGACCGCTTTGTGGCGGTCTGCAAGCCCCTGCAC
TACATGGTCATCATGAACCCCCAGCTCTGCCGGGGCTTGGTGTCAGTGACCTGGGGCTGTGGGGTGGCCAACTCCT
TGGCCATGTCTCCTGTGACCCTGCGCTTACCCCGCTGTGGGCACCACGAGGTGGACCACTTCCTGCGTGAGATGCC
CGCCCTGATCCGGATGGCCTGCGTCAGCACTGTGGCCATNGAAGGCACCGTCTTTGTCCTGGCGGTGGGTGCTGCC
CTGTCCCCCTTGGTGTTTATCATGATATCTTACAGCTACATTGTGAGGGCTGTGTTACAAATTCGGTCAGCATCAG
GAAGGCAGAAGGCCTTCGGCACCTGCGGCTCCCATCTCACTGTGGTCTCCCTTTTCTATGGAAACATCATCTACAT
GTACATGCAGCCAGGAGCCAGTTCTTCCCAGGACCAGGGCAAGTTCCTCACGCTCTTCTACAACATTGTCACCCCC
CTCCTCAATCCTCTCATCTACACCCTCAGAAACAGAGAGGTGAAGGGGGCACTGGGAAGGTTGCTTCTGGGGAAGA
GAGAGCTAGGAAAGGAGTAAAGGCATCTCCACCTGACTTCACCTCCATCCAGGGCCACTGGCAGCATCTGGAACGG
CTGAATTCCAGCTGATATTAGCCCACGACTCCCAACTTGCCTTTTTCTGGACTTTTGTGAGGCTGTTTCAGTTCTG
ACATTATGTGTTTTTGTTGTTGCTCTTAAAATTGAGACGGGGTCTCACTCTGTCACCTAGGGTGGAGTGCAGTGGT
GCCACCATAGCTCCTTCGACTATTGGGCTTAAGCGATCCTCCCCCACCTCAGCCTTCCAAGTAACTGGGACTACAG
GTGTGCATCACTGGCAGTGGGAATTGTGGCTTTTCTGTCTTCTATGGAGACGGGGTCTTGCTGTGTTGACCAGGCT
GGTCCCCAAACTCCTGGCCTCATGTGATCCTCCTGCCATGGCCTCCTAAAGTTCTGGGATTACAAGTGTGAGTCAC
TGTGACTGGCCAACATTATGTGATTTATGTGTGAACTATATAACACAAATCATCCCCAAAACCCATCATGATCTGT
AAAGCAGCTGCAAAGAATGAAGTGAGAGAAACAGTTGTAAAGATGAGTTTCCACCTACTTATACCAGAGTGCTAAG
AGGAAATAACTCTTCTCAATCAGAGCTTTGCTTTGTTTGTTGTTGTTTGCTTTAAAGTCTAACACACCTGACATGT
TTCAGTCAGAATGACCCCAAATGCATCACTGTTCTCCACGTGGTCCAAGTGCCTCTCTGTTTAGGGCCATCAAATC
ATGGAATGCAGCACAGTTTGATATTTTCTATATTCCCAATTCCTACCCAAACCTTTTCATGAAATCGTAGAGTTTG
TTTTACCCTTTATCTGGTGTAAGATTCTGCATAAACCAAGAAGTGAACCTGTAATATCTATC

The nucleic acid sequence of NOV3 maps to chromosome 1 and 629 of 918 bases (68%) identical to a gb:GENBANK-ID:AF098664|acc:AF098664.1 mRNA from Homo sapiens (Homo sapiens olfactory receptor-like protein (OR2C1) gene, complete cds).

The NOV3 polypeptide (SEQ ID NO: 14) is 322 amino acid residues in length and is presented using the one-letter amino acid code in Table 3B. The SignalP, Psort and/or Hydropathy results predict that NOV3a has a signal peptide and is likely to be localized to the plasma membrane with a certainty of 0.6000. In alternative embodiments, a NOV3a polypeptide is located to the Golgi body with a certainty of 0.4000, the endoplasmic reticulum (membrane) with a certainty of 0.3000, or the microbody (peroxisome) with a certainty of 0.3000. The SignalP predicts a likely cleavage site for a NOV3a peptide between amino acid positions 58 and 59, i.e., at the dash in the sequence VSR-LD.

TABLE 3B
Encoded NOV3 Protein Sequence

(SEQ ID NO:14)

MSSSGAVEMGATNDSTFSHFILIGFSDRPELERVLFAIILPAYLLTLLGNSIIILVSRLDPHLHTPMYFFLTH
LSFLDLSFTSSSIPQLLYNLSGPDKTISYVGCALQLVLFLGLGGVECLLLAVMAYDRFVAVCKPLHYMVIMNP
QLCRGLVSVTWGCGVANSLAMSPVTLRLPRCGHHEVDHFLREMPALIRMACVSTVAXEGTVFVLAVGAALSPL
VFIMISYSYIVRAVLQIRSASGRQKAFGTCGSHLTVVSLFYGNIIYMYMQPGASSSQDQGKFLTLFYNIVTPL
LNPLIYTLRNREVKGALGRLLLGKRELGKE

The NOV3 amino acid sequence has 281 of 314 amino acid residues (89%) identical to, and 295 of 314 amino acid residues (93%) similar to, the 314 amino acid residue gi|17445344|refXP_{—}060558.1|XM_{—}060558 protein from Homo sapiens (Human) (similar to OLFACTORY RECEPTOR) (E=e−149).

NOV3 is expressed in at least the following tissues: liver, spleen. This information was derived by determining the tissue sources of the sequences that were included in the invention including but not limited to SeqCalling sources, Public EST sources, Literature sources, and/or RACE sources.

NOV3 has homology to the amino acid sequences shown in the BLASTP data listed in Table 3C.

TABLE 3C
BLAST results for NOV3

| Gene Index/ | Protein/ | Length | Identity | Positives | |
|---|---|---|---|---|---|
| Identifier | Organism | (aa) | (%) | (%) | Expect |
| gi\|17445344\|ref\|XP_ | similar to | 314 | 281/314 | 295/314 | e−149 |
| 060558.1\| | olfactory | | (89%) | (93%) | |
| (XM_060558) | receptor (H. | | | | |
| | sapiens) | | | | |
| | [Homo sapiens] | | | | |
| gi\|5901478\|gb\| | olfactory | 237 | 196/237 | 216/237 | e−102 |
| AAD55304.1\|AF044033_1 | receptor | | (82%) | (90%) | |
| (AF044033) | [Marmota | | | | |
| | marmota] | | | | |
| gi\|13624329\|ref\|NP_ | olfactory | 320 | 178/305 | 236/305 | 3e−97 |
| 112165.1\| | receptor, | | (58%) | (77%) | |
| (NM_030903) | family 2, | | | | |
| | subfamily W, | | | | |
| | member 1 | | | | |
| gi\|12054431\|emb\| | olfactory | 320 | 178/305 | 236/305 | 4e−97 |
| CAC20523.1(AJ302603) | receptor | | (58%) | (77%) | |
| | [Homo sapiens] | | | | |
| gi\|12054429\|emb\| | olfactory | 320 | 178/305 | 236/305 | 5e−97 |
| CAC20522.1(AJ302602) | receptor | | (58%) | (77%) | |
| | [Homo sapiens] | | | | |

The homology of these sequences is shown graphically in the ClustalW analysis shown in Table 3D.

TABLE 3D
ClustalW for NOV3

1) NOV3 (SEQ ID NO:14)
2) gi 17445344 ref XP 060558.1 (XM_060558) similar to olfactory re-
ceptor (H. sapiens) [Homo sapiens] (SEQ ID NO:44)
3) gi|5901478|gb AAD55304.1 AF044033 1 (AF044033) olfactory receptor
[Marmota marmota] (SEQ ID NO:45)
4) gi|13624329|ref|NP 112165.1| (NM_030903) olfactory receptor, fam-
ily 2, subfamily W, member 1 [Homo sapiens] (SEQ ID NO:46)
5) gi|12054431 emb CAC20523.1| (AJ302603) olfactory receptor [Homo
sapiens] (SEQ ID NO:47)
6) gi|12054429|emb|CAC20522.1| (AJ302602) olfactory receptor [Homo
sapiens] (SEQ ID NO:48)

| | |
|---|---|
| | 10 20 30 40 50 |
| | ....\|....\|....\|....\|....\|....\|....\|....\|....\|....\| |
| NOV3 COR100339661 | MSSSGAVE GAT D TFSH I DR EL RV FAIILPA L L |
| gi\|17445344\| | -------- GT G TQTH DR HL R FV IL A L |
| gi\|5901478\| | -------------------------------------------------- |
| gi\|13624329\| | -------- QS Y SLHG NH KM M SG VA F I |
| gi\|12054431\| | -------- QS Y SLHG NH KM M SG VA F I |
| gi\|12054429\| | -------- QS Y SLHG NH KM M SG VA F I |
| | 60 70 80 90 100 |
| | ....\|....\|....\|....\|....\|....\|....\|....\|....\|....\| |
| NOV3 COR100339661 | SI V R PH TH S S S L Y S |
| gi\|17445344\| | T V R PH AH S S L Y N C |
| gi\|5901478\| | --------------- L G S S L H S R |
| gi\|13624329\| | A A L SQ R C I M V W |
| gi\|12054431\| | A A L SQ R C I V V W |
| gi\|12054429\| | A A L SQ R C I M V W |
| | 110 120 130 140 150 |
| | ....\|....\|....\|....\|....\|....\|....\|....\|....\|....\| |
| NOV3 COR100339661 | AL VLFLG G A V V M I Q R |
| gi\|17445344\| | M FLFLG G A CV M I R R |
| gi\|5901478\| | VV FLFLG G A V V T I SSR |
| gi\|13624329\| | I YVYMW S S T F V H |
| gi\|12054431\| | I YVYMW S T F V H |
| gi\|12054429\| | I YVYMW S S T F V H |
| | 160 170 180 190 200 |
| | ....\|....\|....\|....\|....\|....\|....\|....\|....\|....\| |
| NOV3 COR100339661 | GLVSVT GCGV LAMSPV R R HHEV R M IRM S |
| gi\|17445344\| | GLVSVT GCGV LAMSPV R R HHEV R M IRM S |
| gi\|5901478\| | GLVSVA GCGM L MSPV Q R H KV M IRM N |
| 9i\|13624329\| | KMIIMI SISL V LCTL N T N IL L VKI D |
| gi\|12054431\| | KMIIMI SISL V LCTL N T N IL L VKI D |
| gi\|12054429\| | KMIIMI SISL V LCTL N T N IL L VKI D |
| | 210 220 230 240 250 |
| | ....\|....\|....\|....\|....\|....\|....\|....\|....\|....\| |
| NOV3 COR100339661 | VAX GT V AVGAA S VF M S VR QIR ASGRQ FG |
| gi\|17445344\| | VAI GT V KKGV S VF L S VR QIR ASGRQ FG |
| gi\|5901478\| | VAI GT V AVG S VF V H VR F IQ SSGRHRIF |
| gi\|13624329\| | TTV MS A GII T IL AK TK KASQR M |
| gi\|12054431\| | TTV MS A GII T IL AK TK KASQR M |
| gi\|12054429\| | TTV MS A GII T IL AK TK KASQR M |
| | 260 270 280 290 300 |
| | ....\|....\|....\|....\|....\|....\|....\|....\|....\|....\| |
| NOV3 COR100339661 | L N M ASS Q NIV L |
| gi\|17445344\| | L N M ASS Q M M NIV L |
| gi\|5901478\| | L N M S S Q NIV L F S |
| gi\|13624329\| | M T L N A K TVI S |
| gi\|12054431\| | M T L N A K TVI S |
| gi\|12054429\| | M T L N A K TVI S |
| | 310 320 |
| | ....\|....\|....\|....\|....\|... |
| NOV3 COR100339661 | REV G GR LLGKRELG E------ |
| gi\|17445344\| | REV G GR LLGKRELG E------ |
| gi\|5901478\| | -------------------------- |
| gi\|13624329\| | KDM D KK MRFHHKST IKRNCKS |
| gi\|12054431\| | KDM D KK MRFHHKST IKRNCKS |
| gi\|12054429\| | KNM D KK MRFHHKST IKRNCKS |

Table 3E lists the domain description from DOMAIN analysis results against NOV3. This indicates that the NOV3 sequence has properties similar to those of other proteins known to contain these domains.

TABLE 3E
Domain Analysis of NOV3

gnl|Pfam|pfam00001 7tm_1, 7 transmembrane receptor (rhodopsin family).
(SEQ ID NO:49)
CD-Length = 254 residues, 100.0% aligned
Score = 82.0 bits (201), Expect = 5e−17

| | | | |
|---|---|---|---|
| NOV 3: | 49 | GNSIIILVSRLDPHLHTPMYFFLTHLSFLDLSFTSSSIPQLLYNLSGPDKTISYVGCALQ | 108 |
| | | \|\|++\|\|\| \|\|\| \|\|+\|+ \|\|\| + \| \|\|\|\|\| \|\| | |
| Sbjct: | 1 | GNLLVILVTLRTKKLRTPTNIFLLNLAVADLLFLLTLPPWALYYLVGGDWVFGDALCKLV | 60 |
| NOV 3: | 109 | LVLFLGLGGVECLLLAVMAYDRFVAVCKPLHYMVIMNPQLCRGLVSVTWGCGVANSLAMS | 168 |
| | | \|\|+ \| \|\|\| ++\|\|++\|+ \|\|\| \| \|+ +\|++\| + \|\| | |
| Sbjct: | 61 | GALFVVNGYASILLLTAISIDRYLAIVHPLRYRRIRTPRRAKVLILLVWVLALLLSLPPL | 120 |
| NOV 3: | 169 | -PVTLRLPRCGHHEVDHFLREMPALIRMACVSTVAXEGTVFVLAVGAALSPLVFIMISYS | 227 |
| | | \|\| \|+ \| ++\| \|+ + \|\|\|+\|++\|+ | |
| Sbjct: | 121 | LFSWLRTVEEGNTTVCLIDFPEESVKR----------SYVLLSTLVGFVLPLLVILVCYT | 170 |
| NOV 3: | 228 | YIVRAV---------LQIRSASGRQKAFGTCGSHLTVVSLFYG----NIIYMYMQPGASS | 274 |
| | | \|+\|+ \|+\|\|+\|\|+\| + \| + ++ | |
| Sbjct: | 171 | RILRTLRKRARSQRSLKRRSSSERKAAKMLLVVVVVFVLCWLPYHIVLLLDSLCLLSIWR | 230 |
| NOV 3: | 275 | SQDQGKFLTLFYNIVTPLLNPLIY 298 | |
| | | +\|\|+ \| \|\|\|+\|\| | |
| Sbjct: | 231 | VLPTALLITLWLAYVNSCLNPIIY 254 | |

G-protein-coupled receptors (GPCRs) constitute a vast protein family that encompasses a wide range of functions (including various autocrine, paracrine and endocrine processes). They show considerable diversity at the sequence level, on the basis of which they can be separated into distinct groups. The term “clan” is used to describe the GPCRs, as they embrace a group of families for which there are indications of evolutionary relationship, but between which there is no statistically significant similarity in sequence. The currently known clan members include the rhodopsin-like GPCRs, the secretin-like GPCRs, the cAMP receptors, the fungal mating pheromone receptors, and the metabotropic glutamate receptor family. The metabotropic glutamate receptors are functionally and pharmacologically distinct from the ionotropic glutamate receptors. They are coupled to G-proteins and stimulate the inositol phosphate/Ca2+intracellular signalling pathway. The amino acid sequences of the receptors contain high proportions of hydrophobic residues grouped into seven domains, in a manner reminiscent of the rhodopsins and other receptors believed to interact with G-proteins. However, while a similar 3D framework has been proposed to account for this, there is no significant sequence identity between these and receptors of the rhodopsin-type family: the metabotropic glutamate receptors thus bear their own distinctive ‘7TM’ signature. This 7TM signature is also shared by the calcium-sensing receptors, and GABA (gamma-amino-butyric acid) type B (GABA(B)) receptors.

The protein similarity information, expression pattern, and map location for the NOV3 protein and nucleic acid disclosed herein suggest that it may have important structural and/or physiological functions characteristic of the GPCR family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The NOV3 nucleic acid and protein are useful in potential diagnostic and therapeutic applications implicated in various diseases and disorders described below and/or other pathologies. For example, the compositions of the present invention will have efficacy for treatment of patients suffering from: Cardio-vascular diseases, Von Hippel-Lindau (VHL) syndrome, Cirrhosis,Transplantation, Hemophilia, Hypercoagulation, Idiopathic thrombocytopenic purpura, Immunodeficiencies, Graft versus host disorders and other diseases, disorders and conditions of the like.

NOV4

NOV4 includes two novel ankyrin repeat containing proteins. The disclosed proteins have been named NOV4a and NOV4b.

NOV4a

A disclosed NOV4a nucleic acid (designated as CuraGen Acc. No. COR87934767), encodes a novel ankyrin repeat containing protein and includes the 2381 nucleotide sequence (SEQ ID NO: 15) shown in Table 4A. An open reading frame for the mature protein was identified beginning with an ATG codon at nucleotides 849-851 and ending with a TAA codon at nucleotides 1965-1967. Putative untranslated regions downstream from the termination codon and upstream from the initiation codon are underlined in Table 4A, and the start and stop codons are in bold letters.

TABLE 4A
NOV4a Nucleotide Sequence

(SEQ ID NO:15)

GGGAAAATTGACGGGAGGGAAGAGGGTGGAGAGCAGGACAGAGAGGGCGGTGCAGAAGGGGAATATCCCTCCTGAG
TTCCCTGGAAGAGCGTCAGCCTGGACCCTGGTCTTGGGCTTCTCTGCTGGAATCCTGGGCAGCCCCGGGTGCTGCG
GCGAGGGTCAAGGCCACACAAAGGGCAAGGCAGGCAGACGAGCCAGTCACATGGGGCAGTCGAGCTGCCTGCGTGA
ATGCTAGGCGCGGGACAATGGCAACTCCGGGACAAAGTGCAGGGAGACTCCTGAAGAGATAAGAGGGAAGGGCGAA
GGAAGGGGGCGGGGAGCCAGAGCCTCGGAGCTCCAGGACCGCGCTTTGGGAGACCGTGGCTGGAAGCCGAGCTCGG
CCCGCTGCGGAAGGGGCGCCCTCGCGCCTCTACACTCTAGCCCCGGCTGGGATGCTGAGAACCGCGGCTTCCAGGG
CCGCAGGCGAGCTCCCAGCCAGTCCCCGCGCCCGCCCTTCGGTGCTGGAGGCGGGGCTGCCGAGCTCACCTGGCCG
TTTGGGGTGGGACCGCCCGCGACCCGGGGGAGCTGCAGAGGCGGCGGTACCCAGGGAAGTGGAGCTGGGCTTGCCC
TGGGGACTTGGCTGGAGCTCACACCCCTCCACGCCCCCCAAGGCCTGCGCGGGGGCCCTCCCCTAGCTCCCTCCCT
CCTCCTCCTCCTCCTCCTCCTCCTCTCCTTTGCTCCCTCCCTCCGAACCCAATTGCTCAAGCAGCTTCCTTCCCCA
ACGCCAGCGCCAGTTCCTCTCCCGTTGGGGCCCGGGAAGGGCAGCTAACGCTGGACACTGGGACGGCCGCGCCGGC
AGCTTCAAGACCATGGCCCAGCTCGGAGGGGCCGCGAACCGGGCACCCACGGCCTCTCTCGCGCCGACCTCGCAGA
GCCTGCGGTGCGCCCCGCAGCCCCGCCCCTCGAGAGCGGACACTGGTAGCCTGGGCAGGTACTGGGGCAAAGCCGC
AGCCGCCGCCTCCCGGGAGCACCCCTTCCCAGGCACGCTGATGCACTCTGCAGCGGGCTCAGGGCGCCGGCGGGGA
GCGCTGCGGGAACTGCTGGGGCTGCAGCGGGCGGCTCCTGCGGGGTGGCTGTCGGAGGAGCGCGCCGAGGAGCTGG
GCGGGCCGAGTGGGCCGGGCAGCAGCAGGCTGTGCCTGGAACCGCGGGAGCACGCGTGGATTCTGGCAGCCGCCGA
GGGCCGCTATGAGGTGCTGCGGGAGCTGCTGGAGGCTGAGCCGGAGCTGCTGCTGAGGGGCGACCCGATCACCGGC
TACTCGGTTCTGCACTGGCTGGCCAAGCACGGGCGCCACGAGGAGCTCATTCTGGTACACGACTTCGCCCTACGCC
GGGGGCTGAGGCTCGACGTGAGCGCCCCAGGCAGCGGCGGCCTCACGCCCCTCCACCTGGCGGCCCTTCAGGGCCA
CGACATGGTCATCAAGGTGCTGGTGGGCGCCCTGGGTGCTGACGCTACGCGCCGCGACCACAGCGGCCACCGGGCC
TGCCACTACCTGCGGCCCGACGCGCCTTGGAGGTTGCGGGAGCTGTCGGGAGCCGAGGAATGGGAGATGGAGAGCG
GCAGCGGGTGCACCAACCTGAACAACAACAGCAGCGGCACCACTGCGTGGAGGGCCGCGAGCGCAGTGGGGCGCGA
ACGGCTGTGGAGACAAGCAGGAGAGTGGCAGCGTCGCGGACCAAGGCGAAGGACACCGCGGGCAGCCGGGTGGCGC
AAATGCATAGCCTTTTCCGCCATCTGTTCCCCTCATTCCAGGACCGTTGACAGGGACAGAGACTGGAGAGCTAGGA
GGGGCTGTGACACTGTGGCGATGGCTAGGTCCTGGGTTGTCCCGGGTTCCACCGAAGGAGAGGCGCCTTGGACGCT
GCTTGGGCCTGCAAGGAACAGAACACGTCGGGGTCCGACTCAGGTACTTGTCTCAGGTCTCCTGTAACCACCGGCC
TGGAGGACCCGGGGACTCGGGCACCACNTCACCAAGAGAGAGTGAAGGACCAAGCTGGCCTGGCTCCGAGTTCCAA
AGCTACAGGACTAAGGAGTTGGGAGCAGGGAGCGTGGTCCTGCTTGGGAGAGGGCAAGTTAAGCTTCCAGGGGCCA
TTTCTGGGCAGGCCGACGCGCTGGGTTTATTAGGAAACATTCGCTAGAAGAATGAGTTAAGATTGTAAACCACCAA
TGCAGAGAAAACGCCTAACTCTGCCGGCCTCGCTCGGCCATTAATGGGTCTTGGGGTGCGGGTAGAGTCAGCCTCT
GACAACCTCCTCCTGAGACGACCCAGCCTTACTGGTACTTTTTCTCATGTATCACAGGTTACTTCTTATGTATATT
AAAGTGGAATATGTGTTCTTTTCAC

The nucleic acid sequence of NOV4a maps to chromosome X and has 764 of 1297 bases (58%) identical to a gb:GENBANK-ID:AK025523|acc:AK025523.1 mRNA from Homo sapiens (Homo sapiens cDNA: FLJ21870 fis, clone HEP02445).

The NOV4a polypeptide (SEQ ID NO: 16) is 372 amino acid residues in length and is presented using the one-letter amino acid code in Table 4B. The SignalP, Psort and/or Hydropathy results predict that NOV4a has a signal peptide and is likely to be localized to the microbody (peroxisome) with a certainty of 0.4763. In alternative embodiments, a NOV4a polypeptide is located to the nucleus with a certainty of 0.3000, the lysosome (lumen) with a certainty of 0.2592, or the mitochondrial matrix space with a certainty of 0.1000.

TABLE 4B
Encoded NOV4a Protein Sequence

(SEQ ID NO:16)

MAQLGGAANRAPTASLAPTSQSLRCAPQPRPSRADTGSLGRYWGKAAAAASREHPFPGTLMHSAAGSGRRRGA
LRELLGLQRAAPAGWLSEERAEELGGPSGPGSSRLCLEPREHAWILAAAEGRYEVLRELLEAEPELLLRGDPI
TGYSVLHWLAKHGRHEELILVHDFALRRGLRLDVSAPGSGGLTPLHLAALQGHDMVIKVLVGALGADATRRDH
SGHRACHYLRPDAPWRLRELSGAEEWEMESGSGCTNLNNNSSGTTAWRAASAVGRERLWRQAGEWQRRGPRRR
TPRAAGWRKCIAFSAICSPHSRTVDRDRDWRARRGCDTVAMARSWVVPGSTEGEAPWTLLGPARNRTRRGPTQ
VLVSGLL

The NOV4a amino acid sequence has 273 of 273 amino acid residues (100%) identical to, and 273 of 273 amino acid residues (100%) similar to, the 314 amino acid residue gi|17486018|ref|XP_{—}066736.1|XM_{—}066736 protein (similar to LD31582p, H. sapiens) (E=e−¹²⁵).

NOV4a is predicted to be expressed in the following tissues because of the expression pattern of (GENBANK-ID: gb:GENBANK-ID :AK025523|acc:AK025523.1) a closely related Homo sapiens cDNA: FLJ21870 fis, clone HEP02445 homolog in species Homo sapiens: uterus, lung, kidney, brain and placenta.

NOV4b

A disclosed NOV4b nucleic acid (designated as CuraGen Acc. No. CG57238-01), a variant of NOV4a, includes the 1209 nucleotide sequence (SEQ ID NO: 17) shown in Table 4C.

TABLE 4C
NOV4b Nucleotide Sequence

(SEQ ID NO:17)

AGCTAACGCTGGACACTGGGACGGCCGCGGCGGCAGCTTCAAGACCATGGCCCAGCTCGGAGGGGCCGCG
AACCGGGCACCCACGGCCTCTCTCGCGCCGACCTCGCAGAGCCTGCGGTGCGCCCCGCAGCCCCGCCCCT
CGAGAGCGGACACTGGTAGCCTGGGCAGGTACTGGGGCAAAGCCGCAGCCGCCGCCTCCCGGGAGCACCC
CTTCCCAGGCACGCTGATGCACTCTGCAGCGGGCTCAGGGCGCCGGCGGGGAGCGCTGCGGGAACTGCTG
GGGCTGCAGCGGGCGGCTCCTGCCGGGTGGCTGTCGGAGGAGCGCGCCGAGGAGCTGGGCGGGCCGAGTG
GGCCGGGCAGCAGCAGGCTGTGCCTGGAACCGCGGGAGCACGCGTGGATTCTGGCAGCCGCCGAGGGCCG
CTATGAGGTGCTGCGGGAGCTGCTGGAGGCTGAGCCGGAGCTGCTGCTGAGGGGCGACCCGATCACCGGC
TACTCGGTTCTGCACTGGCTGGCCAAGCACGGGCGCCACGAGGAGCTCATTCTGGTACACGACTTCGCCC
TACGCCGGGGGCTGAGGCTCGACGTGAGCGCCCCAGGCAGCGGCGGCCTCACGCCCCTCCACCTGGCGGC
CCTTCAGGGCCACGACATGGTCATCAAGGTGCTGGTGGGCGCCCTGGGTGCTGACGCTACGCGCCGCGAC
CACAGCGGCCACCGGGCCTGCCACTACCTGCGGCCCGACGCGCCTTGGAGGTTGCGGGAGCTGTCGGGAG
CCGAGGAaTGGGAGATGGAGAGCGGCAGCGGGTGCACCAACCTGAACAACAACAGCAGCGGCACCACTGC
GTGGAGGGCCGCGAGCGCAGTGGGCGCGACGGCTGTGGAGACAAGCAGGAGAGTGGCAGCGTCGCGGACC
AAGGCGAAGGACACCGCGGGCAGCCGGGTGGCGCAAATGCATAGCCTTTTCCGCCATCTGTTCCCCTCAT
TCCAGGACCGTTGACAGGGACAGAGACTGGAGAGCTAGGAGGGGCTGTGACACTGTGGCGATGGCTAGGT
CCTGGGTTGCCCCGGGTTCCACCGAAGGAGAGGCGCCTTGGACGCTGCTTGGGCCTGCAAGGAACAGAAC
ACGTCGGGGTCCGACTCAGGTACTTGTCTCAGGTCTCCTGTAACCACCGGCCTGGAGGACCCGGGGACTC
GGGCACCACTTCACCAAGA

The nucleic acid sequence of NOV4b maps to chromosome X and has has 764 of 1297 bases (58%) identical to a gb:GENBANK-ID:AK025523|acc:AK025523.1 mRNA from Homo sapiens (Homo sapiens cDNA: FLJ21870 fis, clone HEP02445).

The NOV4a polypeptide (SEQ ID NO: 18) is 315 amino acid residues in length and is presented using the one-letter amino acid code in Table 4D. The SignalP, Psort and/or Hydropathy results predict that NOV4b has a signal peptide and is likely to be localized to the microbody (peroxisome) with a certainty of 0.4763. In alternative embodiments, a NOV4b polypeptide is located to the nucleus with a certainty of 0.3000, the lysosome (lumen) with a certainty of 0.2592, or the mitochondrial matrix space with a certainty of 0.1000.

TABLE 4D
Encoded NOV4b Protein Sequence

(SEQ ID NO:18)

MAQLGGAANRAPTASLAPTSQSLRCAPQPRPSRADTGSLGRYWGKAAAAASREHPFPGTLMHSAAGSGRRRGALRE
LLGLQRAAPAGWLSEERAEELGGPSGPGSSRLCLEPREHAWILAAAEGRYEVLRELLEAEPELLLRGDPITGYSVL
HWLAKHGRHEELILVHDFALRRGLRLDVSAPGSGGLTPLHLAALQGHDMVIKVLVGALGADATRRDHSGHRACHYL
RPDAPWRLRELSGAEEWEMESGSGCTNLNNNSSGTTAWRAASAVGATAVETSRRVAASRTKAKDTAGSRVAQMHSL
FRHLFPSFQDR

The NOV4b amino acid sequence has 273 of 273 amino acid residues (100%) identical to, and 273 of 273 amino acid residues (100%) similar to, the 314 amino acid residue gi|17486018|refXP_{—}066736.1|XM_{—}066736 protein (similar to LD31582p, H. sapiens) (E=e−125).

NOV4b is predicted to be expressed in the following tissues because of the expression pattern of (GENBANK-ID: gb:GENBANK-ID:AK025523|acc:AK025523.1) a closely related Homo sapiens cDNA: FLJ21870 fis, clone HEP02445 homolog in species Homo sapiens: uterus, lung, kidney, brain and placenta.

NOV4a and NOV4b are very closely homologous as is shown in the amino acid alignment in Table 4E.

TABLE 4E
Amino Acid Alignment of NOV4a and NOV4b

| | |
|---|---|
| | 10 20 30 40 50 |
| | ....\|....\|....\|....\|....\|....\|....\|....\|....\|....\| |
| COR87934767 | |
| CG57238-01 | |
| | 60 70 80 90 100 |
| | ....\|....\|....\|....\|....\|....\|....\|....\|....\|....\| |
| CQR87934767 | |
| CG57238-01 | |
| | 110 120 130 140 150 |
| | ....\|....\|....\|....\|....\|....\|....\|....\|....\|....\| |
| COR87934767 | |
| CG57238-01 | |
| | 160 170 180 190 200 |
| | ....\|....\|....\|....\|....\|....\|....\|....\|....\|....\| |
| COR87934767 | |
| CG57238-01 | |
| | 210 220 230 240 250 |
| | ....\|....\|....\|....\|....\|....\|....\|....\|....\|....\| |
| COR87934767 | |
| CG57238-01 | |
| | 260 270 280 290 300 |
| | ....\|....\|....\|....\|....\|....\|....\|....\|....\|....\| |
| COR87934767 | RERLWRQ G WQ GPRR PRAAGWR |
| CG57238-01 | -----AT V TS VAAS ------- |
| | 310 320 330 340 350 |
| | ....\|....\|....\|....\|....\|....\|....\|....\|....\|....\| |
| COR87934767 | KCIAFSAICSPHSRTVDRDR WRAR GCDT MAR WVVPGSTEGEAPWT |
| CG57238-01 | -----------------KAK TAGS ---- QMH LFR----------H |
| | 360 370 |
| | ....\|....\|....\|....\|.. |
| COR87934767 | LGPARN TRRGPTQVLVSGLL |
| CG57238-01 | FPSFQD -------------- |

Homologies to any of the above NOV4 proteins will be shared by the other NOV4 proteins insofar as they are homologous to each other as shown above. Any reference to NOV4 is assumed to refer to both of the NOV4 proteins in general, unless otherwise noted.

NOV4a also has homology to the amino acid sequence shown in the BLASTP data listed in Table 4F.

TABLE 4F
BLAST results for NOV4

| Gene Index/ | Protein/ | Length | Identity | Positives | |
|---|---|---|---|---|---|
| Identifier | Organism | (aa) | (%) | (%) | Expect |
| gi\|17486018\|ref\|XP_ | similar to | 315 | 273/273 | 273/273 | e−125 |
| 066736.1\| | LD31582p (H. | | (100%) | (100%) | |
| (XM_066736) | sapiens) | | | | |
| | [Homo sapiens] | | | | |

The homology of these sequences is shown graphically in the ClustalW analysis shown in Table 4G.

TABLE 4G
ClustalW Analysis for NOV4

1)NOV4a (SEQ ID NOV16)
2)NOV4b (SEQ ID NO:18)
3) gi|17486018 ref XP 066736.1| (XM_066736) similar to LD31582p (H.
sapiens) [Homo sapiens] (SEQ ID NO:50)

| | |
|---|---|
| | 10 20 30 40 50 |
| | ....\|....\|....\|....\|....\|....\|....\|....\|....\|....\| |
| NOV4a COR87934767 | |
| NOV4b CG57238-01 | |
| gi\|17486018\| | |
| | 60 70 80 90 100 |
| | ....\|....\|....\|....\|....\|....\|....\|....\|....\|....\| |
| NOV4a COR87934767 | |
| NOV4b CG57238-01 | |
| gi\|17486018\| | |
| | 110 120 130 140 150 |
| | ....\|....\|....\|....\|....\|....\|....\|....\|....\|....\| |
| NOV4a COR87934767 | |
| NOV4b CG57238-01 | |
| gi\|17486018\| | |
| | 160 170 180 190 200 |
| | ....\|....\|....\|....\|....\|....\|....\|....\|....\|....\| |
| NOV4a COR87934767 | |
| NOV4b CG57238-01 | |
| gi\|17486018\| | |
| | 210 220 230 240 250 |
| | ....\|....\|....\|....\|....\|....\|....\|....\|....\|....\| |
| NOV4a COR87934767 | |
| NOV4b CG57238-01 | |
| gi\|17486018\| | |
| | 260 270 280 290 300 |
| | ....\|....\|....\|....\|....\|....\|....\|....\|....\|....\| |
| NOV4a COR87934767 | RERLWRQ G WQ GPRR PRAAGWR |
| NOV4b CG57238-01 | ----- ------- |
| gi\|17486018\| | ----- ------- |
| | 310 320 330 340 350 |
| | ....\|....\|....\|....\|....\|....\|....\|....\|....\|....\| |
| NOV4a COR87934767 | KCIAFSAICSPHSRTVDRDR WRAR GCDT MAR WVVPGSTEGEAPWT |
| NOV4b CG57238-01 | ----------------- ---- ---------- |
| gi\|17486018\| | ----------------- ---- ---------- |
| | 360 370 |
| | ....\|....\|....\|....\| |
| NOV4a COR87934767 | LGPARN TRRCPTQVLVSGLL |
| NOV4b CG57238-01 | -------------- |
| gi\|17486018\| | -------- ----- |

Table 4H lists the domain description from DOMAIN analysis results against NOV4. This indicates that the NOV4 sequence has properties similar to those of other proteins known to contain these domains.

TABLE 4H
Domain Analysis of NOV4

gn1|Pfam|pfam00023, ank, Ank repeat. Ankyrin re-
peats generally consist of a beta, alpha, alpha,
beta order of secondary structures. The repeats
associate to form a higher order structure.
(SEQ ID NO:5l)
CD-Length = 33 residues, 97.0% aligned
Score = 35.4 bits (80), Expect = 0.006

| | | | |
|---|---|---|---|
| NOV4: | 187 | GLTPLHLAALQGHDMVIKVLVGALGADATRRDH | 219 |
| | | \|\|\|\|\|\|\|\| \|\| \|+\|+\|+\|\|\|\| \|\| | |
| Sbjct: | 2 | GNTPLHLAARNGHLEVVKLLLEA-GADVNARDK | 33 |

The protein similarity information, expression pattern, and map location for the NOV4 proteins and nucleic acids disclosed herein suggest that it may have important structural and/or physiological functions characteristic of the family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: Cardio-vascular disorders, Cardiomyopathy, Atherosclerosis, Hypertension, Congenital heart defects, Aortic stenosis, Atrial septal defect (ASD), Atrioventricular (A-V) canal defect, Ductus arteriosus , Pulmonary stenosis , Subaortic stenosis, Ventricular septal defect (VSD), valve diseases, Tuberous sclerosis, Scleroderma, Obesity, Transplantation, Systemic lupus erythematosus, Autoimmune disease, Asthma, Emphysema, Scleroderma, allergy, Diabetes, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome and other diseases, disorders and conditions of the like.Ankyrin repeats are tandemly repeated modules of about 33 amino acids. They occur in a large number of functionally diverse proteins mainly from eukaryotes. The few known examples from prokaryotes and viruses may be the result of horizontal gene transfers. The conserved fold of the ankyrin repeat unit is known from several crystal and solution structures, e.g., from: p53-binding protein 53BP2, cyclin-dependent kinase inhibitor p19Ink4d, transcriptional regulator GABP-beta, and NF-kappaB inhibitory protein IkB-alpha. It has has been described as an L-shaped structure consisting of a beta-hairpin and two alpha-helices. Many ankyrin repeat regions are known to function as protein-protein interaction domains.

NOV5

A disclosed NOV5 nucleic acid (designated as CuraGen Acc. No. COR100396092), encodes a novel ankyrin repeat containing protein and includes the 6272 nucleotide sequence (SEQ ID NO: 19) shown in Table 5A. An open reading frame for the mature protein was identified beginning with an ATG codon at nucleotides 7-9 and ending with a TGA codon at nucleotides 6181-6183. Putative untranslated regions downstream from the termination codon and upstream from the initiation codon are underlined in Table 5A, and the start and stop codons are in bold letters.

TABLE 5A
NOV5 Nucleotide Sequence

(SEQ ID NO:19)

AGGACGATGCCCAAGGGTGGGTGCCCTAAAGCACCACAGCAGGAAGAGCTTCCCCTCAGCAGCGACATGGTGGAGA
AGCAGACTGGGAAAAAGAAAGATAAAGTTTCTCTAACCAAGACCCCAAAACTGGAGCGTGGCGATGGCGGGAAGGA
GGTGAGGGAGCGAGCCAGCAAGCGGAAGCTGCCCTTCACCGCGGGCGCCAATGGGGAGCAGAAGGACTCGGACACA
GGTACCAGCCCGACAGCCTTACCTCTGTGTGACCCCTTCACATACACTGCGGAAGAAGCCAAAGCTGAAAGGCAGA
AGCAGGGCCCTGAGCGGAAGAGGATTAAGAAGGAGCCTGTCACCCGGAAGGCCGGGCTGTCTGGAATCCGAGCCGG
CTACCCCCTCTCCGAGCGCCAGCAGGTGGCCCTTCTCATGCAGATGACGGCCGAGGAGTCTGCCAACAGCCCAGTG
GACACAACACCAAAGCACCCCTCCCAGTCTACAGTGTGTCAGAAGGGAACGCCCAACTCTGCCTCAAAAACCAAAG
ATAAAGTGAACAAGAGAAACGAGCGTGGAGAGACCCGCCTGCACCGAGCCGCCATCCGCGGGGACGCCCGGCGCAT
CAAAGAGCTCATCAGCGAGGGGGCAGACGTCAACGTCAAGGACTTCGCAGGCTGGACGGCGCTGCACGAGGCCTGT
AACCGGGGCTACTACGACGTCGCGAAGCAGCTGCTGGCTGCAGGTGCGGAGGTGAACACCAAGGGCCTAGATGACG
ACACGCCTTTGCACGACGCTGCCAACAACGGGCACCAGGTGGTGAAGCTGCTGCTGCGGTACGGAGGGAACCCGCA
GCAGAGCAACAGGAAAGGCGAGACGCCGCTGAAAGTGGCCAACTCCCCCACGATGGTGAACCTCCTGTTAGGCAAA
GGCACTTACACTTCCAGCGAGGAGAGCAGCTCAGAAGAGGAAGACGCACCATCCTTCGCACCTTCCAGTTCAGTCG
ACGGCAACAACACGGACTCCGAGTTCGAAAAAGGCCTCAAGCACAAGGCCAAGAACCCAGAGCCACAGAAGGCCAC
GGCCCCCGTCAAGGACGAGTATGAGTTTGATGAGGACGACGAGCAGGACAGGGTTCCTCCGGTGGACGACAAGCAC
CTATTGAAAAAGGACTACAGAAAAGAAACGAAATCCAATAGTTTTATCTCTATACCCAAAATGGAGGTTAAAAGTT
ACACTAAAAATAACACGATTGCACCAAAGAAAGCGTCCCATCGTATCCTGTCAGACACGTCGGACGAGGAGGACGC
GAGTGTCACCGTGGGGACAGGAGAGAAGCTGAGACTCTCGGCACATACGATATTGCCTGGTAGTAAGACACGAGAG
CCTTCTAATGCCAAGCAGCAGAAGGAAAAAAATAAAGTGAAAAAGAAGCGAAAGAAAGAAACAAAAGGCAGAGAGG
TTCGCTTCGGAAAGCGGAGCGACAAGTTCTGCTCCTCGGAGTCGGAGAGCGAGTCCTCAGAGAGTGGGGAGGATGA
CAGGGACTCTCTGGGGAGCTCTGGCTGCCTCAAGGGGTCCCCGCTGGTGCTGAAGGACCCCTCCCTGTTCAGCTCC
CTCTCTGCCTCCTCCACCTCGTCTCACGGGAGCTCTGCCGCCCAGAAGCAGAACGACCAGCACACCAAGCACTGGA
AAACCATTTCTTCCCCGGCTTGGTCAGAGGTCAGTTCTTTATCAGACTCCACAAGGACGAGACTGACAAGCGAGTC
TGACTACTCCTCTGAGGGCTCCAGTGTGGAATCGCTGAAGCCAGTGAGGAAGAGGCAGGAGCACAGGAAGCGAGCC
TCCCTGTCGGAGAAGAAGAGCCCCTTCCTGTCCAGCGCGGAGGGCGCTGTCCCCAAACTGGACAAGGAGGGGAAAG
TTGTCAAAAAACATAAAACAAAACACAAACACAAAAACAAGGAGAAGATCAGCCAAGAGCTGAAGTTGAAAAGTTT
TACTTACGAATATGAGGACTCCAAGCAGAAGTCAGATAAGGCTATACTGTTAGAGAATGATCTTTCCACTGAAAAC
AAGCTAAAAGTGTTAAAGCACGATCGCGACCACTTTAAAAAAGAAGAGAAACTTAGCAAAATGAAATTAGAAGAAA
AAGAATGGCTCTTTAAAGATGAAAAATCACTGAAGAGAATCAAAGACAAACTGAGACTGTACAAAGAGGAGAGAGA
CAAAATTTCAAAAGAGAAGGAGAAGATTTTTAAAGAAGATAAAGAAAAACTCAAAAAAGAAAAGGTTTATAGGGAA
GATAGCCTTTCTGACCGGGATTCATCCTTTGATTTCAAAGGGGCCAAGCTCATCTTGGAGACGGTGAAGGAGGACA
GCAAGGAGAGGAGGCGGGACAGCCGGGCCCGGGAGAAGCACCCAGCACGAGAGAAGGAGAAGCCCGATAAGAGGAA
GAGATACAAAGAGAAAGACAAGGACAAAAGTGAGAAATCAATCCTGGAAAAATGTCAGAAGGACAAAGAGAAAAAA
GAAAAACATAAAGACACACATGGCAAAGACAAAGAAAGGAAAGCGTCTGTCTTTGAAAAGCACAAGGAGAAGAAGG
ATAAAGAGTCCACAGAAAAGTACAAGGACAGAGCCTCAGTGGACTCCACGCAAGATAAGAAAAATAAACAGGAGAA
GGCTGAAAAGAAGCACGCTGCCGAAGACAAGGCTAAAAGCAAACACAAAGAGAAGTCGGACAAAGAACATTCCAAG
GAGAGGAAGTCCTCGAGAAGTGCCGACGCGGAATACAGAGAAAGCGAGGTCTCCTCTGACAGCTTCACGGACCGAG
AGGACGACAAGAGCGCCTGCCTCCCTGAGAAGCTGAAAGAGAAGAGGCACAGACACTCCTCATCTTCATCCAAGAA
GAGCCACGACCGAGAGGAGAAGAAAGAGGATTACAAGGAGGGCAGGAAGGGCCAGTACGAAAAGGACCTGGAGGCG
GATGCTTACGGAGTTTCTTACAACATGAAAGCTATTGAATTGTTTGAAAAGAAAGATAAAAATGATGAACCTCTAA
AAGAGAAGAAGAAGAGAGAGAAACACAGGGAGAAATGGAGAGACGAGAAGGAGAGGCACCGGGACAGGCATGCGGA
TAGGCCGAAGCCATCCAAAGACCCAGGCAAGAAAGACGCCAGGCCCAGGGAGAAGCTCCTGGGGGACGGCGACCTG
ATGATGACCAGCTTCGAGAGGATGCTGTCCCAGAAGGACCTGGAGATCGAGGAGCGCCACAAGCGGCACAAGGAGA
GGATGAAGCAAATGGAGAAGCTGAGGCACCGGTCCGGAGACCCCAAGCTCAAGGAGAAGGCGAAGCCGGCAGACGA
CGGGCGGAAGAAGGGTCTGGACATTCCTGCTAAGAAACCGCCGGGGCTGGACCCTCCATTTAAAGACAAAAAGCTC
AAAGAGTCGACTCCTATTCCACCTGCCGCGGAAAATAAGCTACACCCAGCATCAGGTGCAGACTCCAAAGACTGGC
TGGCAGGCCCTCACATGAAAGAGGTCCTGCCTGCGTCCCCCAGGCCTGACCAGAGCCGGCCCACTGGCGTGCCCAC
CCCTACGTCGGTGCTATCCTGCCCCAGCTACGAGGAGGTGATGCACACGCCCAGGACCCCGTCCTGCAGCGCCGAT
GACTACGCGGACCTCGTGTTCGACTGCGCCGACTCGCAGCACTCCACGCCCGTGCCCACCGCTCCCACCAGCGCCT
GCTCCCCCTCCTTTTTCGACAGGTTCTCCGTGGCTTCAAGTGGGCTTTCGGAAAACGCCAGCCAGGCTCCTGCCAG
GCCTCTCTCCACAAACCTTTACCGCTCGGTCTCTGTCGACATTGACAAGCTCTTCAGGCAGCAGAGCGTTCCTGCT
GCCTCCAGCTACGACTCTCCCATGCCACCCTCGATGGAAGACAGGGCGCCCCTGCCCCCGGTTCCCGCGGAGAAGT
TTGCCTGCTTGTCGCCAGGGTACTACTCCCCAGACTATGGCCTCCCGTCGCCCAAAGTCGACGCTTTGCACTGCCC
ACCGGCTGCCGTTGTCACTGTCACCCCGTCTCCAGAGGGCGTCTTCTCAAGTTTACAAGCAAAACCTTCCCCTTCC
CCCCCTTCCCTGGACACCTCCGAGGACCAGCAGGCGACGGCCGCCATCATCCCCCCGGAGCCCAGCTACCTGGAGC
CGCTGGACGAGGGTCCCTTCAGCGCCGTCATCACCGAGCAGCCCGTTGAGTGGGCCCACCCCTCCGAGCAGGCGCT
TGCCTCTAGCCTGATCGGGGGCACCTCTGAAAACCCTGTGAGCTGGCCTGTGGGCTCGGACCTCCTGCTGAAGTCT
CCACAGAGATTCCCCGAGTCCCCAAAGCGTTTCTGCCCCGCGGACCCCCTCCACTCTGCCGCCCCAGGGCCCTTCA
GCGCCTCGGAGGCGCCGTACCCCGCCCCTCCCGCCTCTCCTGCCCCGTACGCTCTGCCCGTCGCTGAGCTGGAGGA
CGTCAAGGACGTCCCCGCCGCCATCTCCACCTCAGAGGCGGCTCCCTACGCCCCTCCCTCCGGGCTGGAGTCCTTC
TTCAGCAACTGCAAGTCACTTCCGGAAGCCCCGCTGGACGTGGCCCCCGAGGCTCTGGGGCCCCTGGAAAATAGCT
TCCTGGACGGCAGCCGCGGCCTGTCTCACCTCGGCCAGGTGGAGCCGGTGCCCTGGGCGGACGCCTTCGCCGGCCC
CGAGGACGACCTGGACCTGGGGCCCTTCTCCCTGCCGGAGCTTCCCCTGCAGACTAAAGATGCCGCAGATGGTGAA
GCGGAACCCGTGGAAGAAAGTCTTGCTCCTCCAGAAGAGATGCCTCCAGGGGCCCCCCGGGAGCTCGAGCCTGAGC
CCTCAGGGGAGCCAAAGCTGGACGTGGCTCTAGAAGCTGCGGTGGAGGCGGAGACGGTGCCGGAAGAGAGGGCCCG
TGGGGATCCGGACTCCAGCGTGGAGCCCGCGCCCGTTCCCCCAGAACAGCTGGGGAGCGGAGACCCCTCCCTCTGT
GCCCCTGACGGCCCCGCCCCGAACACTGTGGCACAAGCTCAGGCCGCAGACGGTGCCGGCCCCGAGGACGACACTG
AGGCCTCCCGTGCCGCCGCCCCAGCCGAAGGCCCTCCTGGCCAGCCGGAAGCCGCAGAACCAAAACCCACGGCCGA
AGCCCCGAAGGCCCCCCGAGAGATCCCTCAGCGCATGACCAGGAACCGGGCGCAGATGCTCGCGAACCAGAGCAAG
CAGGGCCCGCCCCCCTCCGAGAAGGAGTGCGCCCCCACCCCTGCCCCGGTCACCAGGGCCAAGGCCCGCGGCTCCG
AGGACGACGACGCCCAGGCCCAGCATCCGCGCAAACGCCGCTTTCAGCGCTCCACCCAGCAGCTGCAGCTGAACAC
GTCCACGCAGCAGACGCGGGAGGTGATCCAGCAGACGCTGGCCGCCATCGTGGACGCCATCAAGCTGGATGCCATC
GAGCCCTACCACAGCGACAGGGCCAACCCCTACTTCGAATACCTGCAGATCAGGAAGAAGATCGAGGAGAAGCGCA
AGATCCTGTGCTGTATCACGCCGCAGGCGCCCCAGTGCTACGCCGAGTACGTCACCTACACGGGCTCCTACCTCCT
GGACGGCAAGCCCCTCAGCAAGCTCCACATCCCCGTGATCGCACCCCCTCCCTCCCTGGCGGAGCCCCTGAAGGAG
CTGTTCAGCCAGCAGGAGGCCGTCCGGGGAAAGCTGCGTCTACAGCACAGCATCGAGCGGGAGAAGCTGATCGTAT
CCTGTGAGCAGGAGATTCTGCGGGTTCACTGCCGGGCGGCCAGGACCATCGCCAACCAGGCAGTGCCATTCAGCGC
CTGCACGATGCTGCTGGACTCCGAGGTCTACAACATGCCCCTGGAGAGCCAGGGTGACGAGAACAAGTCAGTGCGC
GACCGTTTCAACGCCCGCCAGTTCATCTCCTGCCTCCAGGACGTGGATGACAAGTATGACCGCATGAAGGTCTGCC
TCCTCATGCGGCAGCAGCACGAGGCCGCGGCCCTGAACGCCGTGCAGAGGATGGAGTGGCAGCTGAAGGTGCAGGA
ACTGGACCCCGCCGGGCACAAGTCCCTGTGCGTGAACGAGGTGCCCTCCTTCTACGTGCCCATGGTCGACGTCAAC
GACGACTTTGTATTGTTGCCGGCATGACACCGCGGGACGGCCGCAGGACGCAGGCGAGGGCCGCACGGCTGCCCAG
GACTGCTGCTGAGCCCCAGGGGCGGAGGAGGGAGCGCCCT

The nucleic acid sequence of NOV5 maps to chromosome 16 and has 555 of 857 bases (64% ) identical to gb:GENBANK-ID:AF317425|acc:AF317425.1 mRNA from Homo sapiens (Homo sapiens GAC-1 (GAC-1) mRNA, complete cds).

The NOV5 polypeptide (SEQ ID NO: 20) is 2058 amino acid residues in length and is presented using the one-letter amino acid code in Table 5B. The SignalP, Psort and/or Hydropathy results predict that NOV5a has a signal peptide and is likely to be localized in the nucleus with a certainty of 0.9800. In alternative embodiments, a NOV5a polypeptide is located to the microbody (peroxisome) with a certainty of 0.3000, the mitochondrial matrix space with a certainty of 0.1000, or the lysosome (lumen) with a certainty of 0.1000.

TABLE 5B
Encoded NOV5 Protein Sequence

(SEQ ID NO:20)

MPKGGCPKAPQQEELPLSSDMVEKQTGKKKDKVSLTKTPKLERGDGGKEVRERASKRKLPFTAGANGEQKDSD
TGTSPTALPLCDPFTYTAEEAKAERQKQGPERKRIKKEPVTRKAGLSGIRAGYPLSERQQVALLMQMTAEESA
NSPVDTTPKHPSQSTVCQKGTPNSASKTKDKVNKRNERGETRLHRAAIRGDARRIKELISEGADVNVKDFAGW
TALHEACNRGYYDVAKQLLAAGAEVNTKGLDDDTPLHDAANNGHQVVKLLLRYGGNPQQSNRKGETPLKVANS
PTMVNLLLGKGTYTSSEESSSEEEDAPSFAPSSSVDGNNTDSEFEKGLKHKAKNPEPQKATAPVKDEYEFDED
DEQDRVPPVDDKHLLKKDYRKETKSNSFISIPKMEVKSYTKNNTIAPKKASHRILSDTSDEEDASVTVGTGEK
LRLSAHTILPGSKTREPSNAKQQKEKNKVKKKRKKETKGREVRFGKRSDKFCSSESESESSESGEDDRDSLGS
SGCLKGSPLVLKDPSLFSSLSASSTSSHGSSAAQKQNDQHTKHWKTISSPAWSEVSSLSDSTRTRLTSESDYS
SEGSSVESLKPVRKRQEHRKRASLSEKKSPFLSSAEGAVPKLDKEGKVVKKHKTKHKHKNKEKISQELKLKSF
TYEYEDSKQKSDKAILLENDLSTENKLKVLKHDRDHFKKEEKLSKMKLEEKEWLFKDEKSLKRIKDKLRLYKE
ERDKISKEKEKIFKEDKEKLKKEKVYREDSLSDRDSSFDFKGAKLILETVKEDSKERRRDSRAREKHPAREKE
KPDKRKRYKEKDKDKSEKSILEKCQKDKEKKEKHKDTHGKDKERKASVFEKHKEKKDKESTEKYKDRASVDST
QDKKWKQEKAEKKHAAEDKAKSKHKEKSDKEHSKERKSSRSADAEYRESEVSSDSFTDREDDKSACLPEKLKE
KRHRHSSSSSKKSHDREEKKEDYKEGRKGQYEKDLEADAYGVSYNMKAIELFEKKDKNDEPLKEKKKREKHRE
KWRDEKERHRDRHADRPKPSKDPGKKDARPREKLLGDGDLMMTSRERMLSQKDLEIEERHKRHKERMKQMEKL
RHRSGDPKLKEKAKPADDGRKKGLDIPAKKPPGLDPPFKDKKLKESTPIPPAAENKLHPASGADSKDWLAGPH
MKEVLPASPRPDQSRPTGVPTPTSVLSCPSYEEVMHTPRTPSCSADDYADLVFDCADSQHSTPVPTAPTSACS
PSFFDRFSVASSGLSENASQAPARPLSTNLYRSVSVDIDKLFRQQSVPAASSYDSPMPPSMEDRAPLPPVPAE
KFACLSPGYYSPDYGLPSPKVDALHCPPAAVVTVTPSPEGVFSSLQAKPSPSPPSLDTSEDQQATAAIIPPEP
SYLEPLDEGPFSAVITEEPVEWAHPSEQALASSLIGGTSENPVSWPVGSDLLLKSPQRFPESPKRFCPADPLH
SAAPGPFSASEAPYPAPPASPAPYALPVAELEDVKDVPAAISTSEAAPYAPPSGLESFFSNCKSLPEAPLDVA
PEALGPLENSFLDGSRGLSHLGQVEPVPWADAFAGPEDDLDLGPFSLPELPLQTKDAADGEAEPVEESLAPPE
EMPPGAPRELEPEPSGEPKLDVALEAAVEAETVPEERARGDPDSSVEPAPVPPEQLGSGDPSLCAPDGPAPNT
VAQAQAADGAGPEDDTEASRAAAPAEGPPGQPEAAEPKPTAEAPKAPREIPQRMTRNRAQMLANQSKQGPPPS
EKECAPTPAPVTRAKARGSEDDDAQAQHPRKRRFQRSTQQLQLNTSTQQTREVTQQTLAAIVDAIKLDAIEPY
HSDRANPYFEYLQIRKKIEEKRKILCCITPQAPQCYAEYVTYTGSYLLDGKPLSKLHIPVIAPPPSLAEPLKE
LFRQQEAVRGKLRLQHSIEREKLIVSCEQEILRVHCRAARTIANQAVPFSACTMLLDSEVYNMPLESQGDENK
SVRDRFNARQFISWLQDVDDKYDRMKVCLLMRQQHEAAALNAVQRMEWQLKVQELDPAGHKSLCVNEVPSFYV
PMVDVNDDFVLLPA

The NOV5 amino acid sequence has 373 of 398 amino acid residues (93%) identical to, and 376 of 398 amino acid residues (93%) similar to, the 399 amino acid residue gi|17486077|refXP_{—}066756.1|XM_{—}066756 protein from Homo sapiens (Human) (similar to KIAA0874 PROTEIN) (E=0.0).

NOV5 is expressed in at least the following tissues: Heart, liver, Blood, Gall Bladder, Adrenal Gland/Suprarenal gland, Amygdala, Ascending Colon, Bone, Bone Marrow, Brain, Cervix, Dermis, Hippocampus, Kidney, Lung, Lymph node, Lymphoid tissue, Mammary gland/Breast, Ovary, Parotid Salivary glands, Pituitary Gland, Placenta, Prostate, Small Intestine, Spinal Chord, Spleen, Synovium/Synovial membrane, Testis, Thymus, Thyroid, Urinary Bladder, Vulva. This information was derived by determining the tissue sources of the sequences that were included in the invention including but not limited to SeqCalling sources, Public EST sources, Literature sources, and/or RACE sources.

NOV5 has homology to the amino acid sequences shown in the BLASTP data listed in Table 5C.

TABLE 5C
BLAST results for NOV5

| Gene Index/ | Protein/ | Length | Identity | Positives | |
|---|---|---|---|---|---|
| Identifier | Organism | (aa) | (%) | (%) | Expect |
| gi\|14140238\|ref\|NP_ | KIAA0874 | 2062 | 804/2109 | 1142/2109 | 0.0 |
| 056023.1\| | protein [Homo | | (38%) | (54%) | |
| (NM_015208) | sapiens] | | | | |
| gi\|17486077\|ref\|XP_ | similar to | 399 | 373/398 | 376/398 | 0.0 |
| 066756.1\| | KIAA0874 | | (93%) | (93%) | |
| (XM_066756) | protein (H. | | | | |
| | sapiens) | | | | |
| | [Homo sapiens] | | | | |
| gi\|7019449\|ref\|NP_ | nasopharyngeal | 366 | 308/366 | 315/366 | e−141 |
| 037407.1\| | carcinoma | | (84%) | (85% | |
| (NM_013275) | susceptibility | | | | |
| | protein | | | | |
| | [Homo sapiens] | | | | |
| gi\|6690397\|gb\|AAF24125.1 | nasopharyngeal | 366 | 308/366 | 315/366 | e−141 |
| \|AF121775_1 | carcinoma | | (84%) | (85% | |
| (AF121775) | susceptibility | | | | |
| | protein | | | | |
| | LZ16 [Homo | | | | |
| | sapiens] | | | | |
| gi\|4240237\|dbj\| | KIAA0874 | 601 | 283/600 | 365/600 | e−120 |
| BAA74897.1\|(AB020681) | protein [Homo | | (47%) | (60%) | |
| | sapiens] | | | | |
| gi\|17445427\|ref\|XP_ | similar to | 999 | 248/517 | 301/517 | 8e-80 |
| 065620.1\| | putative (H. | | (47%) | (57%) | |
| (XM_065820) | sapiens) | | | | |
| | [Homo sapiens] | | | | |

The homology of these sequences is shown graphically in the ClustalW analysis shown in Table 5D.

TABLE 5D
ClustalW Analysis of NOV5

| | |
|---|---|
| 1) NOV5 | (SEQ ID NO:20) |
| 2) gi\|14140238 ref NP 056023.1 (NM_015208) KIAA0874 | (SEQ ID NO:52) |
| protein [Homo sapiens] | |
| 3) gi\|17486077 ref XP 066756.1 (XM_066756) similar to | (SEQ ID NO:53) |
| KIAA0874 protein (H. sapiens) [Homo sapiens] | |
| 4) gi\|7019449\|ref\|NP 037407.1\| (NM_013275) naso- | (SEQ ID NO:54) |
| pharyngeal carcinoma susceptibility protein [Homo | |
| sapiens] | |
| 5) regi\|6690397L\|gb\|AAF24125.1 AF121775 1 (AF121775) | (SEQ ID NO:55) |
| nasopharyngeal carcinoma susceptibility protein LZ16 | |
| [Homo sapiens] | |
| 6) gi\|4240237\|dbj\|BAA74897.1\| (AB020681) KIAA0874 | (SEQ ID NO:56) |
| protein [Homo sapiens] | |
| 7) gi\|17445427 ref XP 065820.1 (XM_065820) similar to | (SEQ ID NO:57) |
| putative (H. sapiens) [Homo sapiens] | |

| | |
|---|---|
| | 10 20 30 40 50 |
| | .....\|....\|....\|....\|....\|....\|....\|....\|....\|....\| |
| NOV5 COR100396092 | -------------------------------------------------- |
| gi\|14140238\| | MPKSGFTKPIQSENSDSDSNMVEKPYGRKSKDKIASYSKTPKIERSDVSK |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 60 70 80 90 100 |
| | .....\|....\|....\|....\|....\|....\|....\|....\|....\|....\| |
| NOV5 COR100396092 | -------------------------------------------------- |
| gi\|14140238\| | EMKEKSSMKRKLPFTISPSRNEERDSDTDSDPGHTSENWGERLISSYRTY |
| gi\|17486077\| | -------------------------------------------------- |
| gi\|70194491\| | -------------------------------------------------- |
| gi\|4240237\| | -------------------------------------------------- |
| gi\|17445427\| | -------------------------------------------------- |
| | 110 120 130 140 150 |
| | .....\|....\|....\|....\|....\|....\|....\|....\|....\|....\| |
| NOV5 COR100396092 | -------------------------------------------------- |
| gi\|14140238\| | SEKEGPEKKKTKKEAGNKKSTPVSTLFGYPLSEKQMALLQMTARDNSP |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 160 170 180 190 200 |
| | .....\|....\|....\|....\|....\|....\|....\|....\|....\|....\| |
| NOV5 COR100396092 | -------------------------------------------------- |
| gi\|14140238\| | DSTPNHPSQTTPAQKKTPSSSSRQKDKVNKRNERGETPLHMAAIRGDVKQ |
| gi\|17488077\| | -------------------------------------------------- |
| gi\|7019449\| | -------------------------------------------------- |
| gi\|4240237\| | -------------------------------------------------- |
| gi\|17445427\| | -------------------------------------------------- |
| | 210 220 230 240 250 |
| | .....\|....\|....\|....\|....\|....\|....\|....\|....\|....\| |
| NOV5 COR100396092 | -------------------------------------------------- |
| gi\|14140238\| | VKELISLGANVNVKDFAGWTPLHEACNVGYYDVAKILIAAGADVNTQGLD |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 260 270 280 290 300 |
| | .....\|....\|....\|....\|....\|....\|....\|....\|....\|....\| |
| NOV5 COR100396092 | -------------------------------------------------- |
| gi\|14140238\| | DDTPLHDSASSGHRDIVKLLLRHGGNPFQANKHGERPVDVAETEELELLL |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 310 320 330 340 350 |
| | .....\|....\|....\|....\|....\|....\|....\|....\|....\|....\| |
| NOV5 COR100396092 | -------------------------------------------------- |
| gi\|14140238\| | KREVPLSDDDESYTDSEEAQSVNPSSVDENIDSETEKDSLICESKQILPS |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 360 370 380 390 400 |
| | .....\|....\|....\|....\|....\|....\|....\|....\|....\|....\| |
| NOV5 COR100396092 | -------------------------------------------------- |
| gi\|1410238\| | KTPLPSALDEYEFKDDDDEEINKMIDDRHILRKEQRKENEPEAEKTHLFA |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 410 420 430 440 450 |
| | .....\|....\|....\|....\|....\|....\|....\|....\|....\|....\| |
| NOV5 COR100396092 | -------------------------------------------------- |
| gi\|14140238\| | KQEKAFYPKSFKSKKQKPSRVLYSSTESSDEEALQNKKISTSCSVIPETS |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 460 470 480 490 500 |
| | .....\|....\|....\|....\|....\|....\|....\|....\|....\|....\| |
| NOV5 COR100396092 | -------------------------------------------------- |
| gi\|14140238\| | NSDMQTKKEYVVSGEHKQKGKVKRKLKNQNKNKENQELKQEKEGKENTRI |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 510 520 530 540 550 |
| | .....\|....\|....\|....\|....\|....\|....\|....\|....\|....\| |
| NOV5 COR100396092 | -------------------------------------------------- |
| gi\|1414023\| | TNLTVNTGLDCSEKTREEGNFRKSFSPKDDTSLHLFHISTGKSPKHSCGL |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 560 570 580 590 600 |
| | .....\|....\|....\|....\|....\|....\|....\|....\|....\|....\| |
| NOV5 COR100396092 | -------------------------------------------------- |
| gi\|14140238\| | SEKQSTPLKQEHTKTCSPGSSEMSLQPDLVRYDNTESEFLPESSSVKSC |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 610 620 630 640 650 |
| | .....\|....\|....\|....\|....\|....\|....\|....\|....\|....\| |
| NOV COR100396092 | -------------------------------------------------- |
| gi\|14140238\| | KHKEKSKHQKDFHLEFGEKSNAKIKDEDHSPTFENSDCTLKMDKEGKTL |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 660 670 680 690 700 |
| | .....\|....\|....\|....\|....\|....\|....\|....\|....\|....\| |
| NOV 5 COR100396092 | -------------------------------------------------- |
| gi\|14140238\| | KKHKLKHKEREKEKHKKEIEGEKEKYKTKDSAKELQRSVEFDREFWKENF |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 710 720 730 740 750 |
| | .....\|....\|....\|....\|....\|....\|....\|....\|....\|....\| |
| NOV5 COR100396092 | -------------------------------------------------- |
| gi\|14140238\| | FKSDETEDLFLNMEHESLTLEKKSKLEKNIKDDKSTKEKHVSKERNFKEE |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 760 770 780 790 800 |
| | .....\|....\|....\|....\|....\|....\|....\|....\|....\|....\| |
| NOV5 COR100396092 | -------------------------------------------------- |
| gi\|14140238\| | RDKIKKESEKSFREEKIKDLKEERENIPTDKDSEFTSLGMSAIEESIGLH |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 810 820 830 840 850 |
| | .....\|....\|....\|....\|....\|....\|....\|....\|....\|....\| |
| NOV5 COR100396096 | -------------------------------------------------- |
| gi\|14140238\| | LVEKEIDIEKQEKHIKESKEKPEKRSQIKEKDIEKMERKTFEKEKKIKHE |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 860 870 880 890 900 |
| | .....\|....\|....\|....\|....\|....\|....\|....\|....\|....\| |
| NOV5 COR100396092 | -------------------------------------------------- |
| gi\|14140238\| | HKSEKDKLDLSECVDKIKEKDKLYSHHTEKCHKEGEKSKNTAAIKKTDDR |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 910 920 930 940 950 |
| | .....\|....\|....\|....\|....\|....\|....\|....\|....\|....\| |
| NOV5 COR100396092 | -------------------------------------------------- |
| gi\|14140238\| | EKSREKMDRKHDKEKPEKERHLAESKEKHLMEKKNKQSDNSEYSKSEKGK |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 960 970 980 990 1000 |
| | .....\|....\|....\|....\|....\|....\|....\|....\|....\|....\| |
| NOV5 COR100396092 | -------------------------------------------------- |
| gi\|14140238\| | NKEKDRELDKKEKSRDKESINITNSKHIQEEKKSSIVDGNKAQHEKPLSL |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 1010 1020 1030 1040 1050 |
| | .....\|....\|....\|....\|....\|....\|....\|....\|....\|....\| |
| NOV5 COR100396092 | -------------------------------------------------- |
| gi\|14140238\| | KEKTKDEPLKTPDGKEKDKKDKDIDRYKERDKHKDKIQINSLLKLKSEAD |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 1060 1070 1080 1090 1100 |
| | .....\|....\|....\|....\|....\|....\|....\|....\|....\|....\| |
| NOV5 COR100396092 | -------------------------------------------------- |
| gi\|14140238\| | KPKPKSSPASKDTRPKEKRLVNDDLMQTSFERMLSLKDLEIEQWHKKHKE |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 1110 1120 1130 1140 1150 |
| | .....\|....\|....\|....\|....\|....\|....\|....\|....\|....\| |
| NOV5 COR100396092 | -------------------------------------------------- |
| gi\|14140238\| | KIKQKEKERLRNRNCLELKIKDKEKTKHTPTESKNKELTRSKSSEVTDAY |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 1160 1170 1180 1190 1200 |
| | .....\|....\|....\|....\|....\|....\|....\|....\|....\|....\| |
| NOV5 COR100396092 | -------------------------------------------------- |
| gi\|14140238\| | TKEKQPKDAVSNRSQSVDTKNVMTLGKSSFVSDNSLNRSPRSENEKPGLS |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 1210 1220 1230 1240 1250 |
| | .....\|....\|....\|....\|....\|....\|....\|....\|....\|....\| |
| NOV5 COR100396092 | -------------------------------------------------- |
| gi\|14140238\| | SRSVSMISVASSEDSCHTTVTTPRPPVEYDSDFMLESSESQMSFSQSPFL |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 1260 1270 1280 1290 1300 |
| | .....\|....\|....\|....\|....\|....\|....\|....\|....\|....\| |
| NOV5 COR100396092 | -------------------------------------------------- |
| gi\|14140238\| | SIAKSPALHERELDSLADLPERIKPPYANRLSTSHLRSSSVEDVKLIISE |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 1310 1320 1330 1340 1350 |
| | .....\|....\|....\|....\|....\|....\|....\|....\|....\|....\| |
| NOV5 COR100396092 | -------------------------------------------------- |
| gi\|14140238\| | GRPTIEVRRCSMPSVICEHTKQEQTISEESNQGSLLTVPGDTSPSPKPEV |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 1360 1370 1380 1390 1400 |
| | .....\|....\|....\|....\|....\|....\|....\|....\|....\|....\| |
| NOV5 COR100396092 | -------------------------------------------------- |
| gi\|14140238\| | FSNVPERDLSNVSNIHSSFATSPTGASNSKYVSADRNLIKNTAPVNTVMD |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 1410 1420 1430 1440 1450 |
| | .....\|....\|....\|....\|....\|....\|....\|....\|....\|....\| |
| NOV5 COR100396092 | -------------------------------------------------- |
| gi\|14140238\| | SPVHLEPSSQVGVIQNKSWEPVDRLETLSTRDFICPNSNIPDQESSLOS |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 1460 1470 1480 1490 1500 |
| | .....\|....\|....\|....\|....\|....\|....\|....\|....\|....\| |
| NOV5 COR100396092 | -------------------------------------------------- |
| gi\|14140238\| | FCNSENKVLKENADFLSLRQTELPGNSCAQDPASFMPPQQPCSFPSQSLS |
| gi\|17486077\| | -------------------------------------------------- |
| gi\|7019449\| | -------------------------------------------------- |
| gi\|4240237\| | -----------NADFLSLRQTELPGNSCAQDPASFMPPQQPCSFPSQSLS |
| g1\|17445427\| | ---------MISEEKEWLFKDEIIKVSKDEKSLKRIKGMNKDISRSFQEE |
| | 1510 1520 1530 1540 1550 |
| | .....\|....\|....\|....\|....\|....\|....\|....\|....\|....\| |
| NOV5 COR100396092 | ------MPKGGCPK P QEELPLSSDM EMQTGKK-K KV LTK PKLER |
| gi\|14140238\| | DAESISKHMSLSYV N EPGILQQKNA QIISSALDT NE TKD ENTFV |
| gi\|17486077\| | -------------------------------------------------- |
| gi\|70194491\| | ------MPKGGCPK P QEELPLSSDM EKQTGKKDK KV LTK PKLER |
| gi\|4240237\| | DAESISKHMSLSYV N EPGILQQKNA QIISSALDT NE TKD ENTFV |
| gi\|17445427\| | KDCSNTAEKEKSLKEKSSKEEKLRLYKEERKTPKRQK KEPKDKRKDTGA |
| | 1560 1570 1580 1590 1600 |
| | .....\|....\|....\|....\|....\|....\|....\|....\|....\|....\| |
| NOV5 COR100396092 | GDGGKE-----------------VRERASKRK FTAG------------ |
| gi\|14140238\| | LGDVQKTDAFVP-VYSDSTIQEASPNFEKAYT VLPSE DFNGSDASTQ |
| gi\|17486077\| | -------------------------------M QS-SA DYLG ----- |
| gi\|7019449\| | GDGGKE--------------- VRERASKRK FTAG ------- |
| gi\|4240237\| | LGDVQKTDAFVP-VYSDSTIQEASPNFEKAYT VLPSE DFNGSDASTQ |
| gi\|17445427\| | ADGVTDKKEKVLEKHKEKKVEYQKNKKNKQK EKAEK QSAEDK----- |
| | 1610 1620 1630 1640 1650 |
| | .....\|....\|....\|....\|....\|....\|....\|....\|....\|....\| |
| NOV5 COR100396092 | ------NGEQ DSDTGTSPTAL----------------PLCDPFTYTAE |
| gi\|14140238\| | LNTHY FSKLTYKSSSGHEV NSTTDTQVISEKENKLESLVLTHLSRCD |
| gi\|17486077\| | -----EYCIL AQAADGAGP DDTEASRAAAPAE--------------- |
| gi\|7019449\| | ------NGEQ DSDT---------------------------------- |
| gi\|4240237\| | LNTHY FSKLTYKSSSGHEV NSTTDTQVISHEKENKLESLVLTHLSRCD |
| gi\|17445427\| | ------NSKH EKSDKEYSK RKSLRSADMEKSLLEKLEEALHEYRDDSS |
| | 1660 1670 1680 1690 1700 |
| | .....\|....\|....\|....\|....\|....\|....\|....\|....\|....\| |
| NOV5 COR100396092 | EAKAERQKQG ERKRIKKE VTRKA-------------GLSG RAGYPLS |
| gi\|14140238\| | SDLCEMNAGM KGNLNEQD KHCPES-EKCLLSIEDEESQQS LSSLENH |
| gi\|17486077\| | --------GP GG--------------------IQPEAAE --------- |
| gi\|7019449\| | ------EKQG ERKRIKKE VTRKAG-------LLFGMGLSG RAGYPLS |
| gi\|4240237\| | SDLCEMNAGM KGNLNEQD KHCPES-EKCLLSIEDEESQQS LSSLENH |
| gi\|17445427\| | DKITTTERDSQERKVPEEKGRDYKEGGSRKDTGQYEKDFLEMVAYGVSYN |
| | 1710 1720 1730 1740 1750 |
| | .....\|....\|....\|....\|....\|....\|....\|....\|....\|....\| |
| NOV5 COR100396092 | ER QVALL QMTAEESANSPVDTTPKHPSQSTVC KG P S SKTKDKVN |
| gi\|14140238\| | SQ STQPE HKYGQLVKVELEENAEDDKTENQIP RM R K NTMANQSK |
| gi\|17486077\| | ________P_________KPTAEAPKAPRVEEIP RM R R QMLANQSK |
| gi\|7019449\| | ER QVALL QMTAEESANSPVDTTPKHPSQSTVC KG P S SKTKDKLN |
| gi\|4240237\| | SQ STQPE HKYGQLVKVELEENAEDDKTENQIP RM R K NTMANQSK |
| gi\|17445428\| | MKAVIEDRLNKTVELFSTEKKDKNDSERETSKKIEKELKPYGSRTQKPT |
| | 1760 1770 1780 1790 1800 |
| | .....\|....\|....\|....\|....\|....\|....\|....\|....\|....\| |
| NOV5 COR100396092 | KRNERGETRLHRAAIRGDA--------KELISEG DVNVKDFAGWTALHE |
| gi\|14140238\| | QILASCTLLSEKDESSSP------G RLTED DPQIHHPRKRKVSRVQ |
| gi\|17486077\| | QGPPPSEKECAP-TPAPVT-----AKARGSED D QAQHPRKRRFQRSTO |
| gi\|7019449\| | KRNERGETRLHRAAIRGDA--------KELISEG DVNVKDFAGWTALHE |
| gi\|4240237\| | QILASCTLLSEKDESSSP-----G RLTED DPQIHHPRKRKVSRVPQ |
| gi\|17445427\| | ARDKDSPPRALKDKSRDEDPRLRKAKLKEKFK S EKEKDDSVKMSKGDD |
| | 1810 1820 1830 1840 1850 |
| | .....\|....\|....\|....\|....\|....\|....\|....\|....\|....\| |
| NOV5 COR100396092 | ACN----------------------------------------------- |
| gi\|14140238\| | PVQVSP-------------------------------------------- |
| gi\|17486077\| | QLQQLN-------------------------------------------- |
| gi\|7019449\| | ACN----------------------------------------------- |
| gi\|4240237\| | PVQVSP-------------------------------------------- |
| gi\|17445427\| | KVSPSKDPGKKNARPREKLRGDGDMMIISFQRMFSQKDLEIEERHKGHKE |
| | 1860 1870 1880 1890 1900 |
| | .....\|....\|....\|....\|....\|....\|....\|....\|....\|....\| |
| NOV5 COR10396092 | -----------------------------RGYYDVA QLLAAG E NTKG |
| gi\|14140238\| | -----------------------------SLLQAKE TQQS A I DSL |
| gi\|17486077\| | ----------------------------TSTQQTREVIQQT A I DAI |
| gi\|7019449\| | -----------------------------RGYYDVA QLLAAG E NTKG |
| gi\|4240237\| | -----------------------------SLLQAKE TQQS A I DSL |
| gi\|17445427\| | RMKQMEKLRHQSRDPNLKERAKPADDGRKKGLEIPA KPPG DPPFKDK |
| | 1910 1920 1930 1940 1950 |
| | .....\|....\|....\|....\|....\|....\|....\|....\|....\|....\| |
| NOV5 COR100396092 | DDT LHDAAN GH--------------QVV LL RYGGN OS-- |
| gi\|14140238\| | EIQ YSSERA PYFEYLHIRK------IEE RK LCSVI AP-- |
| gi\|17486077\| | AIE YHSDRA PYFEYLQIRK------IEE RKILCCIT AP-- |
| gi\|7019449\| | DDT LHDAAN GHY-------------KVV LL RYGGN QS------ |
| gi\|4240237\| | EIQ YSSERA PYFEYLHIRK------IEE RK LCSVI AP------ |
| gi\|17445427\| | KELT IPPAAE KPRPGSGADS DWLAGPHM EV PASPR DQSRPVCP |
| | 1960 1970 1980 1990 2000 |
| | .....\|....\|....\|....\|....\|....\|....\|....\|....\|....\| |
| NOV5 COR100396092 | ----------NRK ETP KVANS--------------------------- |
| gi\|14140238\| | ---QYYDEYV FN SYL D NPLSKICIPTIT ----------------- |
| gi\|17486077\| | ---QWYAQYV YT SYL D KPLSKLHIPVIA ----------------- |
| gi\|7019449\| | ----------NRK ETP KVANS--------------------------- |
| gi\|4240237\| | ---QYYDEYV FN SYL D NPLSKICIPTIT ----------------- |
| gi\|17445427\| | PLRRCCPASA RR HSPAP RHRGPAGYSPHH GAQLPGAAGRGLIGSA |
| | 2010 2020 2030 2040 2050 |
| | .....\|....\|....\|....\|....\|....\|....\|....\|....\|....\| |
| NOV5 COR100396092 | -------TMVNL LG------------------------KGTYTSSE--- |
| gi\|14140238\| | -------SLSDP KELFRQ-----------QEVVRMKLRLOH IEREK I |
| gi\|17486077\| | -------SLAEP KELFRQ-----------QEAVRGKLRLQH IEREK I |
| gi\|7019449\| | -------TMVNL LG------------------------KGTYTSSE--- |
| gi\|4240237\| | -------SLSDP KELFRQ-----------QEVVRMKLRLQH IEREK I |
| gi\|17445427\| | SENPVSW VGSEL LKSPQRFPESPEYFCSADSLHSAAPGPF ASENT L |
| | 2060 2070 2080 2090 2100 |
| | .....\|....\|....\|....\|....\|....\|....\|....\|....\|....\| |
| NOV5 COR100396092 | -------------ES---SSEEED PS APSSSVDGNN------------ |
| gi\|14140238\| | VSN--- QEVL HYRA RTLANQTL SACTV LDAEVYNVP D___ |
| gi\|17486077\| | VSC--- QEIL HCRA RTIANQ V STCTM LDSEVYNMP E--- |
| gi\|7019449\| | ------------ESSTESSEEED PS APSSSVDGNN------------- |
| gi\|4240237\| | VSN--- QEVL HYRA TRLANQTL SACTV LDAEVYNVP D-- |
| gi\|17445427\| | IAEPGL DVKD EAIP TISTSE A YAPPSG ESFFNNCKS PESLLD |
| | 2110 2120 2130 2140 2150 |
| | .....\|....\|....\|....\|....\|....\|....\|....\|....\|....\| |
| NOV5 COR100396092 | ----------------TD EFE GLKHKAKNPEPQKATAPVKDEYEFDED |
| gi\|14140238\| | -----------------Q DS T-------------------------- |
| gi\|17486077\| | -----------------QG EN --------------------------- |
| gi\|7019449\| | ----------------TD EFE G-------------------------- |
| gi\|4240237\| | -----------------Q DS T-------------------------- |
| gi\|17445427\| | MAPEACNHCGSDAFAG ED LDLGSFSLPELPLQTKDVPDVETEPTEESL |
| | 2160 2170 2180 2190 2200 |
| | .....\|....\|....\|....\|....\|....\|....\|....\|....\|....\| |
| NOV5 COR100396092 | DEODRVPP---------------------------VDDHKLLKKDYRKET |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | APSEKIPGAPVVLPTELEPEPSEEKPKLDVALEATEAEAVPEERSAGDLD |
| | 2210 2220 2230 2240 2250 |
| | .....\|....\|....\|....\|....\|....\|....\|....\|....\|....\| |
| NOV5 COR100396092 | K NSFISIPMEVKSYTKNN--------------------TIAPKKASHRI |
| gi\|14140238\| | - VR-------DRFNA-----------------------QFMS L DVDD |
| gi\|17486077\| | - VR-------DRFNA-----------------------QFIS L DVDD |
| gi\|7019449\| | - ---------S---T--------------------------PRT SHR- |
| gi\|4240237\| | - VR-------DRFNA-----------------------QFMS L DVDD |
| gi\|17445427\| | S MEPTPVRPEQCQLGS DQGAEAEHLLPPAASLCAPDTPCPP TLWHKP |
| | 2260 2270 2280 2290 2300 |
| | .....\|....\|....\|....\|....\|....\|....\|....\|....\|....\| |
| NOV5 COR100396092 | LS TSDEEDASVTVGTGEKLRLSAHT----ILPGSKTREPSN KQQKEKN |
| gi\|14140238\| | KF KLKT------------------------CLLM QQHE A LN VQ L |
| gi\|17486077\| | KY RMKT------------------------CLLM QQHE A LN VQ M |
| gi\|7019449\| | ------------------------------------PRPPS--------T |
| gi\|4240237\| | KF KLKT------------------------CLLM QQHE A LN VO L |
| gi\|17445427\| | RLRTVLAPTTTLRASRAAAPAEGPPCGIDPEATESEPKPT E PK PRHS |
| | 2310 2320 2330 2340 2350 |
| | .....\|....\|....\|....\|....\|....\|....\|....\|....\|....\| |
| NOV5 COR100396092 | KVKK RKKETKGREVRFGKRSDKFCSSESESESSESGEDDRDSLGSSGC |
| gi\|14140238\| | EW LQELDP-------------------------------ATYKSI |
| gi\|17486077\| | EW VQELDP-------------------------------AGHKS |
| gi\|7019449\| | SMS MRTTS----------R--------------------------TGF |
| gi\|4240237\| | EW LQELDP-------------------------------ATYKSI |
| gi\|17445427\| | TQ NTSTQQT-----REVIQQTLATIVDAIKLDAIPYHSDRANPYFEF |
| | 2360 2370 2380 2390 2400 |
| | .....\|....\|....\|....\|....\|....\|....\|....\|....\|....\| |
| NOV5 COR100396092 KGSPLVLKDPSLFSSLSASSTSSHSSSAAQKQNDQHTKHWKTISSPAWSE | |
| gi\|14140238\| | SIY---------------EIQEF VPL DVNDDFE TPI----------- |
| gi\|17486077\| | CVN---------------EVPSF VPM DVNDDFV LPA----------- |
| gi\|7019449\| | R-----------------WTTST--------------------------- |
| gi\|4240237\| | SIY---------------EIQEF VPL DVNDDFE TPI----------- |
| gi\|17445427\| | HIRKKIEEKRKILCCITPQATQW AEY TYTGSYL DGKSLSKLHMPMIA |
| | 2410 2420 2430 2440 2450 |
| | .....\|....\|....\|....\|....\|....\|....\|....\|....\|....\| |
| NOV5 COR100396092 | VSSLSDSTRTRLTSESDYSSEGSSVESLKPVRKRQEHRKRASLSEKKSPF |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | PPPSLRASATRTSQCATGSTPASSSPGSMTWTTIQPHEDLLTWQQHEAAA |
| | 2460 2470 2480 2490 2500 |
| | .....\|....\|....\|....\|....\|....\|....\|....\|....\|....\| |
| NOV5 COR100396092 | LSSAEGAVPKLDKEGKVVKKHKTKHKHKNKEKISQELKLKSFTYEYEDSK |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | LNAMQRMEWQLKVQKLDPAGH----------------------------- |
| | 2510 2520 2530 2540 2550 |
| | .....\|....\|....\|....\|....\|....\|....\|....\|....\|....\| |
| NOV5 COR100396092 | QKSDKAILLENDLSTENKLKVLKHDRDHFKKEEKLSKMKLEEKEWLFKDE |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 2560 2570 2580 2590 2600 |
| | .....\|....\|....\|....\|....\|....\|....\|....\|....\|....\| |
| NOV5 COR100396092 | KSLKRIKDKLRLYKEERDKISKEKEKIFKEDKEKLKKEKVYREDSLSDRD |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 2610 2620 2630 2640 2650 |
| | .....\|....\|....\|....\|....\|....\|....\|....\|....\|....\| |
| NOV COR100396092\| | SSFDFKGAKLILETVKEDSKERRRDSRAREKHPAREKEKPDKRKRYKEKD |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 2660 2670 2680 2690 2700 |
| | .....\|....\|....\|....\|....\|....\|....\|....\|....\|....\| |
| NOV5 COR100396092 | KDKSEKSILEKCQKDKEKKEKHKKDTHGKDKERKASVFEKHKEKKDKESTE |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 2710 2720 2730 2740 2750 |
| | .....\|....\|....\|....\|....\|....\|....\|....\|....\|....\| |
| NOV5 COR10039609 | KYKDRASVDSTQDKKNKQEKAEKKHAAEDKAKSKHKEKSDKEHSKERKSS |
| gi\|17486077\| | -------------------------------------------------- |
| gi\|17486077\| | -------------------------------------------------- |
| gi\|7019449\| | -------------------------------------------------- |
| gi\|4249237\| | -------------------------------------------------- |
| gi\|17445427\| | -------------------------------------------------- |
| | 2760 2770 2780 2790 2800 |
| | .....\|....\|....\|....\|....\|....\|....\|....\|....\|....\| |
| NOV5 COR100396092 | RSADAEYRESEVSSDSFTDREDDKSACLPEKLKEKRHRHSSSSSKKSHDR |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 2810 2820 2830 2840 2850 |
| | .....\|....\|....\|....\|....\|....\|....\|....\|....\|....\| |
| NOV5 COR100396092 | EEKKEDYKEGRKGQYEKDLEADAYGVSYNMKAIELFEKKDKNDEPLKEKK |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 2860 2870 2880 2890 2900 |
| | .....\|....\|....\|....\|....\|....\|....\|....\|....\|....\| |
| NOV5 COR100396092 | KREKHREKWRDEKERHRDRHADRPKPSKDPGKKDARPREKLLGDGDLMMT |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 2910 2920 2930 2940 2950 |
| | .....\|....\|....\|....\|....\|....\|....\|....\|....\|....\| |
| NOV5 COR100396092 | SFERMLSQKDLEIEERHKRHKERMKQMEKLRHRSGDPKLKEKAKPADDGR |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 2960 2970 2980 2990 3000 |
| | .....\|....\|....\|....\|....\|....\|....\|....\|....\|....\| |
| NOV5 COR100396092 | KKGLDIPAKKPPGLDPPFKDKKLKESTPIPPAAENKLHPASGADSKDWLA |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3010 3020 3030 3040 3050 |
| | .....\|....\|....\|....\|....\|....\|....\|....\|....\|....\| |
| NOV5 COR100396092 | GPHMKEVLPASPRPDQSRPTGVPTPTSVLSCPSYEEVMHTPRTPSCSADD |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3060 3070 3080 3090 3100 |
| | .....\|....\|....\|....\|....\|....\|....\|....\|....\|....\| |
| NOV5 COR100396092 | YADLVFDCADSQHSTPVPTAPTSACSPSFFDRFSVASSGLSENASQAPAR |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3110 3120 3130 3140 3150 |
| | .....\|....\|....\|....\|....\|....\|....\|....\|....\|....\| |
| NOV5 COR100396092 | PLSTNLYRSVSVDIDKLFRQQSVPAASSYDSPMPPSMEDRAPLPPVPAEK |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3160 3170 3180 3190 3200 |
| | .....\|....\|....\|....\|....\|....\|....\|....\|....\|....\| |
| NOV5 COR100396092 | FACLSPGYYSPDYGLPSPKVDALHCPPAAVVTVTPSPEGVFSSLQAKPSP |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3210 3220 3230 3240 3250 |
| | .....\|....\|....\|....\|....\|....\|....\|....\|....\|....\| |
| NOV5 COR100396092 | SPPSLDTSEDQQATAAIIPPEPSYLEPLDEGPFSAVTTEEPVEWAHPSEQ |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3260 3270 3280 3290 3300 |
| | .....\|....\|....\|....\|....\|....\|....\|....\|....\|....\| |
| NOV5 COR100396092 | ALASSLIGGTSENPVSWPVGSDLLLRSPQRFPESPKRFCPADPLHSAAPG |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3310 3320 3330 3340 3350 |
| | .....\|....\|....\|....\|....\|....\|....\|....\|....\|....\| |
| NOV5 COR100396092 | PFSASEAPYPAPPASPAPYALPVAELEDVKDVPAAISTSEAAPYAPPGL |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3360 3370 3380 3390 3400 |
| | .....\|....\|....\|....\|....\|....\|....\|....\|....\|....\| |
| NOV5 COR100396092 | ESFFSNCKSLPEAPLDVAPEALGPLENSFLDGRSGLSHLGQVEPVPWADA |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3410 3420 3430 3440 3450 |
| | .....\|....\|....\|....\|....\|....\|....\|....\|....\|....\| |
| NOV5 COR100396092 | FAGPEDDLDLGPFSLPELPLQTKDAADGEAEPVEESLAPPEEMPPGAPRE |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3460 3470 3480 3490 3500 |
| | .....\|....\|....\|....\|....\|....\|....\|....\|....\|....\| |
| NOV5 COR100396092 | LEPESGEPKLDVALEAAVEAETVPEERARGDPDSSVEPAPVPPEQLGSG |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3510 3520 3530 3540 3550 |
| | .....\|....\|....\|....\|....\|....\|....\|....\|....\|....\| |
| NOV5 COR100396092 | DPSLCAPDGPAPNTVAQAQAADGAGPEDDTEASRAAAPAEGPPGQPEAAE |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3560 3570 3580 3590 3600 |
| | .....\|....\|....\|....\|....\|....\|....\|....\|....\|....\| |
| NOV5 COR100396092 | PKPTAEAPKAPREIPQRMTRNRAQMLANQSKQGPPPSEKECAPTPAPVTR |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3610 3620 3630 3640 3650 |
| | .....\|....\|....\|....\|....\|....\|....\|....\|....\|....\| |
| NOV5 COR100396092 | AKARGSEDDDAQAQHPRKRRFQRSTQQLQLNTSTQQTREVIQQTLAAIVD |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3660 3670 3680 3690 3700 |
| | .....\|....\|....\|....\|....\|....\|....\|....\|....\|....\| |
| NOV5 COR100396092 | AIKLDAIEPYHSDPANPYFEYLQIRKKIEEKRKTLCCITPQAPQCYAEYV |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3710 3720 3730 3740 3750 |
| | .....\|....\|....\|....\|....\|....\|....\|....\|....\|....\| |
| NOV5 COR100396092 | TYTGSYLLDGKPLSKLHIPVIAPPPSLAEPLKELFRQQEAVRGKLRLQHS |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3760 3770 3780 3790 3800 |
| | .....\|....\|....\|....\|....\|....\|....\|....\|....\|....\| |
| NOV5 COR100396092 | IEREKLIVSCEQEILRVHCRAARTIANQAVPFSACTMLLDSEVYNMPLES |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3810 3820 3830 3840 3850 |
| | .....\|....\|....\|....\|....\|....\|....\|....\|....\|....\| |
| NOV5 COR100396092 | QGDENKSVRDRFNARQFISWLQDVDDKYDRMKVCLLMRQQHEAAALNAVQ |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |
| | 3860 3870 3880 3890 |
| | .....\|....\|....\|....\|....\|....\|....\|....\|....\|....\| |
| NOV5 COR100396092 | RMEWQLKVQELDPAGHKSLCVNEVPSFYVPMVDVNDDFVLLPA |
| gi\|14140238\| | -------------------------------------------------- |
| gi\|17486077\| | -------------------------------------------------- |
| gi\|7019449\| | -------------------------------------------------- |
| gi\|4240237\| | -------------------------------------------------- |
| gi\|17445427\| | -------------------------------------------------- |

Tables 5E, 5F, 5G, 5H, 5I and 5J list the domain description from DOMAIN analysis results against NOV5. This indicates that the NOV5 sequence has properties similar to those of other proteins known to contain these domains.

TABLE 5E
Domain Analysis of NOV5

gn1|Pfam|pfam0023, ank, Ank repeat. Ankyrin
repeats generally consist of a beta, alpha, alpha,
beta order of secondary structures. The repeats
associate to form a higher order structure.
(SEQ ID NO:58)
CD-Length = 33 residues, 84.8% aligned
Score = 45.8 bits (107), Expect = 2e−05

| | | | |
|---|---|---|---|
| NOV 5: | 218 | GWTALHEACNRGYYDVAKQLLAAGAEVN | 245 |
| | | \| \| \|\| \| \|++\| \| \|\| \|\|\|+\|\| | |
| Sbjct: 2 | GNTPLHLAARNGHLEVVKLLLEAGADVN | 29 | |

TABLE 5F
Domain Analysis of NOV5

gn1|Pfam|pfam00023, ank, Ank repeat. Ankyrin
repeats generally consist of a beta, alpha, alpha,
beta order of secondary structures. The repeats
associate to form a higher order structure.
(SEQ ID NO:59)
CD-Length = 33 residues, 100.0% aligned
Score = 43.1 bits (100), Expect = 2e−04

| | | | |
|---|---|---|---|
| NOV 5: | 250 | DDDTPLHDAANNGH-QVVKLLLRYGGNPQQSNR | 281 |
| | | \| +\|\|\|\| \|\| \|\|\| +\|\|\|\|\|\| \| + ++ | |
| Sbjct: | 1 | DGNTPLHLAANGHLEVVKLLLEAGADVNARDK | 33 |

TABLE 5G
Domain Analysis of NOV5

gn1|Pfam|pfam00023, ank, Ank repeat. Ankyrin
repeats generally consist of a beta, alpha, alpha,
beta order of secondary structures. The repeats
associate to form a higher order structure.
(SEQ ID NO:60)
CD-Length = 33 residues, 93.9% aligned
Score = 42.0 bits (97), Expect = 3e−04

| | | | |
|---|---|---|---|
| NOV 5: | 185 | GETRLHRAAIRGDARRIKELISEGADVNVKD | 215 |
| | | \| \| \|\| \|\| \| +\| \|+ \|\|\|\|\| +\| | |
| Sbjct: | 2 | GNTPLHLAARNGHLEVVKLLLEAGADVNARD | 32 |

TABLE 5H
Domain Analysis of NOV5

gn1|Smart|smart00248+EE, ANK, ankyrin repeats; Ankyrin
repeats are about 33 amino acids long and occur in
at least four consecutive copies. They are
involved in protein-protein interactions. The core
of the repeat seems to be an helix-loop-helix
structure.
(SEQ ID NO:61)
CD-Length = 30 residues, 93.3% aligned
Score = 43.1 bits (100). Expect = 2e−04

| | | | |
|---|---|---|---|
| NOV 5: | 218 | GWTALHEACNRGYYDVAKQLLAAGAEVN | 245 |
| | | \| \| \|\| 51 \| +\| \| \|\| \|\|++\| | |
| Sbjct: | 2 | GRTPLHLAAENGNLEVVKLLLDKGADIN | 29 |

TABLE 5I
Domain Analysis of NOV5

gn1|Smart smart00248+EE, ANK, ankyrin repeats; Ankyrin
repeats are about 33 amino acids long and occur in
at least four consecutive copies. They are
involved in protein-protein interactions. The core
of the repeat seems to be an helix-loop-helix
structure.
(SEQ ID NO:62)
CD-Length = 30 residues, 93.3% aligned
Score = 41.2 bits (95), Expect = 6e−04

| | | | |
|---|---|---|---|
| NOV 5: | 250 | DDDTPLHDAANNGH-QVVKLLLRYGGNP | 276 |
| | | \| \|\|\|\| \|\| \|\|+ +\|\|\|\|\|\| \| + | |
| Sbjct: | 1 | DGRTPLHLAAENGNLEVVKLLLDKGADI | 28 |

TABLE 5J
Domain Analysis of NOV5

gn1|Smart|smart00248, ANK, ankyrin repeats;
Ankyrin repeats are about 33 amino acids long and
occur in at least four consecutive copies. They
are involved in protein-protein interactions. The
core of the repeat seems to be an helix-loop-helix
structure.
(SEQ ID NO:63)
CD-Length = 30 residues, 96.7% aligned
Score = 39.3 bits (90), Expect = 0.002

| | | | |
|---|---|---|---|
| NOV 5: | 185 | GETRLHRAAIRGDARRIKELISEGADVNV | 213 |
| | | \| \| \|\| \|\| \|+ +\| \|++\|\|\|+\|+ | |
| Sbjct: | 2 | GRTPLHLAAENGNLEVVKLLLDKGADINL | 30 |

Ankyrin repeats are tandemly repeated modules of about 33 amino acids. They occur in a large number of functionally diverse proteins mainly from eukaryotes. The few known examples from prokaryotes and viruses may be the result of horizontal gene transfers. The conserved fold of the ankyrin repeat unit is known from several crystal and solution structures, e.g., from: p53-binding protein 53BP2, cyclin-dependent kinase inhibitor p19Ink4d, transcriptional regulator GABP-beta, and NF-kappaB inhibitory protein IkB-alpha. It has has been described as an L-shaped structure consisting of a beta-hairpin and two alpha-helices. Many ankyrin repeat regions are known to function as protein-protein interaction domains.

The protein similarity information, expression pattern, and map location for the NOV5 protein and nucleic acid disclosed herein suggest that it may have important structural and/or physiological functions characteristic of the family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: Cardio-vascular disorders, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Systemic lupus erythematosus, Autoimmune disease, Asthma, Emphysema, Scleroderma, allergy, Diabetes, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome and other diseases, disorders and conditions of the like.

NOV6

A disclosed NOV6 nucleic acid (designated as CuraGen Acc. No. COR87941483), encodes a novel TNF intracellular domain interacting protein and includes the 1749 nucleotide sequence (SEQ ID NO: 21) shown in Table 6A. An open reading frame for the mature protein was identified beginning with an ATG codon at nucleotides 103-105 and ending with a TAG codon at nucleotides 1579-1581. Putative untranslated regions downstream from the termination codon and upstream from the initiation codon are underlined in Table 6A, and the start and stop codons are in bold letters.

TABLE 6A
NOV6a Nucleotide Sequence

(SEQ ID NO:21)

AGAACGCGGAGAGTCGCCGCCTGGCCGGGCGTAGACGCGGTGGCAGAGCCCGCGCGGCGCTGGAA
GCGAGTGGCGGAGCGGCGGGACCTCGGCGGACTCGCCATGGAGGAGGAGGGTGTGAAGGAAGCCG
GTGAGAAGCCTCGGGGAGCACAGATGGTGGACAAGGCTGGCTGGATCAAGAAGAGCAGTGGGGGC
CTCCTGGGTTTCTGGAAAGACCGATATCTGCTCCTCTGCCAGGCCCAGCTGCTGGTCTATGAGAATG
AGGATGATCAGAAGTGTGTGGAGACTGTGGAGCTGGGCAGCTATGAGAAGTGCCAGGACCTTCGTG
CCCTCCTCAAGCGAAAACACCGCTTTATCCTGCTGCGATCCCCAGGGAACAAGGTCAGCGACATCA
AATTCCAGGCACCCACCGGGGAGGAGAAGGAATCCTGGATCAAAGCCCTCAATGAAGGGATTAAC
CGAGGCAAAAACAAGGCTTTCGATGAGGTAAAGGTGGACAAGAGCTGCGCCCTGGAGCATGTGAC
ACGGGACCGGGTGCGAGGGGGCCAGCGACGCCGGCCACCAACGAGAGTCCACCTGAAGGAGGTGG
CCAGTGCAGCTTCTGACGGTCTTCTGCGCCTGGATCTTGATGTTCCGGACAGTGGGCCACCAGTGTT
TGCCCCCAGCAATCATGTCAGTGAAGCCCAACCTCGGGAGACACCCCGGCCCCTCATGCCTCCTACC
AAGCCTTTCCTAGCACCTGAGACCACCAGCCCTGGTGACAGGGTGGAGACCCCTGTGGGGGAGAGA
GCCCCAACCCCTGTCTCAGCAAGCTCTGAGGTCTCCCCTGAGAGCCAAGAGGACTCAGAGACCCCA
GCAGAGGAGGACAGTGGCTCTGAGCAGCCTCCCAACAGCGTCCTGCCTGACAAACTGAAGGTGAGC
TGGGAGAACCCCAGCCCCCAGGAGGCCCCTGCTGCAGAGAGTGCAGAACCGTCCCAGGCACCCTGT
TCTGAGACTTCTGAGGCTGCCCCCAGGGAGGGTGGGAAGCCCCCTACACCCCCACCCAAGATCTTA
TCAGAAGAACACTTGAAAGCCTCCATGGGTGAGATGCAGGCTTCTGGGCCACCTGCTCCAGGCACA
GTGAAAGGTCTCAGTCAAATGGCAAGAATGGAAGGACTGAGCATTGCCAAGCACTCTAAGGCTGA
AGGCACCCAAAGAACTTCTCCAAAGGATGCACTAACACACCAAGCACTGCCCCCCTGGGACCTGCC
ACCTCAGTTCCATCACCGCTGCTCCTCCCTTGGGGACTTGCTTGGGGAAGGCCCGCGGCATCCCTTG
CAGCCCAGGCAACGGCTATATCGGGCCCAGCTGGAGGTGAAGGTGGCCTCGGAACAGACGGAGAA
ACTGTTGAACAAGGTGCTGGGCAGTGAGCCGGCCCCTGTTAGTGCCGAAACATTGCTCAGCCAGGC
TGTGGAGCAGCTGAGGCAGGCCACCCAGGTCCTGCAGGAAATGAGAGATTTGGGAGAGCTGAGCC
AGGAAGCACCTGGGCTAAGGGAGAAGCGGAAGGAGCTGGTGACCCTCTACAGGAGAAGTGCACCC
TAGGGCCTTCTGGGCCAGAGGCACCATCCCTTCTGGCCATCCATCAAGTCCATCAAGGCCCAGCCCT
GCTGAGAAATGTGCTTCTGCTTCTACAGCAATGGCTGCAGGAGGGCCATTGGGCATGTCAGGGTTT
GGCCATGACCCGAAGAGACTCCTGGCGTCCTTCCTACT

The nucleic acid sequence of NOV6 maps to chromosome 15 and has 360 of 631 bases (57%) identical to a gb:GENBANK-ID:AF168676|acc:AF168676.1 mRNA from Homo sapiens (Homo sapiens TNF intracellular domain-interacting protein mRNA, complete cds).

The NOV6 polypeptide (SEQ ID NO: 22) is 492 amino acid residues in length and is presented using the one-letter amino acid code in Table 6B. The SignalP, Psort and/or Hydropathy results predict that NOV6 has a signal peptide and is likely to be localized to the nucleus with a certainty of 0.7000. In alternative embodiments, a NOV6 polypeptide is located to the mitochondrial matrix spacewith a certainty of 0.1000 or the lysosome (lumen) with a certainty of 0.1000.

TABLE 6B
Encoded NOV6 Protein Sequence

(SEQ ID NO:22)

MEEEGVKEAGEKPRGAQMVDKAGWIKKSSGGLLGFWKDRYLLLCQAQLLVYENEDDQKCVETVE
LGSYEKCQDLRALLKRKHRIFILLRSPGNKVSDIKFQAPTGEEKESWIKALNEGINRGKNKAFDEVKV
DKSCALEHVTRDRVRGGQRRRPPTRVHLKEVASAASDGLLRLDLDVPDSGPPVFAPSNHVSEAQPRE
TPRPLMPPTKPFLAPETTSPGDRVETPVGERAPTPVSASSEVSPESQEDSETPAEEDSGSEQPPNSVLPD
KLKVSWENPSPQEAPAAESAEPSQAPCSETSEAAPREGGKPPTPPPKILSEEHLKASMGEMQASGPPA
PGTVKGLSQMARMEGLSIAKHSKAEGTQRTSPKDALTHQALPPWDLPPQFHHRCSSLGDLLGEGPR
HPLQPRQRLYRAQLEVKVASEQTEKLLNKVLGSEPAPVSAETLLSQAVEQLRQATQVLQEMRDLGE
LSQEAPGLREKRKELVTLYRRSAP

The NOV6 amino acid sequence has 263 of 289 amino acid residues (91%) identical to, and 269 of 289 amino acid residues (93%) similar to, the 399 amino acid residue gi|18027838|gb|AAL55880.1 AF3 8373_{—}1 AF318373 protein from Homo sapiens (Human) (UNKNOWN) (E=e⁻¹⁰²).

NOV6 has homology to the amino acid sequences shown in the BLASTP data listed in Table 6C.

TABLE 6C
BLAST results for NOV6

| | | | | | |
|---|---|---|---|---|---|
| Gene Index/ | Protein | Length | Identity | Positives | |
| Identifier | Organism | (aa) | (%) | (%) | Expect |

| | | | | |
|---|---|---|---|---|
| gi 18027838\|gb AAL5 | unknown [Homo 287 | 263/289 | 269/289 | e−102 |
| 5880.1 AF318373 1 | sapiens] | (91%) | (93%) | |
| (AF318373) | | | | |

The homology of this sequence is shown graphically in the ClustalW analysis shown in Table 6D.

TABLE 6D
ClustalW Analysis of NOV6

| | |
|---|---|
| 1) NOVG | (SEQ ID NO:22) |
| 2) gi\|18027838 gb\|AAL55880.1\|AF318373 1 (AF318373) | (SEQ ID NO:64) |
| unknown [Homo sapiens] | |

| | |
|---|---|
| | 10 20 30 40 50 |
| | ....\|....\|....\|....\|....\|....\|....\|....\|....\|....\| |
| NOV6 COR87941483 | MEEEGVKEACEKPRGAQMVDKAGWIKKSSGGLLCFWKDRYLLLCQAQLLV |
| gi\|18027838\| | -------------------------------------------------- |
| | 60 70 80 90 100 |
| | ....\|....\|....\|....\|....\|....\|....\|....\|....\|....\| |
| NOV6 COR87941483 | YENEDDQKCVETVELGSYEKCQDLRALLKRKHRFILLRSPGNKVSDIKFQ |
| gi\|18027838\| | -------------------------------------------------- |
| | 110 120 130 140 150 |
| | ....\|....\|....\|....\|....\|....\|....\|....\|....\|....\| |
| NOV6 COR87941483 | APTGEEKESWIKALNEGINRGKNKAFDEVKVDKSCALEHVTRDRVRGGQR |
| gi\|18027838\| | -------------------------------------------------- |
| | 160 170 180 190 200 |
| | ....\|....\|....\|....\|....\|....\|....\|....\|....\|....\| |
| NOV6 COR87941483 | RRPPTRVHLKEVASAASDGLLRLDLDVPDSGPPVFAPSNHVSEAQPRETP |
| gi\|18027838\| | -------------------------------------------------- |
| | 210 220 230 240 250 |
| | ....\|....\|....\|....\|....\|....\|....\|....\|....\|....\| |
| NOV6 COR87941483 | RPL |
| gi\|18027838\| | --- |
| | 260 270 280 290 300 |
| | ....\|....\|....\|....\|....\|....\|....\|....\|....\|....\| |
| NOV6 COR87941483 | P |
| gi\|18027838\| | S |
| | 310 320 330 340 350 |
| | ....\|....\|....\|....\|....\|....\|....\|....\|....\|....\| |
| NOV6 COR87941483 | EH KGL QMARME |
| gi\|18027838\| | K- Q-V VNGMDD |
| | 360 370 380 390 400 |
| | ....\|....\|....\|....\|....\|....\|....\|....\|....\|....\| |
| NOV6 COR87941483 | GLSI H K QR S L HQ H |
| gi\|18027838\| | SPEP P Q PG P T ST P |
| | 410 420 430 440 450 |
| | ....\|....\|....\|....\|....\|....\|....\|....\|....\|....\| |
| NOV6 COR87941483 | Q |
| gi\|18027838\| | E |
| | 460 470 480 490 |
| | ....\|....\|....\|....\|....\|....\|....\|....\|.. |
| NOV6 COR87941483 | |
| gi\|18027838\| | |

Tables 6E and 6F list the domain description from DOMAIN analysis results against NOV6. This indicates that the NOV6 sequence has properties similar to those of other proteins known to contain these domains.

TABLE 6E
Domain Analysis of NOV6

gn1|Pfam|pam00169, PH, PH domain. PH stands for pleckstrin homology
(SEQ ID NO:65)
CD-Length = 100 residues, 99.0% aligned
Score = 57.8 bits (138), Expect = 1e−09

| | | | |
|---|---|---|---|
| NOV 6: | 19 | VDKAGWIKKSSGGLLGFWKDRYLLLCQAQLLVYENE-DDQKCVETVELGSYEKCQDLRAL | 77 |
| | | + \| \|\|+ \| \| \|\| \|\| \| \|+ \|+++ + ++ \| + | |
| Sbjct: | 1 | IVKEGWLLKKSTVKKKRWKKRYFFLFNDVLIYYKDKKKSYEPKGSIPLSCCSVEDVPDSE | 60 |
| NOV6: | 78 | LKRKHRFILLRSPGNKVSDIKFQAPTGEEKESWIKALNEGI | 118 |
| | | \|\| + \| \| \|+ \|\| + \|\|++ \|\|\|\|+ \| | |
| Sbjct: | 61 | FKRPNCFQLRSRDGKET--FILQAESEEERQDWIKAIQSAI | 99 |

TABLE 6F
Domain Analysis of NOV6

gn1|Smart|smart00233, PH, Pleckstrin homology domain.; Domain commonly
found (SEQ ID NO:66) in eukaryotic signalling proteins. The domain family
possesses multiple functions including the abilities to bind inositol
phosphates, and various proteins. PH domains have been found to possess
inserted domains (such as in PLC gamma, syntrophins) and to be inserted
within other domains. Mutations in Brutons tyro sine kinase (Btk) within
its PH domain cause X-linked agammaglobulinaemia (XLA) in patients. Point
mutations cluster into the positively charged end of the molecule around
the predicted binding site for phosphatidylinositol lipids.
CD-Length = 104 residues, 99.0% aligned
Score = 57.8 bits (138), Expect = 1e−09

| | | | |
|---|---|---|---|
| NOV 6: | 19 | VDKAGWIKKSSGGLLGFWKDRYLLLCQAQLLVYENE---DDQECVETVELGSYEKCQDLR | 75 |
| | | \| \| \|\|+ \| \| \| \|\| \|\| +\| \|\| \|+++ \| ++ \| + | |
| Sbjct: | 1 | VIKEGWLLKKSSGGKKSWKKRYFVLFNGVLLYYKSKKKKSSSKPKGSIPLSGCTVREAPD | 60 |
| NOV 6: | 76 | ALLKRKHRFILLRSPGNKVSDIKFQAPTGEEKESWIKALNEGINR | 120 |
| | | + +\| + +\| \| + \|\| +=10 \|\|++ \|++\|\| + \| + | |
| Sbjct: | 61 | SDSDKKKNCFEIVTPDRKT--LLLQAESEEERKEWVEALEKAIAK | 103 |

The protein similarity information, expression pattern, and map location for the NOV6 protein and nucleic acid disclosed herein suggest that it may have important structural and/or physiological functions characteristic of the family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The NOV6 nucleic acid and protein are useful in potential diagnostic and therapeutic applications implicated in various diseases and disorders described below and/or other pathologies. For example, the compositions of the present invention will have efficacy for treatment of patients suffering from: Cardio-vascular disorders, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Systemic lupus erythematosus, Autoimmune disease, Asthma, Emphysema, Scleroderma, allergy, Diabetes, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome and other diseases, disorders and conditions of the like.

The ‘pleckstrin homology’ (PH) domain is a domain of about 100 residues that occurs in a wide range of proteins involved in intracellular signaling or as constituents of the cytoskeleton. The function of this domain is not clear, several putative functions have been suggested:

binding to the beta/gamma subunit of heterotrimeric G proteins,

binding to lipids, e.g. phosphatidylinositol-4,5-bisphosphate,

binding to phosphorylated Ser/Thr residues,

attachment to membranes by an unknown mechanism.

It is possible that different PH domains have totally different ligand requirements. The 3D structure of several PH domains has been determined. All known cases have a common structure consisting of two perpendicular anti-parallel beta sheets, followed by a C-terminal amphipathic helix. The loops connecting the beta-strands differ greatly in length, making the PH domain relatively difficult to detect. There are no totally invariant residues within the PH domain.

Proteins reported to contain one more PH domains belong to the following families:

Pleckstrin, the protein where this domain was first detected, is the major substrate of protein kinase C in platelets. Pleckstrin is one of the rare proteins to contains two PH domains.

Ser/Thr protein kinases such as the Act/Rac family, the beta-adrenergic receptor kinases, the mu isoform of PKC and the trypanosomal NrkA family.

Tyrosine protein kinases belonging to the Btk/Itk/Tec subfamily.

Insulin Receptor Substrate 1 (IRS-1).

Regulators of small G-proteins like guanine nucleotide releasing factor

NOV7

A disclosed NOV7 nucleic acid (designated as CuraGen Acc. No. COR101716725) encodes a novel secretory protein and includes the 1491 nucleotide sequence (SEQ ID NO: 23) shown in Table 7A. An open reading frame for the mature protein was identified beginning with an ATG codon at nucleotides 31-33 and ending with a TGA codon at nucleotides 1324-1326. Putative untranslated regions are underlined in Table 7A, and the start and stop codons are in bold letters.

TABLE 7A
NOV7 Nucleotide Sequence

(SEQ ID NO:23)

GGGCCCGCGCAGCCCCGGCCGGAACCCACCATGCGGCGGCTGCGGCGCCTGGCGCACCTGGTGCTC
TTCTGCCCCTTCTCCAAGCGCCTGCAGGGCCGGCTCCCAGGCCTCAGGGTCCGCTGCATCTTCCTGG
CCTGGCTGGGCGTCTTTGCAGGCAGCTGGCTGGTGTACGTGCACTACTCGTCCTACTCGGAGCGCTG
TCGCGGCCATGTCTGCCAGGTGGTCATTTGTGACCAGTACCGCAAGGGGATCATCTCGGGCTCCGTC
TGCCAGGACCTGTGTGAGCTGCATATGGTGGAGTGGAGGACCTGCCTCTCGGTGGCCCCGGGCCAG
CAGGTGTACAGCGGGCTCTGGCGGGACAAGGATGTAACCATCAAGTGTGGCATTGAGGAGACCCTC
GACTCCAAGGCCCGGTCGGATGCGGCCCCCCGGCGGGAGCTGGTACTGTTTGACAAGCCCACCCGG
GGCACCTCCATCAAGGAATTCCGGGAGATGACCCTCGGCTTCCTCAAGGCGAACCTGGGAGACCTG
CCTTCCCTGCCGGCGCTGGYFGGCCAGGTCCTGCTCATGGCTGACTTCAACAAGGACAACCGGGTGT
CCCTGGCGGAAGCCAAGTCCGTGTGGGCCCTGCTGCAGCGTAACGAGTTCCTGCTGCTGCTGTCCCT
GCAGGAGAAGGAGCACGCCTCCAGACTGCTGGGCTACTGTGGGGACCTCTACCTCACCGAGGGCGT
GCCGCATGGCGCCTGGCACGCGGCCGCCCTTCCACCCCTGTTGCGCCCACTGCTGCCGCCTGCCCTG
CAGGGTGCTCTCCAGCAGTGGCTGGGGCCTGCGTGGCCTTGGCGGGCCAAGATCGCCATCGGCCTG
CTGGAGTTCGTGGAGGAGCTCTTCCACGGCTCTTACGGGACTTTCTACATGTGTGAGACCACACTGG
CCAACGTGGGCTACACAGCCACCTACGACTTCAAGATGGCCGACCTGCAGCAGGTGGCACCCGAGG
CCACCGTGCGCCGCTTCCTGCAGGGCCGCCGCTGCGAGCACAGCACCGACTGCACCTACGGGCGCG
ACTGCAGGCCCCCGTGTGACAGGCTCATGAGGCAGTGCAAGGGCGACCTCATCCAGCCCAACCTGG
CCAAGGTGTGCGCACTGCTACGGGGCTACCTGCTGCCTGGCGCGCCCGCCGACCTCCGCGAGGAGC
TGGGCACACAGCTGCGCACCTGTACCACGCTGAGCGGGCTGGCCAGCCAGGTGGAGGCCCATCACT
CGCTGGTGCTCAGCCACCTCAAGACTCTGCTCTGGAAGAAGATCTCCAACACCAAGTACTCTTGAT
GGGGCAGTGAGGGGCCTGGCCACCCTTCCTGGAGCTGGCCAGGTGCCAGGGTCCAACCCTCCCTCA
AGGAGAGTCCTCCAAGGGGGTTTGTTACTCTGAAGAACGTAATGTCAATAAACAGCTTTTATGTAAT
GCCCAGGGCTGAGCACCCTGAGCCCCCATCA

The nucleic acid sequence of NOV7 has 1137 of 1347 bases (84%) identical to a gb:GENBANK-ID:AB030186|acc:AB030186.1 mRNA from Mus musculus (Mus musculus mRNA, complete cds, clone:1-82).

The NOV7 polypeptide (SEQ ID NO: 24) is 431 amino acid residues in length and is presented using the one-letter amino acid code in Table 7B. The SignalP, Psort and/or Hydropathy results predict that NOV7 has a signal peptide and is likely to be located outside of the cell with a certainty of 0.6615. In alternative embodiments, a NOV7 polypeptide is located to the microbody (peroxisome) with a certainty of 0.1215, the endoplasmic reticulum (membrane) with a certainty of 0.1000, or the endoplasmic reticulum (lumen) with a certainty of 0.1000. The SignalP predicts a likely cleavage site for a NOV7 peptide between amino acid positions 59 and 60, i.e., at the dash in the sequence CRG-HV.

TABLE 7B
Encoded NOV7 Protein Sequence

(SEQ ID NO:24)

MRRLRRLAHLVLFCPFSKRLQGRLPGLRVRCIFLAWLGVFAGSWLVYVHYSSYSERCRGHVCQVVI
CDQYRKGIISGSVCQDLCELHMVEWRTCLSVAPGQQVYSGLWRDKDVTIKCGIEETLDSKARSDAA
PRRELVLFDKPTRGTSIKEFREMTLGFLKANLGDLPSLPALVGQVLLMADFNKDNRVSLAEAKSVW
ALLQRNEFLLLLSLQEKEHASRLLGYCGDLYLTEGVPHGAWHAAALPPLLRPLLPPALQGALQQWL
GPAWPWRAKIAGLLEFVEELFHGSYGTFYMCETTLANVGYTATYDFKMADLQQVAPEATVRRFLQ
GRRCEHSTDCTYGRDCRAPCDRLMRQCKGDLIQPNLAKVCALLRGYLLPGAPADLREELGTQLRTC
TTLSGLASQVEAHHSLVLSHLKTLLWKKISNTKYS

The NOV7 amino acid sequence has 255 of 256 amino acid residues (99%) identical to, and 255 of 266 amino acid residues (99%) similar to, the 266 amino acid residue gi|18027802|gb|AAL55862.1|AF318355_{—}1AF318355 protein from Homo sapiens (Human) (UNKNOWN) (E=e⁻¹³⁶).

NOV7 is expressed in at least the following tissues: Adipose, Adrenal Gland/Suprarenal gland, Amygdala, Aorta, Bone, Bone Marrow, Brain, Cerebral Medulla/Cerebral white matter, Cervix, Chorionic Villus, Colon, Coronary Artery, Dermis, Epidermis, Foreskin, Frontal Lobe, Heart, Hippocampus, Kidney, Liver, Lung, Lymph node, Lymphoid tissue, Mammary gland/Breast, Muscle, Ovary, Pancreas, Parathyroid Gland, Parotid Salivary glands, Peripheral Blood, Pineal Gland, Pituitary Gland, Placenta, Prostate, Respiratory Bronchiole, Retina, Skin, Small Intestine, Spinal Chord, Stomach, Substantia Nigra, Synovium/Synovial membrane, Testis, Thalamus, Thyroid, Tonsils, Umbilical Vein, Uterus and Vein. This information was derived by determining the tissue sources of the sequences that were included in the invention including but not limited to SeqCalling sources, Public EST sources, Literature sources, and/or RACE sources.

NOV7 also has homology to the amino acid sequences shown in the BLASTP data listed in Table 7C.

TABLE 7C
BLAST results for NOV7

| Gene Index/ | Protein/ | Length | Identity | Positives | |
|---|---|---|---|---|---|
| Identifier | Organism | (aa) | (%) | (%) | Expect |
| gi\|13272520\|gb\| | induced | 431 | 382/431 | 397/431 | 0.0 |
| AAK17190.1\|AF332189_1 | protein 49 | | (88%) | (91%) | |
| (AF332189) | [Mus | | | | |
| pancreatitis- | musculus] | | | | |
| gi\|9790001\|ref\| | hypothetical | 428 | 313/348 | 322/348 | e−176 |
| NP_062807.1\| | protein 1-82 | | (89%) | (91%) | |
| (NM_019833) | [Mus | | | | |
| | musculus] | | | | |
| gi\|18027802\|gb\| | unknown | 266 | 255/256 | 255/256 | e−136 |
| AAL55862.1\|AF318355_1 | [Homo sapiens] | | (99%) | (99%) | |
| (AF318355) | | | | | |
| gi\|12850997\|dbj\| | putative [Mus | 428 | 199/412 | 280/412 | e−121 |
| BAB28914.1\|(AK013580) | musculus] | | (48%) | (67%) | |
| gi\|17433824\|ref\|XP_ | hypothetical | 403 | 194/403 | 275/403 | e−119 |
| 028387.2\| | protein | | (48%) | (68%) | |
| (XM_028387) | XP_028387 | | | | |
| | [Homo sapiens] | | | | |

The homology of these sequences is shown graphically in the ClustalW analysis shown in Table 7D.

TABLE 7D
ClustalW Analysis of NOV7

| | |
|---|---|
| 1) NOV7 | (SEQ ID NO:24) |
| 2) gi 13272520 gb\|AAK17190.1\|AF332189 1 (AF332189) | (SEQ ID NO:67) |
| pancreatitis-induced protein 49 [Mus musculus] | |
| 3) gi\|9790001\|ref\|NP 062807.1\| (NM_019833) | (SEQ ID NO:68) |
| hypothetical protein 1-82 [Mus musculus] | |
| 4) gi\|18027802\|gb\|AAL55861.1 AF318355 1 (AF318355) | (SEQ ID NO:69) |
| unknown [Homo sapiens] | |
| 5) gi\|12850997 dbj BAB28914.1\| (AK013580) putative [Mus | (SEQ ID NO:70) |
| musculus] | |
| 6) gi\|17433824 ref XP 028387.2 (XM_028387) hypothetical | (SEQ ID NO:71) |
| protein XP_028387 [Homo sapiens] | |
| | 10 20 30 40 50 |
| | ....\|....\|....\|....\|....\|....\|....\|....\|....\|....\| |
| NOV7 COR101716725 | MRRLRRLAHLVLFCPFSKRLQGRLPGLRVRCIFLA GV A LV H |
| gi\|13272520\| | MRRLRRLVHLVLLCPFSKGLQGRLPGLRVKYVLLV GI V MV H |
| gi\|9790001\| | --------------------------------------------MV H |
| gi\|18027802\| | -------------------------------------------------- |
| gi\|12850997\| | ---MARSLCAGAWLRKPHLQARLSYMRVKYLFFS VV V II Q |
| gi\|17433824\| | ---------------------------MKYLFFS VV V II Q |
| | 60 70 80 90 100 |
| | ....\|....\|....\|....\|....\|....\|....\|....\|....\|....\| |
| NOV7 COR101716725 | S S R HV QVV Q RK I S SV QD ELHM V |
| gi\|13272520\| | S S HV QVV Q RK I S SV QD ELQK S |
| gi\|9790001\| | S S HV QVV Q RK I S SV QD ELQK S |
| gi\|18027802\| | -------------------------------------M V |
| gi\|12850997\| | T T KD KKI K KT V D PA NS VTETLYFGK NK S |
| gi\|17433824\| | T T KD KKI K KT V D PA NS VTETLYFGK TK N |
| | 110 120 130 140 150 |
| | ....\|....\|....\|....\|....\|....\|....\|....\|....\|....\| |
| NOV7 COR101716725 | R D T D RS |
| gi\|13272520\| | Q E N WP |
| gi\|9790001\| | Q E N WP |
| gi\|18027802\| | R D T D RS |
| gi\|12850997\| | N M L V DNLPGVV QM Q HLDFGTELE K I TVQ |
| gi\|17433824\| | N M L I DNLPGVV QM Q HLDFGTELE K I TVQ |
| | 160 170 180 190 200 |
| | ....\|....\|....\|....\|....\|....\|....\|....\|....\|....\| |
| NOV7 COR101716725 | G G V N |
| gi\|13272520\| | D S |
| gi\|9790001\| | D S |
| gi\|18027802\| | G G V N |
| gi\|12850997\| | K K VY LF K QGN SE NL TV GDR GQ G A |
| gi\|17433824\| | K K VY LF K QGN SE NL TV GD GQ G A |
| | 210 220 230 240 250 |
| | ....\|....\|....\|....\|....\|....\|....\|....\|....\|....\| |
| NOV7 COR101716725 | A A A L |
| gi\|13272520\| | I V L A |
| gi\|9790001\| | I V L A |
| gi\|18027802\| | A A A L |
| gi\|12850997\| | L MVI D TPK M F 10 VM S EYT LY IS WVMEL |
| gi\|17433824\| | L MVI D TPK M F 10 VM S EYT LY IS WVMEL |
| | 260 270 280 290 300 |
| | ....\|....\|....\|....\|....\|....\|....\|....\|....\|....\| |
| NOV7 COR101716725 | PA QG L |
| gi\|13272520\| | V H |
| gi\|9790001\| | V H |
| gi\|18027802\| | PA QG L |
| gi\|12850997\| | FI GFR SMD L T S RK DV P N L D SA |
| gi\|17433824\| | FI GFR SMD L T S RK DV P N L D SA |
| | 310 320 330 340 350 |
| | ....\|....\|....\|....\|....\|....\|....\|....\|....\|....\| |
| NOV7 COR101716725 | R H T T R AP |
| gi\|13272520\| | Q S I R AP |
| gi\|9790001\| | Q S I R AP |
| gi\|18027802\| | R H T TT--------- |
| gi\|12850997\| | K L NEK L V MRKIV TNLKELIKD SDL V T TS |
| gi\|17433824\| | K L NDK L V MRKIV TNLKELIKD SDL V T TS |
| | 360 370 380 390 400 |
| | ....\|....\|....\|....\|....\|....\|....\|....\|....\|....\| |
| NOV7 COR101716725 | RLMRQ KGDL V A RG P ADLR GT RT TT S |
| gi\|13272520\| | RLMRQ KGDL V E R P AGLY G RT TT S |
| gi\|9790001\| | RLMRQ KGDL V E R P AGLY G CAPAPQKV |
| gi\|18027802\| | --------------------TAGPRVTGS--------------------- |
| gi\|12850997\| | LSTMK TSEV A Q K H SEIR E YS IA K |
| gi\|17433824\| | QSTMK TSEV A Q K R SEIR E YS IA K |
| | 410 420 430 440 450 |
| | ....\|....\|....\|....\|....\|....\|....\|....\|....\|....\| |
| NOV7 COR101716725 | GL S V AH AH V SH KK N----------KY---------- |
| gi\|13272520\| | GL S I AH AH V SH RE N----------NY---------- |
| gi\|9790001\| | DWPARLRLTIHWC AT RPYSGGRSPTPTTPRAAGSRHY SQVAPPHSLQ |
| gi\|18027802\| | -------------------------------------------------- |
| gi\|12850997\| | VT N M ME I NN KK Y----------ND---------- |
| gi\|17433824\| | VT N M ME I NN KK Y----------ND---------- |
| | 460 470 |
| | ....\|....\|....\|....\|.. |
| NOV7 COR101716725 | ---------------------- |
| gi\|13272520\| | ---------------------- |
| gi\|9790001\| | QLSRGARGPYQRSPTGPNPPNM |
| gi\|18027802\| | ---------------------- |
| gi\|12850997\| | ---------------------- |
| gi\|17433824\| | ---------------------- |

Many calcium-binding proteins belong to the same evolutionary family and share a type of calcium-binding domain known as the EF-hand. This type of domain consists of a twelve residue loop flanked on both side by a twelve residue alpha-helical domain. In an EF-hand loop the calcium ion is coordinated in a pentagonal bipyramidal configuration. The six residues involved in the binding are in positions 1, 3, 5, 7, 9 and 12; these residues are denoted by X, Y, Z, -Y, -X and -Z. The invariant Glu or Asp at position 12 provides two oxygens for liganding Ca (bidentate ligand).

The protein similarity information, expression pattern, and map location for the NOV7 protein and nucleic acid disclosed herein suggest that it may have important structural and/or physiological functions characteristic of the EF-hand family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The NOV7 nucleic acid and protein are useful in potential diagnostic and therapeutic applications implicated in various diseases and disorders described below and/or other pathologies. For example, the compositions of the present invention will have efficacy for treatment of patients suffering from: Cardio-vascular diseases, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Systemic lupus erythematosus, Autoimmune disease, Asthma, Emphysema, Scleroderma, allergy, Diabetes, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome and other diseases, disorders and conditions of the like.

NOV8

NOV8 includes two GPCR-like proteins. They have been designated NOV8a and NOV8b.

NOV8a

A disclosed NOV8a nucleic acid (designated as CuraGen Acc. No. CG56663-01), encodes a novel GPCR-like protein and includes the 1062 nucleotide sequence (SEQ ID NO: 25) shown in Table 8A. An open reading frame for the mature protein was identified beginning with an ATG codon at nucleotides 10-12 and ending with a TAA codon at nucleotides 948-950. Putative untranslated regions are underlined in Table 8A, and the start and stop codons are in bold letters.

TABLE 8A
NOV8a Nucleotide Sequence

(SEQ ID NO:25)

TAGAGATGGATGGAACCAATGGCAGCACCCAAACCCATTTCATCCTACTGGGATTCTCTGACCGAC
CCCATCTGGAGAGGATCCTCTTTGTGGTCATCCTGATCGCGTACCTCCTGACCCTCGTAGGCAACAC
CACCATCATCCTGGTGTCCCGGCTGGACCCCCACCTCCACACCCCCATGTACTTCTTCCTCGCCCACC
TTTCCTTCCTGGACCTCAGTTTCACCACCAGCTCCATCCCCCAGCTGCTCTACAACCTTAATGGATGT
GACAAGACCATCAGCTACATGGGCTGTGCCATCCAGCTCTTCCTGTTCCTGGGTCTGGGTGGTGTGG
AGTGCCTGCTTCTGGCTGTCATGGCCTATGACCGGTGTGTGGCTATCTGCAAGCCCCTGCACTACAT
GGTGATCATGAACCCCAGGCTCTGCCGGGGCTTGGTGTCAGTGACCTGGGGCTGTGGGGTGGCCAA
CTCCTTGGCCATGTCTCCTGTGACCCTGCGCTTACCCCGCTGTGGGCACCACGAGGTGGACCACTTC
CTGCGTGAGATGCCCGCCCTGATCCGGATGGCCTGCGTCAGCACTGTGGCCATCGAAGGCACCGTC
TTTGTCCTGAAAAAAGGTGTTGTGCTGTCCCCCTTGGTGTTTATCCTGCTCTCTTACAGCTACATTGT
GAGGGCTGTGTTACAAATTCGGTCAGCATCAGGAAGGCAGAAGGCCTTCGGCACCTGCGGCTCCCA
TCTCACTGTGGTCTCCCTTTTCTATGGAAACATCATCTACATGTACATGCAGCCAGGAGCCAGTTCTT
CCCAGGACCAGGGCATGTTCCTCATGCTCTTCTACAACATTGTCACCCCCCTCCTCAATCCTCTCATC
TACACCCTCAGAAACAGAGAGGTGAAGGGGGCACTGGGAAGGTTGCTTCTGGGGAAGAGAGAGCT
AGGAAAGGAGTAAAGGCATCTCCACCTGACTTCACTTCCATCCAGGGCCACTGGCAGCATCTGGAA
CGGCTGAATTCCAGCTGATATTAGCCCACGACTCCCAACTTGCCTTTTTCTGGACTTTT

The NOV8a polypeptide (SEQ ID NO: 26) is 314 amino acid residues in length and is presented using the one-letter amino acid code in Table 8B.

TABLE 8B
Encoded NOV8a Protein Sequence

(SEQ ID NO:26)

MDGTNGSTQTHFILLGFSDRPHLERILFVVILIAYLLTLVGNTTIILVSRLDPHLHTPMYFFLAHLSFLD
LSFTTSSIPQLLYNLNGCDKTISYMGCAIQLFLFLGLGGVECLLLAVMAYDRCVAICKPLHYMVIMNP
RLCRGLVSVTWGCGVANSLAMSPVTLRLPRCGHHEVDHFLREMPALIRMACVSTVAIEGTVFVLKK
GVVLSPLVFILLSYSYIVRAVLQIRSASGRQKAFGTCGSHLTVVSLFYGNIIYMYMQPGASSSQDQGM
FLMLFYNIVTPLLNPLIYTLRNREVKGALGRLLLGKRELGKE

NOV8b

A disclosed NOV8b nucleic acid (designated as CuraGen Acc. No. CG56663-02), which is a variant of NOV8a, includes the 1062 nucleotide sequence (SEQ ID NO: 27) shown in Table 8C. An open reading frame for the mature protein was identified beginning with an ATG codon at nucleotides 6-8 and ending with a TAA codon at nucleotides 948-950. The start and stop codons of the open reading frame are highlighted in bold type. Putative untranslated regions are underlined and found upstream from the initiation codon and downstream from the termination codon.

TABLE 8C
NOV8b Nucleotide Sequence

| | |
|---|---|
| TAGAGATGGATGGAACCAATGGCAGCACCCAAACCCATTTCATCCTACTGGGATTCTCTGAC | (SEQ ID NO:27) |
| CGACCCCATCTGGAGAGGATCCTCTTTGTGGTCATCCTGATCGCGTACCTCCTGACCCTCGTA | |
| GGCAACACCACCATCATCCTGGTGTCCCGGCTGGACCCCCACCTCCACACCCCCATGTACTT | |
| CTTCCTCGCCCACCTTTCCTTCCTGGACCTCAGTTTCACCACCAGCTCCATCCCCCAGCTGCTC | |
| TACAACCTTAATGGATGTGACAAGACCATCAGCTACATGGGCTGTGCCATCCAGCTCTTCCT | |
| GTTCCTGGGTCTGGGTGGTGTGGAGTGCCTGCTTCTGGCTGTCATGGCCTATGACCGGTGTGT | |
| GGCTATCTGCAAGCCCCTGCACTACATGGTGATCATGAACCCCAGGCTCTGCCGGGGCTTGG | |
| TGTCAGTGACCTGGGGCTGTGGGGTGGCCAACTCCTTGGCCATGTCTCCTGTGACCCTGCGCT | |
| TACCCCGCTGTGGGCACCACGAGGTGGACCACTTCCTGCGTGAGATGCCCGCCCTGATCCGG | |
| ATGGCCTGCGTCAGCACTGTGGCCATCGACGGCACCGTCTTTGTCCTGGCGGTGGGTGTTGT | |
| GCTGTCCCCCTTGGTGTTTATCCTGCTCTCTTACAGCTACATTGTGAGGGCTGTGTTACAAAT | |
| TCGGTCAGCATCAGGAAGGCAGAAGGCCTTCGGCACCTGCGGCTCCCATCTCACTGTGGTCT | |
| CCCTTTTCTATGGAAACATCATCTACATGTACATGCAGCCAGGAGCCAGTTCTTCCCAGGAC | |
| CAGGGCATGTTCCTCATGCTCTTCTACAACATTGTCACCCCCCTCCTCAATCCTCTCATCTAC | |
| ACCCTCAGAAACAGAGAGGTGAAGGGGGCACTGGGAAGGTTGCTTTTGGGGAAGAGAGAGC | |
| TAGGAAAGGAGTAAAGGCATCTCCACCTGACTTCACTTCCATCCAGGGCCACTGGCAGCATC | |
| TGGAACGGCTGAATTCCAGCTGATATTAGCCCACGACTCCCAACTTGCCTTTTTCTGGACTTT | |
| T | |

A NOV8b polypeptide (SEQ ID NO: 28) is 314 amino acid residues and is presented using the one letter code in Table 8D.

TABLE 8D
Encoded NOV8b Protein Sequence

(SEQ ID NO:28)

MDGTNGSTQTHFILLGFSDRPHLERILFVVILIAYLLTLVGNTTIILVSRLDPHLHTPMYFFLAHLSFLDLSF
TTSSIPQLLYNLNGCDKTISYMGCAIQLFLFLGLGGVECLLLAVMAYDRCVAICKPLHYMVIMNPRLCR
GLVSVTWGCGVANSLAMSPVTLRLPRCGHHEVDHFLREMPALIRMACVSTVAIDGTVFVLAVGVVLSP
LVFILLSYSYLVRAVLQIRSASGRQKAFGTCGSHLTVVSLFYGNIIYMYMQPGASSSQDQGMFLMLFYNI
VTPLLNPLIYTLRNREVKGALGRLLLGKRELGKE

The nucleic acid sequence of NOV8 has 600 of 710 bases (84%) identical to a gb:GENBANK-ID:AX008326|acc:AX008326.1 mRNA from Marmota marmota (Sequence 24 from Patent WO9967282).

A NOV8 amino acid sequence has 314 of 314 amino acids (100%) identical to, and 314 of 314 amino acids (100%) similar to, a gi|17445344|ref|XP_{—}060558.1|XM_{—}060558 protein from Homo sapiens (Human) (similar to OLFACTORY RECEPTOR) (E=e−¹⁶⁴).

NOV8 is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to SeqCalling sources, Public EST sources, Literature sources, and/or RACE sources.

NOV8a and NOV8b are very closely homologous as is shown in the amino acid alignment in Table 8E.

TABLE 8E
Amino Acid Alignment of NOV8a and NOV8b

| | |
|---|---|
| | 10 20 30 40 50 |
| | ....\|....\|....\|....\|....\|.....\|....\|....\|....\|....\| |
| NOV8a CG56663-01 | |
| NOV8b CG56663-02 | |
| | 60 70 80 90 100 |
| | ....\|....\|....\|....\|....\|....\|....\|....\|....\|....\| |
| NOV8a CG56663-01 | |
| NOV8b CG56663-02 | |
| | 110 120 130 140 150 |
| | ....\|....\|....\|....\|....\|....\|....\|....\|....\|....\| |
| NOV8a CG56663-01 | |
| NOV8b CG56663-02 | |
| | 160 170 180 190 200 |
| | ....\|....\|....\|....\|....\|....\|....\|....\|....\|....\| |
| NOV8a CG56683-01 | E |
| NOV8b CG56663-02 | D |
| | 210 220 230 240 250 |
| | ....\|....\|....\|....\|....\|....\|....\|....\|....\|....\| |
| NOV8a CG56663-01 | KK |
| NOV8b 8G56663-02 | AV |
| | 260 270 280 290 300 |
| | ....\|....\|....\|....\|....\|....\|....\|....\|....\|....\| |
| NOV8a CG56663-01 | |
| NOV8b CG56663-02 | |
| | 310 |
| | ....\|....\|.... |
| NOV8a CG56663-01 | |
| NOV8b CG56663-02 | |

Homologies to any of the above NOV8 proteins will be shared by the other NOV8 proteins insofar as they are homologous to each other as shown above. Any reference to NOV8 is assumed to refer to both of the NOV8 proteins in general, unless otherwise noted.

The SignalP, Psort and/or Hydropathy results predict that a NOV8 has a signal peptide and is likely to be localized to the plasma membrane with a certainty of 0.6000. In alternative embodiments, a NOV8 polypeptide is located to the Golgi body with a certainty of 0.4000, the endoplasmic reticulum (membrane) with a certainty of 0.3000, or the microbody (peroxisome) with a certainty of 0.3000. The SignalP predicts a likely cleavage site for a NOV8 peptide between amino acid positions 41 and 42, i.e., at the dash in the sequence LVG-NT.

NOV8a also has homology to the amino acid sequences shown in the BLASTP data listed in Table 8F.

TABLE 8F
BLAST results for NOV8a

| Gene Index/ | Protein/ | Length | Identity | Positives | |
|---|---|---|---|---|---|
| Identifier | Organism | (aa) | (%) | (%) | Expect |
| gi\|17445344\|ref\|XP_ | similar to | 314 | 314/314 | 314/314 | e−164 |
| 060558.1\| | olfactory | | (100%) | (100%) | |
| (XM_060558) | receptor (H. | | | | |
| | sapiens) | | | | |
| | [Homo sapiens] | | | | |
| gi\|5901478\|gb\| | olfactory | 237 | 194/237 | 215/237 | 2e-99 |
| AAD55304.1\|AF044033_1 | receptor | | (81%) | (89%) | |
| (AF044033) | [Marmota | | | | |
| | marmota] | | | | |
| gi\|13624329\|ref\|NP_ | olfactory | 320 | 184/305 | 236/305 | 1e-94 |
| 112165.1\| | receptor, | | (60%) | (77%) | |
| (NM_030903) | family 2, | | | | |
| | subfamily W, | | | | |
| | member 1 | | | | |
| | [Homo sapiens] | | | | |
| gi\|12054431\|emb\| | olfactory | 320 | 184/305 | 236/305 | 1e-94 |
| CAC20523.1\|(AJ302603) | receptor | | (60%) | (77%) | |
| | [Homo sapiens] | | | | |
| gi\|12054429\|emb\| | olfactory | 320 | 184/305 | 236/305 | 2e-94 |
| CAC20522.1\|(AJ302602) | receptor | | (60%) | (77%) | |
| | [Homo sapiens] | | | | |

The homology of these sequences is shown graphically in the ClustalW analysis shown in Table 8G.

TABLE 8G
ClustalW Analysis for NOV8a

1) NOV8a (SEQ ID NO:26)
2) NOV8b (SEQ ID NO:28)
3) gi|17443344 ref XP C60558.1 (XM_060553) similar to olfactory receptor (H.
sapiens) [Homo sapiens] (SEQ ID NO:72)
4) gi|590l478|gbAAD55304.1 AF044033 1 (AF044033) olfactory receptor [Marmota
marmota] (SEQ ID NO:73)
5) gi|13624329 ref NP 112165.1 (NM_030903) olfactory receptor, family 2, sub-
family W, member 1 [Homo sapiens] (SEQ ID NO:74)
6) gi 12054431 emc CAC20523.1 (AJ302603) olfactory receptor [Homo sapiens] (SEQ
ID NO:75)
7) gi/12054429 emb CAC20522.1| (AJ302602) olfactory receptor [Homo sapiens] (SEQ
ID NO:76)

| | |
|---|---|
| | 10 20 30 40 50 |
| | ....\|....\|....\|. ..\|....\|....\| ...\|.. .\| . \|...\| |
| NOV8a Cura 559 CG56663-01 | GT G TQTH DR HL R FV IL A L T V R |
| Nov8b Cura-559B CG56663-02 | GT G TQTH DR HL R FV IL A L T V R |
| gi\|17445344\| | GT G TQTH DR HL R FV IL A L T V R |
| gi\|5901478\| | -------------------------------------------------- |
| gi\|13624329\| | QS Y SLHG NH KM M SG VA F I A A L |
| gi\|12054429\| | QS Y SLHG NH KM M SG VA F I A A L |
| gi\|12054429\| | QS Y SLHG NH KM M SG VA F I A A L |
| | 60 70 80 90 100 |
| | ....\|....\|....\|....\|....\|....\|....\|....\|....\|....\| |
| NOV8a Cura 559 CG56663-01 | PH AH S S L Y N C M A |
| NOV8bS Cura-559B CG56663-02 | PH AH S S L Y N C M A |
| gi\|17445344\| | PH AH S S L Y N C M A |
| gi\|5901478\| | ------- L GN S S L H S R V VV |
| gi\|13624329\| | SQ RN C I M V W P V I |
| gi\|12054431\| | SQ RN C I V V W P V I |
| gi\|12054429\| | SQ RN C I M V W P V I |
| | 110 120 130 140 150 |
| | ....\|....\|....\|....\|....\|....\|....\|....\|....\|....\| |
| NOV8a Cura 559 CG56663-01 | FLFLG G A CV M I R RGLVSVT G |
| NOV8b Cura-559B CG56663-02 | FLFLG G A CV M I R RGLVSVT G |
| gi\|17445344\| | FLFLG G A CV M I R RGLVSVT G |
| gi\|5901478\| | FLFLG G A FV V T I SSR LGLVSVA G |
| gi\|13624329\| | YVYMW S S FT F V H LKMIIMI S |
| gi12054431\| | YVYMW S S FT F V H LKMIIMI S |
| gi\|12054429\| | YVYMW S S FT F V H LKMIIMI S |
| | 160 170 180 190 200 |
| | ....\|....\|....\|....\|....\|....\|....\|....\|....\|....\| |
| NOV8a Cura 559 CG56663-01 | CGV LAMSPV R R HHEV R M IRM S VAI GT |
| NOV8b Cura-559B CG56663-02 | CGV LAMSPV R R HHEV R M IRM S VAIDGT |
| gi\|17445344\| | CGV LAMSPV R R HHEV R M IRM S VAI GT |
| gi\|5901478\| | CGM LVMSPV Q R HNKV C M IRM N VAI GT |
| gi\|13624329\| | ISL VVLCTL N T NNIL C L VKI D TTV MS |
| gi\|12054431\| | ISL VVLCTL N T NNIL C L VKI D TTV MS |
| gi\|12054429\| | ISL VVLCTL N T NNIL C L VKI D TTV MS |
| | 210 220 230 240 250 |
| | ....\|....\|....\|....\|....\|....\|....\|....\|....\|....\| |
| NOV8a Cura 559 CG56663-01 | V KKGV S VF L S VR QIR ASGRQ FG L |
| NOV8b Cura 559B CG56663-02 | V AVGV S VF L S VR QIR ASGRQ FG L |
| gi\|17445344\| | V KKGV S VF L S VR QIR ASGRQ FG L |
| gi\|5901478\| | V AVGI S VF V GH VR FRIQ SSGRHRIFN L |
| gi\|13624329\| | A GIII T IL I G AK RTK KASQR MN M |
| gi\|12054431\| | A GIII T IL I G AK RTK KASQR MN M |
| gi\|12054429\| | A GIII T IL I G AK RTK KASQR MN M |
| | 260 270 280 290 300 |
| | ....\|....\|....\|....\|....\|....\|....\|....\|....\|....\| |
| NOV8a Cura 559 CG56663-01 | N M ASS Q M M NIV L REV G |
| NOV8b Cura-559B CG56663-02 | N M ASS Q M M NIV L REV G |
| gi\|17445344\| | N M ASS Q M M NIV L REV G |
| gi\|5901478\| | N M SRS Q K T NIV L F S ------ |
| gi\|13624329\| | T L NRA K K T TVI S KDM D |
| gi\|12054431\| | T L NRA K K T TVI S KDM D |
| gi\|12054429\| | T L NRA K K T TVI S KNM D |
| | 310 320 |
| | ....\|....\|....\|....\| |
| NOV8a Cura 559 CG56663-01 | GR LLGKRELG E------ |
| NOV8b Cura-559B CG56663-02 | GR LLGKRELG E------ |
| gi\|17445344\| | GR LLGKRELG E------ |
| gi\|5901478\| | -------------------- |
| gi\|13624329\| | KK MRFHHKST IKRNCKS |
| gi\|12054431\| | KK MRFHHKST IKRNCKS |
| gi\|12054429\| | KK MRFHHKST IKRNCKS |

Table 8H lists the domain description from DOMAIN analysis results against NOV8. This indicates that the NOV8 sequence has properties similar to those of other proteins known to contain these domains.

TABLE 8H
Domain Analysis of NOV8

gnl Pfam pfam00001, 7tm_1, 7 transmembrane receptor (rhodopsin family).
(SEQ ID NO:77)
CD-Length=254 residues, 100.0% aligned
Score = 95.1 bits (235), Expect = 5e−21

| | | | |
|---|---|---|---|
| NOV 8: | 41 | GNTTIILVSRLDPHLHTPMYFFLAHLSFLDLSFTTSSIPQLLYNLNGCDKTISYMGCAIQ | 100 |
| | | \|\| +\|\|\| \| \|\| \|\| +\|+ \|\| + \| \|\| \| \| \| + | |
| Sbjct: | 1 | GNLLVILVILRTKKLRTPTNIFLLNLAVADLLFLLTLPPWALYYLVGGDWVFGDALCKLV | 60 |
| NOV8: | 101 | LFLFLGLGGVECLLLAVMAYDRCVAICKPLHYMVIMNPRLCRGLVSVTWGCGVANSLAMS | 160 |
| | | \|\|+ \| \|\|\| ++ \|\| +\|\| \|\| \| \| \|\| + \|++ \| + \|\| | |
| Sbjct: | 61 | GALFVVNGYASILLLTAISIDRYLAIVHPLRYRRIRTPRRAKVLILLVWVLALLLSLP-- | 118 |
| NOV8: | 161 | PVTLRLPRCGHHEVDHFLREMPALIRMACVSTVAIEGTVFVLKKGVVLSPLVFILLSYSY | 220 |
| | | \|+ \| + + \|\|\| \|\|+ \|\|+ \|+ | |
| Sbjct: | 119 | PLLFSWLRTVEEGNTTVCLIDFPEESVKRSYVLLSTLVGFVL-------PLLVILVCYTR | 171 |
| NOV 8: | 221 | IVRAV---------LQIRSASGRQKAFGTCGSHLTVVSLFYG----NIIYMYMQPGASSS | 267 |
| | | \|+\| + \|+ \|\|+\| + \| + \| + ++ | |
| Sbjct: | 172 | ILRTLRKRARSQRSLKRRSSSERKAAKMLLVVVVVFVLCWLPYHIVLLLDSLCLLSIWRV | 231 |
| NOV8: | 268 | QDQGMFLMLFYNIVTPLLNPLIY 290 | |
| | | + + \|+ \| \|\|\|+\|\| | |
| Sbjct: | 232 | LPTALLITLWLAYVNSCLNPIIY 254 | |

G-Protein Coupled Receptor (GPCRs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of various signals. Previously, GPCR genes cloned in different species were from random locations in the respective genomes. The human GPCR genes are intron less and belong to four different gene subfamilies, displaying great sequence variability. These genes are dominantly expressed in olfactory epithelium.

Olfactory receptors (ORs) have been identified as extremely large subfamily of G protein-coupled receptors in a number of species. These receptors share a seven transmembrane domain structure with many neurotransmitter and hormone receptors, and are likely to underlie the recognition and G-protein-mediated transduction of odorant signals. Previously, OR genes cloned in different species were from random locations in the respective genomes. The human OR genes are intron less and belong to four different gene subfamilies, displaying great sequence variability. These genes are dominantly expressed in olfactory epithelium.

The protein similarity information, expression pattern, and map location for the NOV8 proteins and nucleic acids disclosed herein suggest that it may have important structural and/or physiological functions characteristic of the GPCR family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The NOV8 nucleic acid and protein are useful in potential diagnostic and therapeutic applications implicated in various diseases and disorders described below and/or other pathologies. For example, the compositions of the present invention will have efficacy for treatment of patients suffering from: developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders; Cell Shape disorders, Feeding disorders; control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy(DRPLA) Hypophosphatemic rickets, autosomal dominant (2) Acrocallosal syndrome and dyskinesias, such as Huntington's disease or Gilles de la Tourette syndrome and/or other pathologies and disorders of the like.. The polypeptides can be used as immunogens to produce antibodies specific for the invention, and as vaccines. They can also be used to screen for potential agonist and antagonist compounds. For example, a cDNA encoding the OR-like protein may be useful in gene therapy, and the OR-like protein may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The novel nucleic acid encoding OR-like protein, and the OR-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.and other diseases, disorders and conditions of the like.

NOV9

A disclosed NOV9 is nucleic acid (designated as CuraGen Acc. No. CG56787-01, encodes a novel dual specificity phosphatase and includes the 624 nucleotide sequence (SEQ ID NO: 29) shown in Table 9A. An open reading frame for the mature protein was identified beginning at nucleotide 1 and ending with a TAA codon at nucleotides 805-807. Putative untranslated regions downstream from the termination codon are underlined in Table 9A, and the stop codon is in bold letters.

TABLE 9A
NOV9 Nucleotide Sequence

| | |
|---|---|
| CTTTGAGCTTCTCTGACTGCTGACCACTGACCCACCGACTTGATGACAGCACCCTCGTGTGCCTTCC | (SEQ ID NO:29) |
| CAGTTCAAATCCGGCAGCCCTCAGTCAGCGGCCTCTCGCAGATAACCAAAAGCCTGTATATCAGCA | |
| ATGGTGTGGCCGCCAACAACAAGCTCATGCTGTCTAGCAACCAGATCACCATGGTCATCAATGTCTC | |
| AGTGGAGGTAGTGAACACCTTGTATGAGGATATCCAGTACATGCAGGTACCTGTGGCTGACTCCCC | |
| TAACTCACGTCTCTGTGACTTCTTTGACCCTATTGCTGACCATATCCACAGCGTGGAGATGAAGCAG | |
| GGCCGTACTTTGCTGCACTGTGCTGCTGGTGTGAGCCGCTCAGCTGCCCTGTGCCTCGCCTACCTCA | |
| TGAAGTACCACGCCATGTCCCTGCTGGACGCCCACACGTGGACCAAGTCATGCCGGCCCATCATCC | |
| GACCCAACAGCGGCTTTTGGGAGCAGCTCATCCACTATGAGTTCCAATTGTTTGGCAAGAACACTGT | |
| GCACATGGTCAGTTCCCCAGTGGGAATGATCCCTGACATCTATGAGAAGGAAGTCCGTTTGATGATT | |
| CCACTGTGAGCCATCCCACGAGCC | |

The nucleic acid sequence of NOV9 maps to chromosome 22 and has 363 of 563 bases (64%) identical to a gb:GENBANK-ID:AF120032|acc:AF120032.1 mRNA from Homo sapiens (Homo sapiens MAP kinase phosphatase 6 (MKP6) mRNA, complete cds).

The NOV9 polypeptide (SEQ ID NO: 30) is 188 amino acid residues in length and is presented using the one-letter amino acid code in Table 9B. The SignalP, Psort and/or Hydropathy results predict that NOV9 has a signal peptide and is likely to be localized to the cytoplasm with a certainty of 0.4500. In alternative embodiments, a NOV9 polypeptide is located to the microbody (peroxisome) with a certainty of 0.3000, the lysosome (lumen) with a certainty of 0.1955, or the mitochondrial matrix space with a certainty of 0.1000.

TABLE 9B
Encoded NOV9 Protein Sequence

| | |
|---|---|
| MTAPSCAFPVQIRQPSVSGLSQITKSLYISNGVAANNKLMLSSNQITMVINVSVEVVNTLYEDIQYMQ | (SEQ ID NO:30) |
| VPVADSPNSRLCDFFDPIADHIHSVEMKQGRTLLHCAAGVSRSAALCLAYLMKYHAMSLLDAHTWT | |
| KSCRPIIRPNSGFWEQLIHYEFQLFGKNTVHMVSSPVGMIPDIYEKEVRLMIPL | |

The NOV9 amino acid sequence has 187 of 188 amino acid residues (99%) identical to, and 187 of 188 amino acid residues (99%) similar to, the 188 amino acid residue gi|17485142|ref|XP_{—}038481 .2|XM_{—}038481 protein from Homo sapiens (Human) (HYPOTHETICAL PROTEIN XP_{—}038481) (E=e⁻¹⁰²).

NOV9 is expressed in at least the following tissues: Brain, Brown adipose, Cartilage, Colon, Dermis, Epidermis, Hair Follicles, Hippocampus, Hypothalamus, Kidney, Lung, Lymph node, Lymphoid tissue, Ovary, Oviduct/Uterine Tube/Fallopian tube, Parotid Salivary glands, Peripheral Blood, Pituitary Gland, Prostate, Right Cerebellum, Skin, Substantia Nigra, Testis, Thyroid, Tonsils, Umbilical Vein, Uterus, Vulva, Whole Organism. Expression information was derived from the tissue sources of the sequences that were included in the derivation of the sequence of NOV9.The sequence is predicted to be expressed in the following tissues because of the expression pattern of (GENBANK-ID: gb:GENBANK-ID:AF120032|acc:AF120032.1) a closely related Homo sapiens MAP kinase phosphatase 6 (MKP6) mRNA, complete cds homolog in species Homo sapiens: breast and ovarian tissue, pancreas, brain, liver, kidney, spleen, testis, ovary, and peripheral blood leukocytes.

NOV9 has homology to the amino acid sequences shown in the BLASTP data listed in Table 9C.

TABLE 9C
BLAST results for NOV9

| Gene Index/ | Protein/ | Length | Identity | Positives | |
|---|---|---|---|---|---|
| Identifier | Organism | (aa) | (%) | (%) | Expect |
| gi\|17485142\|ref\|XP_ | hypothetical | 188 | 187/188 | 187/188 | e−102 |
| 038481.2\| | protein | | (99%) | (99%) | |
| (XM_038481) | XP_038481 | | | | |
| | [Homo sapiens] | | | | |
| gi\|18043293\|gb\| | Unknown | 188 | 156/188 | 171/188 | 4e-86 |
| AAH20036.1\|AAH20036 | (protein for | | (82%) | (89%) | |
| (BC020036) | MGC:28218) | | | | |
| | [Mus | | | | |
| | musculus] | | | | |
| gi\|13278657\|gb\| | Unknown | 151 | 148/148 | 148/148 | 2e-81 |
| AAH04110.1\|AAH04110 | (protein for | | (100%) | (100%) | |
| (BC004110) | IMAGE: | | | | |
| | 3689593) | | | | |
| | [Homo sapiens] | | | | |
| gi\|12840422\|dbj\| | putative [Mus | 189 | 137/186 | 158/186 | 1e-76 |
| BAB24847.1\|(AK007061) | musculus] | | (73%) | (84%) | |
| gi\|10334445\|emb\| | bA386N14.1 | 190 | 131/190 | 164/190 | 6e-72 |
| CAC10195.1\|(AL133545) | (novel | | (68%) | (85%) | |
| | protein | | | | |
| | similar to a | | | | |
| | dual | | | | |
| | specificity | | | | |
| | phosphatase) | | | | |
| | [Homo sapiens] | | | | |

The homology of these sequences is shown graphically in the ClustalW analysis shown in Table 9D.

TABLE 9D
ClustalW Analysis of NOV9

1) NOV9 (SEQ ID NO:38)
2) gi 27485142 ref XP 038481.2 (XM_038481) hypothetical protein
XP_038481 [Homo sapiens] (SEQ ID NO:78)
3) gi|18043293 gb|AAH20036.1 AAH20036 (BC020036) Unknown (protein
for MGC:28218) [Mus musculus] (SEQ ID NO:79)
4) gi|13278657|gb|AAH04110.1 (BC004110) Unknown (protein for
IMAGE:3689593) [Homo sapiens] (SEQ ID NO:80)
5) gi|12840422 dbj BAB24847.1| (AK007061) putative [Mus
musculus] (SEQ ID NO:81)
6) gi|10334445 emb CAC10195.1| (AL133545) bA386N14.1 (novel pro-
tein similar to a dual specificity phosphatase) [Homo sapiens]
(SEQ ID NO:82)

| | |
|---|---|
| | 10 20 30 40 50 |
| | ....\|....\|....\|....\|....\|....\|....\|...\|....\|....\| |
| NOV9 | APSCA -V IR P-VS K Y N M M |
| gi\|17485142\| | APSCA -V FR P- VS K Y N M M |
| gi\|18043293\| | SPWSA -V IP P- IR K F N L T |
| gi\|13278657\| | -----------------------------------RG---- M |
| gi\|12840422\| | TASCI -S AT QDNIY A F SAV D T N H T |
| gi\|10334445\| | ASASS SSS GV QP IYSF R FL D L R A |
| | 60 70 80 90 100 |
| | ....\|....\|....\|....\|....\|....\|....\|....\|....\|....\| |
| NOV9 | L M A S C K |
| gi\|17485142\| | L M A S C K |
| gi\|18043293\| | A F V V A VA SN SV R QK |
| gi\|13278657\| | L M A S C K |
| gi\|12840422\| | I A FF V S A Y Y G RN |
| gi\|10334445\| | IV A VFF G IK T ARD Y L TID R |
| | 110 120 130 140 150 |
| | ....\|....\|....\|....\|....\|....\|....\|....\|....\|....\| |
| NOV9 | |
| gi\|17483142\| | |
| gi\|18043293\| | V |
| gi\|13278637\| | |
| gi\|12840422\| | T N T T N |
| gi\|10334445\| | M S S R N |
| | 160 170 180 190 |
| | ....\|....\|....\|....\|....\|....\|....\|....\| |
| NOV9 | H VS M V |
| gi\|17485142\| | H VS M V |
| gi\|18043293\| | L MQ MD M R T |
| gi\|13278657\| | H VS M V |
| gi\|12840422\| | K SR R IY I L N AY ELM |
| gi\|10334445\| | N K NN R IN N DL M SM |

Tables 9E, 9F and 9G list the domain description from DOMAIN analysis results against NOV9. This indicates that the NOV9 sequence has properties similar to those of other proteins known to contain these domains.

TABLE 9E
Domain Analysis of NOV9

gnl Smart smart00195ee DSPc, Dual specificity phosphatase, catalytic domain
(SEQ ID NO:83)
CD-Length = 139 residues, 100.0% aligned
Score=134 bits (336), Expect = 6e−33

| | | | |
|---|---|---|---|
| NOV 9: | 19 | GLSQITKSLYISNGVAANNKLMLSSNQITMVINVSVEVVNTLYEDIQYMQVPVADSPNSR | 78 |
| | | \| \|+\| \|\|+ + \|+\| +\| \|\| + \|\| \|+ \|+ +\|\| \|+ ++ | |
| Sbjct: | 1 | GPSEILPHLYLGSYSDASNLALLKKLGITHVINVTEEVPNSNKSGFLYLGIPVDDNTETK | 60 |
| NOV 9: | 79 | LCDFFDPIADHIHSVEMKQGRTLLHCAAGVSRSAALCLAYLMKYHAMSLLDAHTWTKSCR | 138 |
| | | + + + \| \| \| \|+\|+\|\| \|\|\|\|\|\|\| \| +\|\|\|\|\|\| \|\|\| \|\|+ + \| \| | |
| Sbjct: | 61 | ISPYLPEAVEFIEDAEKKGGKVLVHCQAGVSRSATLIIAYLMKYRNMSLNDAYDFVKEER | 120 |
| NOV9: | 139 | PIIRPNSGFWEQILIHYEFQ 157 | |
| | | \|\|\| \|\| \|\| \|\|\| \|\| + | |
| Sbjct: | 121 | PIISPNFGPLRQLIEYERK 139 | |

TABLE 9F
Domain Analysis of NOV9

gnlPfam|pfam00782, DSPc, Dual specificity phosphatase, catalytic domain.
Ser/Thr and Tyr protein phosphatases. The enzyme's tertiary fold is highly
similar to that of tyrosine-specific phosphatases, except for a “recogni-
tion” region.
(SEQ ID NO:84)
CD-Length = 139 residues, 100.0% aligned
Score = 134 bits (336), Expect=6e−33

| | | | |
|---|---|---|---|
| NOV 9: | 19 | GLSQITKSLYISNGVAANKLMLSSNQITMVINVSVEVVNTLYEDIQYMQVPVADSPNSR | 78 |
| | | \| \|+\| \|\|+ + \|+\| \|\| \|\| \|\|\|\|+ \|\| \|+ \|+ +\|\| \|+ + | |
| Sbjct: | 1 | GPSEILPHLYLGSYPTASNLAFLSKLGITHVINVTEEVPNSKNSGFLYLHIPVDDNHETD | 60 |
| NOV 9: | 79 | LCDFFDPIADMIHSVEMKQGRTLLHCAAGVSRSAALCLAYLMKYHAMSLLDAETWTKSCR | 138 |
| | | + + \| + \| \| \|+ \|+\|\| \|\|+\|\|\|\| \| +\|\|\|\|\| +\|\| +\|+++ \| \| | |
| Sbjt: | 61 | ISPYLDEAVEFIEDARQKGGKVLVHCQAGISRSATLIIAYLMKTRNLSLNEAYSFVKERR | 120 |
| NOV 9: | 139 | PIIRPNSGFWEQLIHYEFQ 157 | |
| | | \|\|\| \|\| \|\| \|\|\| \|\| + | |
| Sbjct: | 121 | PIISPNFGFKRQLIEYERK 139 | |

TABLE 9G
Domain Analysis of NOV9

gnl Smart smart00194, PTPc, Protein tyrosine phos-
phatase, catalytic domain (SEQ ID NO:85)
CD-Length = 264 residues, 12.5% aligned
Score = 35.0 bits (79), Expect = 0.004

| | | | |
|---|---|---|---|
| NOV 9: | 88 | DHIHSVEMKQGRTLLHCAAGVSRSAALCLAYLM | 120 |
| | | \| \| ++\|\|+\|\|\| \|+ ++ | |
| Sbjct: | 187 | RKSQSTLRNSGPIVVHCSAGVGRTGTFIAIDIL | 219 |

Mitogen-activated protein (MAP) kinase phosphatases constitute a growing family of dual specificity phosphatases thought to play a role in the dephosphorylation and inactivation of MAP kinases and are therefore likely to be important in the regulation of diverse cellular processes such as proliferation, differentiation, and apoptosis. For this reason it has been suggested that MAP kinase phosphatases may be tumor suppressors. DUSP6 (alias PYST1), one of the dual-specificity tyrosine phosphatases, is localized on 12q21, one of the regions of frequent allelic loss in pancreatic cancer. This gene is composed of three exons, and two forms of alternatively spliced transcripts are ubiquitously expressed. Although no mutations were observed in 26 pancreatic cancer cell lines, reduced expressions of the full-length transcripts were observed in some cell lines, which may suggest some role for DUSP6 in pancreatic carcinogenesis. PMID: 9858808

The mitogen-induced gene, DUSP2, encodes a nuclear protein, PAC1, that acts as a dual-specific protein phosphatase with stringent substrate specificity for MAP kinase. MAP kinase phosphorylation and consequent enzymatic activation is a central and often obligatory component in signal transduction initiated by growth factor stimulation or resulting from various types of oncogenic transformation. DUSP2 downregulates intracellular signal transduction through the dephosphorylation/inactivation of MAP kinases. PMID: 7590752

Keyse and Emslie (1992) isolated and characterized a cDNA, which they designated CL100, corresponding to an mRNA that is highly inducible by oxidative stress and heat shock in human skin cells. The cDNA was obtained by differential screening of a library made from normal human skin fibroblasts stressed for 2 hours in a solution of hydrogen peroxide. The cDNA contains an open reading frame specifying a 367-residue protein of 39.3 kD predicted molecular mass with the structural features of a nonreceptor type protein-tyrosine phosphatase. It has significant amino acid sequence similarity to a tyr/ser-protein phosphatase encoded by the late gene H1 of vaccinia virus. The purified protein encoded by the open reading frame expressed in bacteria has intrinsic phosphatase activity. Given the relationship between the levels of protein-tyrosine phosphorylation, receptor activity, cellular proliferation, and cell-cycle control, Keyse and Emslie (1992) concluded that induction of this gene may play an important regulatory role in the human cellular response to environmental stress. Alessi et al. ( 1993) found that the phosphatase encoded by CL100 has dual specificity for tyrosine and threonine and that it specifically inactivates mitogen-activated protein kinase in vitro. Brondello et al. (1999) determined that DUSP1, which they called MKP1, is a labile protein with a half-life of approximately 45 minutes in CCL39 hamster fibroblasts. Its degradation was attenuated by inhibitors of the ubiquitin-directed proteasome complex. MKP1 was a target in vivo and in vitro for p42MAPK (176948) or p44MAPK (601795), which phosphorylates MKP1 on 2 C-terminal serine residues, ser359 and ser364. This phosphorylation did not modify MKP1's intrinsic ability to dephosphorylate p44MAPK, but led to stabilization of the protein. Brondello et al. (1999) concluded that these results illustrated the importance of regulated protein degradation in the control of mitogenic signaling.

The protein similarity information, expression pattern, and map location for the NOV9 protein and nucleic acid disclosed herein suggest that it may have important structural and/or physiological functions characteristic of the family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: brain disorders including epilepsy, eating disorders, schizophrenia, ADD, and cancer; heart disease; blood disorders, kidney disorders, liver diseases, inflammation and autoimmune disorders including Crohn's disease, IBD, allergies, rheumatoid and osteoarthritis, inflammatory skin disorders, allergies, blood disorders; psoriasis; colon-, ovarian-, testicular-, lymphatic-, brain-, and pancreatic cancers; leukemia AIDS; thalamus disorders; metabolic disorders including diabetes and obesity; lung diseases such as asthma, emphysema, cystic fibrosis, and cancer; pancreatic disorders including pancreatic insufficiency; and prostate disorders including prostate cancer and other diseases, disorders and conditions of the like.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS: 1, 3, 5, 7, 9, 11, 13, 15, 17, 19, 21, 23, 25, 27 and 29, or a complement of this aforementioned nucleotide sequence, can be isolated using standard molecular biology techniques and the sequence information provided herein. Using all or a portion of the nucleic acid sequence of SEQ ID NOS: 1, 3, 5, 7, 9, 11, 13, 15, 17, 19, 21, 23, 25, 27 and 29 as a hybridization probe, NOVX molecules can be isolated using standard hybridization and cloning techniques (erg., as described in Sambrook, el al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS: 1, 3, 5, 7, 9, 11, 13, 15, 17, 19, 21, 23, 25, 27 and 29, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 1, 3, 5, 7, 9, 11, 13, 15, 17, 19, 21, 23, 25, 27 and 29, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown NOS: 1, 3, 5, 7, 9, 11, 13, 15, 17, 19, 21, 23, 25, 27, 29, 31, 33, 35, 37, 39 or 41 is one that is sufficiently complementary to the nucleotide sequence shown NOS: 1, 3, 5, 7, 9, 11, 13, 15, 17, 19, 21, 23, 25, 27, 29, 31, 33, 35, 37, 39 or 41 that it can hydrogen bond with little or no mismatches to the nucleotide sequence shown SEQ ID NOS: 1, 3, 5, 7, 9, 11, 13, 15, 17, 19, 21, 23, 25, 27 and 29, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic aids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS: 1, 3, 5, 7, 9, 11, 13, 15, 17, 19, 21, 23, 25, 27 and 29, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS: 1, 3, 5, 7, 9, 11, 13, 15, 17, 19, 21, 23, 25, 27 and 29; or an anti-sense strand nucleotide sequence of SEQ ID NOS: 1, 3, 5, 7, 9, 11, 13, 15, 17, 19, 21, 23, 25, 27 and 29; or of a naturally occurring mutant of SEQ ID NOS: 1, 3, 5, 7, 9, 11, 13, 15, 17, 19, 21, 23, 25, 27 and 29.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS: 1, 3, 5, 7, 9, 11, 13, 15, 17, 19, 21, 23, 25, 27 and 29, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS: 1, 3, 5, 7, 9, 11, 13, 15, 17, 19, 21, 23, 25, 27 and 29due to degeneracy of the genetic code and thus encode the same NOVX proteins as that encoded by the nucleotide sequences shown in SEQ ID NOS: 1, 3, 5, 7, 9, 11, 13, 15, 17, 19, 21, 23, 25, 27 and 29. In another embodiment, an isolated nucleic acid molecule of the invention has a nucleotide sequence encoding a protein having an amino acid sequence shown in SEQ ID NOS: 2, 4, 6, 8, 10, 12, 14, 16, 18, 20, 22, 24, 26, 28 and 30.

In addition to the human NOVX nucleotide sequences shown in SEQ ID NOS: 1, 3, 5, 7, 9, 11, 13, 15, 17, 19, 21, 23, 25, 27 and 29, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS: 1 3, 5, 7, 9, 11, 13, 15, 17, 19, 21, 23, 25, 27 and 29 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS: 1, 3, 5, 7, 9, 11, 13, 15, 17, 19, 21, 23, 25, 27 and 29. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS: 1, 3, 5, 7, 9, 11, 13, 15, 17, 19, 21, 23, 25, 27 and 29, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS: 1, 3, 5, 7, 9, 11, 13, 15, 17, 19, 21, 23, 25, 27 and 29, or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, N.Y., and Kriegler, 1990; GENE TRANSFER AND EXPRESSION, A LABORATORY MANUAL, Stockton Press, N.Y.

In a third embodiment, a nucleic acid that is hybridizable to the nucleic acid molecule comprising the nucleotide sequences SEQ ID NOS: 1, 3, 5, 7, 9, 11, 13, 15, 17, 19, 21, 23, 25, 27 and 29, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (H 7.4), 5 mM EDTA, and 0.1% SDS at 50° C. Other conditions of low stringency that may be used are well known in the art (e.g., as employed for cross-species hybridizations). See, e.g. Ausubel, et al. (eds.), 1993, CURRENT PROTOCOLS IN MOLECULAR BIOLOGY, John Wiley & Sons, N.Y., and Kriegler, 1990, GENE TRANSFER AND EXPRESSION, A LABORATORY MANUAL, Stockton Press, N.Y.; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS: 1, 3, 5, 7, 9, 11, 13, 15, 17, 19, 21, 23, 25, 27 and 29, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS: 2, 4, 6, 8, 10, 12, 14, 16, 18, 20, 22, 24, 26, 28 and 30. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS: 1, 3, 5, 7, 9, 11, 13, 15, 17, 19, 21, 23, 25, 27 and 29 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS: 2, 4, 6, 8, 10, 12, 14, 16, 18, 20, 22, 24, 26, 28 and 30. Preferably, the protein encoded by the nucleic acid molecule is at least about 60% homologous to SEQ ID NOS: 2, 4, 6, 8, 10, 12, 14, 16, 18, 20, 22, 24, 26, 28 and 30; more preferably at least about 70% homologous SEQ ID NOS: 2, 4, 6, 8, 10, 12, 14, 16, 18, 20, 22, 24, 26, 28 and 30; still more preferably at least about 80% homologous to SEQ ID NOS: 2, 4, 6, 8, 10, 12, 14, 16, 18, 20, 22, 24, 26, 28 and 30; even more preferably at least about 90% homologous to SEQ ID NOS: 2, 4, 6, 8, 10, 12, 14, 16, 18, 20, 22, 24, 26, 28 and 30; and most preferably at least about 95% homologous to SEQ ID NOS: 2, 4, 6, 8, 10, 12, 14, 16, 18, 20, 22, 24, 26, 28 and 30.

An isolated nucleic acid molecule encoding an NOVX protein homologous to the protein of SEQ ID NOS: 2, 4, 6, 8, 10, 12, 14, 16, 18, 20, 22, 24, 26, 28 and 30 can be created by introducing one or more nucleotide substitutions, additions or deletions into the nucleotide sequence of SEQ ID NOS: 1, 3, 5, 7, 9, 11, 13, 15, 17, 19, 21, 23, 25, 27 and 29, such that one or more amino acid substitutions, additions or deletions are introduced into the encoded protein.

Mutations can be introduced into SEQ ID NOS: 1, 3, 5, 7, 9, 11, 13, 15, 17, 19, 21, 23, 25, 27 and 29 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS: 1, 3, 5, 7, 9, 11, 13, 15, 17, 19, 21, 23, 25, 27 and 29, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS: 1, 3, 5, 7, 9, 11, 13, 15, 17, 19, 21, 23, 25, 27 and 29, or fragments, analogs or derivatives thereof An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS: 2, 4, 6, 8, 10, 12, 14, 16, 18, 20, 22, 24, 26, 28 and 30, or antisense nucleic acids complementary to an NOVX nucleic acid sequence of SEQ ID NOS: 1, 3, 5, 7, 9, 11, 13, 15, 17, 19, 21, 23, 25, 27 and 29, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxyrnethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS: 1, 3, 5, 7, 9, 11, 13, 15, 17, 19, 21, 23, 25, 27 and 29). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAS” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS: 2, 4, 6, 8, 10, 12, 14, 16, 18, 20, 22, 24, 26, 28 and 30. The invention also includes a mutant or variant protein any of whose residues may be changed from the corresponding residues shown in SEQ ID NOS: 2, 4, 6, 8, 10, 12, 14, 16, 18, 20, 22, 24, 26, 28 and 30 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS: 2, 4, 6, 8, 10, 12, 14, 16, 18, 20, 22, 24, 26, 28 and 30) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS: 2, 4, 6, 8, 10, 12, 14, 16, 18, 20, 22, 24, 26, 28 and 30. In other embodiments, the NOVX protein is substantially homologous to SEQ ID NOS: 2, 4, 6, 8, 10, 12, 14, 16, 18, 20, 22, 24, 26, 28 and 30, and retains the functional activity of the protein of SEQ ID NOS: 2, 4, 6, 8, 10, 12, 14, 16, 18, 20, 22, 24, 26, 28 and 30, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS: 2, 4, 6, 8, 10, 12, 14, 16, 18, 20, 22, 24, 26, 28 and 30, and retains the functional activity of the NOVX proteins of SEQ ID NOS: 2, 4, 6, 8, 10, 12, 14, 16, 18, 20, 22, 24, 26, 28 and 30.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 11, 13, 15, 17, 19, 21, 23, 25, 27 and 29.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS: 2, 4, 6, 8, 10, 12, 14, 16, 18, 20, 22, 24, 26, 28 and 30, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et a., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with SI nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′}, and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 1:0 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

Fab Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g.

F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, 131I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif), and picZ (Invitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banecji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS: 1, 3, 5, 7, 9, 11, 13, 15, 17, 19, 21, 23, 25, 27 and 29can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS: 1, 3, 5, 7, 9, 11, 13, 15, 17, 19, 21, 23, 25, 27 and 29), but more preferably, is a non-human homologue of a human NOVX gene. For example, a mouse homologue of human NOVX gene of SEQ ID NOS: 1, 3, 5, 7, 9, 11, 13, 15, 17, 19, 21, 23, 25, 27 and 29can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage Pi. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerelvisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries: synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding, of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl—N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et a., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS: 1, 3, 5, 7, 9, 11, 13, 15, 17, 19, 21, 23, 25, 27 and 29, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS: 1, 3, 5, 7, 9, 11, 13, 15, 17, 19, 21, 23, 25, 27 and 29are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS: 1, 3, 5, 7, 9, 11, 13, 15, 17, 19, 21, 23, 25, 27 and 29, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with SI nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

### Identification of NOVX Nucleic Acids

TblastN using CuraGen Corporation's sequence file for polypeptides or homologs was run against the Genomic Daily Files made available by GenBank or from files downloaded from the individual sequencing centers. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The novel NOVX target sequences identified in the present invention were subjected to the exon linking process to confirm the sequence. PCR primers were designed by starting at the most upstream sequence available, for the forward primer, and at the most downstream sequence available for the reverse primer. PCR primer sequences were used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

Physical clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

### Example 2

### Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8) 1249-1265, 2000).

### Example 3

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune diseases), Panel CNSD.01 (containing central nervous system samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First. the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1×TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58′-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No.4324020), following the manufacturer's instructions. PCR amplification was performed as follows: 95° C 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1 .3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1. 1, 1.2 and 1.3D, the following abbreviations are used:

ca.=carcinoma,

*=established from metastasis,

met=metastasis,

s cell var=small cell variant,

non-s=non-sm=non-small,

squam=squamous,

p1. eff=p1 effusion=pleural effusion,

glio=glioma,

astro=astrocytoma, and

neuro=neuroblastoma.

General_Screening_Panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶cells/ml in DMEM 5% FCS (Hyclone), 100 μM non essential amino acids (Gibco), 1 mM sodium pymivate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 nM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_Comprehensive Panel_v1.0

The plates for AI_comprehensive panel_v1.0 include two control wells and 89 test samples comprised of eDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1 anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22 /Rep20=individual patients

RA=Rheumatoid arthritis

Backus=From Backus Hospital

OA=Osteoarthritis

(SS)(BA)(MF)=Individual patients

Adj=Adjacent tissue

Match control=adjacent tissues

−M=Male

−F=Female

COPD=Chronic obstructive pulmonary disease

Panels 5D and 5I

The plates for Panel 5D and 51 include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

| | |
|---|---|
| Patient 2 | Diabetic Hispanic, overweight, not on insulin |
| Patient 7-9 | Nondiabetic Caucasian and obese (BMI > 30) |
| Patient 10 | Diabetic Hispanic, overweight, on insulin |
| Patient 11 | Nondiabetic African American and overweight |
| Patient 12 | Diabetic Hispanic on insulin |

Adipocyte differentiation was induced in donor progenitor cells obtained from Osirus (a division of Clonetics/Bio Whittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multi lineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

A. NOV1a: Delta Serrate Ligand Receptor (Also Known as MEGF)

Expression of the NOV1 a gene (COR87920446_A) was assessed using the primer-probe set Ag3978, described in Table A1. Results of the RTQ-PCR runs are shown in Tables A2, A3 and A4.

TABLE A1

Probe Name Ag3978

| | | | | |
|---|---|---|---|---|
| Primers | Sequences | Length | Start Position | Seq ID No. |

| | | | | |
|---|---|---|---|---|
| Forward | 5′-ctggaccgaagctacagctata-3′ | 22 | 2605 | 86 |
| Probe | TET-5′-atggcccaggcccattctacaataaa-3′-TAMRA | 26 | 2636 | 87 |
| Reverse | 5′-cgagctcctcttcagagatga-3′ | 21 | 2666 | 88 |

TABLE A2
CNS neurodegeneration v1.0

| | Rel. Exp.(%) Ag3978, | | Rel. Exp.(%) Ag3978, |
|---|---|---|---|
| Tissue Name | Run 206880050 | Tissue Name | Run 206880050 |
| AD 1 Hippo | 21.2 | Control (Path) 3 | 17.9 |
| | | Temporal Ctx | |
| AD 2 Hippo | 43.5 | Control (Path) 4 | 42.9 |
| | | Temporal Ctx | |
| AD 3 Hippo | 7.5 | AD 1 Occipital Ctx | 22.4 |
| AD 4 Hippo | 8.9 | AD 2 Occipital Ctx | 0.0 |
| | | (Missing) | |
| AD 5 Hippo | 20.2 | AD 3 Occipital Ctx | 6.4 |
| AD 6 Hippo | 100.0 | AD 4 Occipital Ctx | 8.7 |
| Control 2 Hippo | 13.6 | AD 5 Occipital Ctx | 65.1 |
| Control 4 Hippo | 8.3 | AD 6 Occipital Ctx | 40.3 |
| Control (Path) 3 Hippo | 10.4 | Control 1 Occipital | 6.6 |
| | | Ctx | |
| AD 1 Temporal Ctx | 21.6 | Control 2 Occipital | 20.7 |
| | | Ctx | |
| AD 2 Temporal Ctx | 26.2 | Control 3 Occipital | 18.0 |
| | | Ctx | |
| AD 3 Temporal Ctx | 0.0 | Control 4 Occipital | 4.2 |
| | | Ctx | |
| AD 4 Temporal Ctx | 9.2 | Control (Path) 1 | 36.1 |
| | | Occipital Ctx | |
| AD 5 Inf Temporal Ctx | 24.5 | Control (Path) 2 | 30.6 |
| | | Occipital Ctx | |
| AD 5 Sup Temporal Ctx | 17.7 | Control (Path) 3 | 16.5 |
| | | Occipital Ctx | |
| AD 6 Inf Temporal Ctx | 84.7 | Control (Path) 4 | 60.7 |
| | | Occipital Ctx | |
| AD 6 Sup Temporal Ctx | 79.6 | Control 1 Parietal Ctx | 6.7 |
| Control 1 Temporal Ctx | 2.5 | Control 2 Parietal Ctx | 13.7 |
| Control 2 Temporal Ctx | 17.9 | Control 3 Parietal Ctx | 23.2 |
| Control 3 Temporal Ctx | 17.8 | Control (Path) 1 | 32.8 |
| | | Parietal Ctx | |
| Control 3 Temporal Ctx | 9.2 | Control (Path) 2 | 41.5 |
| | | Parietal Ctx | |
| Control (Path) 1 | 79.6 | Control (Path) 3 | 24.8 |
| Temporal Ctx | | Parietal Ctx | |
| Control (Path) 2 | 23.3 | Control (Path) 4 | 31.0 |
| Temporal Ctx | | Parietal Ctx | |

TABLE A3
General screening panel v1.4

| | Rel. Exp.(%) Ag3978, | | Rel. Exp.(%) Ag3978, |
|---|---|---|---|
| Tissue Name | Run 217525358 | Tissue Name | Run 217525358 |
| Adipose | 3.1 | Renal ca. TK-10 | 5.4 |
| Melanoma* | 3.9 | Bladder | 1.2 |
| Hs688(A).T | | | |
| Melanoma* | 10.0 | Gastric ca. (liver met.) | 0.2 |
| Hs688(B).T | | NCI-N87 | |
| Melanoma* M14 | 0.0 | Gastric ca. KATO III | 0.3 |
| Melanoma* | 3.2 | Colon ca. SW-948 | 0.4 |
| LOXIMVI | | | |
| Melanoma* SK- | 0.0 | Colon ca. SW480 | 1.1 |
| MEL-5 | | | |
| Squamous cell | 1.1 | Colon ca.* (SW480 | 0.1 |
| carcinoma SCC-4 | | met) SW620 | |
| Testis Pool | 2.4 | Colon ca. HT29 | 0.1 |
| Prostate ca.* (bone | 4.8 | Colon ca. HCT-116 | 1.6 |
| met) PC-3 | | | |
| Prostate Pool | 1.1 | Colon ca. CaCo-2 | 0.3 |
| Placenta | 1.8 | Colon cancer tissue | 2.0 |
| Uterus Pool | 1.1 | Colon ca. SW1116 | 0.6 |
| Ovarian ca. OVCAR-3 | 0.1 | Colon ca. Colo-205 | 0.0 |
| Ovarian ca. SK-OV-3 | 92.7 | Colon ca. SW-48 | 0.1 |
| Ovarian ca. OVCAR-4 | 0.1 | Colon Pool | 4.2 |
| Ovarian ca. OVCAR-5 | 11.5 | Small Intestine Pool | 5.6 |
| Ovarian ca. IGROV-1 | 0.1 | Stomach Pool | 92.7 |
| Ovarian ca. OVCAR-8 | 0.5 | Bone Marrow Pool | 1.7 |
| Ovary | 1.1 | Fetal Heart | 1.5 |
| Breast ca. MCF-7 | 0.1 | Heart Pool | 1.3 |
| Breast ca. MDA-MB- | 1.0 | Lymph Node Pool | 6.5 |
| 231 | | | |
| Breast ca. BT 549 | 0.0 | Fetal Skeletal Muscle | 1.8 |
| Breast ca. T47D | 21.3 | Skeletal Muscle Pool | 1.5 |
| Breast ca. MDA-N | 0.0 | Spleen Pool | 2.1 |
| Breast Pool | 100.0 | Thymus Pool | 3.1 |
| Trachea | 1.9 | CNS cancer (glio/astro) | 0.1 |
| | | U87-MG | |
| Lung | 0.5 | CNS cancer (glio/astro) | 0.3 |
| | | U-118-MG | |
| Fetal Lung | 4.9 | CNS cancer (neuro;met) | 3.8 |
| | | SK-N-AS | |
| Lung ca. NCI-N417 | 91.4 | CNS cancer (astro) SF- | 0.0 |
| | | 539 | |
| Lung ca. LX-1 | 1.1 | CNS cancer (astro) | 0.2 |
| | | SNB-75 | |
| Lung ca. NCI-H146 | 0.0 | CNS cancer (glio) SNB- | 0.0 |
| | | 19 | |
| Lung ca. SHP-77 | 0.1 | CNS cancer (glio) SF- | 0.0 |
| | | 295 | |
| Lung ca. A549 | 0.5 | Brain (Amygdala) Pool | 0.3 |
| Lung ca. NCI-H526 | 0.0 | Brain (cerebellum) | 0.3 |
| Lung ca. NCI-H23 | 0.1 | Brain (fetal) | 0.8 |
| Lung ca. NCI-H460 | 88.3 | Brain (Hippocampus) | 0.6 |
| | | Pool | |
| Lung ca HOP-62 | 0.5 | Cerebral Cortex Pool | 0.5 |
| Lung ca. NCI-H522 | 0.3 | Brain (Substantia nigra) | 0.8 |
| | | Pool | |
| Liver | 0.3 | Brain (Thalamus) Pool | 0.5 |
| Fetal Liver | 2.9 | Brain (whole) | 95.9 |
| Liver ca. HepG2 | 0.0 | Spinal Cord Pool | 0.6 |
| Kidney Pool | 8.0 | Adrenal Gland | 2.3 |
| Fetal Kidney | 2.9 | Pituitary gland Pool | 0.6 |
| Renal ca. 786-0 | 0.0 | Salivary Gland | 0.4 |
| Renal ca. A498 | 0.5 | Thyroid (female) | 1.1 |
| Renal ca. ACHN | 2.0 | Pancreatic ca. CAPAN2 | 0.8 |
| Renal ca. UO-31 | 2.2 | Pancreas Pool | 3.0 |

TABLE A4
Panel 4.1D

| | Rel. Exp.(%) | | Rel. Exp.(%) |
|---|---|---|---|
| | Ag3978, Run | | Ag3978, Run |
| Tissue Name | 170737278 | Tissue Name | 170737278 |
| Secondary Th1 act | 0.0 | HUVEC IL-1beta | 33.4 |
| Secondary Th2 act | 0.0 | HUVEC IFN gamma | 28.1 |
| Secondary Tr1 act | 0.0 | HUVEC TNF alpha + IFN | 25.9 |
| | | gamma | |
| Secondary Th1 rest | 0.0 | HUVEC TNF alpha + IL4 | 46.7 |
| Secondary Th2 rest | 0.0 | HUVEC IL-11 | 23.3 |
| Secondary Tr1 rest | 0.0 | Lung Microvascular EC | 100.0 |
| | | none | |
| Primary Th1 act | 0.0 | Lung Microvascular EC | 39.0 |
| | | TNFalpha + IL-1beta | |
| Primary Th2 act | 0.0 | Microvascular Dermal EC | 66.9 |
| | | none | |
| Primary Tr1 act | 0.0 | Microsvasular Dermal EC | 36.3 |
| | | TNFalpha + IL-1beta | |
| Primary Th1 rest | 0.0 | Bronchial epithelium | 7.8 |
| | | TNFalpha + IL1beta | |
| Primary Th2 rest | 0.0 | Small airway epithelium | 1.5 |
| | | none | |
| Primary Tr1 rest | 0.0 | Small airway epithelium | 22.7 |
| | | TNFalpha + IL-1beta | |
| CD45RA CD4 | 1.2 | Coronery artery SMC rest | 14.5 |
| lymphocyte act | | | |
| CD45RO CD4 | 0.0 | Coronery artery SMC | 9.0 |
| lymphocyte act | | TNFalpha + IL-1beta | |
| CD8 lymphocyte act | 0.0 | Astrocytes rest | 0.9 |
| Secondary CD8 | 0.0 | Astrocytes TNFalpha + IL- | 1.4 |
| lymphocyte rest | | 1beta | |
| Secondary CD8 | 0.0 | KU-812 (Basophil) rest | 2.3 |
| lymphocyte act | | | |
| CD4 lymphocyte none | 0.0 | KU-812 (Basophil) | 1.6 |
| | | PMA/ionomycin | |
| 2ry Th1/Th2/Tr1_anti- | 0.0 | CCD1106 (Keratinocytes) | 0.3 |
| CD95 CH11 | | none | |
| LAK cells rest | 0.0 | CCD1106 (Keratinocytes) | 0.0 |
| | | TNFalpha + IL-1beta | |
| LAK cells IL-2 | 0.0 | Liver cirrhosis | 0.9 |
| LAK cells IL-2 + IL-12 | 0.0 | NCI-H292 none | 1.3 |
| LAK cells IL-2 + IFN | 0.0 | NCI-H292 IL-4 | 3.4 |
| gamma | | | |
| LAK cells IL-2 + IL-18 | 0.0 | NCI-H292 IL-9 | 3.1 |
| LAK cells | 0.3 | NCI-H292 IL-13 | 3.4 |
| PMA/ionomycin | | | |
| NK Cells IL-2 rest | 0.0 | NCI-H292 IFN gamma | 2.9 |
| Two Way MLR 3 day | 0.0 | HPAEC none | 49.7 |
| Two Way MLR 5 day | 0.0 | HPAEC TNF alpha + IL-1 | 40.9 |
| | | beta | |
| Two Way MLR 7 day | 0.0 | Lung fibroblast none | 4.8 |
| PBMC rest | 0.0 | Lung fibroblast TNF | 2.7 |
| | | alpha + IL-1beta | |
| PBMC PWM | 0.0 | Lung fibroblast IL-4 | 2.8 |
| PBMC PHA-L | 0.0 | Lung fibroblast IL-9 | 7.0 |
| Ramos (B cell) none | 0.0 | Lung fibroblast IL-13 | 2.0 |
| Ramos (B cell) | 0.0 | Lung fibroblast IFN gamma | 2.4 |
| ionomycin | | | |
| B lymphocytes PWM | 0.0 | Dermal fibroblast | 5.1 |
| | | CCD1070 rest | |
| B lymphocytes CD40L | 0.0 | Dermal fibroblast | 0.5 |
| and IL-4 | | CCD1070 TNF alpha | |
| EOL-1 dbcAMP | 0.6 | Dermal fibroblast | 1.6 |
| | | CCD1070 IL-1beta | |
| EOL-1 dbcAMP | 1.1 | Dermal fibroblast IFN | 0.0 |
| PMA/ionomycin | | gamma | |
| Dendritic cells none | 0.3 | Dermal fibroblast IL-4 | 0.0 |
| Dendritic cells LPS | 0.0 | Dermal Fibroblasts rest | 0.4 |
| Dendritic cells anti- | 0.0 | Neutrophils TNFa+LPS | 0.3 |
| CD40 | | | |
| Monocytes rest | 0.0 | Neutrophils rest | 0.6 |
| Monocytes LPS | 0.0 | Colon | 0.2 |
| Macrophages rest | 0.3 | Lung | 4.5 |
| Macrophages LPS | 0.0 | Thymus | 2.1 |
| HUVEC none | 42.9 | Kidney | 2.2 |
| HUVEC starved | 56.6 | | |

CNS_neurodegeneration_v1.0 Summary: Ag3978 This panel confirms expression of the COR87920446_A gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in the treatment of central nervous system disorders.

General_screening_panel_v1.4 Summary: Ag3978 Expression of the COR87920446_A gene is highest in samples derived from normal breast, stomach and brain tissues (CTs=26.6). Thus, the expression of this gene could be used to distinguish these samples from the other samples in the panel. In addition, there is substantial expression of this gene associated with an ovarian cancer cell line and two lung cancer cell lines. Therefore, therapeutic modulation of the activity of this gene or its protein product, through the use of small molecule drugs, protein therapeutics or antibodies, might be beneficial in the treatment of lung cancer or ovarian cancer.

In addition, this gene is expressed at low levels in all CNS regions examined, including amygdala, cerebellum, hippocampus, cerebral cortex, substantia nigra, thalamus and spinal cord (CTs=33-35). Interestingly, COR87920446_A gene expression is significantly higher in adult brain (CT=26.6) than in fetal brain (CT=33.5), suggesting that expression of this gene may be used to distinguish between adult and fetal brain. This gene encodes a protein with homology to the MEGF protein, and may therefore possibly interact with Notch receptors in neurodevelopment. This protein could therefore be of use in directing compensatory synaptogenesis in clinical conditions involving neuronal death such as stroke and head trauma, and neurodegenerative diseases such as Alzheimer's, Parkinson's, and Huntington's diseases.

This gene is also expressed at low to moderate levels in a number of tissues with metabolic or endocrine function, including adipose, adrenal gland, gastrointestinal tract, pancreas, skeletal muscle and thyroid. Therefore, therapeutic modulation of the activity of this gene may prove useful in the treatment of endocrine/metabolically related diseases, such as Type II diabetes.

Panel 4.1D Summary: Ag3978 The COR87920446_A gene is expressed at low to moderate levels in endothelial cells (HUVEC, HPAEC) as well as in epithelium (CTs=30-32). Activation with a variety of cytokines does not significantly change expression. This gene may encode a ligand for Notch; Notch-ligand interactions play an essential role during limb, craniofacial, and thymic development in mice. Multiple ligands that activate Notch and related receptors have been identified, including Serrate and Delta in Drosophila and JAG1 in vertebrates [602570; OMIM]. This family of molecules is also important in fate determination and development. Therefore, therapeutics designed with the protein encoded for by this transcript could be important for wound healing and organogenesis. Such therapeutics could be important in the treatment of emphysema, psoriasis, arthritis, cirrhosis and inflammatory bowel disease, where there is considerable damage due to inflammation or aberrant would healing.

References:

1. Shutter J R, Scully S, Fan W, Richards W G, Kitajewski J, Deblandre G A, Kintner C R, Stark K L. D114, a novel Notch ligand expressed in arterial endothelium. Genes Dev Jun. 1, 2000 ;14(11):1313-8

We report the cloning and characterization of a new member of the Delta family of Notch ligands, which we have named D114. Like other Delta genes, D114 is predicted to encode a membrane-bound ligand, characterized by an extracellular region containing several EGF-like domains and a DSL domain required for receptor binding. In situ analysis reveals a highly selective expression pattern of D114 within the vascular endothelium. The activity and expression of D114 and the known actions of other members of this family suggest a role for D114 in the control of endothelial cell biology. PMID: 10837024

B. NOV2: Novel Kinase

Expression of the NOV2 gene (COR87940554) was assessed using the primer-probe set Ag3979, described in Table B1. Results of the RTQ-PCR runs are shown in Tables B2, B3, and B4.

TABLE B1

Probe Name Ag3979

| | | | | |
|---|---|---|---|---|
| Primers | Sequences | Length | Start Position | Seq ID No. |

| | | | | |
|---|---|---|---|---|
| Forward | 5′-gctccttcaagacggtgtatc-3′ | 21 | 612 | 89 |
| Probe | TET-5′-ctagacaccgacaccacagtggaggt-3′-TAMRA | 26 | 638 | 90 |
| Reverse | 5′-ccgctcagctctagacagttt-3′ | 21 | 689 | 91 |

TABLE B2
General screening panel v1.4

| | Rel. Exp.(%) Ag3979, | | Rel. Exp.(%) Ag3979, |
|---|---|---|---|
| Tissue Name | Run 217534174 | Tissue Name | Run 217534174 |
| Adipose | 1.3 | Renal ca. TK-10 | 14.5 |
| Melanoma* | 20.7 | Bladder | 0.6 |
| Hs688(A).T | | | |
| Melanoma* | 91.4 | Gastric ca. (liver met.) | 6.7 |
| Hs688(B).T | | NCI-N87 | |
| Melanoma* M14 | 8.6 | Gastric ca. KATO III | 0.3 |
| Melanoma* | 4.2 | Colon ca. SW-948 | 3.4 |
| LOXIMVI | | | |
| Melanoma* SK- | 0.8 | Colon ca. SW480 | 4.5 |
| MEL-5 | | | |
| Squamous cell | 0.5 | Colon ca.* (SW480 | 5.9 |
| carcinoma SCC-4 | | met) SW620 | |
| Testis Pool | 0.8 | Colon ca. HT29 | 24.3 |
| Prostate ca.* (bone | 100.0 | Colon ca. HCT-116 | 5.1 |
| met) PC-3 | | | |
| Prostate Pool | 15.4 | Colon ca. CaCo-2 | 39.8 |
| Placenta | 0.0 | Colon cancer tissue | 24.1 |
| Uterus Pool | 0.3 | Colon ca. SW1116 | 0.6 |
| Ovarian ca. OVCAR-3 | 0.5 | Colon ca. Colo-205 | 0.2 |
| Ovarian ca. SK-OV-3 | 0.7 | Colon ca. SW-48 | 15.8 |
| Ovarian ca. OVCAR-4 | 0.5 | Colon Pool | 0.0 |
| Ovarian ca. OVCAR-5 | 11.5 | Small Intestine Pool | 2.9 |
| Ovarian ca. IGROV-1 | 1.3 | Stomach Pool | 0.5 |
| Ovarian ca. OVCAR-8 | 2.4 | Bone Marrow Pool | 0.6 |
| Ovary | 3.2 | Fetal Heart | 0.5 |
| Breast ca. MCF-7 | 0.8 | Heart Pool | 0.1 |
| Breast ca. MDA-MB- | 6.1 | Lymph Node Pool | 0.3 |
| 231 | | | |
| Breast ca. BT 549 | 21.8 | Fetal Skeletal Muscle | 0.0 |
| Breast ca. T47D | 16.0 | Skeletal Muscle Pool | 0.0 |
| Breast ca. MDA-N | 0.9 | Spleen Pool | 0.3 |
| Breast Pool | 0.2 | Thymus Pool | 0.1 |
| Trachea | 6.3 | CNS cancer (glio/astro) | 0.3 |
| | | U87-MG | |
| Lung | 1.2 | CNS cancer (glio/astro) | 0.3 |
| | | U-118-MG | |
| Fetal Lung | 2.9 | CNS cancer (neuro;met) | 0.1 |
| | | SK-N-AS | |
| Lung ca. NCI-N417 | 0.0 | CNS cancer (astro) SF- | 0.0 |
| | | 539 | |
| Lung ca. LX-1 | 6.9 | CNS cancer (astro) | 1.7 |
| | | SNB-75 | |
| Lung ca. NCI-H146 | 0.1 | CNS cancer (glio) SNB- | 0.7 |
| | | 19 | |
| Lung ca. SHP-77 | 1.0 | CNS cancer (glio) SF- | 11.4 |
| | | 295 | |
| Lung ca. A549 | 5.4 | Brain (Amygdala) Pool | 0.3 |
| Lung ca. NCI-H526 | 0.1 | Brain (cerebellum) | 1.7 |
| Lung ca. NCI-H23 | 3.1 | Brain (fetal) | 2.1 |
| Lung ca. NCI-H460 | 0.8 | Brain (Hippocampus) | 1.3 |
| | | Pool | |
| Lung ca. HOP-62 | 12.8 | Cerebral Cortex Pool | 0.5 |
| Lung ca. NCI-H522 | 5.6 | Brain (Substantia nigra) | 0.2 |
| | | Pool | |
| Liver | 0.0 | Brain (Thalamus) Pool | 0.3 |
| Fetal Liver | 52.5 | Brain (whole) | 1.0 |
| Liver ca. HepG2 | 28.7 | Spinal Cord Pool | 0.4 |
| Kidney Pool | 0.0 | Adrenal Gland | 0.2 |
| Fetal Kidney | 24.5 | Pituitary gland Pool | 0.6 |
| Renal ca. 786-0 | 0.9 | Salivary Gland | 0.6 |
| Renal ca. A498 | 1.6 | Thyroid (female) | 0.0 |
| Renal ca. ACHN | 32.1 | Pancreatic ca. CAPAN2 | 1.5 |
| Renal ca. UO-31 | 20.7 | Pancreas Pool | 1.6 |

TABLE B3
Panel 2.1

| | Rel. Exp.(%) Ag3979, | | Rel. Exp.(%) Ag3979, |
|---|---|---|---|
| Tissue Name | Run 170721574 | Tissue Name | Run 170721574 |
| Normal Colon | 9.9 | Kidney Cancer | 0.0 |
| | | 9010320 | |
| Colon cancer (OD06064) | 0.2 | Kidney margin | 44.4 |
| | | 9010321 | |
| Colon cancer margin | 2.6 | Kidney Cancer | 1.5 |
| (OD06064) | | 8120607 | |
| Colon cancer (OD06159) | 3.5 | Kidney margin | 8.7 |
| | | 8120608 | |
| Colon cancer margin | 2.4 | Normal Uterus | 0.0 |
| (OD06159) | | | |
| Colon cancer (OD06298- | 30.8 | Uterus Cancer | 0.6 |
| 08) | | | |
| Colon cancer margin | 12.7 | Normal Thyroid | 0.0 |
| (OD06298-018) | | | |
| Colon Cancer Gr.2 ascend | 4.7 | Thyroid Cancer | 0.0 |
| colon (ODO3921) | | | |
| Colon Cancer margin | 4.1 | Thyroid Cancer | 0.0 |
| (ODO3921) | | A302152 | |
| Colon cancer metastasis | 0.8 | Thyroid margin | 0.0 |
| (OD06104) | | A302153 | |
| Lung margin (OD06104) | 1.2 | Normal Breast | 3.2 |
| Colon mets to lung | 9.5 | Breast Cancer | 3.4 |
| (OD04451-01) | | | |
| Lung margin (OD04451- | 0.0 | Breast Cancer | 2.9 |
| 02) | | | |
| Normal Prostate | 9.3 | Breast Cancer | 1.3 |
| | | (OD04590-01) | |
| Prostate Cancer (OD04410) | 6.4 | Breast Cancer Mets | 2.3 |
| | | (OD04590-03) | |
| Prostate margin (OD04410) | 9.0 | Breast Cancer | 100.0 |
| | | Metastasis | |
| Normal Lung | 0.2 | Breast Cancer | 0.0 |
| Invasive poor diff. lung | 0.3 | Breast Cancer | 2.8 |
| adeno 1 (ODO4945-01) | | 9100266 | |
| Lung margin (ODO4945- | 0.0 | Breast margin | 2.9 |
| 03) | | 9100265 | |
| Lung Malignant Cancer | 2.8 | Breast Cancer | 0.5 |
| (OD03126) | | A209073 | |
| Lung margin (OD03126) | 0.6 | Breast margin | 3.5 |
| | | A2090734 | |
| Lung Cancer (OD05014A) | 0.0 | Normal Liver | 0.0 |
| Lung margin (OD05014B) | 0.0 | Liver Cancer 1026 | 2.6 |
| Lung Cancer (OD04237- | 0.0 | Liver Cancer 1025 | 0.3 |
| 01) | | | |
| Lung margin (OD04237- | 0.0 | Liver Cancer 6004-T | 0.6 |
| 02) | | | |
| Ocular Mel Met to Liver | 3.9 | Liver Tissue 6004-N | 1.4 |
| (ODO4310) | | | |
| Liver margin (ODO4310) | 0.0 | Liver Cancer 6005-T | 4.4 |
| Melanoma Mets to Lung | 13.9 | Liver Tissue 6005-N | 0.0 |
| (OD04321) | | | |
| Lung margin (OD04321) | 0.0 | Liver Cancer | 1.7 |
| Normal Kidney | 19.9 | Normal Bladder | 0.0 |
| Kidney Ca, Nuclear grade 2 | 76.8 | Bladder Cancer | 6.7 |
| (OD04338) | | | |
| Kidney margin (OD04338) | 1.5 | Bladder Cancer | 0.0 |
| Kidney Ca Nuclear grade | 0.7 | Normal Ovary | 1.8 |
| 1/2 (OD04339) | | | |
| Kidney margin (OD04339) | 19.1 | Ovarian Cancer | 0.0 |
| Kidney Ca, Clear cell type | 0.0 | Ovarian cancer | 0.0 |
| (OD04340) | | (OD06145) | |
| Kidney margin (OD04340) | 15.4 | Ovarian cancer | 0.0 |
| | | margin (OD06145) | |
| Kidney Ca, Nuclear grade 3 | 0.0 | Normal Stomach | 1.8 |
| (OD04348) | | | |
| Kidney margin (OD04348) | 20.7 | Gastric Cancer | 2.5 |
| | | 9060397 | |
| Kidney Cancer (OD04450- | 1.4 | Stomach margin | 1.2 |
| 01) | | 9060396 | |
| Kidney margin (OD04450- | 42.9 | Gastric Cancer | 1.0 |
| 03) | | 9060395 | |
| Kidney Cancer 8120613 | 0.0 | Stomach margin | 2.2 |
| | | 9060394 | |
| Kidney margin 8120614 | 9.9 | Gastric Cancer | 1.0 |
| | | 064005 | |

TABLE B4
Panel 4.1D

| | Rel. Exp.(%) | | Rel. Exp.(%) |
|---|---|---|---|
| | Ag3979, Run | | Ag3979, Run |
| Tissue Name | 170721251 | Tissue Name | 170721251 |
| Secondary Th1 act | 0.0 | HUVEC IL-1beta | 1.2 |
| Secondary Th2 act | 0.0 | HUVEC IFN gamma | 1.8 |
| Secondary Tr1 act | 0.0 | HUVEC TNF alpha + IFN | 0.2 |
| | | gamma | |
| Secondary Th1 rest | 0.0 | HUVEC TNF alpha + IL4 | 0.3 |
| Secondary Th2 rest | 0.0 | HUVEC IL-11 | 0.9 |
| Secondary Tr1 rest | 0.0 | Lung Microvascular EC | 100.0 |
| | | none | |
| Primary Th1 act | 0.1 | Lung Microvascular EC | 58.2 |
| | | TNF alpha + IL-1beta | |
| Primary Th2 act | 0.0 | Microvascular Dermal EC | 72.2 |
| | | none | |
| Primary Tr1 act | 0.0 | Microsvasular Dermal EC | 48.0 |
| | | TNF alpha + IL-1beta | |
| Primary Th1 rest | 0.0 | Bronchial epithelium | 3.4 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.7 |
| | | TNF alpha + IL-1beta | |
| CD45RA CD4 | 5.2 | Coronery artery SMC rest | 39.5 |
| lymphocyte act | | | |
| CD45RO CD4 | 0.0 | Coronery artery SMC | 40.6 |
| lymphocyte act | | TNF alpha + IL-1beta | |
| CD8 lymphocyte act | 0.0 | Astrocytes rest | 12.1 |
| Secondary CD8 | 0.0 | Astrocytes TNF alpha + IL- | 27.5 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 1.7 |
| CD95 CH11 | | none | |
| LAK cells rest | 0.0 | CCD1106 (Keratinocytes) | 1.3 |
| | | TNF alpha + IL-1beta | |
| LAK cells IL-2 | 0.0 | Liver cirrhosis | 0.6 |
| LAK cells IL-2 + IL-12 | 0.0 | NCI-H292 none | 0.5 |
| LAK cells IL-2 + IFN | 0.0 | NCI-H292 IL-4 | 0.8 |
| gamma | | | |
| LAK cells IL-2 + IL-18 | 0.0 | NCI-H292 IL-9 | 1.2 |
| LAK cells | 0.0 | NCI-H292 IL-13 | 0.3 |
| PMA/ionomycin | | | |
| NK Cells IL-2 rest | 0.0 | NCI-H292 IFN gamma | 0.6 |
| Two Way MLR 3 day | 0.0 | HPAEC none | 3.4 |
| Two Way MLR 5 day | 0.0 | HPAEC TNF alpha + IL-1 | 7.5 |
| | | beta | |
| Two Way MLR 7 day | 0.2 | Lung fibroblast none | 0.3 |
| PBMC rest | 0.0 | Lung fibroblast | 0.1 |
| | | TNF alpha + IL-1beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.6 |
| Ramos (B cell) | 0.4 | Lung fibroblast IFN gamma | 0.9 |
| ionomycin | | | |
| B lymphocytes PWM | 0.0 | Dermal fibroblast | 14.3 |
| | | CCD1070 rest | |
| B lymphocytes CD40L | 0.0 | Dermal fibroblast | 9.9 |
| and IL-4 | | CCD1070 TNF alpha | |
| EOL-1 dbcAMP | 0.9 | Dermal fibroblast | 11.2 |
| | | CCD1070 IL-1beta | |
| EOL-1 dbcAMP | 1.8 | Dermal fibroblast IFN | 0.9 |
| PMA/ionomycin | | gamma | |
| Dendritic cells none | 0.0 | Dermal fibroblast IL-4 | 0.2 |
| Dendritic cells LPS | 0.0 | Dermal Fibroblasts rest | 0.0 |
| Dendritic cells anti- | 0.3 | Neutrophils TNFa + LPS | 0.3 |
| CD40 | | | |
| Monocytes rest | 0.0 | Neutrophils rest | 0.3 |
| Monocytes LPS | 0.0 | Colon | 2.0 |
| Macrophages rest | 0.0 | Lung | 0.0 |
| Macrophages LPS | 0.0 | Thymus | 4.5 |
| HUVEC none | 3.5 | Kidney | 29.1 |
| HUVEC starved | 1.5 | | |

CNS_neurodegeneration_v1.0 Summary: Ag3979 Expression of the COR87940554 gene is low/undetectable (CTs>35) across all of the samples on this panel (data not shown).

General_screening_panel_v1.4 Summary: Ag3979 Expression of the COR87940554 gene is highest in prostate cancer cell line PC-3 and a melanoma cell line (CT=28). Thus, the expression of this gene could be used to distinguish these cells from the other samples in the panel. In addition, there is substantial expression of this gene associated with kidney cancer cell lines and colon cancer cell lines. Therefore, therapeutic modulation of the activity of this gene or its protein product, through the use of small molecule drugs, protein therapeutics or antibodies, might be of benefit in the treatment of kidney cancer, prostate cancer, colon cancer or melanoma. Finally, expression of this gene is much higher in fetal liver (CT=29) than adult liver (CT=40), as well as in fetal kidney (CT=30) than adult kidney (CT=40). This observation suggests that expression of this gene may be used to distinguish fetal from adult liver or kidney.

This gene encodes a protein with homology to kinases and is expressed at very low levels in the fetal brain, hippocampus, and cerebellum. This gene is predominantly expressed in fetal tissues and in cancer cell lines, suggesting that it plays a role in cell division or differentiation. Thus, this gene may therefore be of use in regulation of the cell cycle in stem cell research or therapy.

Panel 2.1 Summary: Ag3979 Expression of the COR87940554 gene is highest in a sample derived from a metastatic breast cancer (CT=30.9). Thus, the expression of this gene could be used to distinguish this metastatic breast cancer specimen from other samples in the panel. In addition, there appears to be substantial expression of this gene associated with a number of normal kidney tissue samples adjacent to malignant kidney. Therefore, therapeutic modulation of the activity of this gene or its protein product, through the use of small molecule drugs, protein therapeutics or antibodies, might be of benefit in the treatment of breast and kidney cancer.

Panel 4.1D Summary: Ag3979 Expression of this gene is highest in lung microvascular endothelial cells (CT=29.7). The COR87940554 gene is also expressed in fibroblasts, endothelium, and smooth muscle cells. This gene encodes a putative protein kinase that localizes to the nucleus based on PSORT analysis. The protein encoded for by this transcript may be important in the normal function of the fibroblasts, endothelial cells and smooth muscle cells. Therefore, therapies designed with the protein encoded for by this transcript could be used to regulate fibroblast, endothelium and smooth muscle cell function and may be important in the treatment of asthma, emphysema, arthritis, and inflammatory bowel disease.

C. NOV8a and NOV8b: GPCR

Expression of the NOV8a gene (CG56663-01) and its variant NOV8b (CG56663-02) was assessed using the primer-probe set Ag2971, described in Table C1. Results of the RTQ-PCR runs are shown in Tables C2, C3 and C4. NOV8b represents a full-length physical clone of the NOV8a gene, validating the prediction of the gene sequence.

TABLE C1

Probe Name Ag2971

| | | | | |
|---|---|---|---|---|
| Primers | Sequences | Length | Start Position | Seq ID No. |

| | | | | |
|---|---|---|---|---|
| Forward | 5′-gtaaaggcatctccacctgact-3′ | 22 | 947 | 92 |
| Probe | TET-5′-tcacttccatccagggccactgg-3′-TAMRA | 23 | 969 | 93 |
| Reverse | 5′-gggctaatatcagctggaattc-3′ | 22 | 1009 | 94 |

TABLE C2
CNS neurodegeneration v1.0

| | Rel. Exp.(%) Ag2971, | | Rel. Exp.(%) Ag2971, |
|---|---|---|---|
| Tissue Name | Run 209778983 | Tissue Name | Run 209778983 |
| AD 1 Hippo | 6.5 | Control (Path) 3 | 1.2 |
| | | Temporal Ctx | |
| AD 2 Hippo | 31.6 | Control (Path) 4 | 51.4 |
| | | Temporal Ctx | |
| AD 3 Hippo | 1.8 | AD 1 Occipital Ctx | 14.9 |
| AD 4 Hippo | 15.1 | AD 2 Occipital Ctx | 0.0 |
| | | (Missing) | |
| AD 5 hippo | 52.9 | AD 3 Occipital Ctx | 0.8 |
| AD 6 Hippo | 19.6 | AD 4 Occipital Ctx | 20.9 |
| Control 2 Hippo | 18.9 | AD 5 Occipital Ctx | 11.9 |
| Control 4 Hippo | 3.9 | AD 6 Occipital Ctx | 26.1 |
| Control (Path) 3 | 3.8 | Control 1 Occipital | 2.8 |
| Hippo | | Ctx | |
| AD 1 Temporal Ctx | 9.5 | Control 2 Occipital | 46.3 |
| | | Ctx | |
| AD 2 Temporal Ctx | 22.1 | Control 3 Occipital | 13.2 |
| | | Ctx | |
| AD3 Temporal Ctx | 3.1 | Control 4 Occipital | 4.4 |
| | | Ctx | |
| AD 4 Temporal Ctx | 28.5 | Control (Path) 1 | 100.0 |
| | | Occipital Ctx | |
| AD 5 Inf Temporal | 82.4 | Control (Path) 2 | 7.3 |
| Ctx | | Occipital Ctx | |
| AD 5 Sup Temporal | 46.0 | Control (Path) 3 | 5.5 |
| Ctx | | Occipital Ctx | |
| AD 6 Inf Temporal | 30.4 | Control (Path) 4 | 23.3 |
| Ctx | | Occipital Ctx | |
| AD 6 Sup Temporal | 28.3 | Control 1 Parietal | 8.4 |
| Ctx | | Ctx | |
| Control 1 Temporal | 3.6 | Control 2 Parietal | 25.3 |
| Ctx | | Ctx | |
| Control 2 Temporal | 33.7 | Control 3 Parietal | 22.2 |
| Ctx | | Ctx | |
| Control 3 Temporal | 32.3 | Control (Path) 1 | 81.8 |
| Ctx | | Parietal Ctx | |
| Control 4 Temporal | 7.1 | Control (Path) 2 | 9.2 |
| Ctx | | Parietal Ctx | |
| Control (Path) 1 | 80.7 | Control (Path) 3 | 3.2 |
| Temporal Ctx | | Parietal Ctx | |
| Control (Path) 2 | 10.5 | Control (Path) 4 | 80.7 |
| Temporal Ctx | | Parietal Ctx | |

TABLE C3
Panel 1.3D

| | Rel. Exp.(%) Ag2971, | | Rel. Exp.(%) Ag2971, |
|---|---|---|---|
| Tissue Name | Run 166219829 | Tissue Name | Run 166219829 |
| Liver adenocarcinoma | 4.2 | Kidney (fetal) | 1.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 3.4 | Renal ca. ACHN | 0.0 |
| Salivary gland | 0.0 | Renal ca. UO-31 | 0.0 |
| Pituitary gland | 4.3 | Renal ca. TK-10 | 0.0 |
| Brain (fetal) | 33.4 | Liver | 0.0 |
| Brain (whole) | 45.7 | Liver (fetal) | 21.0 |
| Brain (amygdala) | 18.2 | Liver ca. | 1.9 |
| | | (hepatoblast) HepG2 | |
| Brain (cerebellum) | 1.5 | Lung | 0.0 |
| Brain (hippocampus) | 7.5 | Lung (fetal) | 1.1 |
| Brain (substantia nigra) | 17.8 | Lung ca. (small cell) | 0.0 |
| | | LX-1 | |
| Brain (thalamus) | 14.9 | Lung ca. (small cell) | 0.0 |
| | | NCI-H69 | |
| Cerebral Cortex | 22.1 | Lung ca. (s.cell var.) | 0.0 |
| | | SHP-77 | |
| Spinal cord | 4.9 | Lung ca. (large | 0.0 |
| | | cell) NCI-H460 | |
| glio/astro U87-MG | 0.0 | Lung ca. (non-sm. | 0.0 |
| | | cell) A549 | |
| glio/astro U-118-MG | 0.0 | Lung ca. (non-s.cell) | 0.0 |
| | | NCI-H23 | |
| astrocytoma SW1783 | 0.0 | Lung ca. (non-s.cell) | 3.9 |
| | | HOP-62 | |
| neuro*; met SK-N-AS | 0.9 | Lung ca. (non-s.cl) | 0.0 |
| | | NCI-H522 | |
| astrocytoma SF-539 | 0.0 | Lung ca. (squam.) | 0.0 |
| | | SW 900 | |
| astrocytoma SNB-75 | 0.0 | Lung ca. (squam.) | 0.0 |
| | | NCI-H596 | |
| glioma SNB-19 | 0.0 | Mammary gland | 5.9 |
| glioma U251 | 0.0 | Breast ca.* (pl.ef) | 0.0 |
| | | MCF-7 | |
| glioma SF-295 | 0.0 | Breast ca.* (pl.ef) | 0.0 |
| | | MDA-MB-231 | |
| Heart (fetal) | 15.1 | Breast ca.* (pl.ef) | 0.0 |
| | | T47D | |
| Heart | 0.0 | Breast ca. BT-549 | 0.0 |
| Skeletal muscle (fetal) | 3.3 | Breast ca. MDA-N | 0.0 |
| Skeletal muscle | 0.8 | Ovary | 0.5 |
| Bone marrow | 100.0 | Ovarian ca. OVCAR-3 | 0.0 |
| Thymus | 0.0 | Ovarian ca. OVCAR-4 | 0.0 |
| Spleen | 2.0 | Ovarian ca. OVCAR-5 | 0.0 |
| Lymph node | 0.0 | Ovarian ca. OVCAR-8 | 7.3 |
| Colorectal | 4.2 | Ovarian ca. IGROV-1 | 0.0 |
| Stomach | 0.0 | Ovarian ca.* (ascites) | 57.0 |
| | | SK-OV-3 | |
| Small intenstine | 1.1 | Uterus | 0.9 |
| Colon ca. SW480 | 0.9 | Placenta | 27.9 |
| Colon ca.* | 0.0 | Prostate | 0.0 |
| SW620(SW480 met) | | | |
| Colon ca. HT29 | 0.0 | Prostate ca.* (bone | 4.5 |
| | | met)PC-3 | |
| Colon ca. HCT-116 | 0.0 | Testis | 12.8 |
| Colon ca. CaCo-2 | 0.0 | Melanoma | 0.0 |
| | | Hs688(A).T | |
| Colon ca. | 0.0 | Melanoma* (met) | 0.0 |
| tissue(ODO3866) | | Hs688(B).T | |
| Colon ca. HCC-2998 | 0.0 | Melanoma UACC-62 | 9.0 |
| Gastric ca.* (liver met) | 0.0 | Melanoma M14 | 1.1 |
| NCI-N87 | | | |
| Bladder | 0.0 | Melanoma LOX | 0.0 |
| | | IMVI | |
| Trachea | 0.0 | Melanoma* (met) | 0.0 |
| | | SK-MEL-5 | |
| Kidney | 0.0 | Adipose | 2.5 |

TABLE C4
Panel 4D

| | Rel. Exp.(%) | | Rel. Exp.(%) |
|---|---|---|---|
| | Ag2971, Run | | Ag2971, Run |
| Tissue Name | 164403109 | Tissue Name | 164403109 |
| Secondary Th1 act | 0.6 | HUVEC IL-1beta | 0.0 |
| Secondary Th2 act | 0.4 | HUVEC IFN gamma | 0.7 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.3 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 1.5 | Bronchial epithelium | 0.0 |
| | | TNF alpha + IL1beta | |
| Primary Th2 rest | 2.2 | Small airway epithelium | 0.0 |
| | | none | |
| Primary Tr1 rest | 0.4 | Small airway epithelium | 0.0 |
| | | TNF alpha + IL-1beta | |
| CD45RA CD4 | 0.0 | Coronery artery SMC rest | 3.1 |
| lymphocyte act | | | |
| CD45RO CD4 | 0.8 | Coronery artery SMC | 0.5 |
| lymphocyte act | | TNF alpha + IL-1beta | |
| CD8 lymphocyte act | 0.0 | Astrocytes rest | 0.0 |
| Secondary CD8 | 0.4 | Astrocytes TNF alpha + IL- | 0.0 |
| lymphocyte rest | | 1beta | |
| Secondary CD8 | 0.0 | KU-812 (Basophil) rest | 47.0 |
| lymphocyte act | | | |
| CD4 lymphocyte none | 0.1 | KU-812 (Basophil) | 100.0 |
| | | PMA/ionomycin | |
| 2ry Th1/Th2/Tr1_anti- | 0.1 | CCD1106 (Keratinocytes) | 0.0 |
| CD95 CH11 | | none | |
| LAK cells rest | 0.2 | CCD1106 (Keratinocytes) | 0.0 |
| | | TNF alpha + IL-1beta | |
| LAK cells IL-2 | 1.1 | Liver cirrhosis | 2.3 |
| LAK cells IL-2 + IL-12 | 1.4 | Lupus kidney | 0.0 |
| LAK cells IL-2 + IFN | 0.5 | NCI-H292 none | 0.0 |
| gamma | | | |
| LAK cells IL-2 + IL-18 | 0.6 | NCI-H292 IL-4 | 0.0 |
| LAK cells | 0.0 | NCI-H292 IL-9 | 0.0 |
| PMA/ionomycin | | | |
| NK Cells IL-2 rest | 0.5 | NCI-H292 IL-13 | 0.0 |
| Two Way MLR 3 day | 0.2 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.6 | Lung fibroblast none | 0.3 |
| PBMC PWM | 1.4 | Lung fibroblast | 0.2 |
| | | TNF alpha + IL-1beta | |
| PBMC PHA-L | 1.1 | Lung fibroblast IL-4 | 0.2 |
| Ramos (B cell) none | 0.0 | Lung fibroblast IL-9 | 0.8 |
| Ramos (B cell) | 0.0 | Lung fibroblast IL-13 | 0.0 |
| ionomycin | | | |
| B lymphocytes PWM | 0.8 | Lung fibroblast IFN gamma | 0.2 |
| B lymphocytes CD40L | 1.1 | Dermal fibroblast | 0.0 |
| and IL-4 | | CCD1070 rest | |
| EOL-1 dbcAMP | 1.1 | Dermal fibroblast | 4.5 |
| | | CCD1070 TNF alpha | |
| EOL-1 dbcAMP | 0.0 | Dermal fibroblast | 0.0 |
| PMA/ionomycin | | CCD1070 IL-1 beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.3 | IBD Colitis 2 | 0.2 |
| CD40 | | | |
| Monocytes rest | 2.5 | IBD Crohn's | 0.0 |
| Monocytes LPS | 0.2 | Colon | 0.9 |
| Macrophages rest | 0.2 | Lung | 0.3 |
| Macrophages LPS | 0.2 | Thymus | 0.9 |
| HUVEC none | 0.0 | Kidney | 2.0 |
| HUVEC starved | 0.0 | | |

CNS_neurodegeneration_v1.0 Summary: Ag2971 This panel confirms the expression of the CG56663-01 gene at low levels in the brain in an independent group of individuals. However, no differential expression of this gene was detected between Alzheimer's diseased postmortem brains and those of non-demented controls in this experiment. Please see Panel 1.3D for a discussion of the potential utility of this gene in treatment of central nervous system disorders.

Panel 1.3D Summary: Ag2971 Expression of the CG56663-01 gene is highest in bone marrow (CT=31.6). Interestingly, expression of this gene is significantly higher in fetal heart (CT=34.3) than adult heart (CT=40) as well as in fetal liver (CT=33.8) than adult liver (CT=40). This observation suggests that expression of this gene may be used to distinguish fetal from adult heart and liver.

This gene is also expressed at low levels in several regions of the CNS examined, including amygdala, substantia nigra, thalamus and cerebral cortex. This gene encodes a novel G-protein coupled receptor (GPCR). The GPCR family of receptors contains a large number of neurotransmitter receptors, including the dopamine, serotonin, □ and □-adrenergic, acetylcholine muscarinic, histamine, peptide, and metabotropic glutamate receptors. GPCRs are excellent drug targets in various neurologic and psychiatric diseases. All antipsychotics have been shown to act at the dopamine D2 receptor; similarly novel antipsychotics also act at the serotonergic receptor, and often the muscarinic and adrenergic receptors as well. While the majority of antidepressants can be classified as selective serotonin reuptake inhibitors, blockade of the 5-HT1A and □2 adrenergic receptors increases the effects of these drugs. The GPCRs are also of use as drug targets in the treatment of stroke. Blockade of the glutamate receptors may decrease the neuronal death resulting from excitotoxicity; further more the purinergic receptors have also been implicated as drug targets in the treatment of cerebral ischemia. The □-adrenergic receptors have been implicated in the treatment of ADHD with Ritalin, while the □-adrenergic receptors have been implicated in memory. Therefore, this gene may be of use as a small molecule target for the treatment of any of the described diseases.

References:

1. E1 Yacoubi M, Ledent C, Parmentier M, Bertorelli R, Ongini E, Costentin J, Vaugeois J M. Adenosine A2A receptor antagonists are potential antidepressants: evidence based on pharmacology and A2A receptor knockout mice. Br J Pharmacol 2001 September;134(1):68-77

1. Adenosine, an ubiquitous neuromodulator, and its analogues have been shown to produce ‘depressant’ effects in animal models believed to be relevant to depressive disorders, while adenosine receptor antagonists have been found to reverse adenosine-mediated ‘depressant’ effect. 2. We have designed studies to assess whether adenosine A2A receptor antagonists, or genetic inactivation of the receptor would be effective in established screening procedures, such as tail suspension and forced swim tests, which are predictive of clinical antidepressant activity. 3. Adenosine A2A receptor knockout mice were found to be less sensitive to ‘depressant’ challenges than their wildtype littermates. Consistently, the adenosine A2A receptor blockers SCH 58261 (1-10 mg kg(−1), i.p.) and KW 6002 (0.1-10 mg kg(−1), p.o.) reduced the total immobility time in the tail suspension test. 4. The efficacy of adenosine A2A receptor antagonists in reducing immobility time in the tail suspension test was confirmed and extended in two groups of mice. Specifically, SCH 58261 (1-10 mg kg(−1)) and ZM 241385 (15-60 mg kg(−1)) were effective in mice previously screened for having high immobility time, while SCH 58261 at 10 mg kg(−1) reduced immobility of mice that were selectively bred for their spontaneous ‘helplessness’ in this assay. 5. Additional experiments were carried out using the forced swim test. SCH 58261 at 10 mg kg(−1) reduced the immobility time by 61%, while KW 6002 decreased the total immobility time at the doses of 1 and 10 mg kg(−1) by 75 and 79%, respectively. 6. Administration of the dopamine D2 receptor antagonist haloperidol (50-200 microg kg(−1) i.p.) prevented the antidepressant-like effects elicited by SCH 58261 (10 mg kg(−1) i.p.) in forced swim test whereas it left unaltered its stimulant motor effects. 7. In conclusion, these data support the hypothesis that A2A receptor antagonists prolong escape-directed behaviour in two screening tests for antidepressants. Altogether the results support the hypothesis that blockade of the adenosine A2A receptor might be an interesting target for the development of effective antidepressant agents.

2. Blier P. Pharmacology of rapid-onset antidepressant treatment strategies. Clin Psychiatry 2001;62 Suppl 15:12-7

Although selective serotonin reuptake inhibitors (SSRIs) block serotonin (5-HT) reuptake rapidly, their therapeutic action is delayed. The increase in synaptic 5-HT activates feedback mechanisms mediated by 5-HT1A (cell body) and 5-HT1B (terminal) autoreceptors, which, respectively, reduce the firing in 5-HT neurons and decrease the amount of 5-HT released per action potential resulting in attenuated 5-HT neurotransmission. Long-term treatment desensitizes the inhibitory 5-HT1 autoreceptors, and 5-HT neurotransmission is enhanced. The time course of these events is similar to the delay of clinical action. The addition of pindolol, which blocks 5-HT1A receptors, to SSRI treatment decouples the feedback inhibition of 5-HT neuron firing and accelerates and enhances the antidepressant response. The neuronal circuitry of the 5-HT and norepinephrine (NE) systems and their connections to forebrain areas believed to be involved in depression has been dissected. The firing of 5-HT neurons in the raphe nuclei is driven, at least partly, by alpha1-adrenoceptor-mediated excitatory inputs from NE neurons. Inhibitory alpha2-adrenoceptors on the NE neuroterminals form part of a feedback control mechanism. Mirtazapine, an antagonist at alpha2-adrenoceptors, does not enhance 5-HT neurotransmission directly but disinhibits the NE activation of 5-HT neurons and thereby increases 5-HT neurotransmission by a mechanism that does not require a time-dependent desensitization of receptors. These neurobiological phenomena may underlie the apparently faster onset of action of mirtazapine compared with the SSRIs.

3. Tranquillini M E, Reggiani A. Glycine-site antagonists and stroke. Expert Opin Investig Drugs 1999 November;8(11):1837-1848

The excitatory amino acid, (S)-glutamic acid, plays an important role in controlling many neuronal processes. Its action is mediated by two main groups of receptors: the ionotropic receptors (which include NMDA, AMPA and kainic acid subtypes) and the metabotropic receptors (mGluR(1-8)) mediating G-protein coupled responses. This review focuses on the strychnine insensitive glycine binding site located on the NMDA receptor channel, and on the possible use of selective antagonists for the treatment of stroke. Stroke is a devastating disease caused by a sudden vascular accident. Neurochemically, a massive release of glutamate occurs in neuronal tissue; this overactivates the NMDA receptor, leading to increased intracellular calcium influx, which causes neuronal cell death through necrosis. NMDA receptor activation strongly depends upon the presence of glycine as a co-agonist. Therefore, the administration of a glycine antagonist can block overactivation of NMDA receptors, thus preserving neurones from damage. The glycine antagonists currently identified can be divided into five main categories depending on their chemical structure: indoles, tetrahydroquinolines, benzoazepines, quinoxalinediones and pyrida-zinoquinolines.

4. Monopoli A, Lozza G, Forlani A, Mattavelli A, Ongini E. Blockade of adenosine A2A receptors by SCH 58261 results in neuroprotective effects in cerebral ischaemia in rats. Neuroreport Dec. 1, 1998;9(17):3955-9

Blockade of adenosine receptors can reduce cerebral infarct size in the model of global ischaemia. Using the potent and selective A2A adenosine receptor antagonist, SCH 58261, we assessed whether A2A receptors are involved in the neuronal damage following focal cerebral ischaemia as induced by occluding the left middle cerebral artery. SCH 58261 (0.01 mg/kg either i.p. or i.v.) administered to normotensive rats 10 min after ischaemia markedly reduced cortical infarct volume as measured 24 h later (30% vs controls, p<0.05). Similar effects were observed when SCH 58261 (0.01 mg/kg, i.p.) was administered to hypertensive rats (28% infarct volume reduction vs controls, p<0.05). Neuroprotective properties of SCH 58261 administered after ischaemia indicate that blockade of A2A adenosine receptors is a potentially useful biological target for the reduction of brain injury.

Panel 4D Summary: Ag2971 The CG56663-01 gene is expressed exclusively in the basophil cell line KU-812, irrespective of treatment with PMA and ionomycin. Thus, expression of this gene may be used to distinguish basophils from the other samples on this panel. This gene encodes a putative GPCR and it is known that GPCR-type receptors are important in multiple physiological responses mediated by basophils (ref. 1). Therefore, antibody or small molecule therapies designed with the protein encoded for by this gene could block or inhibit inflammation or tissue damage due to basophil activation in response to asthma, allergies, hypersensitivity reactions, psoriasis, and viral infections.

Reference:

1. Heinemann A., Hartnell A., Stubbs V. E., Murakami K., Soler D., LaRosa G., Askenase P. W., Williams T. J., Sabroe I. (2000) Basophil responses to chemokines are regulated by both sequential and cooperative receptor signaling. J. Immunol. 165: 7224-7233.

To investigate human basophil responses to chemokines, we have developed a sensitive assay that uses flow cytometry to measure leukocyte shape change as a marker of cell responsiveness. PBMC were isolated from the blood of volunteers. Basophils were identified as a single population of cells that stained positive for IL-3Ralpha (CDw 123) and negative for HLA-DR, and their increase in forward scatter (as a result of cell shape change) in response to chemokines was measured. Shape change responses of basophils to chemokines were highly reproducible, with a rank order of potency: monocyte chemoattractant protein (MCP) 4 (peak at/=eotaxin-2=eotaxin-3>/=eotaxin>MCP-1=MCP-3>macrophage-inflammatory protein-1alpha >RANTES=MCP-2=IL-8. The CCR4-selective ligand macrophage-derived chemokine did not elicit a response at concentrations up to 10 nM. Blocking mAbs to CCR2 and CCR3 demonstrated that responses to higher concentrations (>10 nM) of MCP-1 were mediated by CCR3 rather than CCR2, whereas MCP-4 exhibited a biphasic response consistent with sequential activation of CCR3 at lower concentrations and CCR2 at 10 nM MCP-4 and above. In contrast, responses to MCP-3 were blocked only in the presence of both mAbs, but not after pretreatment with either anti-CCR2 or anti-CCR3 mAb alone. These patterns of receptor usage were different from those seen for eosinophils and monocytes. We suggest that cooperation between CCRs might be a mechanism for preferential recruitment of basophils, as occurs in tissue hypersensitivity responses in vivo.

PMID:11120855

D. NOV9: Dual Specificity Phosphatase

Expression of the NOV9 gene (CG56787-01) was assessed using the primer-probe set Ag3021, described in Table D1. Results of the RTQ-PCR runs are shown in Tables D2, D3 and D4.

TABLE D1

Probe Name Ag3O21

| | | | | |
|---|---|---|---|---|
| Primers | Sequences | Length | Start Position | Seq ID No. |

| | | | | |
|---|---|---|---|---|
| Forward | 5′-aattgtttggcaagaacactgt-3′ | 22 | 512 | 95 |
| Probe | TET-5′-ccagtgggaatgatccctgacatcta-3′-TAMRA | 26 | 550 | 96 |
| Reverse | 5′-atcatcaaacggacttccttct-3′ | 22 | 578 | 97 |

TABLE D2
CNS neurodegeneration v1.0

| | Rel. Exp.(%) Ag3021, | | Rel. Exp.(%) Ag3021, |
|---|---|---|---|
| Tissue Name | Run 209821073 | Tissue Name | Run 209821073 |
| AD 1 Hippo | 3.3 | Control (Path) 3 | 1.0 |
| | | Temporal Ctx | |
| AD 2 Hippo | 5.8 | Control (Path) 4 | 5.5 |
| | | Temporal Ctx | |
| AD 3 Hippo | 2.7 | AD 1 Occipital Ctx | 3.0 |
| AD 4 Hippo | 1.2 | AD 2 Occipital Ctx | 0.0 |
| | | (Missing) | |
| AD 5 Hippo | 12.6 | AD 3 Occipital Ctx | 2.5 |
| AD 6 Hippo | 10.3 | AD 4 Occipital Ctx | 100.0 |
| Control 2 Hippo | 3.3 | AD 5 Occipital Ctx | 4.2 |
| Control 4 Hippo | 3.9 | AD 6 Occipital Ctx | 5.3 |
| Control (Path) 3 | 2.7 | Control 1 Occipital | 1.4 |
| Hippo | | Ctx | |
| AD 1 Temporal Ctx | 4.3 | Control 2 Occipital | 5.0 |
| | | Ctx | |
| AD 2 Temporal Ctx | 5.1 | Control 3 Occipital | 2.7 |
| | | Ctx | |
| AD 3 Temporal Ctx | 1.8 | Control 4 Occipital | 3.0 |
| | | Ctx | |
| AD 4 Temporal Ctx | 4.2 | Control (Path) 1 | 13.4 |
| | | Occipital Ctx | |
| AD 5 Inf Temporal | 18.8 | Control (Path) 2 | 1.6 |
| Ctx | | Occipital Ctx | |
| AD 5 Sup Temporal | 13.3 | Control (Path) 3 | 1.1 |
| Ctx | | Occipital Ctx | |
| AD 6 Inf Temporal | 13.0 | Control (Path) 4 | 3.0 |
| Ctx | | Occipital Ctx | |
| AD 6 Sup Temporal | 12.2 | Control 1 Parietal | 1.5 |
| Ctx | | Ctx | |
| Control 1 Temporal | 1.4 | Control 2 Parietal | 8.2 |
| Ctx | | Ctx | |
| Control 2 Temporal | 3.1 | Control 3 Parietal | 2.9 |
| Ctx | | Ctx | |
| Control 3 Temporal | 2.2 | Control (Path) 1 | 9.1 |
| Ctx | | Parietal Ctx | |
| Control 3 Temporal | 0.2 | Control (Path) 2 | 5.9 |
| Ctx | | Parietal Ctx | |
| Control (Path) 1 | 10.7 | Control (Path) 3 | 0.6 |
| Temporal Ctx | | Parietal Ctx | |
| Control (Path) 2 | 4.5 | Control (Path) 4 | 6.8 |
| Temporal Ctx | | Parietal Ctx | |

TABLE D3
Panel 1.3D

| | Rel. Exp.(%) Ag3021, | | Rel. Exp.(%) Ag3021, |
|---|---|---|---|
| Tissue Name | Run 167966916 | Tissue Name | Run 167966916 |
| Liver adenocarcinoma | 14.2 | Kidney (fetal) | 24.3 |
| Pancreas | 4.4 | Renal ca. 786-0 | 15.0 |
| Pancreatic ca. CAPAN 2 | 6.0 | Renal ca. A498 | 10.0 |
| Adrenal gland | 2.6 | Renal ca. RXF 393 | 15.2 |
| Thyroid | 5.7 | Renal ca. ACHN | 2.8 |
| Salivary gland | 3.4 | Renal ca. UO-31 | 10.2 |
| Pituitary gland | 15.7 | Renal ca. TK-10 | 13.5 |
| Brain (fetal) | 60.7 | Liver | 2.1 |
| Brain (whole) | 11.5 | Liver (fetal) | 0.7 |
| Brain (amygdala) | 11.9 | Liver ca. | 2.5 |
| | | (hepatoblast) HepG2 | |
| Brain (cerebellum) | 5.8 | Lung | 10.7 |
| Brain (hippocampus) | 10.0 | Lung (fetal) | 19.1 |
| Brain (substantia nigra) | 22.1 | Lung ca. (small cell) | 21.8 |
| | | LX-1 | |
| Brain (thalamus) | 10.9 | Lung ca. (small cell) | 20.2 |
| | | NCI-H69 | |
| Cerebral Cortex | 10.7 | Lung ca. (s.cell var.) | 31.6 |
| | | SHP-77 | |
| Spinal cord | 11.5 | Lung ca. (large | 1.8 |
| | | cell) NCI-H460 | |
| glio/astro U87-MG | 26.8 | Lung ca. (non-sm. | 20.0 |
| | | cell) A549 | |
| glio/astro U-118-MG | 23.5 | Lung ca. (non-s.cell) | 16.0 |
| | | NCI-H23 | |
| astrocytoma SW1783 | 24.0 | Lung ca. (non-s.cell) | 9.3 |
| | | HOP-62 | |
| neuro*; met SK-N-AS | 9.7 | Lung ca. (non-s.cl) | 12.0 |
| | | NCI-H522 | |
| astrocytoma SF-539 | 9.5 | Lung ca. (squam.) | 17.6 |
| | | SW 900 | |
| astrocytoma SNB-75 | 20.4 | Lung ca. (squam.) | 24.3 |
| | | NCI-H596 | |
| glioma SNB-19 | 6.5 | Mammary gland | 11.0 |
| glioma U251 | 16.2 | Breast ca.* (pl.ef) | 16.6 |
| | | MCF-7 | |
| glioma SF-295 | 29.9 | Breast ca.* (pl.ef) | 8.7 |
| | | MDA-MB-231 | |
| Heart (fetal) | 6.6 | Breast ca.* (pl.ef) | 40.1 |
| | | T47D | |
| Heart | 6.1 | Breast ca. BT-549 | 7.0 |
| Skeletal muscle (fetal) | 4.6 | Breast ca. MDA-N | 0.9 |
| Skeletal muscle | 4.0 | Ovary | 9.5 |
| Bone marrow | 6.4 | Ovarian ca. OVCAR-3 | 8.4 |
| Thymus | 17.2 | Ovarian ca. OVCAR-4 | 7.3 |
| Spleen | 6.6 | Ovarian ca. OVCAR-5 | 100.0 |
| Lymph node | 17.1 | Ovarian ca. OVCAR-8 | 5.9 |
| Colorectal | 20.7 | Ovarian ca. IGROV-1 | 4.3 |
| Stomach | 6.7 | Ovarian ca.* (ascites) | 47.3 |
| | | SK-OV-3 | |
| Small intestine | 5.5 | Uterus | 8.1 |
| Colon ca. SW480 | 5.3 | Placenta | 0.0 |
| Colon ca.* | 27.7 | Prostate | 1.6 |
| SW620 (SW480 met) | | | |
| Colon ca. HT29 | 12.5 | Prostate ca.* (bone | 9.8 |
| | | met) PC-3 | |
| Colon ca. HCT-116 | 12.1 | Testis | 14.2 |
| Colon ca. CaCo-2 | 20.4 | Melanoma | 3.8 |
| | | Hs688(A).T | |
| Colon ca. | 18.3 | Melanoma* (met) | 2.7 |
| tissue (ODO3866) | | Hs688(B).T | |
| Colon ca. HCC-2998 | 19.8 | Melanoma UACC-62 | 11.5 |
| Gastric ca.* (liver met) | 44.4 | Melanoma M14 | 3.5 |
| NCI-N87 | | | |
| Bladder | 10.9 | Melanoma LOX | 9.7 |
| | | IMVI | |
| Trachea | 12.4 | Melanoma* (met) | 15.1 |
| | | SK-MEL-5 | |
| Kidney | 5.8 | Adipose | 11.0 |

TABLE D4
Panel 4D

| | Rel. Exp.(%) | | Rel. Exp.(%) |
|---|---|---|---|
| | Ag3021, Run | | Ag3021, Run |
| Tissue Name | 164528127 | Tissue Name | 164528127 |
| Secondary Th1 act | 15.1 | HUVEC IL-1beta | 11.1 |
| Secondary Th2 act | 16.5 | HUVEC IFN gamma | 20.0 |
| Secondary Tr1 act | 15.5 | HUVEC TNF alpha + IFN | 25.9 |
| | | gamma | |
| Secondary Th1 rest | 4.5 | HUVEC TNF alpha + IL4 | 22.2 |
| Secondary Th2 rest | 8.2 | HUVEC IL-11 | 18.3 |
| Secondary Tr1 rest | 7.6 | Lung Microvascular EC | 24.3 |
| | | none | |
| Primary Th1 act | 6.6 | Lung Microvascular EC | 27.7 |
| | | TNF alpha + IL-1beta | |
| Primary Th2 act | 10.1 | Microvascular Dermal EC | 33.2 |
| | | none | |
| Primary Tr1 act | 15.6 | Microsvasular Dermal EC | 27.7 |
| | | TNF alpha + IL-1beta | |
| Primary Th1 rest | 34.4 | Bronchial epithelium | 16.4 |
| | | TNF alpha + IL1beta | |
| Primary Th2 rest | 14.3 | Small airway epithelium | 8.4 |
| | | none | |
| Primary Tr1 rest | 7.1 | Small airway epithelium | 100.0 |
| | | TNF alpha + IL-1beta | |
| CD45RA CD4 | 4.4 | Coronery artery SMC rest | 10.4 |
| lymphocyte act | | | |
| CD45RO CD4 | 16.3 | Coronery artery SMC | 4.9 |
| lymphocyte act | | TNF alpha + IL-1beta | |
| CD8 lymphocyte act | 9.0 | Astrocytes rest | 8.7 |
| Secondary CD8 | 15.4 | Astrocytes TNF alpha + IL- | 13.3 |
| lymphocyte rest | | 1beta | |
| Secondary CD8 | 6.7 | KU-812 (Basophil) rest | 3.9 |
| lymphocyte act | | | |
| CD4 lymphocyte none | 6.4 | KU-812 (Basophil) | 19.8 |
| | | PMA/ionomycin | |
| 2ry Th1/Th2/Tr1_anti- | 11.6 | CCD1106 (Keratinocytes) | 6.8 |
| CD95 CH11 | | none | |
| LAK cells rest | 18.2 | CCD1106 (Keratinocytes) | 4.6 |
| | | TNF alpha + IL-1beta | |
| LAK cells IL-2 | 16.6 | Liver cirrhosis | 3.1 |
| LAK cells IL-2 + IL-12 | 11.7 | Lupus kidney | 3.0 |
| LAK cells IL-2 + IFN | 25.9 | NCI-H292 none | 7.1 |
| gamma | | | |
| LAK cells IL-2 + IL-18 | 18.3 | NCI-H292 IL-4 | 7.2 |
| LAK cells | 8.8 | NCI-H292 IL-9 | 8.8 |
| PMA/ionomycin | | | |
| NK Cells IL-2 rest | 12.2 | NCI-H292 IL-13 | 5.0 |
| Two Way MLR 3 day | 15.4 | NCI-H292 IFN gamma | 4.0 |
| Two Way MLR 5 day | 11.8 | HPAEC none | 20.2 |
| Two Way MLR 7 day | 8.3 | HPAEC TNF alpha + IL-1 | 27.2 |
| | | beta | |
| PBMC rest | 7.1 | Lung fibroblast none | 4.9 |
| PBMC PWM | 55.5 | Lung fibroblast | 4.1 |
| | | TNF alpha + IL-1 beta | |
| PBMC PHA-L | 25.9 | Lung fibroblast IL-4 | 19.1 |
| Ramos (B cell) none | 9.9 | Lung fibroblast IL-9 | 12.5 |
| Ramos (B cell) | 33.4 | Lung fibroblast IL-13 | 12.8 |
| ionomycin | | | |
| B lymphocytes PWM | 38.7 | Lung fibroblast IFN gamma | 28.7 |
| B lymphocytes CD40L | 15.0 | Dermal fibroblast | 16.4 |
| and IL-4 | | CCD1070 rest | |
| EOL-1 dbcAMP | 5.8 | Dermal fibroblast | 37.9 |
| | | CCD1070 TNF alpha | |
| EOL-1 dbcAMP | 17.0 | Dermal fibroblast | 6.2 |
| PMA/ionomycin | | CCD1070 IL-1beta | |
| Dendritic cells none | 26.8 | Dermal fibroblast IFN | 14.8 |
| | | gamma | |
| Dendritic cells LPS | 16.2 | Dermal fibroblast IL-4 | 29.1 |
| Dendritic cells anti- | 21.8 | IBD Colitis 2 | 0.5 |
| CD40 | | | |
| Monocytes rest | 30.8 | IBD Crohn's | 1.4 |
| Monocytes LPS | 24.1 | Colon | 23.5 |
| Macrophages rest | 35.1 | Lung | 14.8 |
| Macrophages LPS | 23.0 | Thymus | 19.6 |
| HUVEC none | 27.9 | Kidney | 33.0 |
| HUVEC starved | 40.1 | | |

CNS_neurodegeneration_v1.0 Summary: Ag3021 This panel confirms the expression of the CG56787-01 gene at low levels in the brain in an independent group of individuals. However, no differential expression of this gene was detected between Alzheimer's diseased postmortem brains and those of non-demented controls in this experiment. Please see Panel 1.3D for a discussion of the potential utility of this gene in treatment of central nervous system disorders.

Panel 1.3D Summary: Ag3021 Expression of the CG56787-01 gene is highest in a sample derived from ovarian cancer cell line OVCAR-5 (CT=30). In addition, there is substantial expression of this gene associated with other ovarian cancer cell lines as well as a breast cancer cell line. Thus, the expression of this gene could be used to distinguish OVCAR-5 cells from other samples in the panel. Moreover, therapeutic modulation of the activity of this gene or its protein product, through the use of small molecule drugs, protein therapeutics or antibodies, might be beneficial for the treatment of ovarian or breast cancer.

This gene is expressed at low levels in all regions of the CNS examined, including amygdala, cerebellum, hippocampus, substantia nigra, cerebral cortex, thalamus and spinal cord. This gene encodes a protein with homology to dual-specificity phosphatases. Dual-specificity phosphatases comprise a family of MAP kinase-regulating enzymes that are upregulated in brains subjected to insults such as ischemia and seizure activity. MAP kinases are known to regulate neurotrophic and neurotoxic pathways. Consequently, agents that modulate the activity of CG56787-01 may have utility in attenuating the apoptotic and neurodegenerative processes following brain insults.

This gene is also expressed at low levels (CTs=33-34) in pancreas, thyroid, pituitary gland, adult and fetal heart, adult and fetal skeletal muscle, and adipose. Thus, this novel protein phosphatase may be a target for small molecule drugs in the treatment of metabolic and endocrine diseases, including obesity and diabetes.

References:

1. Wiessner C. The dual specificity phosphatase PAC-1 is transcriptionally induced in the rat brain following transient forebrain ischemia. Brain Res Mol Brain Res 1995 February;28(2):353-6

PAC-1 mRNA has previously been found only in activated T-cells in vitro and in vivo. The gene encodes a dual specificity protein phosphatase that regulates MAP kinase activity. Here, I describe that PAC-1 mRNA is induced also in neurons in the rat brain following 30 min of forebrain ischemia. At 6, 12 and 24 h after ischemia, PAC-1 mRNA was found most prominently in hippocampal cells which are resistant to 30 min of forebrain ischemia, but not in the selectively vulnerable CA1 sector. At later time points and in control animals no PAC-1 mRNA could be detected in any brain region. The protein-tyrosine/threonine phosphatase PAC-1, therefore, may be involved in adaptational responses of hippocampal cells resistant to ischemic injury.

2. Boschert U, Muda M, Camps M, Dickinson R, Arkinstall S. Induction of the dual specificity phosphatase PAC1 in rat brain following seizure activity. Neuroreport Sep. 29, 1997 29;8(14):3077-80

Recurrent seizure activity leads to delayed neuronal death as well as to inflammatory responses involving microglia in hippocampal subfields CA1, CA3 and CA4. Since mitogen activated protein (MAP) kinases control neuronal apoptosis and trigger generation of inflammatory cytokines, their activation state could determine seizure-related brain damage. PAC1 is a dual specificity protein phosphatase inactivating MAP kinases which we have found to be undetectable in normal brain. Despite this, kainic acid-induced seizure activity lead to rapid (approximately 3 h) but transient appearance of PAC1 mRNA in granule cells of the dentate gyrus as well as in pyramidal CA1 neurons. This pattern changed with time and after 2-3 days PAC1 was induced in dying CA1 and CA3 neurons. At this time PAC1 mRNA was also expressed in white matter microglia as well as in microglia invading the damaged hippocampus. PAC1 may play an important role controlling MAP kinase involvement in both neuronal death and neuro-inflammation following excitotoxic damage.

3. Muda M, Boschert U, Dickinson R, Martinou J C, Martinou I, Camps M, Schlegel W, Arkinstall S. MKP-3, a novel cytosolic protein-tyrosine phosphatase that exemplifies a new class of mitogen-activated protein kinase phosphatase. J Biol Chem Feb. 23, 1996;271(8):4319-26

MKP-1 (also known as CL100, 3CH134, Erp, and hVH-1) exemplifies a class of dual-specificity phosphatase able to reverse the activation of mitogen-activated protein (MAP) kinase family members by dephosphorylating critical tyrosine and threonine residues. We now report the cloning of MKP-3, a novel protein phosphatase that also suppresses MAP kinase activation state. The deduced amino acid sequence of MKP-3 is 36% identical to MKP-1 and contains the characteristic extended active-site sequence motif VXVHCXXGXSRSXTXXXAYLM (where X is any amino acid) as well as two N-terminal CH2 domains displaying homology to the cell cycle regulator Cdc25 phosphatase. When expressed in COS-7 cells, MKP-3 blocks both the phosphorylation and enzymatic activation of ERK2 by mitogens. Northern analysis reveals a single mRNA species of 2.7 kilobases with an expression pattern distinct from other dual-specificity phosphatases. MKP-3 is expressed in lung, heart, brain, and kidney, but not significantly in skeletal muscle or testis. In situ hybridization studies of MKP-3 in brain reveal enrichment within the CA1, CA3, and CA4 layers of the hippocampus.

Panel 4D Summary: Ag3021 The CG56787-01 gene is expressed at low to moderate levels in all tissues examined except IBD colitis and Crohn's. This gene encodes a putative dual specificity phosphatase that may be important in maintaining normal cellular homeostasis in a wide range of tissues. Therapies designed with the protein encoded for by this transcript could be important in the treatment of diseases, such as IBD and Crohn's disease that show reduce the expression of this transcript.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 28 and 30;
(b) a variant of a mature form of an amino acid sequence selected from the group consisting of SEQ ID NOS: 2, 4, 6, 8, 10, 12, 14, 16, 18, 20, 22, 24, 26, 28 and 30, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, 26, 28 and 30; and
(d) a variant of an amino acid sequence selected from the group consisting of SEQ ID NOS: 2, 4, 6, 8, 10, 12, 14, 16, 18, 20, 22, 24, 26, 28 and 30 wherein one or more amino acid residues in said variant differs from the amino acid sequence of said mature form, provided that said variant differs in no more than 15% of amino acid residues from said amino acid sequence.

2 The polypeptide of claim 1, wherein said polypeptide comprises the amino acid sequence of a naturally-occurring allelic variant of an amino acid sequence selected from the group consisting of SEQ ID NOS: 2, 4, 6, 8, 10, 12, 14, 16, 18, 20, 22, 24, 26, 28 and 30.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS: 1, 3, 5, 7, 9, 11, 13, 15, 17, 19, 21, 23, 25, 27 and 29.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 28 and 30;
(b) a variant of a mature form of an amino acid sequence selected from the group consisting of SEQ ID NOS: 2, 4, 6, 8, 10, 12, 14, 16, 18, 20, 22, 24, 26, 28 and 30, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, 26, 28 and 30;
(d) a variant of an amino acid sequence selected from the group consisting of SEQ ID NOS: 2, 4, 6, 8, 10, 12, 14, 16, 18, 20, 22, 24, 26, 28 and 30, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS: 2, 4, 6, 8, 10, 12, 14, 16, 18, 20, 22, 24, 26, 28 and 30, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS: 1, 3,5,7,9, 11, 13, 15, 17, 19,21,23,25,27 and 29.

9. The nucleic acid molecule of claim 5, wherein said nucleic acid molecule comprises a nucleotide sequence selected from the group consisting of
(a) a nucleotide sequence selected from the group consisting of SEQ ID NOS: 1, 3, 5, 7, 9, 11, 13, 15, 17, 19, 21, 23, 25, 27 and 29;
(b) a nucleotide sequence differing by one or more nucleotides from a nucleotide sequence selected from the group consisting of SEQ ID NOS: 1, 3, 5, 7, 9, 11, 13, 15, 17, 19, 21, 23, 25, 27 and 29, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NOS: 1, 3, 5, 7, 9, 11, 13, 15, 17, 19, 21, 23, 25, 27 and 29, or a complement of said nucleotide sequence.

11. The nucleic acid molecule of claim 5, wherein the nucleic acid molecule comprises a nucleotide sequence selected from the group consisting of
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that immunospecifically-binds to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. A method of identifying an agent that binds to a polypeptide of claim 1, the method comprising:
(a) contacting said polypeptide with said agent; and
(b) determining whether said agent binds to said polypeptide.

21. A method for identifying an agent that modulates the expression or activity of the polypeptide of claim 1, the method comprising:
(a) providing a cell expressing said polypeptide;
(b) contacting the cell with said agent; and
(c) determining whether the agent modulates expression or activity of said polypeptide,
whereby an alteration in expression or activity of said peptide indicates said agent modulates expression or activity of said polypeptide.

22. A method for modulating the activity of the polypeptide of claim 1, the method comprising contacting a cell sample expressing the polypeptide of said claim with a compound that binds to said polypeptide in an amount sufficient to modulate the activity of the polypeptide.

23. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

24. The method of claim 23, wherein said subject is a human.

25. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

26. The method of claim 25, wherein said subject is a human.

27. A method of treating or preventing a NOVX-associated disorder, said method comprising administering to a subject in which such treatment or prevention is desired the antibody of claim 15 in an amount sufficient to treat or prevent said NOVX-associated disorder in said subject.

28. The method of claim 27, wherein the subject is a human.

29. A pharmaceutical composition comprising the polypeptide of claim 1 and a pharmaceutically-acceptable carrier.

30. A pharmaceutical composition comprising the nucleic acid molecule of claim 5 and a pharmaceutically-acceptable carrier.

31. A pharmaceutical composition comprising the antibody of claim 15 and a pharmaceutically-acceptable carrier.

32. A kit comprising in one or more containers, the pharmaceutical composition of claim 29.

33. A kit comprising in one or more containers, the pharmaceutical composition of claim 30.

34. A kit comprising in one or more containers, the pharmaceutical composition of claim 31.

35. The use of a therapeutic in the manufacture of a medicament for treating a syndrome associated with a human disease, the disease selected from a NOVX-associated disorder, wherein said therapeutic is selected from the group consisting of a NOVX polypeptide, a NOVX nucleic acid, and a NOVX antibody.

36. A method for screening for a modulator of activity or of latency or predisposition to a NOVX-associated disorder, said method comprising:
(a) administering a test compound to a test animal at increased risk for a NOVX-associated disorder, wherein said test animal recombinantly expresses the polypeptide of claim 1;
(b) measuring the activity of said polypeptide in said test animal after administering the compound of step (a);
(c) comparing the activity of said protein in said test animal with the activity of said polypeptide in a control animal not administered said polypeptide, wherein a change in the activity of said polypeptide in said test animal relative to said control animal indicates the test compound is a modulator of latency of or predisposition to a NOVX-associated disorder.

37. The method of claim 36, wherein said test animal is a recombinant test animal that expresses a test protein transgene or expresses said transgene under the control of a promoter at an increased level relative to a wild-type test animal, and wherein said promoter is not the native gene promoter of said transgene.

38. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

39. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

40. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS: 2, 4, 6, 8, 10, 12, 14, 16, 18, 20, 22, 24, 26, 28 and 30, or a biologically active fragment thereof

41. A method of treating a pathological state in a mammal, the method comprising administering to the mammal the antibody of claim 15 in an amount sufficient to alleviate the pathological state.
